SEQUENCE LISTING (295 sequences)

SEQ ID NO: 1 - protein, 9 aa, synthetic construct
FTFSSYSMN

SEQ ID NO: 2 - protein, 17 aa, synthetic construct
YISSSSSTIYYADSVKG

SEQ ID NO: 3 - protein, 126 aa, synthetic construct
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARDLTSMQSRDYYYYGMDVWGQGTTVTVSS

SEQ ID NO: 4 - protein, 11 aa, synthetic construct
RASQGISSWLA

SEQ ID NO: 5 - protein, 7 aa, synthetic construct
AASSLQS

SEQ ID NO: 6 - protein, 9 aa, synthetic construct
QQVPIFPYT

SEQ ID NO: 7 - protein, 9 aa, synthetic construct
FTFGSYAMN

SEQ ID NO: 8 - protein, 17 aa, synthetic construct
YISSSSSFIDYADSVKG

SEQ ID NO: 9 - protein, 19 aa, synthetic construct
ARDLTSMQSRDYYYYGMDV

SEQ ID NO: 10 - protein, 126 aa, synthetic construct
EVQLVESGGGLVQPGGSLRLSCAASGFTFGSYAMNWVRQAPGKGLEWVSYISSSSSFIDYADSVKGRFTISRDNAKNSLYLQMNSLRAEDTAVYYCARDLTSMQSRDYYYYGMDVWGQGTTVTVSS

SEQ ID NO: 11 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQVPIFPYTFGGGTKVEIK

SEQ ID NO: 12 - protein, 9 aa, synthetic construct
FTFEHYSMN

SEQ ID NO: 13 - protein, 17 aa, synthetic construct
YISSSSSYIDYADSVKG

SEQ ID NO: 14 - protein, 126 aa, synthetic construct
EVQLVESGGGLVQPGGSLRLSCAASGFTFEHYSMNWVRQAPGKGLEWVSYISSSSSYIDYADSVKGRFTISRDNAKNSLYLQMNSLRAEDTAVYYCARDLTSMQSRDYYYYGMDVWGQGTTVTVSS

SEQ ID NO: 15 - protein, 9 aa, synthetic construct
FTFNDYSMN

SEQ ID NO: 16 - protein, 126 aa, synthetic construct
EVQLVESGGGLVQPGGSLRLSCAASGFTFNDYSMNWVRQAPGKGLEWVSYISSSSSYIDYADSVKGRFTISRDNAKNSLYLQMNSLRAEDTAVYYCARDLTSMQSRDYYYYGMDVWGQGTTVTVSS

SEQ ID NO: 17 - protein, 19 aa, synthetic construct
ARDLTDRQSRDYYYYGMDV

SEQ ID NO: 18 - protein, 126 aa, synthetic construct
EVQLVESGGGLVQPGGSLRLSCAASGFTFEHYSMNWVRQAPGKGLEWVSYISSSSSYIDYADSVKGRFTISRDNAKNSLYLQMNSLRAEDTAVYYCARDLTDRQSRDYYYYGMDVWGQGTTVTVSS

SEQ ID NO: 19 - protein, 11 aa, synthetic construct
GSISSSSYYWG

SEQ ID NO: 20 - protein, 16 aa, synthetic construct
SISYSGSTYYNPSLKS

SEQ ID NO: 21 - protein, 16 aa, synthetic construct
ARGEYDRSYYYYGMDV

SEQ ID NO: 22 - protein, 12 aa, synthetic construct
RASQSVSSSYLA

SEQ ID NO: 23 - protein, 7 aa, synthetic construct
GASSRAT

SEQ ID NO: 24 - protein, 124 aa, synthetic construct
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGEYDRSYYYYGMDVWGQGTTVTVSS

SEQ ID NO: 25 - protein, 11 aa, synthetic construct
GSIPSSSYYWA

SEQ ID NO: 26 - protein, 16 aa, synthetic construct
NIYYSGSTYYNPSLKS

SEQ ID NO: 27 - protein, 16 aa, synthetic construct
ARGEYDRSALYYGMDV

SEQ ID NO: 28 - protein, 124 aa, synthetic construct
QLQLQESGPGLVKPSETLSLTCTVSGGSIPSSSYYWAWIRQPPGKGLEWIGNIYYSGSTYYNPSLKSRVTISVDTSKNQFSLKLSSVTAADTAVYYCARGEYDRSALYYGMDVWGQGTTVTVSS

SEQ ID NO: 29 - protein, 107 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQAFSSPTFGGGTKVEIK

SEQ ID NO: 30 - protein, 11 aa, synthetic construct
GSIPSSSYYWS

SEQ ID NO: 31 - protein, 16 aa, synthetic construct
ARGEYDRSYYFWGMDV

SEQ ID NO: 32 - protein, 124 aa, synthetic construct
QLQLQESGPGLVKPSETLSLTCTVSGGSIPSSSYYWSWIRQPPGKGLEWIGNIYYSGSTYYNPSLKSRVTISVDTSKNQFSLKLSSVTAADTAVYYCARGEYDRSYYFWGMDVWGQGTTVTVSS

SEQ ID NO: 33 - protein, 16 aa, synthetic construct
ARGEYDRSYYYWGLDV

SEQ ID NO: 34 - protein, 8 aa, synthetic construct
QQAFSSPT

SEQ ID NO: 35 - protein, 8 aa, synthetic construct
QQAFSSPS

SEQ ID NO: 36 - protein, 124 aa, synthetic construct
QLQLQESGPGLVKPSETLSLTCTVSGGSIPSSSYYWSWIRQPPGKGLEWIGNIYYSGSTYYNPSLKSRVTISVDTSKNQFSLKLSSVTAADTAVYYCARGEYDRSYYYWGLDVWGQGTTVTVSS

SEQ ID NO: 37 - protein, 107 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQAFSSPSFGGGTKVEIK

SEQ ID NO: 38 - protein, 9 aa, synthetic construct
YTFTGYYMH

SEQ ID NO: 39 - protein, 17 aa, synthetic construct
GIIPISGTANYAQKFQG

SEQ ID NO: 40 - protein, 20 aa, synthetic construct
ARGSASGYYDRSYYYYGMDV

SEQ ID NO: 41 - protein, 9 aa, synthetic construct
QQVSSFPYT

SEQ ID NO: 42 - protein, 127 aa, synthetic construct
QVQLVQSGAEVKKPGASVKVSCKASGYTFTGYYMHWVRQAPGQGLEWMGGIIPISGTANYAQKFQGRVTITADESTSTAYMELSSLRSEDTAVYYCARGSASGYYDRSYYYYGMDVWGQGTTVTVSS

SEQ ID NO: 43 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQVSSFPYTFGGGTKVEIK

SEQ ID NO: 44 - protein, 9 aa, synthetic construct
GTFSGYSIS

SEQ ID NO: 45 - protein, 17 aa, synthetic construct
LIFPISGTANYAQKFQG

SEQ ID NO: 46 - protein, 127 aa, synthetic construct
QVQLVQSGAEVKKPGSSVKVSCKASGGTFSGYSISWVRQAPGQGLEWMGLIFPISGTANYAQKFQGRVTITADESTSTAYMELSSLRSEDTAVYYCARGSASGYYDRSYYYYGMDVWGQGTTVTVSS

SEQ ID NO: 47 - protein, 9 aa, synthetic construct
GTFNNYHIS

SEQ ID NO: 48 - protein, 17 aa, synthetic construct
GIIPPFGTAHYAQKFQG

SEQ ID NO: 49 - protein, 127 aa, synthetic construct
QVQLVQSGAEVKKPGSSVKVSCKASGGTFNNYHISWVRQAPGQGLEWMGGIIPPFGTAHYAQKFQGRVTITADESTSTAYMELSSLRSEDTAVYYCARGSASGYYDRSYYYYGMDVWGQGTTVTVSS

SEQ ID NO: 50 - protein, 20 aa, synthetic construct
ARGEASGYYDRSYYYYGLDV

SEQ ID NO: 51 - protein, 127 aa, synthetic construct
QVQLVQSGAEVKKPGSSVKVSCKASGGTFNNYHISWVRQAPGQGLEWMGGIIPPFGTAHYAQKFQGRVTITADESTSTAYMELSSLRSEDTAVYYCARGEASGYYDRSYYYYGLDVWGQGTTVTVSS

SEQ ID NO: 52 - protein, 20 aa, synthetic construct
ARQSASGYYDRSYYRYGMDV

SEQ ID NO: 53 - protein, 127 aa, synthetic construct
QVQLVQSGAEVKKPGSSVKVSCKASGGTFSGYSISWVRQAPGQGLEWMGLIFPISGTANYAQKFQGRVTITADESTSTAYMELSSLRSEDTAVYYCARQSASGYYDRSYYRYGMDVWGQGTTVTVSS

SEQ ID NO: 54 - protein, 9 aa, synthetic construct
GTFSGYVIS

SEQ ID NO: 55 - protein, 17 aa, synthetic construct
GIFPISGYAVYAQKFQG

SEQ ID NO: 56 - protein, 20 aa, synthetic construct
ARASASGYYDRSYYYYGMDV

SEQ ID NO: 57 - protein, 9 aa, synthetic construct
QQVSSFPQT

SEQ ID NO: 58 - protein, 127 aa, synthetic construct
QVQLVQSGAEVKKPGSSVKVSCKASGGTFSGYVISWVRQAPGQGLEWMGGIFPISGYAVYAQKFQGRVTITADESTSTAYMELSSLRSEDTAVYYCARASASGYYDRSYYYYGMDVWGQGTTVTVSS

SEQ ID NO: 59 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQVSSFPQTFGGGTKVEIK

SEQ ID NO: 60 - protein, 9 aa, synthetic construct
GTFSNYHIS

SEQ ID NO: 61 - protein, 17 aa, synthetic construct
GIFPTFGTANYAQKFQG

SEQ ID NO: 62 - protein, 20 aa, synthetic construct
ARGSASGYYDRSYYYDGMDV

SEQ ID NO: 63 - protein, 9 aa, synthetic construct
QQVISFPYT

SEQ ID NO: 64 - protein, 127 aa, synthetic construct
QVQLVQSGAEVRKPGSSVKVSCKASGGTFSNYHISWVRQAPGQGLEWMGGIFPTFGTANYAQKFQGRVTITADESTSTAYMELSSLRSEDTAVYYCARGSASGYYDRSYYYDGMDVWGQGTTVTVSS

SEQ ID NO: 65 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQVISFPYTFGGGTKVEIK

SEQ ID NO: 66 - protein, 9 aa, synthetic construct
GTFSNYAIS

SEQ ID NO: 67 - protein, 17 aa, synthetic construct
WISAYSGDTNYAQKLQG

SEQ ID NO: 68 - protein, 15 aa, synthetic construct
ARAAGYGARHWYFDL

SEQ ID NO: 69 - protein, 11 aa, synthetic construct
RASQSISSYLN

SEQ ID NO: 70 - protein, 7 aa, synthetic construct
GASSLQS

SEQ ID NO: 71 - protein, 10 aa, synthetic construct
DQSYSTPPIT

SEQ ID NO: 72 - protein, 122 aa, synthetic construct
QVQLVQSGAEVKKPGSSVKVSCKASGGTFSNYAISWVRQAPGQGLEWMGWISAYSGDTNYAQKLQGRVTMTTDTSTSTAYMELRSLRSDDTAVYYCARAAGYGARHWYFDLWGQGTMVTVSS

SEQ ID NO: 73 - protein, 108 aa, synthetic construct
DIQMTQSPSSLSASVGDRVTITCRASQSISSYLNWYQQKPGKAPKLLIYGASSLQSGVPSRFSGSGSGTDFTLTISSLQPEDFATYYCDQSYSTPPITFGGGTKVEIK

SEQ ID NO: 74 - protein, 9 aa, synthetic construct
YTFGPYGIS

SEQ ID NO: 75 - protein, 15 aa, synthetic construct
ARAAGYGARWWYFDL

SEQ ID NO: 76 - protein, 10 aa, synthetic construct
DQFYSTPPIT

SEQ ID NO: 77 - protein, 122 aa, synthetic construct
QVQLVQSGAEVKKPGASVKVSCKASGYTFTSEGIHWVRQAPGQGLEWMGSISASSGDTNYAQELQGRVTMTTDTSTSTAYMELRSLRSDDTAVYYCARAAGYGARHWYFDLWGQGTMVTVSS

SEQ ID NO: 78 - protein, 108 aa, synthetic construct
DIQMTQSPSSLSASVGDRVTITCRASQSISSYLNWYQQKPGKAPKLLIYGASSLQSGVPSRFSGSGSGTDFTLTISSLQPEDFATYYCDQFYSTPPITFGGGTKVEIK

SEQ ID NO: 79 - protein, 17 aa, synthetic construct
SISASSGDTNYAQKLQG

SEQ ID NO: 80 - protein, 122 aa, synthetic construct
QVQLVQSGAEVKKPGASVKVSCKASGYTFTSEGIHWVRQAPGQGLEWMGSISASSGDTNYAQKLQGRVTMTTDTSTSTAYMELRSLRSDDTAVYYCARAAGYGARHWYFDLWGQGTMVTVSS

SEQ ID NO: 81 - protein, 10 aa, synthetic construct
DQWYSTPPIT

SEQ ID NO: 82 - protein, 108 aa, synthetic construct
DIQMTQSPSSLSASVGDRVTITCRASQSISSYLNWYQQKPGKAPKLLIYGASSLQSGVPSRFSGSGSGTDFTLTISSLQPEDFATYYCDQWYSTPPITFGGGTKVEIK

SEQ ID NO: 83 - protein, 15 aa, synthetic construct
ARAAGYGARSWYFDL

SEQ ID NO: 84 - protein, 122 aa, synthetic construct
QVQLVQSGAEVKKPGASVKVSCKASGYTFTSEGIHWVRQAPGQGLEWMGSISASSGDTNYAQKLQGRVTMTTDTSTSTAYMELRSLRSDDTAVYYCARAAGYGARSWYFDLWGQGTMVTVSS

SEQ ID NO: 85 - protein, 15 aa, synthetic construct
ARGAGYGARDWYFDL

SEQ ID NO: 86 - protein, 10 aa, synthetic construct
DQRYSTPPIT

SEQ ID NO: 87 - protein, 122 aa, synthetic construct
QVQLVQSGAEVKKPGASVKVSCKASGYTFTSEGIHWVRQAPGQGLEWMGSISASSGDTNYAQKLQGRVTMTTDTSTSTAYMELRSLRSDDTAVYYCARGAGYGARDWYFDLWGQGTMVTVSS

SEQ ID NO: 88 - protein, 108 aa, synthetic construct
DIQMTQSPSSLSASVGDRVTITCRASQSISSYLNWYQQKPGKAPKLLIYGASSLQSGVPSRFSGSGSGTDFTLTISSLQPEDFATYYCDQRYSTPPITFGGGTKVEIK

SEQ ID NO: 89 - protein, 15 aa, synthetic construct
ARSAGYGARHWYFDL

SEQ ID NO: 90 - protein, 11 aa, synthetic construct
GSISSGGYYWS

SEQ ID NO: 91 - protein, 16 aa, synthetic construct
SIYYSGSTYYNPSLKS

SEQ ID NO: 92 - protein, 14 aa, synthetic construct
ARAKQLTTHRYFDY

SEQ ID NO: 93 - protein, 11 aa, synthetic construct
QASQDIANYLN

SEQ ID NO: 94 - protein, 7 aa, synthetic construct
DASNLET

SEQ ID NO: 95 - protein, 9 aa, synthetic construct
QQASDLPFT

SEQ ID NO: 96 - protein, 11 aa, synthetic construct
GSISSGGYYWH

SEQ ID NO: 97 - protein, 107 aa, synthetic construct
DIQMTQSPSSLSASVGDRVTITCQASQDIANYLNWYQQKPGKAPKLLIYDASNLETGVPSRFSGSGSGTDFTFTISSLQPEDIATYYCQQASDLPFTFGGGTKVEIK

SEQ ID NO: 98 - protein, 122 aa, synthetic construct
QVQLQESGPGLVKPSQTLSLTCTVSGGSISSGGYYWSWIRQHPGKGLEWIGSIYYSGSTYYNPSLKSRVTISVDTSKNQFSLKLSSVTAADTAVYYCARAKQLTTHRYFDYWGQGTLVTVSS

SEQ ID NO: 99 - protein, 11 aa, synthetic construct
GSISSGGYSWN

SEQ ID NO: 100 - protein, 16 aa, synthetic construct
SIYRSGSTYYNPSLKS

SEQ ID NO: 101 - protein, 14 aa, synthetic construct
ARAKALTTHRYVDY

SEQ ID NO: 102 - protein, 9 aa, synthetic construct
QQARDLPFT

SEQ ID NO: 103 - protein, 122 aa, synthetic construct
QVQLQESGPGLVKPSQTLSLTCTVSGGSISSGGYSWNWIRQHPGKGLEWIGSIYRSGSTYYNPSLKSRVTISVDTSKNQFSLKLSSVTAADTAVYYCARAKALTTHRYVDYWGQGTLVTVSS

SEQ ID NO: 104 - protein, 107 aa, synthetic construct
DIQMTQSPSSLSASVGDRVTITCQASQDIANYLNWYQQKPGKAPKLLIYDASNLETGVPSRFSGSGSGTDFTFTISSLQPEDIATYYCQQARDLPFTFGGGTKVEIK

SEQ ID NO: 105 - protein, 14 aa, synthetic construct
ARAKQLTLTRYFDY

SEQ ID NO: 106 - protein, 9 aa, synthetic construct
QQASQLPFT

SEQ ID NO: 107 - protein, 122 aa, synthetic construct
QVQLQESGPGLVKPSQTLSLTCTVSGGSISSGGYSWNWIRQHPGKGLEWIGSIYRSGSTYYNPSLKSRVTISVDTSKNQFSLKLSSVTAADTAVYYCARAKQLTLTRYFDYWGQGTLVTVSS

SEQ ID NO: 108 - protein, 107 aa, synthetic construct
DIQMTQSPSSLSASVGDRVTITCQASQDIANYLNWYQQKPGKAPKLLIYDASNLETGVPSRFSGSGSGTDFTFTISSLQPEDIATYYCQQASQLPFTFGGGTKVEIK

SEQ ID NO: 109 - protein, 16 aa, synthetic construct
YIYRSGYTYYNPSLKS

SEQ ID NO: 110 - protein, 14 aa, synthetic construct
ARAKQLTTSRYFDY

SEQ ID NO: 111 - protein, 122 aa, synthetic construct
QVQLQESGPGLVKPSQTLSLTCTVSGGSISSGGYYWHWIRQHPGKGLEWIGYIYRSGYTYYNPSLKSRVTISVDTSKNQFSLKLSSVTAADTAVYYCARAKQLTTSRYFDYWGQGTLVTVSS

SEQ ID NO: 112 - protein, 17 aa, synthetic construct
GIIPIFGTANYAQKFQG

SEQ ID NO: 113 - protein, 20 aa, synthetic construct
ARGAPNYYGSSSSRGIAFDI

SEQ ID NO: 114 - protein, 9 aa, synthetic construct
VQANAFPYT

SEQ ID NO: 115 - protein, 127 aa, synthetic construct
QVQLVQSGAEVKKPGSSVKVSCKASGGTFSNYAISWVRQAPGQGLEWMGGIIPIFGTANYAQKFQGRVTITADESTSTAYMELSSLRSEDTAVYYCARGAPNYYGSSSSRGIAFDIWGQGTMVTVSS

SEQ ID NO: 116 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCVQANAFPYTFGGGTKVEIK

SEQ ID NO: 117 - protein, 9 aa, synthetic construct
GTFNNYAIS

SEQ ID NO: 118 - protein, 17 aa, synthetic construct
AIIPIFGVANYAQKFQG

SEQ ID NO: 119 - protein, 20 aa, synthetic construct
ARGAPNYYGAQSSRGIAFDI

SEQ ID NO: 120 - protein, 9 aa, synthetic construct
VQANAFPYK

SEQ ID NO: 121 - protein, 127 aa, synthetic construct
QVQLVQSGAEVKKPGSSVKVSCKASGGTFNNYAISWVRQAPGQGLEWMGAIIPIFGVANYAQKFQGRVTITADESTSTAYMELSSLRSEDTAVYYCARGAPNYYGAQSSRGIAFDIWGQGTMVTVSS

SEQ ID NO: 122 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCVQANAFPYKFGGGTKVEIK

SEQ ID NO: 123 - protein, 9 aa, synthetic construct
GTFSSHAIT

SEQ ID NO: 124 - protein, 17 aa, synthetic construct
GIIPIFGFANYAQKFQG

SEQ ID NO: 125 - protein, 20 aa, synthetic construct
ARGAPNYYGSASSRGIAFDI

SEQ ID NO: 126 - protein, 9 aa, synthetic construct
VQANAFPYS

SEQ ID NO: 127 - protein, 127 aa, synthetic construct
QVQLVQSGAEVKKPGSSVKVSCKASGGTFSSHAITWVRQAPGQGLEWMGGIIPIFGFANYAQKFQGRVTITADESTSTAYMELSSLRSEDTAVYYCARGAPNYYGSASSRGIAFDIWGQGTMVTVSS

SEQ ID NO: 128 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCVQANAFPYSFGGGTKVEIK

SEQ ID NO: 129 - protein, 20 aa, synthetic construct
ARGAPTYFGAASHYYYYMDV

SEQ ID NO: 130 - protein, 11 aa, synthetic construct
RASQSISSWLA

SEQ ID NO: 131 - protein, 7 aa, synthetic construct
KASSLES

SEQ ID NO: 132 - protein, 10 aa, synthetic construct
EQYGSYSPLT

SEQ ID NO: 133 - protein, 128 aa, synthetic construct
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGAPTYFGAASHYYYYMDVWGKGTTVTVSS

SEQ ID NO: 134 - protein, 108 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCEQYGSYSPLTFGGGTKVEIK

SEQ ID NO: 135 - protein, 11 aa, synthetic construct
GSISSSPYNWV

SEQ ID NO: 136 - protein, 16 aa, synthetic construct
SILHSGSTYYNPSLKS

SEQ ID NO: 137 - protein, 20 aa, synthetic construct
ARGVPTYWGAASHYYYYMDV

SEQ ID NO: 138 - protein, 10 aa, synthetic construct
EQYGSESPLT

SEQ ID NO: 139 - protein, 128 aa, synthetic construct
QLQLQESGPGLVKPSETLSLTCTVSGGSISSSPYNWVWIRQPPGKGLEWIGSILHSGSTYYNPSLKSRVTISVDTSKNQFSLKLSSVTAADTAVYYCARGVPTYWGAASHYYYYMDVWGKGTTVTVSS

SEQ ID NO: 140 - protein, 108 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCEQYGSESPLTFGGGTKVEIK

SEQ ID NO: 141 - protein, 17 aa, synthetic construct
SINPSSGGTNYAQKFQG

SEQ ID NO: 142 - protein, 21 aa, synthetic construct
ARGGPSTWYGGSAYYYYGMDV

SEQ ID NO: 143 - protein, 12 aa, synthetic construct
RASQSVSSDYLA

SEQ ID NO: 144 - protein, 8 aa, synthetic construct
QQYANLWT

SEQ ID NO: 145 - protein, 128 aa, synthetic construct
QVQLVQSGAEVKKPGASVKVSCKASGYTFTGYYMHWVRQAPGQGLEWMGSINPSSGGTNYAQKFQGRVTMTRDTSISTAYMELSRLRSDDTAVYYCARGGPSTWYGGSAYYYYGMDVWGQGTTVTVSS

SEQ ID NO: 146 - protein, 107 aa, synthetic construct
EIVLTQSPGTLSLSPGERATLSCRASQSVSSDYLAWYQQKPGQAPRLLIYGASSRATGIPDRFSGSGSGTDFTLTISRLEPEDFAVYYCQQYANLWTFGGGTKVEIK

SEQ ID NO: 147 - protein, 9 aa, synthetic construct
YTFAGYDMH

SEQ ID NO: 148 - protein, 17 aa, synthetic construct
SINPTSGRTNYAQKFQG

SEQ ID NO: 149 - protein, 21 aa, synthetic construct
ARGGPSTWYGGSAYTYYGADV

SEQ ID NO: 150 - protein, 8 aa, synthetic construct
QQYAPLWT

SEQ ID NO: 151 - protein, 128 aa, synthetic construct
QVQLVQSGAEVKKPGASVKVSCKASGYTFAGYDMHWVRQAPGQGLEWMGSINPTSGRTNYAQKFQGRVTMTRDTSISTAYMELSRLRSDDTAVYYCARGGPSTWYGGSAYTYYGADVWGQGTTVTVSS

SEQ ID NO: 152 - protein, 107 aa, synthetic construct
EIVLTQSPGTLSLSPGERATLSCRASQSVSSDYLAWYQQKPGQAPRLLIYGASSRATGIPDRFSGSGSGTDFTLTISRLEPEDFAVYYCQQYAPLWTFGGGTKVEIK

SEQ ID NO: 153 - protein, 122 aa, synthetic construct
QVQLQESGPGLVKPSQTLSLTCTVSGGSISSGGYYWHWIRQHPGKGLEWIGYIYRSGYTYYNPSLKSRVTISVDTSKNQFSLKLSSVTAADTAVYYCARAKQLTTHRYFDYWGQGTLVTVSS

SEQ ID NO: 154-197 - skipped (44 entries)

SEQ ID NO: 198 - protein, 10 aa, synthetic construct
DGSYSTPPIT

SEQ ID NO: 199 - protein, 122 aa, synthetic construct
QVQLVQSGAEVKKPGASVKVSCKASGYTFGPYGISWVRQAPGQGLEWMGWISAYSGDTNYAQKLQGRVTMTTDTSTSTAYMELRSLRSDDTAVYYCARAAGYGARWWYFDLWGQGTMVTVSS

SEQ ID NO: 200 - protein, 108 aa, synthetic construct
DIQMTQSPSSLSASVGDRVTITCRASQSISSYLNWYQQKPGKAPKLLIYGASSLQSGVPSRFSGSGSGTDFTLTISSLQPEDFATYYCDGSYSTPPITFGGGTKVEIK

SEQ ID NO: 201 - protein, 17 aa, synthetic construct
SISASSGDTNYAQELQG

SEQ ID NO: 202 - DNA, 378 bp, synthetic construct
  source 1..378: mol_type = other DNA
gaagtgcagcttgtggagtccggaggaggcctcgtgcaaccgggcggaagcctgaggctgagctgcgcagcctcagggttcactttttcctcctactccatgaattgggtcagacaggctcctgggaagggtctggaatgggtgtcctacatctcctcctcgtcatctaccatctactacgccgactccgtcaagggccgcttcacgatttcccgcgacaacgccaagaactcgctgtacctccaaatgaactccttgcgggccgaggataccgcggtgtactactgcgcccgggatctgactagcatgcagagccgggactactactattacggaatggacgtctggggacagggcaccaccgtgaccgtgtcgagc

SEQ ID NO: 203 - DNA, 321 bp, synthetic construct
  source 1..321: mol_type = other DNA
gatattcaaatgacccagtccccgtcgtccgtgagcgcctcagtgggcgacagagtcactattacctgtcgcgctagccagggcatctcctcctggctcgcctggtaccagcagaaacccggaaaggcacccaagctgctgatctacgcggccagctcgcttcagtccggggtgccatctcggttctccggatccggctcaggaaccgactttactctcaccatctcgagcctgcagcccgaggacttcgccacctactactgccaacaagtgccgatcttcccttacactttcgggggtggaaccaaggtcgaaatcaag

SEQ ID NO: 204 - DNA, 378 bp, synthetic construct
  source 1..378: mol_type = other DNA
gaagtgcaactggtggagtctggtggcggactcgtgcagcccggcggctccctgcggctctcctgcgcggccagcggctttaccttcggatcctacgccatgaattgggtcagacaggccccgggaaaagggcttgaatgggtgtcatacatcagctcgtcctcctccttcatcgactacgcggatagcgtgaagggtcgcttcaccatttcgcgggacaacgcaaagaactcactgtacttgcaaatgaactccctgcgggccgaggacaccgccgtgtactactgtgctcgcgacctgaccagcatgcagtcgagggactactactattacggaatggatgtctggggacaggggactactgtgaccgtgtcctcc

SEQ ID NO: 205 - DNA, 378 bp, synthetic construct
  source 1..378: mol_type = other DNA
gaagtgcagctcgtggagtcgggtggcggactggtgcagcccgggggatccctccggctgagctgcgccgcctctggattcaccttcgaacactacagcatgaactgggtcagacaggctccgggaaagggcctggaatgggtgtcctacatttcctcgtcctcatcgtacatcgactacgccgactccgtgaagggccggtttaccatctcccgggataatgccaagaactccctgtaccttcaaatgaactccttgagggccgaggataccgcggtgtactactgtgcacgcgacctgactagcatgcagtcacgcgactattactactacgggatggacgtctggggccagggtaccaccgtgactgtgtccagc

SEQ ID NO: 206 - DNA, 378 bp, synthetic construct
  source 1..378: mol_type = other DNA
gaagtgcagctcgtggagagcggcggcggattggtgcagcccggcggatcactgagactttcctgcgcggccagcggtttcactttcaacgactactctatgaactgggtccgccaagctccgggaaagggtctggaatgggtgtcctacattagctcctcctcctcgtacatcgattacgccgattccgtgaagggccgctttaccatctcccgggacaacgccaagaactccctgtacctccaaatgaattccctgagggccgaggataccgcagtgtactactgtgcccgggacctgactagcatgcagtcacgggactactactattacgggatggacgtctggggacaggggaccaccgtgaccgtgtcgtcg

SEQ ID NO: 207 - DNA, 378 bp, synthetic construct
  source 1..378: mol_type = other DNA
gaagtgcagcttgtggagagcggtggcggactggtgcagcccggcggatccctgcggctctcctgcgccgcgagcggtttcaccttcgaacactactccatgaattgggtcagacaggccccgggaaagggcctggaatgggtgtcctatatctcgagctcctcctcatacatcgactacgctgactccgtgaagggccggtttaccatttcgagggacaacgccaagaactctctgtacctccaaatgaactccctgagagccgaggataccgcagtgtactactgtgcccgcgacttgactgatcgccagtcgcgggattactactactacgggatggacgtctggggacaggggaccaccgtgactgtgtcaagc

SEQ ID NO: 208 - DNA, 372 bp, synthetic construct
  source 1..372: mol_type = other DNA
cagctgcaactgcaggaatccggaccaggcctcgtgaagcccagcgaaactttgtcccttacctgtaccgtcagcggaggttcaatctcctcctcttcctactactggggttggattaggcagcctccgggaaaggggctcgagtggatcggcagcatttcctactcgggctcgacctactacaacccgtccctgaagtcgcgcgtgaccatctcggtggacacctccaagaaccagttcagcctgaaactgtcctcagtgactgccgctgatacggcggtgtattactgcgcaagaggggagtacgaccggtcatactattactacggaatggacgtctggggacagggcaccactgtgaccgtgtccagc

SEQ ID NO: 209 - DNA, 321 bp, synthetic construct
  source 1..321: mol_type = other DNA
gagatcgtgctgacccagtcccccggaaccctgagcctctcccctggcgaaagggccactctgtcgtgccgggcgtcacagtcagtgtccagctcctacctcgcttggtaccagcaaaagcctggacaagccccacgccttctgatctacggcgcctcgagcagagccactggaattcccgaccggttctccggttctgggtccggcaccgatttcaccctgaccatttcccgcttggaaccggaggactttgccgtgtattactgccagcaggcattctcgagcccgaccttcggtggagggactaaggtcgagatcaag

SEQ ID NO: 210 - DNA, 372 bp, synthetic construct
  source 1..372: mol_type = other DNA
cagctgcaactccaagaatccggaccgggtcttgtgaagccatctgagactctgtcactgacttgcaccgtgtcgggtggcagcatcccctcgagctcctactactgggcctggattaggcagccgcctgggaagggactcgagtggattgggaacatctactactccggctccacgtactacaacccctccctgaaatcccgggtcaccatcagcgtggacacctccaagaaccagttcagcctgaagctgtcctcggtgaccgcagccgatactgccgtgtactactgtgctcgcggcgaatatgacagatccgcgttgtactacggaatggacgtctggggacagggcaccaccgtgaccgtgtcgtca

SEQ ID NO: 211 - DNA, 372 bp, synthetic construct
  source 1..372: mol_type = other DNA
cagctgcaactgcaagaatcggggccgggcttggtcaagccaagcgaaaccctctctctgacctgtactgtgtccggcggaagcatcccctcctcctcctattactggtcgtggattaggcagcctcccgggaagggactggagtggatcggcaacatctactactcgggctcgacttactacaacccgagcctgaagtcccgcgtgaccattagcgtggacacctccaaaaaccagttcagccttaagctctcatcagtgacggccgccgataccgctgtgtactactgcgcgcggggagagtacgacagatcctactacttctggggaatggacgtctggggacagggtaccactgtgaccgtgtcctcc

SEQ ID NO: 212 - DNA, 321 bp, synthetic construct
  source 1..321: mol_type = other DNA
gaaatcgtgttgactcagtcccctggtaccctctcactgtccccgggagaaagggccaccctgtcctgtcgggcatcgcagtccgtgtcctcctcgtaccttgcctggtaccagcagaagcccggccaggctccccgcctcctgatctacggcgcgagcagcagagccactggtattcctgaccggttctctgggtccggaagcggaaccgacttcactctgaccatttcacgcctggagccggaagatttcgccgtgtattactgccaacaagccttcagctcgccatcctttggcgggggaaccaaagtcgagatcaag

SEQ ID NO: 213 - DNA, 372 bp, synthetic construct
  source 1..372: mol_type = other DNA
cagctgcaactgcaagaatcgggacccggactcgtgaagcctagcgagactctgagcctcacctgtactgtgtctggcggatccatcccgtcctcgagctactactggtcctggattcggcagccaccggggaagggcctggagtggattgggaacatctactactccggttcgacctactataacccctcgctgaagtcccgcgtcaccatcagcgtggacacctccaagaaccagttctccctgaaattgtccagcgtgaccgccgctgatacggccgtgtactactgcgcgaggggagaatacgacagatcctactactattggggccttgacgtctggggccagggtactaccgtgaccgtgtcatca

SEQ ID NO: 214 - DNA, 381 bp, synthetic construct
  source 1..381: mol_type = other DNA
caggtccagctggtgcaatcgggggccgaagtcaagaagcctggagcgtccgtgaaggtgtcctgcaaagcctccggctacaccttcaccggttactacatgcactgggtcagacaggcacccggacagggactcgaatggatgggcggcatcatcccgatcagcggaaccgccaactatgcccaaaagttccagggtcgcgtgactattacggctgacgagtccacctccactgcctacatggaactgtcctccctgcggagcgaggataccgccgtgtactactgcgcgaggggatcagcctcggggtattacgaccgctcgtactactactacgggatggacgtgtggggacagggcaccaccgtgactgtgtcaagc

SEQ ID NO: 215 - DNA, 321 bp, synthetic construct
  source 1..321: mol_type = other DNA
gacattcagatgactcagtccccatcctccgtgagcgcctcagtgggggatcgggtcaccattacctgtagagcctcccagggcatctccagctggctggcttggtaccagcagaagcctggaaaggcccctaagttgctgatctacgcagcgagcagcctgcagtcaggagtgccgtcccgcttctcgggatccggttctggaaccgatttcaccctcacgatctcgtcgctgcaaccggaggacttcgccacctactactgccaacaagtgtcgtccttcccctatacttttggcggcgggaccaaagtggaaatcaag

SEQ ID NO: 216 - DNA, 381 bp, synthetic construct
  source 1..381: mol_type = other DNA
caggtccaactggtgcaatccggcgccgaagtgaagaagccgggatcgagcgtgaaggtgtcctgcaaagcctccggagggaccttttccggctactccatctcatgggtcagacaggcacctggacagggtctggaatggatgggcctcatcttccccatttcggggaccgccaactacgcccagaagttccagggaagggtcactatcaccgccgacgagagcacttccacggcctacatggagctgagctctctgcggagcgaagataccgcggtgtactattgtgctcggggatcagcgagcggttactacgaccgctcctactactactacgggatggacgtctggggacagggcaccactgtgaccgtgtcctcg

SEQ ID NO: 217 - DNA, 381 bp, synthetic construct
  source 1..381: mol_type = other DNA
caggtccaactggtgcagtcaggcgcagaagtcaagaagcccggaagctcggtgaaagtgtcctgcaaggcctccgggggaacctttaacaactaccacatttcgtgggtccgccaggccccgggacagggtctggagtggatgggcggcatcatccctccattcggaaccgcccattacgcccaaaagttccagggcagagtgacgatcaccgcggacgaatcgacctccaccgcctacatggaactgagctctctccggtccgaggatactgccgtgtactactgtgcgaggggtagcgcttccgggtattacgaccggtcctactactactacggaatggacgtctggggccagggaaccactgtgactgtgtcatcc

SEQ ID NO: 218 - DNA, 381 bp, synthetic construct
  source 1..381: mol_type = other DNA
caagtgcagcttgtgcagtccggggcagaagtcaagaagcccggaagctcggtgaaagtgtcctgcaaggcctccggtggaaccttcaacaactaccacatctcctgggtccgccaggctccgggacagggtctggagtggatgggcggcatcatccctccatttgggaccgcgcattatgcccaaaagttccagggccgcgtgaccattactgccgatgaatccacgagcaccgcctacatggaactgagctcgctgagatcagaggacactgccgtgtactactgtgcgaggggagaggcctccggctactacgaccggtcctactactactacggactcgacgtctggggacagggcaccactgtgaccgtgtcgtca

SEQ ID NO: 219 - DNA, 381 bp, synthetic construct
  source 1..381: mol_type = other DNA
caagtgcagctcgtgcagtccggagccgaagtcaagaagccgggctcatccgtgaaagtgtcctgcaaggcctccggtggaaccttcagcggatactcgatttcctgggtccgccaagcccctggacaaggccttgagtggatgggactgatctttcccatctcggggaccgcaaactatgcgcagaagttccagggcagagtgaccatcactgccgacgaaagcaccagcacggcctacatggaactgtcaagcctgaggtccgaggatactgcggtgtactactgcgcccggcagtcggcttccgggtactacgaccggtcttactaccgctacggaatggacgtctggggccagggtaccactgtgaccgtgtcctcc

SEQ ID NO: 220 - DNA, 375 bp, synthetic construct
  source 1..375: mol_type = other DNA
caactggtgcagtccggtgctgaagtcaagaagcccggctctagcgtgaaagtgtcgtgcaaggcatccgggggaaccttctccggttacgtgattagctgggtcagacaggcgccgggacagggcctcgagtggatgggcgggatcttccctatctcgggatacgccgtgtatgcccagaagtttcagggccgcgtgaccatcactgccgatgaatccacctccacggcctacatggaactgagctccctgcggtccgaggacaccgccgtgtattactgtgcccgggcctcagcgtcgggatactacgaccgctcctactactactacggaatggacgtctggggccagggaactactgtgaccgtgtcaagc

SEQ ID NO: 221 - DNA, 321 bp, synthetic construct
  source 1..321: mol_type = other DNA
gacattcagatgacccagtccccgtcctccgtgtcggcttccgtgggagacagagtcactatcacttgtcgggccagccagggaatttcctcatggctggcctggtatcagcagaagcctggaaaggccccaaagcttctcatctacgccgcgtcgtcactgcaatccggcgtgcccagccgcttctctgggtccggatcggggactgactttaccctgaccatcagcagcctgcagcccgaggatttcgcaacgtactactgccaacaagtgtcctccttcccgcaaaccttcggcggcggtaccaaagtcgaaatcaag

SEQ ID NO: 222 - DNA, 381 bp, synthetic construct
  source 1..381: mol_type = other DNA
caagtgcaactggtgcagagcggagccgaagtccgcaagcccggctcgagcgtgaaagtgtcctgcaaggcctcgggcggcaccttctccaactaccacatctcatgggtcagacaggcgcctggacagggactcgagtggatgggcggcattttcccgaccttcggtaccgccaactacgctcagaagtttcagggtcgggtcactatcaccgcggacgaaagcacctccacggcctacatggaactgtcatcgctgcggtccgaggacaccgccgtgtactactgtgcccgcggaagcgcatccggatattacgataggtcctactactacgacgggatggatgtctggggacaggggactaccgtgactgtgtcctct

SEQ ID NO: 223 - DNA, 321 bp, synthetic construct
  source 1..321: mol_type = other DNA
gatattcaaatgacccagtcaccgtctagcgtgtcagcctccgtgggcgaccgcgtgactatcacctgtcgggccagccagggaatctcctcgtggctggcttggtatcagcagaaacccggaaaggccccgaagctgctcatctacgccgcgagctcgctgcagtccggcgtgccatcgagattttccggaagcggatccgggaccgacttcacgcttactatctcctccctgcaacccgaggacttcgcaacctactactgccaacaagtcatttccttcccttacaccttcggtggcgggactaaggtcgaaatcaag

SEQ ID NO: 224 - DNA, 366 bp, synthetic construct
  source 1..366: mol_type = other DNA
caagtgcagttggtgcagtcaggcgccgaagtcaagaagccgggtagcagcgtgaaagtgtcgtgcaaggcatccggcggcactttctcgaactacgccatctcctgggtcagacaggcgcctggacaaggactggaatggatggggtggatttccgcctactccggggacaccaactacgcccagaagctgcagggacgggtcaccatgacgaccgacacttccacctccaccgcctacatggagctccgctcactgcggtccgacgatactgccgtgtactattgtgcgagagcagccggctacggagctaggcactggtacttcgatctgtggggacagggtaccatggtcactgtgtccagc

SEQ ID NO: 225 - DNA, 324 bp, synthetic construct
  source 1..324: mol_type = other DNA
gacattcagatgacccagagcccctcctccctctccgcatccgtgggcgacagagtcaccatcacttgtcgggctagccagtcgatctcctcatatcttaactggtaccagcaaaagcctggaaaggcgccgaagctgctgatctacggagccagcagcctgcagtccggagtgccatcccgcttctccggctccggatctggaaccgatttcaccctgactatctcctcgctgcaaccggaggactttgccacttactactgcgaccagtcgtactcaacgccccctattaccttcggtggcgggaccaaagtggaaatcaag

SEQ ID NO: 226 - skipped

SEQ ID NO: 227 - DNA, 324 bp, synthetic construct
  source 1..324: mol_type = other DNA
gatatccaaatgacccagtccccgtcatccctttccgcctccgtgggagatagagtgaccatcacttgtcgggcgtcgcagtccatctcgtcgtacctgaactggtaccagcagaaacctgggaaggcccccaagctgctgatctacggagccagcagcctgcagtccggagtgccttcacgcttctccgggtctggaagcggcaccgactttaccttgactatttcctccctccaacccgaggacttcgctacgtactattgcgacggctcgtacagcaccccgccaattaccttcggtggtggcactaaggtcgaaatcaag

SEQ ID NO: 228 - DNA, 366 bp, synthetic construct
  source 1..366: mol_type = other DNA
caagtgcagctggtgcagtccggagcggaagtcaagaagccgggagcttccgtgaaagtgtcctgcaaggcctccgggtacactttcacatcggagggcatccactgggtcagacaggcccctggccaaggactggagtggatggggtcaatttcggccagctccggagacaccaactacgcccaggaacttcagggccgcgtgactatgaccaccgacaccagcacctccactgcctacatggaactgcggtcgctgaggagcgacgataccgccgtgtactactgtgcaagagcggctggttatggtgcccggcattggtacttcgatctctggggacagggcaccatggtcacggtgtcatcc

SEQ ID NO: 229 - DNA, 324 bp, synthetic construct
  source 1..324: mol_type = other DNA
gacattcaaatgactcagtccccctcatctctgtccgcgagcgtgggagacagagtgaccattacgtgtcgcgccagccagagcatctcgtcatacctgaactggtaccagcagaagcctggcaaagccccgaagcttttgatctacggggcttcctccctccaatcgggagtgccttcccggttctccggaagcggatccgggaccgatttcactctgaccatctcctcgctgcaaccagaagatttcgccacctactactgcgaccagttctattccaccccgcccatcacttttggcggcggtaccaaggtcgagatcaag

SEQ ID NO: 230 - DNA, 366 bp, synthetic construct
  source 1..366: mol_type = other DNA
caagtgcagttggtgcagtcaggcgccgaggtcaagaaacccggagccagcgtgaaggtgtcctgcaaggcgtccggttacaccttcactagcgaagggatccactgggtccgccaagctccgggacagggactggaatggatggggtcgatttccgcctcgtcgggagataccaactatgcccagaagctgcagggtcgcgtgaccatgactactgacacctccacctccacggcctacatggagctgcggtcactgcggagcgacgacaccgccgtgtactactgtgcgagagctgccggctacggagcaaggcattggtacttcgatctctggggccagggcactatggtcaccgtgtcctcc

SEQ ID NO: 231 - DNA, 366 bp, synthetic construct
  source 1..366: mol_type = other DNA
caagtgcagctggtgcagagcggggctgaagtcaagaagcctggagccagcgtgaaagtgtcctgcaaggcctcaggctacaccttcacatccgagggcattcactgggtcagacaggccccgggacaaggactggagtggatgggttcgatctcggcctcatccggcgatactaactacgcgcagaagctgcagggtcgcgtgaccatgaccactgacactagcacctcgaccgcttacatggaactgaggtccctccggtccgacgataccgccgtgtactactgtgcgagagccgcaggatatggggcccggcattggtacttcgacctttggggacagggcaccatggtcacggtgtcctcc

SEQ ID NO: 232 - DNA, 324 bp, synthetic construct
  source 1..324: mol_type = other DNA
gacatccaaatgacccagtccccatcttcgctgtcggccagcgtgggagatcgcgtgactattacctgtcgggctagccagtccatctcgtcctatctcaactggtaccagcagaagccgggaaaggcccctaagctgttgatctacggggcgtcatcccttcaatccggggtgccttcaagattctccggatccggttccggcaccgacttcactctgaccattagcagcctgcagcccgaagatttcgccacctactactgcgaccagtggtactccacgccgcccatcacttttggcggcggaaccaaggtcgagatcaaa

SEQ ID NO: 233 - DNA, 366 bp, synthetic construct
  source 1..366: mol_type = other DNA
caagtgcagctggtgcagtccggtgccgaagtgaaaaagccgggtgctagcgtgaaggtgtcctgcaaggcttccgggtacaccttcacatctgagggcattcactgggtcagacaggcccctggacaagggctggagtggatgggatcgatctcagcgtcctccggcgacaccaactacgcccagaagctgcagggacgggtcactatgaccactgacacgagcacttccaccgcctacatggaactccggtcactgcgctcggatgataccgccgtgtactactgtgcaagagcggccggatacggagccaggtcgtggtatttcgacctttggggccagggcaccatggtcaccgtgtccagc

SEQ ID NO: 234 - DNA, 366 bp, synthetic construct
  source 1..366: mol_type = other DNA
caagtgcagctggtgcagtctggcgccgaggtcaagaagccgggagcctcagtgaaagtgtcctgcaaggcctccggatacaccttcacatcggaagggattcactgggtccgccaagctcccggtcaaggacttgagtggatgggctccatcagcgcttcgtccggggacactaactacgcccagaagctgcagggacgcgtgaccatgactaccgacacctccacctcaaccgcctacatggaactgagaagcctgcggtcggacgatactgccgtgtattactgtgcgaggggtgcaggatacggcgcgagagattggtacttcgacctctggggccagggaaccatggtcacggtgtccagc

SEQ ID NO: 235 - DNA, 324 bp, synthetic construct
  source 1..324: mol_type = other DNA
gatatccaaatgactcagagcccatcctccttgtcggcctcagtgggagacagagtgaccattacgtgtcgcgcgagccagtcgatctcctcctacctcaactggtaccagcagaagcccggcaaagctccgaagcttctgatctatggagcctcctccctgcaatcgggcgtgccgtcaaggttctctggttccggatccggcaccgactttaccctgaccattagcagcctgcagcccgaggacttcgccacttactactgcgatcagcggtactccacccctcctatcaccttcggggggggaactaaggtcgaaatcaag

SEQ ID NO: 236 - DNA, 366 bp, synthetic construct
  source 1..366: mol_type = other DNA
caagtgcagctggtgcagagcggagccgaagtcaagaagccgggagcttccgtgaaagtgtcctgcaaggcctccggctacaccttcacatcggagggtattcactgggtccgccaagcacctggacagggcctggagtggatgggctccatctcggcctcatccggggacaccaactacgcccagaagctgcagggacgggtcacgatgactactgacacctccacctcaaccgcctatatggaacttcgcagcctgaggagcgatgacactgccgtgtactactgtgcgagatctgcgggatacggtgcccggcattggtacttcgatctctggggccaggggactatggtcaccgtgtcgtcc

SEQ ID NO: 237 - DNA, 366 bp, synthetic construct
  source 1..366: mol_type = other DNA
caagtgcaactgcaggagtctggacctggtttggtcaagccgagccagacgctttccctcacctgtaccgtgtcggggggatccatctcatcgggaggctactactggtcctggattagacagcatcccggaaaggggctggaatggattggttccatctactactccggctccacttactataacccaagcctcaagtcaagggtcactatctcggtggatacctccaagaaccagttcagcctgaaactgtccagcgtgaccgctgccgacactgcggtgtactactgcgcccgggcaaagcagctgaccacccaccgctacttcgactactggggccagggcactctggtcaccgtgtcgagc

SEQ ID NO: 238 - DNA, 321 bp, synthetic construct
  source 1..321: mol_type = other DNA
gacatccaaatgacccagagcccctcctccttgtcggcttccgtgggagacagggtcactatcacgtgtcaggcgtcacaggacatcgcgaattacctgaactggtaccagcagaagcccggaaaggcacctaagctcctgatctatgacgcctccaacctggaaaccggggtgccgtctagattctccggttccggaagcggcaccgattttactttcaccattagcagcctccagcctgaggatattgccacctactactgccaacaagcctcggaccttccattcaccttcggtggcggcactaaggtcgaaatcaaa

SEQ ID NO: 239 - DNA, 366 bp, synthetic construct
  source 1..366: mol_type = other DNA
caagtgcaactgcaggagtcagggcctggtctggtcaagccaagccagaccctgtctctgacgtgcactgtgtcgggaggatccatcagctcggggggttactactggcattggattaggcagcaccccggaaagggccttgaatggattggctacatctaccggtccggctacacctactacaacccgtccctgaagtcgagagtgaccatctccgtcgacaccagcaaaaaccagttcagcctgaagttgtcatccgtgaccgccgctgacactgcggtgtactattgtgcacgggccaagcagctcactacccaccgctacttcgattactggggacagggcactctcgtgaccgtgtcctcc

SEQ ID NO: 240 - DNA, 366 bp, synthetic construct
  source 1..366: mol_type = other DNA
caggtccaactgcaagaatcagggccgggactcgtgaagccatctcagaccttgtcgctgacctgtactgtgtccgggggttccatttcgtccggcggatactcctggaactggattcggcagcaccccggaaaaggcctcgagtggatcggctccatctaccggtcgggtagcacttattacaaccctagcctgaagtcgagagtgaccatctccgtcgacacgtccaagaaccagttcagcctgaagctgagctcagtgaccgctgccgacactgccgtgtactactgcgcaagggccaaggcgctgactacccatcgctacgtggattactggggacagggcacccttgtgaccgtgtcctcc

SEQ ID NO: 241 - DNA, 321 bp, synthetic construct
  source 1..321: mol_type = other DNA
gacattcaaatgacccagtccccctcctccctgtcggcttccgtgggagacagagtgaccatcacttgccaagccagccaggacatcgccaactacctgaactggtatcagcagaagccagggaaggcaccgaagttgcttatctacgacgcctcgaacctggaaacgggtgtcccctcacggttctctgggagcggaagcggcactgatttcaccttcaccatctcctccctccaaccggaggacattgcgacctactactgtcagcaggcccgcgatctgcctttcacttttggcggaggcaccaaagtggagatcaag

SEQ ID NO: 242 - DNA, 366 bp, synthetic construct
  source 1..366: mol_type = other DNA
caggtccaacttcaagaatccggccctggactcgtgaaaccaagccagactctgagcctgacttgtaccgtgtccggaggttcgatctcctccggcggctactcatggaactggattagacagcacccggggaagggactggagtggattggttcgatctaccggtccggctcaacctactacaacccctcgctgaagtcccgggtcaccatctcggtggatacctccaagaaccagttcagcctgaagctgagctccgtgactgccgccgacactgctgtgtactattgcgcaagggcgaagcagttgaccctgacccgctacttcgactactggggacaggggacgctcgtgaccgtgtcctct

SEQ ID NO: 243 - DNA, 321 bp, synthetic construct
  source 1..321: mol_type = other DNA
gacattcaaatgactcagagcccgagcagcttgtcagcatcggtcggggatcgcgtgactatcacctgtcaagcgtcgcaggacatcgctaactacctgaactggtaccagcagaagccgggaaaggcccctaagctgctgatctatgacgcctccaacctggaaacgggagtgccttcccggttctccggttccggatcgggcaccgacttcaccttcaccatctcctccctccaacccgaggatattgccacctactactgccagcaggcgtcacagcttccattcacttttggcggcgggaccaaagtggaaatcaag

SEQ ID NO: 244 - DNA, 366 bp, synthetic construct
  source 1..366: mol_type = other DNA
caagtgcagctgcaggaatccggcccgggtcttgtgaagccctcacaaaccctgagcttgacctgtaccgtgtctggagggtcgatctcctccggcggctactactggcactggattaggcagcatcctgggaagggtctggagtggatcggatacatctaccggtccggatacacgtactataacccaagcctgaagtcccgcgtgaccattagcgtggacacctccaagaaccagttctcgctgaaactcagctcagtcactgctgccgataccgccgtctactactgcgcaagagcgaagcagctgactacctcgcggtacttcgactactggggccagggaactctcgtgactgtgtcctcc

SEQ ID NO: 245 - DNA, 384 bp, synthetic construct
  source 1..384: mol_type = other DNA
cagcttcaactgcaagagtcgggacccggactcgtgaaaccctcggaaactctgtcgctgacttgcaccgtgtccggagggtccattagctcctcctcatactactggggttggatccggcagccacctggaaagggcctcgaatggatcggctcaatttcctattccggaagcacctactacaacccgagcttgaagtctcgcgtcaccatctccgtggacacctcgaagaaccagttcagcctgaagctgtccagcgtcacggcagccgatactgccgtgtactactgcgccagaggtgctcctacctacttcggagcggcctcacactattactactacatggacgtctgggggaagggcactaccgtgaccgtgtcctcc

SEQ ID NO: 246 - DNA, 324 bp, synthetic construct
  source 1..324: mol_type = other DNA
gacattcaaatgactcagagcccgtccaccctgtccgcctctgtgggagacagagtgaccatcacttgtcgggctagccagtccatttcctcctggctcgcctggtaccagcagaaacctgggaaggccccaaagctgcttatctacaaggcatcgtccctggaatcaggagtgcccagccgcttcagcggatccggatcgggcaccgaattcactctgaccatctcctcactccaacccgacgattttgcgacctactactgcgagcagtacggctcgtattcgccgttgactttcgggggcggtaccaaggtcgagatcaag

SEQ ID NO: 247 - DNA, 384 bp, synthetic construct
  source 1..384: mol_type = other DNA
cagctgcaactgcaggaatccggcccagggcttgtgaagccgtcggagactctctcgctcacttgcaccgtgtccggaggctccatttcctcctccccctacaactgggtctggatccggcagcctcctggaaagggtctggaatggatcggctcaatcctgcactcgggaagcacctactacaacccgagcttgaagtcccgcgtgaccattagcgtggacacctccaagaaccagttctcgctgaaactgtcctctgtgaccgcggcagataccgccgtctactattgtgcgagaggggtgcccacttactggggtgccgctagccattactactactacatggacgtctggggaaagggcacgactgtgaccgtgtcaagc

SEQ ID NO: 248 - DNA, 324 bp, synthetic construct
  source 1..324: mol_type = other DNA
gacatccaaatgacccagagccccagcaccttgtccgcatccgtgggagatagagtgaccattacttgccgggcctcacagtccatttcctcgtggctcgcgtggtaccagcagaagccgggaaaggccccaaagctcctgatctataaggcttcctcgctggaatctggggtcccttcgcgcttctccggatccggctcagggactgagttcacgctgactatctcctccctgcaaccggacgacttcgccacctactactgcgagcagtacggcagcgaaagcccccttacctttggtggaggcaccaaagtggagatcaag

SEQ ID NO: 249 - DNA, 384 bp, synthetic construct
  source 1..384: mol_type = other DNA
caggtccagctggtgcagtcgggagctgaggtcaagaagcctggagcctcagtgaaggtgtcctgcaaagcctcgggctacaccttcaccggctactacatgcactgggtcagacaggcccccggacaaggactggagtggatggggtccatcaacccgagctcaggaggcactaactacgcccagaagttccaaggccgcgtgaccatgactcgggacacctccatttccaccgcgtacatggaactgtccaggctccggagcgacgatacggcggtgtactactgtgcacgcggtggaccatccacttggtacggcggttcggcctactactactatgggatggacgtgtggggacaggggactaccgtgaccgtcagctcc

SEQ ID NO: 250 - DNA, 321 bp, synthetic construct
  source 1..321: mol_type = other DNA
gaaatcgtgctgacccagtcgcctggcactctgtcactgtccccgggagagagagccaccctgagctgtagggcgtcccagtccgtgagctcggactacctggcctggtatcagcagaagccaggacaagcacctcgccttctcatctacggagcctcatcccgggccactgggatccccgaccggttttcgggaagcggttccggcactgatttcaccctgactatctcccgcttggaaccggaggacttcgctgtgtactactgccaacagtacgcgaacctctggaccttcggaggcggtaccaaggtcgagattaag

SEQ ID NO: 251 - DNA, 384 bp, synthetic construct
  source 1..384: mol_type = other DNA
caggtccagctcgtgcagtctggggccgaagtgaagaagccaggagcgtcggtgaaagtgtcctgcaaggcctccggttacaccttcgccggctacgatatgcactgggtcagacaggctcctggccaaggcctggaatggatggggtcgattaacccgacttccggacggaccaactacgcccagaagttccaaggccgcgtgaccatgactcgggacactagcatctcaacggcctacatggagctgtcccgcctgaggtccgacgataccgccgtgtactattgtgcgagaggaggaccctcaacttggtacgggggatccgcctacacatactacggtgcagacgtctggggccagggaaccaccgtgaccgtgtccagc

SEQ ID NO: 252 - DNA, 321 bp, synthetic construct
  source 1..321: mol_type = other DNA
gaaatcgtgctgacccagtcccctgggactctgagcctttcccctggcgaaagggccactctctcatgtcgggcgtcacagtcggtgtccagcgactacttggcttggtaccagcagaagcctggacaagcccccagactcctgatctatggggcctcctcgagagcaactggaatcccggaccggttctccggatcgggaagcggtaccgacttcaccctgacgatttcccgcctggaaccggaggatttcgccgtgtactactgccaacagtacgccccactgtggacctttggcggcggtaccaaggtcgagattaag

SEQ ID NO: 253 - protein, 9 aa, synthetic construct
  VARIANT 4: note = is S, G, E, or N
  VARIANT 5: note = is S, H, or D
  VARIANT 7: note = is S or A
FTFXXYXMN

SEQ ID NO: 254 - protein, 17 aa, synthetic construct
  VARIANT 8: note = is T, F,or Y
  VARIANT 10: note = is Y or D
YISSSSSXIXYADSVKG

SEQ ID NO: 255 - protein, 19 aa, synthetic construct
  VARIANT 6: note = is S or D
  VARIANT 7: note = is M or R
ARDLTXXQSESYYYYGMDV

SEQ ID NO: 256 - protein, 11 aa, synthetic construct
  VARIANT 4: note = is S or P
  VARIANT 11: note = is G, A, or S
GSIXSSSYYWX

SEQ ID NO: 257 - protein, 16 aa, synthetic construct
  VARIANT 1: note = is S or N
  VARIANT 3: note = is S or Y
XIXYSGSTYYNPSLKS

SEQ ID NO: 258 - protein, 16 aa, synthetic construct
  VARIANT 9: note = is Y or A
  VARIANT 10: note = is Y or L
  VARIANT 11: note = is Y or F
  VARIANT 12: note = is Y or W
  VARIANT 14: note = is M or L
ARGEYDRSXXXXGXDV

SEQ ID NO: 259 - protein, 8 aa, synthetic construct
  VARIANT 8: note = is T or S
QQAFSSPX

SEQ ID NO: 260 - skipped

SEQ ID NO: 261 - protein, 17 aa, synthetic construct
  VARIANT 1: note = is G or L
  VARIANT 3: note = is I or F
  VARIANT 5: note = is I, P or T
  VARIANT 6: note = is S or F
  VARIANT 8: note = is T or Y
  VARIANT 10: note = is N, H or V
XIXPXXGXAXYAQKFQG

SEQ ID NO: 262 - protein, 20 aa, synthetic construct
  VARIANT 3: note = is G, Q or A
  VARIANT 4: note = is S, or E
  VARIANT 15: note = is Y or R
  VARIANT 16: note = is Y or D
  VARIANT 18: note = is M or L
ARXXASGYYDRSYYXXGXDV

SEQ ID NO: 263 - protein, 9 aa, synthetic construct
  VARIANT 4: note = is S or I
  VARIANT 8: note = is Y or Q
QQVXSFPXT

SEQ ID NO: 264 - skipped

SEQ ID NO: 265 - protein, 17 aa, synthetic construct
  VARIANT 1: note = is W or S
  VARIANT 5: note = is Y or S
  VARIANT 14: note = is K or E
XISAXSGDTNYAQXLQG

SEQ ID NO: 266 - protein, 15 aa, synthetic construct
  VARIANT 3: note = is A, G, or S
  VARIANT 10: note = is H, W, S, or D
ARXAGYGARXWYFDL

SEQ ID NO: 267 - protein, 10 aa, synthetic construct
  VARIANT 2: note = is Q or G
  VARIANT 3: note = is S, F, W, or R
DXXYSTPPIT

SEQ ID NO: 268 - protein, 11 aa, synthetic construct
  VARIANT 9: note = is Y or S
  VARIANT 11: note = is S, H or N
GSISSGGYXWX

SEQ ID NO: 269 - protein, 16 aa, synthetic construct
  VARIANT 1: note = is S or Y
  VARIANT 4: note = is Y or R
  VARIANT 7: note = is S or Y
XIYXSGXTYYNPSLKS

SEQ ID NO: 270 - protein, 14 aa, synthetic construct
  VARIANT 5: note = is Q or A
  VARIANT 8: note = is T or L
  VARIANT 9: note = is H, T or S
  VARIANT 12: note = is F or V
ARAKXLTXXRYXDY

SEQ ID NO: 271 - protein, 9 aa, synthetic construct
  VARIANT 4: note = is S or R
  VARIANT 5: note = is D or Q
QQAXXLPFT

SEQ ID NO: 272 - protein, 9 aa, synthetic construct
  VARIANT 4: note = is S or N
  VARIANT 5: note = is N or S
  VARIANT 6: note = is Y or H
  VARIANT 9: note = is S or T
GTFXXXAIX

SEQ ID NO: 273 - protein, 17 aa, synthetic construct
  VARIANT 1: note = is G or A
  VARIANT 8: note = is T, V or F
XIIPIFGXANYAQKFQG

SEQ ID NO: 274 - protein, 20 aa, synthetic construct
  VARIANT 10: note = is S or A
  VARIANT 11: note = is S, Q or A
ARGAPNYYGXXSSRGIAFDI

SEQ ID NO: 275 - protein, 9 aa, synthetic construct
  VARIANT 9: note = is T, K or S
VQANAFPYX

SEQ ID NO: 276 - protein, 11 aa, synthetic construct
  VARIANT 7: note = is S or P
  VARIANT 9: note = is Y or N
  VARIANT 11: note = is G or V
GSISSSXYXWX

SEQ ID NO: 277 - protein, 16 aa, synthetic construct
  VARIANT 3: note = is S or L
  VARIANT 4: note = is Y or H
SIXXSGSTYYNPSLKS

SEQ ID NO: 278 - protein, 20 aa, synthetic construct
  VARIANT 4: note = is A or V
  VARIANT 8: note = is F or W
ARGXPTYXGAASHYYYYMDV

SEQ ID NO: 279 - protein, 10 aa, synthetic construct
  VARIANT 6: note = is Y or E
EQYGSXSPLT

SEQ ID NO: 280 - protein, 9 aa, synthetic construct
  VARIANT 4: note = is T or A
  VARIANT 7: note = is Y or D
YTFXGYXMH

SEQ ID NO: 281 - protein, 17 aa, synthetic construct
  VARIANT 5: note = is S or T
  VARIANT 8: note = is G or R
SINPXSGXTNYAQKFQG

SEQ ID NO: 282 - protein, 21 aa, synthetic construct
  VARIANT 15: note = is Y or T
  VARIANT 19: note = is M or A
ARGGPSTWYGGSAYXYYGXDV

SEQ ID NO: 283 - protein, 8 aa, synthetic construct
  VARIANT 5: note = is N or P
QQYAXLWT

SEQ ID NO: 284 - protein, 9 aa, synthetic construct
YTFTSEGIH

SEQ ID NO: 285 - protein, 122 aa, synthetic construct
QVQLVQSGAEVKKPGASVKVSCKASGYTFTSEGIHWVRQAPGQGLEWMGSISASSGDTNYAQKLQGRVTMTTDTSTSTAYMELRSLRSDDTAVYYCARSAGYGARHWYFDLWGQGTMVTVSS

SEQ ID NO: 286 - DNA, 324 bp, synthetic construct
  source 1..324: mol_type = other DNA
gatattcaaatgacccagtcaccgagctccttgagcgcgtccgtgggagacagagtgaccattacttgtcgcgcctcgcaatcgatctcatcctatctcaactggtaccagcagaagcccggcaaagctccaaagcttctgatctacggcgcaagcagcctgcagtctggagtgccttcgcggttctccggttccggatccgggaccgacttcactctgaccatctcctccctgcaacctgaggatttcgccacctactactgcgaccagttttactccaccccgcccatcacgttcggagggggcactaaggtcgaaatcaag

SEQ ID NO: 287 - DNA, 324 bp, synthetic construct
  source 1..324: mol_type = other DNA
gacattcaaatgacccagtccccatcgtccctgagcgcctcagtgggagatagagtgaccatcacctgtcgcgctagccagtccatctcctcgtacctgaactggtaccagcagaagcctggaaaagccccgaagcttctgatctacggtgccagcagcctgcagtctggcgtgccttcccggttctcgggatcaggctccgggaccgacttcactttgactatctcctccctccaacccgaggatttcgcgacctactattgcgaccagttctactccacgccgcccattacctttggcgggggaactaaggtcgaaatcaag

SEQ ID NO: 288 - DNA, 324 bp, synthetic construct
  source 1..324: mol_type = other DNA
gatatccaaatgacccagtccccgagctccctgtccgcctccgtgggagatcgcgtgaccattacgtgtagagcctcccagtcgatcagcagctatctgaactggtaccagcagaagccgggcaaagctcctaagctccttatctacggcgccagctcactgcagtccggagtgccatcgcggttctccggatccggctcaggaaccgacttcaccttgactatttcttcgctgcaacctgaggactttgcgacctactactgcgaccagtggtactccaccccccccatcactttcggggggggtactaaggtcgaaatcaag

SEQ ID NO: 289 - DNA, 381 bp, synthetic construct
  source 1..381: mol_type = other DNA
caggtccagctcgtgcagtcaggcgccgaagtcaagaagccgggttccagcgtgaaggtgtcctgcaaagccagcggaggcactttctccaactatgcgatcagctgggtcagacaggcccccggacaaggactggagtggatgggcgggatcattccaatcttcgggactgcgaactacgcacagaagtttcagggtcgcgtgaccatcactgctgacgagtcgacctctaccgcctacatggagctgtcctcgctgcggtccgaagataccgccgtgtactactgtgcgagaggagcccctaattactacggaagctcatcctcgcgggggattgccttcgacatctggggacaaggcaccatggtcaccgtgtcctcg

SEQ ID NO: 290 - DNA, 321 bp, synthetic construct
  source 1..321: mol_type = other DNA
gacatccaaatgacccagtcgccatcctccgtgagcgcatccgtgggagatcgcgtcaccattacctgtcgggcgtcacagggcattagctcttggctggcctggtaccagcagaagcccggaaaggctcccaaacttctgatctacgccgcgtcctcactgcaaagcggcgtgccttcgagattcagcggatccggttccggcactgactttactctcaccatctcgtccctgcaaccggaggacttcgccacctactactgcgtgcaggccaacgccttcccttacaccttcggaggggggactaaggtcgaaatcaag

SEQ ID NO: 291 - DNA, 381 bp, synthetic construct
  source 1..381: mol_type = other DNA
caagtgcaactggtgcagtcgggtgccgaggtcaagaagcctgggagctccgtgaaagtgtcctgcaaggcctccggaggaactttcaacaactacgcgatcagctgggtcagacaggctccaggccaaggcctcgaatggatgggcgcgattattcccatcttcggagtggccaactacgcccagaagtttcagggacgcgtgactatcaccgccgatgagtccacctccactgcctacatggaactgagctcactgcggtccgaggacaccgcagtgtactactgtgcaaggggtgccccgaattactatggagcccagtcctcacgggggatcgctttcgacatctggggacagggcaccatggtcaccgtgtcgtcg

SEQ ID NO: 292 - DNA, 321 bp, synthetic construct
  source 1..321: mol_type = other DNA
gacattcaaatgacccagtcgccttcgtccgtgagcgcatccgtgggtgatcgcgtgaccatcacttgccgggcttcacaaggcatttcctcctggctggcctggtaccagcagaagcccggaaaagcgcccaagctgctcatctacgccgcctcctctcttcaatccggcgtgccgtcaagattctcgggaagcggatccggcaccgacttcacgctgactatcagcagcctgcagccagaggacttcgcgacctactactgtgtgcaggccaacgcctttccgtataagttcggaggggggaccaaggtcgaaatcaag

SEQ ID NO: 293 - DNA, 381 bp, synthetic construct
  source 1..381: mol_type = other DNA
caagtgcagctcgtgcagtcgggagccgaggtcaagaagcctggcagcagcgtgaaggtgtcctgcaaagcgtccggaggcaccttcagctcccacgccatcacctgggtcagacaggctccgggacagggtctggagtggatgggcgggatcattcccatcttcgggttcgccaactacgctcagaagttccagggacgcgtgactatcaccgcggacgaatcaaccagcactgcctatatggaactgtcctccctgcggtcggaagataccgccgtgtactactgtgcaaggggagcgccaaactactacggctctgcctcctcacggggaatcgcctttgacatttggggtcaaggcactatggtcacggtgtcctcg

SEQ ID NO: 294 - DNA, 321 bp, synthetic construct
  source 1..321: mol_type = other DNA
gacatccaaatgactcagtcaccgtcctccgtgtcggcctccgtgggagatagagtgaccattacttgtcgcgcaagccagggcatcagcagctggctcgcctggtaccagcagaagcccgggaaagcccctaagctgctgatctacgccgcttcctcccttcaatccggagtgccatcccggttctctggttcgggatcaggcaccgactttaccctgaccattagctcgctgcagcccgaggacttcgcgacctactattgcgtgcaagccaacgcgttcccgtactccttcgggggcggaactaaggtcgaaatcaag

SEQ ID NO: 295 - DNA, 366 bp, synthetic construct
  source 1..366: mol_type = other DNA
caggtccaactcgtgcagtccggggctgaagtcaagaagccgggagctagcgtgaaagtgtcctgcaaggcctccggctacaccttcggaccctacggaatttcatgggtcagacaggcgcctggacagggtctggagtggatggggtggatctccgcctactcgggcgacactaactacgcgcagaagctgcaaggacgcgtgaccatgactaccgacacctcgaccagcaccgcctacatggaacttaggtccctgcggtccgacgatacggccgtgtactactgtgcaagagccgccggctatggagcccggtggtggtacttcgatctgtggggccagggtactatggtcaccgtgtcaagc

(19) United States
(12) Patent Application Publication
(10) Pub. No.: US 2026/0258155 A1
(43) Pub. Date: Sep. 3, 2026

(54) KALLIKREIN ANTIBODIES AND USES THEREOF

(71) Applicant: Triveni Bio, Inc., Watertown, MA (US)

(72) Inventors: Jennifer L. Dovey, Auburndale, MA (US); Allan Capili, Somerville, MA (US); Leonard Zon, Brookline, MA (US); Sonya Entova, Arlington, MA (US); Christopher Nordyke, Woburn, MA (US); Jenna Calvino, Newton Upper Falls, MA (US); Todd Borland, Mansfield, MA (US); Ye Tang, Winchester, MA (US); Brantley Herrin, Watertown, MA (US); Bradley Lunde, Lebanon, NH (US); Nathan Sharkey

(73) Assignee: Triveni Bio, Inc., Watertown, MA (US)

(21) Appl. No.: 19/163,226

(22) PCT Filed: Mar. 8, 2024
(86) PCT No.: PCT/US2024/019245

§ 371 Date: Sep. 8, 2025

Related U.S. Application Data
(60) Provisional application No. 63/489,419, filed on Mar. 9, 2023.
(60) Provisional application No. 63/562,304, filed on Mar. 7, 2024.

(51) Int. Cl. C07K 16/40; A61K 9/00; A61K 38/00; A61P 17/00; C12N 15/11

(52) U.S. Cl. CPC C07K 16/40; A61K 9/0019; A61K 38/00; A61P 17/00; C12N 15/11; C07K 2317/31; C07K 2317/565; C07K 2317/76

(57) ABSTRACT

Aspects of the application provide anti-KLK5 antibodies, anti-KLK7 antibodies, and anti-KLK5/KLK7 antibodies (e.g., bispecific antibodies) and methods of using the same for promoting barrier function and reducing inflammation, and treating conditions such as Netherton syndrome, eosinophilic esophagitis and atopic dermatitis.

### RELATED APPLICATIONS

This application claims priority under 35 U.S.C. § 119(e) to U.S. Provisional Application, U.S. Ser. No. 63/489,419, filed Mar. 9, 2023, and U.S. Provisional Application, U.S. Ser. No. 63/562,304, filed Mar. 7, 2024, the entire contents of each of which are incorporated herein by reference.

### REFERENCE TO AN ELECTRONIC SEQUENCE LISTING

The contents of the electronic sequence listing (A14070000W000-SEQ-LJG.xml; Size: 279,377 bytes; and Date of Creation: Mar. 8, 2024) is herein incorporated by reference in its entirety.

### BACKGROUND

Kallikrein (KLK) enzymes regulate desquamation and innate immunity to support skin homeostasis and wound healing. In a healthy skin, the outermost layer of the epidermis is regularly shed through a KLK driven proteolytic cascade resulting in the degradation of corneodesmosomes and desquamation. KLK5 is understood to be a main activator of this proteolytic cascade. Autoactivated KLK5 enzymatically converts proKLK7 and proKLK14 to active forms and stimulates a positive feedback loop that, via KLK14, leads to the production of more proKLK5. These KLK enzymes are kept in check by endogenous serine protease inhibitors, such as lymphoepithelial Kazal-type-related inhibitors. Dysregulation of KLKs, including KLK5 and KLK7, is associated with skin disorders, inflammatory diseases, and cancer. For example, hyperactive kallikrein 5 and 7 cause both genetic and spontaneous epidermal barrier disorders (e.g., Netherton syndrome, eosinophilic esophagitis, atopic dermatitis).

### SUMMARY

Certain aspects of the disclosure relate to a recognition that loss of balance between endogenous KLK proteases and associated protease inhibitors causes barrier dysfunction and induces inflammation (see, e.g., FIG. 1), which can result in inflammatory conditions, such as Netherton syndrome, eosinophilic esophagitis and atopic dermatitis. In some embodiments, methods and related compositions are provided that are useful for inhibition of KLK5 and/or KLK7 for purposes of improving barrier function and reducing inflammation, which ameliorates disease severity. Aspects of the disclosure provide anti-KLK5 antibodies that have high binding affinity and specificity to KLK5 and that inhibit KLK5 activity. Further aspects of the disclosure provide anti-KLK7 antibodies that have high binding affinity and specificity to KLK7 and that inhibit KLK7 activity. Further aspects of the disclosure provide anti-KLK5/KLK7 antibodies (e.g., bispecific antibodies targeting KLK5 and KLK7) that have high binding affinity and specificity to both KLK5 and KLK7 and that inhibit KLK5 and KLK7 activity. Accordingly, in some embodiments, the disclosure provides methods, antibodies for use in methods, and related antibody compositions for treating conditions associated with KLK5 and KLK7 dysregulation, such as Netherton Syndrome, atopic dermatitis (with and without filaggrin mutations), eosinophilic esophagitis, prurigo nodularis, chronic pruritus of unknown origin (CPUO), asthma (e.g., KLK5 related asthma), and ichthyosis vulgaris.

The foregoing and other aspects, implementations, acts, functionalities, features and embodiments of the present teachings can be more fully understood from the following description in conjunction with the accompanying drawings.

### BRIEF DESCRIPTION OF THE DRAWINGS

The accompanying drawings, which are incorporated in and constitute a part of this specification, illustrate certain embodiments, and together with the written description, serve to provide non-limiting examples of certain aspects of the compositions and methods disclosed herein.

FIG. 1 is a diagram showing aberrant protease activation (e.g., aberrant KLK5, KLK7, and KLK14) activation leading to skin barrier defect associated diseases.

FIGS. 2A-2J show relative response curves of anti-KLK5 antibodies binding to either the active form of human KLK5 (hKLK5) or the proform of hKLK5 (FIGS. 2A-2D) and anti-KLK7 antibodies binding to either the active form of human KLK7 (hKLK7) or the proform of hKLK7 (FIGS. 2F-2I). Anti-KLK5 antibodies KLK5-L1-Ab2 (FIG. 2A), KLK5-L2-Ab3 (FIG. 2B), KLK5-L3-Ab5 (FIG. 2C), and KLK5-L2-Ab4 (FIG. 2D) bind only the active form of hKLK5, and anti-KLK7 antibodies KLK7-L2-Ab4 (FIG. 2F), KLK7-L2-Ab5 (FIG. 2G), KLK7-L5-Ab2 (FIG. 2H), and KLK7-L1-Ab7 (FIG. 2I) bind only the active form of hKLK7. The anti-KLK5/anti-KLK7 bispecific antibody KLK5-L2-Ab2 x KLK7-L1-Ab8 is shown binding only to the active forms of hKLK5 (FIG. 2E) and hKLK7 (FIG. 2J).

FIGS. 3A-3B are SDS-PAGE results showing antibodies alone or following incubation with either hKLK5 (FIG. 3A) or hKLK7 (FIG. 3B). Anti-KLK5 antibody KLK5-L2-Ab2 is resistant to cleavage by hKLK5, while KLK5-L3-Ab7 and KLK-L1-Ab5 undergo heavy chain cleavage by hKLK5 (FIG. 3A). Anti-KLK7 antibodies KLK7-L1-Ab8, KLK7-L2-Ab3, KLK7-L3-Ab2, KLK7-L4-Ab2, and KLK7-L5-Ab2 are resistant to cleavage by hKLK7 (FIG. 3B).

FIGS. 4A-4K show binding activity of anti-KLK5 antibodies, relative to an isotype control, of KLK1 (FIG. 4A), KLK2 (FIG. 4B), KLK4 (FIG. 4C), KLK6 (FIG. 4D), KLK12 (FIG. 4E), KLK14 (FIG. 4F), plasma KLK (FIG. 4G), trypsin (FIG. 4H), chymotrypsin (FIG. 4I), urokinase (FIG. 4J), and KLK6 again in follow-up studies (FIG. 4K). The tested antibodies do not specifically inhibit non-KLK5 family members or related proteases, as the relative activity is not higher than the isotype control.

FIGS. 5A-5J show the binding activity of anti-KLK7 antibodies. Relative to an isotype control, of KLK1 (FIG. 5A), KLK2 (FIG. 5B), KLK4 (FIG. 5C), KLK6 (FIG. 5D), KLK12 (FIG. 5E), KLK14 (FIG. 5F), plasma KLK (FIG. 5G), trypsin (FIG. 5H), chymotrypsin (FIG. 5I), and urokinase (FIG. 5J). The tested antibodies do not specifically inhibit non-KLK7 family members or related proteases, as the relative activity is not higher than the isotype control.

FIG. 6 shows binding activity of anti-KLK5/anti-KLK7 bispecific antibodies, compared to an isotype control, of KLK13 at various concentrations.

FIGS. 7A-7B show the relative response of anti-KLK5 antibody KLK5-L1-Ab5 binding hKLK5 alone or in the presence of inhibitors of serine proteases PMSF (FIG. 7A), leupeptin, or SPINK5 (FIG. 7B).

FIGS. 8A-8B show the relative response of anti-KLK5 antibody KLK5-L2-Ab2 binding hKLK5 alone or in the presence of inhibitors of serine proteases PMSF (FIG. 8A), leupeptin, or SPINK5 (FIG. 8B).

FIGS. 9A-9B show the relative response of anti-KLK5 antibody KLK5-L3-Ab7 binding hKLK5 alone or in the presence of inhibitors of serine proteases PMSF (FIG. 9A), leupeptin, or SPINK5 (FIG. 9B).

FIGS. 10A-10B show the relative response of anti-KLK5/anti-KLK7 bispecific antibody KLK5-L2-Ab2 x KLK7-L1-Ab8 binding hKLK5 alone or in the presence of inhibitors of serine proteases PMSF (FIG. 10A), leupeptin, or SPINK5 (FIG. 10B).

FIGS. 11A-11G show the relative response of anti-KLK7 antibodies KLK7-L1-Ab8 (FIG. 11A), KLK7-L2-Ab3 (FIG. 11B), KLK7-L3-Ab2 (FIG. 11C), KLK7-L4-Ab2 (FIG. 11D), KLK7-L5-Ab2 (FIG. 11E), KLK7-L1-Ab8 (FIG. 11F), and anti-KLK7/anti-KLK5 bispecific antibody KLK5-L2-Ab2 x KLK7-L1-Ab8 (FIG. 11G) binding hKLK7 alone or in the presence of inhibitors of serine proteases PMSF or SPINK5.

FIGS. 12A-12F show competitive binding of KLK5 (FIGS. 12A-12D) and KLK7 (FIGS. 12E-12F) between anti-KLK5 or anti-KLK7 antibodies and SPINK5, which binds the active site of KLK5 and KLK7. Anti-KLK5 antibodies KLK5-L1-Ab5 (FIG. 12A), KLK5-L2-Ab (FIG. 12B), or KLK5-L3-Ab7 (FIG. 12C) are bound to a chip and KLK5 is added, resulting in an increase in the binding curve. Addition of a second anti-KLK5 antibody (“mAb #2”) or SPINK5 does not increase binding. Anti-KLK7 antibody KLK7-L1-Ab5 (FIG. 12E) is bound to a chip and KLK7 is added, resulting in an increase in the binding curve. Addition of an isotype control or SPINK5 does not increase binding. Alternatively, SPINK5 is bound to the chip, and either KLK5 (FIG. 12D) or KLK7 (FIG. 12F) is added, resulting in an increase in the binding curve. Addition of an anti-KLK5 antibody (FIG. 12D) or an anti-KLK7 antibody (FIG. 12F) does not increase binding, as SPINK5 is already bound to the active site of KLK5 or KLK7.

FIG. 13 shows the effects of treatment with either anti-KLK5/anti-KLK7 bispecific antibody KLK5-L2-Ab2 x KLK7-L1-Ab8 or Comparator Antibody #1 at 30 mg/kg on the stratum corneum thickness in an MC903 atopic dermatitis mouse model. Treatment with KLK5-L2-Ab2 x KLK7-L1-Ab8 antibody led to a significant reduction in thickness. * p<0.001; ** p<0.01; * p<0.05, via one-way ANOVA with Dunnett's multiple comparisons.

FIGS. 14A-14C represent the treatment efficacy of anti-KLK5/anti-KLK7 bispecific antibody KLK5-L2-Ab2 x KLK7-L1-Ab8 compared to the topical ointment tacrolimus and Comparator Antibody #1 on disease presentation in an Nc/Nga atopic dermatitis mouse model, as measured by clinical ear thickness (FIG. 14A), epidermal area (FIG. 14B), and itch (FIG. 14C). *** p<0.001; ** p<0.01; * p<0.05, via one-way ANOVA with Dunnett's multiple comparisons.

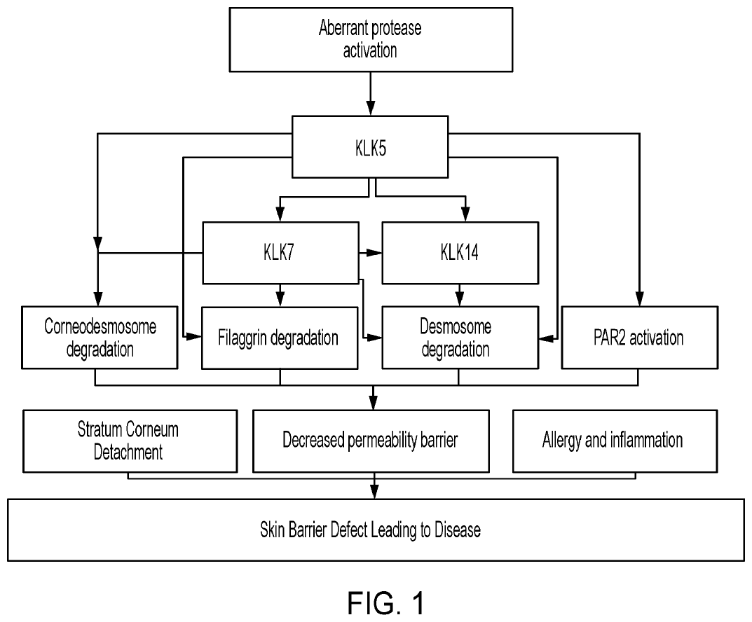

### DETAILED DESCRIPTION

The present disclosure, at least in part, is based on the development of inhibitory anti-KLK5 antibodies and variants thereof that have high binding affinity and specificity to KLK5. Also provided are methods of using the anti-KLK5 antibodies and their variants in research, diagnostic/detection, therapeutic applications, and anti-KLK5 antibodies for use in such methods. The present disclosure is based in further part on the development of inhibitory anti-KLK7 antibodies and variants thereof that have high binding affinity and specificity to KLK7. Also provided are methods of using the anti-KLK7 antibodies and their variants in research, diagnostic/detection, therapeutic applications, and anti-KLK7 antibodies for use in such methods. The present disclosure is based in further part on the development of inhibitory anti-KLK5/KLK7 antibodies (e.g., multi-specific antibodies, e.g., bispecific antibodies) and variants thereof that have high binding affinity and specificity to both KLK5 and KLK7. Also provided are methods of using the anti-KLK5/KLK7 antibodies and their variants in research, diagnostic/detection, and therapeutic applications.

The foregoing and other aspects, implementations, acts, functionalities, features and embodiments of the present teachings can be more fully understood from the following description in conjunction with the accompanying drawings.

### I. DEFINITIONS

Administering: As used herein, the terms “administering” or “administration” means to provide an antibody or a composition thereof to a subject in a manner that is physiologically and/or pharmacologically useful (e.g., to treat a condition in the subject).

Affinity Matured Antibody: “Affinity Matured Antibody” is used herein to refer to an antibody with one or more alterations in one or more CDRs, which result in an improvement in the affinity (e.g., KD, kd or ka) of the antibody for a target antigen compared to a parent antibody, which does not possess the alteration(s). Exemplary affinity matured antibodies may have nanomolar or even picomolar affinities for the target antigen in some embodiments. A variety of procedures for producing affinity matured antibodies are available, including the screening of a combinatory antibody library that has been prepared using bio-display. For example, Marks et al., BioTechnology, 10: 779-783 (1992) describes affinity maturation by VH and VL domain shuffling. Random mutagenesis of CDR and/or framework residues is described by Barbas et al., Proc. Nat. Acad. Sci. USA, 91: 3809-3813 (1994); Schier et al., Gene, 169: 147-155 (1995); Yelton et al., J. Immunol., 155: 1994-2004 (1995); Jackson et al., J. Immunol., 154(7): 3310-3319 (1995); and Hawkins et al, J. Mol. Biol., 226: 889-896 (1992). Selective mutation at selective mutagenesis positions and at contact or hypermutation positions with an activity-enhancing amino acid residue is described in U.S. Pat. No. 6,914,128 B1.

Antibody: As used herein, the term “antibody” refers to a polypeptide that comprises at least one immunoglobulin variable domain, which comprises at least one distinct antigen-specific binding site, or a portion of an immunoglobulin variable domain (such as a paratope or portion thereof) that comprises at least one distinct antigen-specific binding site. In some embodiments, an antibody comprises at least one distinct antigen-specific binding site that specifically binds to the active site of an enzyme. In some embodiments, an antibody is a full-length antibody. In some embodiments, an antibody is a chimeric antibody. In some embodiments, an antibody is a humanized antibody. However, in some embodiments, an antibody is a Fab fragment, a F(ab′)2 fragment, a Fv fragment or a scFv fragment. In some embodiments, an antibody is a multi-specific antibody (e.g., a bispecific antibody). In some embodiments, an antibody is a nanobody derived from a camelid antibody or a nanobody derived from shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (α), delta (Δ), epsilon (ε), gamma (γ) or mu (μ) heavy chain. In some embodiments, the heavy chain of an antibody described herein can comprise a human alpha (α), delta (Δ), epsilon (ε), gamma (γ) or mu (μ) heavy chain. In a particular embodiment, an antibody described herein comprises a human gamma 1 CH1, CH2, and/or CH3 domain. In some embodiments, the amino acid sequence of the V_{H} domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) supra. In some embodiments, the V_{H} domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P., et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Bispecific Antibody: As used herein, the term “bispecific antibody” refers to an antibody that comprises two distinct antigen-specific binding sites or two linked (covalently or non-covalently) antibodies that, combined, comprise two distinct antigen-specific binding sites. Non-limiting examples of bispecific antibody formats or architectures are provided in Labrijn, A F, et al., Bispecific antibodies: a mechanistic review of the pipeline, Nature Reviews Drug Discovery volume 18, pages 585-608 (2019) and Brinkmann U and Kontermann E E, The making of bispecific antibodies, MAbs. 2017 February/March; 9(2):182-212, the entire contents of each of which are incorporated herein by reference in their entireties.

CDR: As used herein, the term “CDR” refers to the complementarity determining region within antibody variable sequences. A typical antibody molecule comprises a heavy chain variable region (VH) and a light chain variable region (VL), which are usually involved in antigen binding. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the IMGT definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242; IMGT®, the international ImMunoGeneTics information System® http://www.imgt.org, Lefranc, M.-P. et al., Nucleic Acids Res., 27:209-212 (1999); Ruiz, M. et al., Nucleic Acids Res., 28:219-221 (2000); Lefranc, M.-P., Nucleic Acids Res., 29:207-209 (2001); Lefranc, M.-P., Nucleic Acids Res., 31:307-310 (2003); Lefranc, M.-P. et al., In Silico Biol., 5, 0006 (2004) [[Epub]], 5:45-60 (2005); Lefranc, M.-P. et al., Nucleic Acids Res., 33:D593-597 (2005); Lefranc, M.-P. et al., Nucleic Acids Res., 37:D1006-1012 (2009); Lefranc, M.-P. et al., Nucleic Acids Res., 43:D413-422 (2015); Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). ee also hgmp.mrc.ac.uk and bioinf.org.uk/abs. As used herein, a CDR may refer to the CDR defined by any method known in the art. Two antibodies having the same CDR means that the two antibodies have the same amino acid sequence of that CDR as determined by the same method, for example, the IMGT definition.

In certain embodiments, there are three CDRs in each of the variable regions of a heavy chain and a light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

CDR-grafted antibody: As used herein, the term “CDR-grafted antibody” refers to antibodies which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

Chimeric antibody: As used herein, the term “chimeric antibody” refers to antibodies which comprise heavy and light chain variable region sequences from one species and constant region sequences from another species, such as antibodies having murine heavy and light chain variable regions linked to human constant regions.

Complementary: As used herein, the term “complementary” refers to the capacity for precise pairing between two nucleotides or two sets of nucleotides. In particular, complementary is a term that characterizes an extent of hydrogen bond pairing that brings about binding between two nucleotides or two sets of nucleotides. For example, if a base at one position of an oligonucleotide is capable of hydrogen bonding with a base at the corresponding position of a target nucleic acid (e.g., an mRNA), then the bases are considered to be complementary to each other at that position. Base pairings may include both canonical Watson-Crick base pairing and non-Watson-Crick base pairing (e.g., Wobble base pairing and Hoogsteen base pairing). For example, in some embodiments, for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

Conservative amino acid substitution: As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Cross-reactive: As used herein, the term “cross-reactive,” refers to a property of the agent being capable of specifically binding to more than one antigen of a similar type or class (e.g., antigens of multiple homologs, paralogs, or orthologs) with similar affinity or avidity. For example, in some embodiments, an antibody that is cross-reactive against human and non-human primate antigens of a similar type or class (e.g., a human KLK5 and non-human primate KLK5, a human KLK7 and non-human primate KLK7) is capable of binding to the human antigen and non-human primate antigens with a similar affinity or avidity. In some embodiments, an antibody is cross-reactive against a human antigen and a rodent antigen of a similar type or class. In some embodiments, an antibody is cross-reactive against a rodent antigen and a non-human primate antigen of a similar type or class. In some embodiments, an antibody is cross-reactive against a human antigen, a non-human primate antigen, and a rodent antigen of a similar type or class.

Effective Amount: As used herein, “an effective amount” refers to the amount of each active agent (e.g., anti-KLK5 antibody, anti-KLK7 antibody, anti-KLK5/KLK7 antibody) required to confer a desired effect (e.g., a therapeutic effect on the subject), either alone or in combination with one or more other active agents. In some embodiments, the therapeutic effect is reduced KLK5 and/or KLK7 activity and/or alleviated disease (e.g., Netherton syndrome, eosinophilic esophagitis and atopic dermatitis) or related symptoms, e.g., improved barrier function.

Framework: As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region. Human heavy chain and light chain acceptor sequences are known in the art. In one embodiment, the acceptor sequences known in the art may be used in the antibodies disclosed herein.

Human antibody: The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

Humanized antibody: As used herein, the term “humanized antibody” refers to antibodies which comprise heavy and light chain variable region sequences from a non-human species (e.g., a mouse) but in which at least a portion of the V_{H} and/or V_{L} sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences. In one embodiment, humanized antibodies are provided. Such antibodies may be generated by obtaining murine monoclonal antibodies using traditional hybridoma technology followed by humanization using in vitro genetic engineering, such as those disclosed in Kasaian et al PCT publication No. WO 2005/123126 A2.

Humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some embodiments, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs derived from one or more CDRs from the original antibody. Humanized antibodies may also involve affinity maturation.

In some embodiments, humanization is achieved by grafting the CDRs (e.g., as shown in Tables 1a, 1b, 2a, or 2b) into the human variable domains (e.g., IGKV1-NL1*01 and IGHV1-3*01 human variable domain). In some embodiments, an antibody of the present disclosure is a humanized variant comprising one or more amino acid substitutions (e.g., in the VH framework region) as compared with any one of the VHs listed in Tables 1a, 1b, 2a, or 2b, and/or one or more amino acid substitutions (e.g., in the VL framework region) as compared with any one of the VLs listed in Tables 1a, 1b, 2a, or 2b.

Isolated antibody: An “isolated antibody”, as used herein, is intended to refer to an antibody that is substantially free of other antibodies having different antigenic specificities (e.g., an isolated antibody that specifically binds KLK5 is substantially free of antibodies that specifically bind antigens other than KLK5, an isolated antibody that specifically binds KLK7 is substantially free of antibodies that specifically bind antigens other than KLK7, an isolated antibody that specifically binds KLK5 and KLK7 is substantially free of antibodies that specifically bind antigens other than KLK5 and KLK7). An isolated antibody may, however, have cross-reactivity to other antigens, in some embodiments. Moreover, an isolated antibody may be substantially free of other cellular material and/or chemicals.

Kabat numbering: As used herein, the terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

Multi-Specific Antigen Binding Molecule: As used herein, the term “multi-specific antigen binding molecule” refers to a molecule that comprises two or more antigen-specific binding sites. In some embodiments, a multi-specific antigen binding molecule is a multi-specific antibody (e.g., a bispecific antibody).

Multi-Specific Antibody: As used herein, the term “multi-specific antibody” refers to an antibody that comprises at least two distinct antigen-specific binding sites or at least two linked (covalently or non-covalently) antibodies that, combined, comprise at least two distinct antigen-specific binding sites. In some embodiments, a multi-specific antibody is a bispecific antibody. Non-limiting examples of multi-specific specific antibody formats or architectures are provided in Sawant M S, et al., Toward Drug-Like Multispecific Antibodies by Design, Int J Mol Sci. 2020 Oct. 12; 21(20):7496; Klein C, et al., The use of CrossMAb technology for the generation of bi- and multispecific antibodies, MAbs 2016 August-September; 8(6):1010-20; and Brinkmann U and Kontermann E E, The making of bispecific antibodies, MAbs. 2017 February/March; 9(2):182-212, the entire contents of each of which are incorporated herein by reference in their entireties.

Recombinant antibody: As used herein, the term “recombinant antibody”, as used herein, is intended to include all antibodies that are prepared, expressed, created or isolated by recombinant means, such as antibodies expressed using a recombinant expression vector transfected into a host cell (described in more details in this disclosure), including, for example, antibodies isolated from a recombinant, combinatorial human antibody library (Hoogenboom H. R., (1997) TIB Tech. 15:62-70; Azzazy H., and Highsmith W. E., (2002) Clin. Biochem. 35:425-445; Gavilondo J. V, and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. In some embodiments, recombinant human antibodies are provided herein. In certain embodiments, such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo. One embodiment of the disclosure provides fully human antibodies, e.g., capable of binding human KLK5 or KLK7, which can be generated using appropriate techniques, such as, but not limited to, using human Ig phage libraries such as those disclosed in Jermutus et al., PCT publication No. WO 2005/007699 A2.

Selective: As used herein, the term “selective” or “selectively” refers to the ability of a molecule to produce an effect (e.g., inhibit, antagonize, agonize, etc) in relation to its target molecule compared to a reference molecule. For example, a molecule that selectively inhibits its target molecule means that this molecule is capable of inhibiting its target molecule with a degree that is distinguishable from a reference molecule in an inhibition assay or other inhibitory context. For example, with respect to an inhibitor, the term, “selectively inhibits”, refers to the ability of the inhibitor to inhibit its target molecule with a degree that is distinguishable from a reference molecule that is not substantially inhibited in an inhibition assay, e.g., to an extent that permit selective inhibition of the target molecule, as described herein. Once the reaction is terminated, the signal produced by inhibiting the target molecule can be measured. The half maximal inhibitor concentration for the target molecule and the reference molecule can be calculated.

Specifically binds: As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, as described herein. In some embodiments, an antibody specifically binds to a target if the antibody has a K_{D} for binding the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, an antibody specifically binds KLK5 or KLK7.

Subject: As used herein, the term “subject” refers to a mammal. In some embodiments, a subject is non-human primate, or rodent. In some embodiments, a subject is a human. In some embodiments, a subject is a patient, e.g., a human patient that has or is suspected of having a disease.

Treatment: As used herein, the term “treating” or “treatment” refers to the application or administration of a composition including one or more active agents (e.g., anti-KLK5 antibodies, anti-KLK7 antibodies, anti-KLK5/KLK7 antibodies) to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder. Alleviating a target disease/disorder includes delaying or preventing the development or progression of the disease, or reducing disease severity. It will be understood that references to treating or treatment may also refer to antibodies.

### II. ANTI-KLK5 ANTIBODIES, ANTI-KLK7 ANTIBODIES AND ANTI-KLK5/KLK7 ANTIBODIES

### (a) Anti-KLK5 Antibodies

In some embodiments, an antibody targeting KLK5 (referred to as an anti-KLK5 antibody) is an antibody specific for Kallikrein-5 (KLK5). Provided herein, in some aspects, are antibodies that bind to KLK5 (e.g., human KLK5, or mouse KLK5) with high specificity and affinity. In some embodiments, the anti-KLK5 antibody described herein specifically binds to an epitope of KLK5 that is exposed or becomes exposed to an antibody. In some embodiments, anti-KLK5 antibodies provided herein bind specifically to KLK5 from human, non-human primates, mouse, rat, etc. In some embodiments, anti-KLK5 antibodies provided herein specifically bind to human KLK5. In some embodiments, anti-KLK5 antibodies provided herein specifically bind to mouse KLK5.

Kallikrein-5, also known as stratum corneum tryptic enzyme (SCTE), is a serine protease expressed in the epidermis, is encoded by the KLK5 gene. The KLK5 gene is one of the fifteen kallikrein subfamily members located in a cluster on chromosome. Its expression is up-regulated by estrogens and progestins. KLK5 is expressed in the stratum granulosum and stratum corneum. In some embodiments, KLK5 regulates epidermal desquamation. In some embodiments, KLK5 regulates epidermal desquamation in conjunction with another member of the Kallikrein family proteases (e.g., KLK7 and/or KLK14). In some embodiments, KLK5 degrades proteins which form the epidermis (e.g., stratum corneum, stratum lucidum, stratum granulosum, stratum spinosum, or stratum basale). In some embodiments, KLK5 degrades proteins which form the stratum corneum and/or stratum granulosum (e.g., Corneodesmosin (CDSN), desmoglein 1 (DSG1), and desmocollin 1 (DSC1), etc). In the epidermis (e.g., stratum granulosum and stratum corneum) KLK5 is expressed in an inactive form (sometimes referred to as the proform or pro-form), proKLK5, and can autoactivate itself. When activated, KLK5 can, through a proteolytic cleavage, convert both proKLK7 and proKLK14 to active forms. Active KLK14 is then able to activate newly produced proKLK5, thus creating a positive feedback loop (see, e.g., Nauroy et al., Kallikreins: Essential epidermal messengers for regulation of the skin microenvironment during homeostasis, repair and disease, Matrix Biol Plus. 2019; 6-7:100019). KLK7 and KLK14 also degrade proteins which form the stratum corneum and/or stratum granulosum (e.g., Corneodesmosin (CDSN), desmoglein 1 (DSG1), and desmocollin 1 (DSC1), etc). Structural proteins, such as CDSN, DSG1, DSC1, are adhesive proteins of the extracellular part of the corneodesmosomes, the junctional structures that mediate corneocyte cohesion. The degradation of these proteins at the epidermis surface lead to desquamation, which may lead to skin barrier defects (e.g., stratum corneum detachment, decreased permeability barrier, allergy and inflammation, etc). KLK5 and KLK7 have been implicated in this process (see, e.g., Caubet et al., Degradation of Corneodesmosome Proteins by Two Serine Proteases of the Kallikrein Family, SCTE/KLK5/hK5 and SCCE/KLK7/hK7, Journal of Investigative Dermatology, Volume 122, Issue 5, May 2004, Pages 1235-1244). Inhibition of KLK5 and/or KLK7 promotes improved skin barrier integrity and reduced inflammation (e.g., Chavarria-Smith et al., Dual antibody inhibition of KLK5 and KLK7 for Netherton syndrome and atopic dermatitis, SCIENCE TRANSLATIONAL MEDICINE, 14 Dec. 2022, Vol 14, Issue 675).

In some embodiments, an anti-KLK5 antibody described herein specifically binds to an epitope on human KLK5. Exemplary amino acid sequences of human KLK5 are set forth in NCBI Accession Numbers NP_001070959.1, NP_001070960.1, or NP_036559.1, and UniProt Accession Numbers: Q8IU55, Q6S9W8, MOQXX2, Q9POG3, A0A2I2MP48, or A0A2I2MP49, the entire sequences of which are incorporated herein by reference.

In some embodiments, an anti-KLK5 antibody described herein specifically binds to an epitope on mouse KLK5. Exemplary amino acid sequences of mouse KLK5 are set forth in NCBI Accession Numbers NP_081082.1, XP_006541213.1, XP_006541214.1, XP_006541215.1, XP_036009294.1, or XP_036009295.1, and UniProt Accession Numbers P15945, or Q9D140, the entire sequences of which are incorporated herein by reference.

In some embodiments, an anti-KLK5 antibody described herein specifically binds to an epitope on KLK5 (e.g., the catalytic domain/pocket of human KLK5 or mouse KLK5). In some embodiments, an anti-KLK5 antibody described herein prevents KLK5 (e.g., human or mouse KLK5) from cleaving its substrates. In some embodiments, an anti-KLK5 antibody described herein bind to a fragment of a KLK5 (e.g., human or mouse KLK5). The fragment of KLK5 (e.g., human or mouse) may be between about 5 and about 425 amino acids, between about 10 and about 400 amino acids, between about 50 and about 350 amino acids, between about 100 and about 300 amino acids, between about 150 and about 250 amino acids, between about 200 and about 300 amino acids, between about 75 and about 150 amino acids, between about 25 and about 100 amino acids, between about 10 and about 30 amino acids in length. Not wishing to be bound to any particular theory, and in some embodiments, a heavy chain (HC) complementarity-determining region 3 (CDR3) of any one of the anti-KLK5 antibodies described herein inhibits KLK5 (e.g., human or mouse KLK5) by binding to the catalytic domain/pocket of KLK5.

In some embodiments, an anti-KLK5 antibody described herein inhibits KLK5 protease activity. In some embodiments the anti-KLK5 antibody inhibits KLK5 cleavage of BOC-Val-Pro-Arg-AMC with an IC50 of less than 30 nM, less than 25 nM, less than 20 nM, less than 15 nM, less than 10 nM, less than 5 nM, less than 3 nM, less than 2.5 nM, less than 2 nM, or less than 1.5 nM, less than 1 nM, less than 0.5 nM, less than 0.3 nM, less than 0.25 nM, less than 0.2 nM, less than 0.1 nM, or less than 0.05 nM.

In some embodiments, an anti-KLK5 antibody described herein binds specifically to the active form of KLK5. In some embodiments, the anti-KLK5 antibody described herein does not bind to the inactive form of KLK5. In some embodiments, an anti-KLK5 antibody described herein binds specifically to the active site of KLK5. The active site of KLK5 is the site at which the KLK5 substrate molecules bind to undergo cleavage. The active site may also be known as the catalytic domain, or catalytic triad. In some embodiments, the active site (i.e., catalytic domain or catalytic triad) of KLK5 consists of amino acids Ser195, His57, and Asp102 of KLK5 (see, e.g., Goettig et al., Natural and synthetic inhibitors of kallikrein-related peptidases (KLKs), Biochimie. 2010 November; 92(11): 1546-1567).

In some embodiments, antibodies described herein are optimized versions (e.g., affinity matured) of the parental antibody. In some embodiments, an antibody described herein specifically binds a KLK5 (e.g., a human or mouse KLK5) with binding affinity (e.g., as indicated by K_{D}) of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, an antibody described herein specifically binds a KLK5 (e.g., a human or mouse KLK5) with binding affinity (e.g., as indicated by K_{D}) of between 1×10⁻¹⁰ M and 5×10⁻⁹ M, between 1×10⁻¹⁰ M and 1×10⁻⁹ M, between 5×10⁻¹⁰ and 1×10⁻⁹ M, between 5×10⁻¹¹ and 1×10⁻¹⁰ M, between 1×10⁻¹¹ and 5×10⁻¹⁰ M, or between 5×10⁻¹³ and 1×10⁻¹² M. For example, an antibody of the present disclosure can bind to a KLK5 protein (e.g., human or mouse KLK5) with an affinity between 1 pM and 500 nM, e.g., between 50 pM and 100 nM, between 500 pM and 50 nM, between 1 pM and 100 pM, between 10 pM and 100 pM, between 50 pM and 100 pM, between 100 pM and 500 pM, between 500 pM and 1 nM, between 1 nM and 5 nM, between 1 nM and 10 nM, between 5 nM and 25 nM, between 10 nM and 50 nM between 50 nM and 100 nM, between 100 nM and 500 nM. The disclosure also includes antibodies that compete with any of the antibodies described herein for binding to a KLK5 protein (e.g., human or mouse KLK5) and that have an affinity of 100 nM or lower (e.g., 80 nM or lower, 50 nM or lower, 20 nM or lower, 10 nM or lower, 1 nM or lower, 500 pM or lower, 50 pM or lower, or 5 pM or lower). The affinity and binding kinetics of an antibody can be tested using any suitable method including but not limited to biosensor technology (e.g., OCTET or BIACORE). In some embodiments, antibodies described herein binds to KLK5 with a K_{D} of sub-nanomolar range.

Binding affinity (or binding specificity) can be determined by a variety of methods including equilibrium dialysis, equilibrium binding, gel filtration, ELISA, surface plasmon resonance (SPR), florescent activated cell sorting (FACS) or spectroscopy (e.g., using a fluorescence assay). Exemplary conditions for evaluating binding affinity are in HBS-P buffer (10 mM HEPES pH7.4, 150 mM NaCl, 0.005% (v/v) surfactant P20) and PBS buffer (10 mM PO4-3, 137 mM NaCl, and 2.7 mM KCl). These techniques can be used to measure the concentration of bound proteins as a function of target protein concentration. The concentration of bound protein ([[Bound]]) is generally related to the concentration of free target protein ([[Free]]) by the following equation:

[[Bound]]=[[Free]]/(Kd+[[Free]])

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

Exemplary anti-KLK5 antibody sequences (e.g., the heavy chain (HC) and light chain (LC) sequences, heavy chain variable domain (VH) and light chain variable domain (VL), CDR sequences are provided in Tables 1a and 1b.

TABLE la
Examples of anti-KLK5 antibodies

| KLK5 Antibody | | Sequences | SEQ ID NO |
|---|---|---|---|
| KLK5-L1- | HC CDR1 | FTFSSYSMN | 1 |
| Ab1 | HC CDR2 | YISSSSSTIYYADSVKG | 2 |
| | HC CDR3 | ARDLTSMQSRDYYYYGMDV | 9 |
| | LC CDR1 | RASQGISSWLA | 4 |
| | LC CDR2 | AASSLQS | 5 |
| | LC CDR3 | QQVPIFPYT | 6 |
| | VH | EVQLVESGGGLVQPGGSLRLSCAASGFTFSSYSMNWVRQAPGK | 3 |
| | | GLEWVSYISSSSSTIYYADSVKGRFTISRDNAKNSLYLQMNSL | |
| | | RAEDTAVYYCARDLTSMQSRDYYYYGMDVWGQGTTVTVSS | |
| | VL | DIQMTQSPSSVSASVGDRVTITCRASQGISSWLAWYQQKPGKA | 11 |
| | | PKLLIYAASSLQSGVPSRFSGSGSGTDFTLTISSLQPEDFATY | |
| | | YCQQVPIFPYTFGGGTKVEIK | |
| KLK5-L1- | HC CDR1 | FTFGSYAMN | 7 |
| Ab2 | HC CDR2 | YISSSSSFIDYADSVKG | 8 |
| | HC CDR3 | ARDLTSMQSRDYYYYGMDV | 9 |
| | LC CDR1 | RASQGISSWLA | 4 |
| | LC CDR2 | AASSLQS | 5 |
| | LC CDR3 | QQVPIFPYT | 6 |
| | VH | EVQLVESGGGLVQPGGSLRLSCAASGFTFGSYAMNWVRQAPGK | 10 |
| | | GLEWVSYISSSSSFIDYADSVKGRFTISRDNAKNSLYLQMNSL | |
| | | RAEDTAVYYCARDLTSMQSRDYYYYGMDVWGQGTTVTVSS | |
| | VL | DIQMTQSPSSVSASVGDRVTITCRASQGISSWLAWYQQKPGKA | 11 |
| | | PKLLIYAASSLQSGVPSRFSGSGSGTDFTLTISSLQPEDFATY | |
| | | YCQQVPIFPYTFGGGTKVEIK | |
| KLK5-L1- | HC CDR1 | FTFEHYSMN | 12 |
| Ab3 | HC CDR2 | YISSSSSYIDYADSVKG | 13 |
| | HC CDR3 | ARDLTSMQSRDYYYYGMDV | 9 |
| | LC CDR1 | RASQGISSWLA | 4 |
| | LC CDR2 | AASSLQS | 5 |
| | LC CDR3 | QQVPIFPYT | 6 |
| | VH | EVQLVESGGGLVQPGGSLRLSCAASGFTFEHYSMNWVRQAPGK | 14 |
| | | GLEWVSYISSSSSYIDYADSVKGRFTISRDNAKNSLYLQMNSL | |
| | | RAEDTAVYYCARDLTSMQSRDYYYYGMDVWGQGTTVTVSS | |
| | VL | DIQMTQSPSSVSASVGDRVTITCRASQGISSWLAWYQQKPGKA | 11 |
| | | PKLLIYAASSLQSGVPSRFSGSGSGTDFTLTISSLQPEDFATY | |
| | | YCQQVPIFPYTFGGGTKVEIK | |
| KLK5-L1- | HC CDR1 | FTFNDYSMN | 15 |
| Ab4 | HC CDR2 | YISSSSSYIDYADSVKG | 13 |
| | HC CDR3 | ARDLTSMQSRDYYYYGMDV | 9 |
| | LC CDR1 | RASQGISSWLA | 4 |
| | LC CDR2 | AASSLQS | 5 |
| | LC CDR3 | QQVPIFPYT | 6 |
| | VH | EVQLVESGGGLVQPGGSLRLSCAASGFTFNDYSMNWVRQAPGK | 16 |
| | | GLEWVSYISSSSSYIDYADSVKGRFTISRDNAKNSLYLQMNSL | |
| | | RAEDTAVYYCARDLTSMQSRDYYYYGMDVWGQGTTVTVSS | |
| | VL | DIQMTQSPSSVSASVGDRVTITCRASQGISSWLAWYQQKPGKA | 11 |
| | | PKLLIYAASSLQSGVPSRFSGSGSGTDFTLTISSLQPEDFATY | |
| | | YCQQVPIFPYTFGGGTKVEIK | |
| KLK5-L1- | HC CDR1 | FTFEHYSMN | 12 |
| Ab5 | HC CDR2 | YISSSSSYIDYADSVKG | 13 |
| | HC CDR3 | ARDLTDRQSRDYYYYGMDV | 17 |
| | LC CDR1 | RASQGISSWLA | 4 |
| | LC CDR2 | AASSLQS | 5 |
| | LC CDR3 | QQVPIFPYT | 6 |
| | VH | EVQLVESGGGLVQPGGSLRLSCAASGFTFEHYSMNWVRQAPGK | 18 |
| | | GLEWVSYISSSSSYIDYADSVKGRFTISRDNAKNSLYLQMNSL | |
| | | RAEDTAVYYCARDLTDRQSRDYYYYGMDVWGQGTTVTVSS | |
| | VL | DIQMTQSPSSVSASVGDRVTITCRASQGISSWLAWYQQKPGKA | 11 |
| | | PKLLIYAASSLQSGVPSRFSGSGSGTDFTLTISSLQPEDFATY | |
| | | YCQQVPIFPYTFGGGTKVEIK | |
| KLK5-L2- | HC CDR1 | GSISSSSYYWG | 19 |
| Ab1 | HC CDR2 | SISYSGSTYYNPSLKS | 20 |
| | HC CDR3 | ARGEYDRSYYYYGMDV | 21 |
| | LC CDR1 | RASQSVSSSYLA | 22 |
| | LC CDR2 | GASSRAT | 23 |
| | LC CDR3 | QQAFSSPT | 34 |
| | VH | QLQLQESGPGLVKPSETLSLTCTVSGGSISSSSYYWGWIRQPP | 24 |
| | | GKGLEWIGSISYSGSTYYNPSLKSRVTISVDTSKNQFSLKLSS | |
| | | VTAADTAVYYCARGEYDRSYYYYGMDVWGQGTTVTVSS | |
| | VL | EIVLTQSPGTLSLSPGERATLSCRASQSVSSSYLAWYQQKPGQ | 29 |
| | | APRLLIYGASSRATGIPDRFSGSGSGTDFTLTISRLEPEDFAV | |
| | | YYCQQAFSSPTFGGGTKVEIK | |
| KLK5-L2- | HC CDR1 | GSIPSSSYYWA | 25 |
| Ab2 | HC CDR2 | NIYYSGSTYYNPSLKS | 26 |
| | HC CDR3 | ARGEYDRSALYYGMDV | 27 |
| | LC CDR1 | RASQSVSSSYLA | 22 |
| | LC CDR2 | GASSRAT | 23 |
| | LC CDR3 | QQAFSSPT | 34 |
| | VH | QLQLQESGPGLVKPSETLSLTCTVSGGSIPSSSYYWAWIRQPP | 28 |
| | | GKGLEWIGNIYYSGSTYYNPSLKSRVTISVDTSKNQFSLKLSS | |
| | | VTAADTAVYYCARGEYDRSALYYGMDVWGQGTTVTVSS | |
| | VL | EIVLTQSPGTLSLSPGERATLSCRASQSVSSSYLAWYQQKPGQ | 29 |
| | | APRLLIYGASSRATGIPDRFSGSGSGTDFTLTISRLEPEDFAV | |
| | | YYCQQAFSSPTFGGGTKVEIK | |
| KLK5-L2- | HC CDR1 | GSIPSSSYYWS | 30 |
| Ab3 | HC CDR2 | NIYYSGSTYYNPSLKS | 26 |
| | HC CDR3 | ARGEYDRSYYFWGMDV | 31 |
| | LC CDR1 | RASQSVSSSYLA | 22 |
| | LC CDR2 | GASSRAT | 23 |
| | LC CDR3 | QQAFSSPS | 35 |
| | VH | QLQLQESGPGLVKPSETLSLTCTVSGGSIPSSSYYWSWIRQPP | 32 |
| | | GKGLEWIGNIYYSGSTYYNPSLKSRVTISVDTSKNQFSLKLSS | |
| | | VTAADTAVYYCARGEYDRSYYFWGMDVWGQGTTVTVSS | |
| | VL | EIVLTQSPGTLSLSPGERATLSCRASQSVSSSYLAWYQQKPGQ | 37 |
| | | APRLLIYGASSRATGIPDRFSGSGSGTDFTLTISRLEPEDFAV | |
| | | YYCQQAFSSPSFGGGTKVEIK | |
| KLK5-L2- | HC CDR1 | GSIPSSSYYWS | 30 |
| Ab4 | HC CDR2 | NIYYSGSTYYNPSLKS | 26 |
| | HC CDR3 | ARGEYDRSYYYWGLDV | 33 |
| | LC CDR1 | RASQSVSSSYLA | 22 |
| | LC CDR2 | GASSRAT | 23 |
| | LC CDR3 | QQAFSSPT | 34 |
| | VH | QLQLQESGPGLVKPSETLSLTCTVSGGSIPSSSYYWSWIRQPP | 36 |
| | | GKGLEWIGNIYYSGSTYYNPSLKSRVTISVDTSKNQFSLKLSS | |
| | | VTAADTAVYYCARGEYDRSYYYWGLDVWGQGTTVTVSS | |
| | VL | EIVLTQSPGTLSLSPGERATLSCRASQSVSSSYLAWYQQKPGQ | 29 |
| | | APRLLIYGASSRATGIPDRFSGSGSGTDFTLTISRLEPEDFAV | |
| | | YYCQQAFSSPTFGGGTKVEIK | |
| KLK5-L3- | HC CDR1 | YTFTGYYMH | 38 |
| Ab1 | HC CDR2 | GIIPISGTANYAQKFQG | 39 |
| | HC CDR3 | ARGSASGYYDRSYYYYGMDV | 40 |
| | LC CDR1 | RASQGISSWLA | 4 |
| | LC CDR2 | AASSLQS | 5 |
| | LC CDR3 | QQVSSFPYT | 41 |
| | VH | QVQLVQSGAEVKKPGASVKVSCKASGYTFTGYYMHWVRQAPGQ | 42 |
| | | GLEWMGGIIPISGTANYAQKFQGRVTITADESTSTAYMELSSL | |
| | | RSEDTAVYYCARGSASGYYDRSYYYYGMDVWGQGTTVTVSS | |
| | VL | DIQMTQSPSSVSASVGDRVTITCRASQGISSWLAWYQQKPGKA | 43 |
| | | PKLLIYAASSLQSGVPSRFSGSGSGTDFTLTISSLQPEDFATY | |
| | | YCQQVSSFPYTFGGGTKVEIK | |
| KLK5-L3- | HC CDR1 | GTFSGYSIS | 44 |
| Ab2 | HC CDR2 | LIFPISGTANYAQKFQG | 45 |
| | HC CDR3 | ARGSASGYYDRSYYYYGMDV | 40 |
| | LC CDR1 | RASQGISSWLA | 4 |
| | LC CDR2 | AASSLQS | 5 |
| | LC CDR3 | QQVSSFPYT | 41 |
| | VH | QVQLVQSGAEVKKPGSSVKVSCKASGGTFSGYSISWVRQAPGQ | 46 |
| | | GLEWMGLIFPISGTANYAQKFQGRVTITADESTSTAYMELSSL | |
| | | RSEDTAVYYCARGSASGYYDRSYYYYGMDVWGQGTTVTVSS | |
| | VL | DIQMTQSPSSVSASVGDRVTITCRASQGISSWLAWYQQKPGKA | 43 |
| | | PKLLIYAASSLQSGVPSRFSGSGSGTDFTLTISSLQPEDFATY | |
| | | YCQQVSSFPYTFGGGTKVEIK | |
| KLK5-L3- | HC CDR1 | GTFNNYHIS | 47 |
| Ab3 | HC CDR2 | GIIPPFGTAHYAQKFQG | 48 |
| | HC CDR3 | ARGSASGYYDRSYYYYGMDV | 40 |
| | LC CDR1 | RASQGISSWLA | 4 |
| | LC CDR2 | AASSLQS | 5 |
| | LC CDR3 | QQVSSFPYT | 41 |
| | VH | QVQLVQSGAEVKKPGSSVKVSCKASGGTFNNYHISWVRQAPGQ | 49 |
| | | GLEWMGGIIPPFGTAHYAQKFQGRVTITADESTSTAYMELSSL | |
| | | RSEDTAVYYCARGSASGYYDRSYYYYGMDVWGQGTTVTVSS | |
| | VL | DIQMTQSPSSVSASVGDRVTITCRASQGISSWLAWYQQKPGKA | 43 |
| | | PKLLIYAASSLQSGVPSRFSGSGSGTDFTLTISSLQPEDFATY | |
| | | YCQQVSSFPYTFGGGTKVEIK | |
| KLK5-L3- | HC CDR1 | GTENNYHIS | 47 |
| Ab4 | HC CDR2 | GIIPPFGTAHYAQKFQG | 48 |
| | HC CDR3 | ARGEASGYYDRSYYYYGLDV | 50 |
| | LC CDR1 | RASQGISSWLA | 4 |
| | LC CDR2 | AASSLQS | 5 |
| | LC CDR3 | QQVSSFPYT | 41 |
| | VH | QVQLVQSGAEVKKPGSSVKVSCKASGGTFNNYHISWVRQAPGQ | 51 |
| | | GLEWMGGIIPPFGTAHYAQKFQGRVTITADESTSTAYMELSSL | |
| | | RSEDTAVYYCARGEASGYYDRSYYYYGLDVWGQGTTVTVSS | |
| | VL | DIQMTQSPSSVSASVGDRVTITCRASQGISSWLAWYQQKPGKA | 43 |
| | | PKLLIYAASSLQSGVPSRFSGSGSGTDFTLTISSLQPEDFATY | |
| | | YCQQVSSFPYTFGGGTKVEIK | |
| KLK5-L3- | HC CDR1 | GTFSGYSIS | 44 |
| Ab5 | HC CDR2 | LIFPISGTANYAQKFQG | 45 |
| | HC CDR3 | ARQSASGYYDRSYYRYGMDV | 52 |
| | LC CDR1 | RASQGISSWLA | 4 |
| | LC CDR2 | AASSLQS | 5 |
| | LC CDR3 | QQVSSFPYT | 41 |
| | VH | QVQLVQSGAEVKKPGSSVKVSCKASGGTFSGYSISWVRQAPGQ | 53 |
| | | GLEWMGLIFPISGTANYAQKFQGRVTITADESTSTAYMELSSL | |
| | | RSEDTAVYYCARQSASGYYDRSYYRYGMDVWGQGTTVTVSS | |
| | VL | DIQMTQSPSSVSASVGDRVTITCRASQGISSWLAWYQQKPGKA | 43 |
| | | PKLLIYAASSLQSGVPSRFSGSGSGTDFTLTISSLQPEDFATY | |
| | | YCQQVSSFPYTFGGGTKVEIK | |
| KLK5-L3- | HC CDR1 | GTFSGYVIS | 54 |
| Ab6 | HC CDR2 | GIFPISGYAVYAQKFQG | 55 |
| | HC CDR3 | ARASASGYYDRSYYYYGMDV | 56 |
| | LC CDR1 | RASQGISSWLA | 4 |
| | LC CDR2 | AASSLQS | 5 |
| | LC CDR3 | QQVSSFPQT | 57 |
| | VH | QVQLVQSGAEVKKPGSSVKVSCKASGGTFSGYVISWVRQAPGQ | 58 |
| | | GLEWMGGIFPISGYAVYAQKFQGRVTITADESTSTAYMELSSL | |
| | | RSEDTAVYYCARASASGYYDRSYYYYGMDVWGQGTTVTVSS | |
| | VL | DIQMTQSPSSVSASVGDRVTITCRASQGISSWLAWYQQKPGKA | 59 |
| | | PKLLIYAASSLQSGVPSRFSGSGSGTDFTLTISSLQPEDFATY | |
| | | YCQQVSSFPQTFGGGTKVEIK | |
| KLK5-L3- | HC CDR1 | GIFSNYHIS | 60 |
| Ab7 | HC CDR2 | GIFPTFGTANYAQKFQG | 61 |
| | HC CDR3 | ARGSASGYYDRSYYYDGMDV | 62 |
| | LC CDR1 | RASQGISSWLA | 4 |
| | LC CDR2 | AASSLQS | 5 |
| | LC CDR3 | QQVISFPYT | 63 |
| | VH | QVQLVQSGAEVRKPGSSVKVSCKASGGTFSNYHISWVRQAPGQ | 64 |
| | | GLEWMGGIFPTFGTANYAQKFQGRVTITADESTSTAYMELSSL | |
| | | RSEDTAVYYCARGSASGYYDRSYYYDGMDVWGQGTTVTVSS | |
| | VL | DIQMTQSPSSVSASVGDRVTITCRASQGISSWLAWYQQKPGKA | 65 |
| | | PKLLIYAASSLQSGVPSRFSGSGSGTDFTLTISSLQPEDFATY | |
| | | YCQQVISFPYTFGGGTKVEIK | |

In some embodiments, certain amino acid positions in an antibody described herein (e.g., amino acids in the VH/VL regions and/or CDR regions) are substitutable and the substitution results in an antibody with substantially similar binding and biological activities (e.g., substantially similar binding affinity, binding specificity, protease activity inhibitory activity, anti-inflammatory activity, or a combination thereof) as the reference antibody. In order to identify a substitutable position of an antibody, the amino acid sequence of that antibody is compared to the sequences of other antibodies belonging to the same group as that antibody. If the identity of that amino acid varies between the different related antibodies of a group at any particular position, that position is a substitutable position of the antibody. In other words, a substitutable position is a position in which the identity of the amino acid varies between the related antibodies. Positions that contain a constant amino acid are not substitutable positions.

In some embodiments, the above method may be employed to provide a consensus antibody sequence. In such a consensus sequence, a non-substitutable position is indicated by the amino acid present at that position, and a substitutable position is indicated as an “X”.

Depending on how the antibodies are to be employed, X may be a) any amino acid, b) any amino acid present at that position in any of the related antibodies in the group or a conservatively substituted variant thereof or c) any amino acid present at that position in any of the related antibodies in the group. Any antibody having a sequence that is encompassed by the consensus should bind to the same antigen as any of the related antibodies.

In some embodiments, the method described above may be employed in methods of designing and making a variant of a parental antibody that at least maintains (e.g., maintains or increases) the antigen binding activity of the parental antibody. Because antibodies containing substitutions at substitutable positions have already been produced and tested, substitutions at those positions can be made in the knowledge that they should not significantly decrease the binding activity of the antibody. In general, an antibody variant of a parental antibody has an antigen binding affinity that is at least 10%, at least 20%, at least 30%, at least 40%, at least 50%, at least 60%, at least 70%, at least 80%, at least 90% or at least 100% (e.g., at least 150%, at least 200%, at least 500%, at least 1000%, usually up to at least 10,000%) of the binding affinity of the parental antibody to a particular antigen.

In some embodiments, a substitutable position of a parental antibody may be substituted by a) any of the 20 naturally occurring amino acids to produce random substitutions, b) an amino acid having biochemical properties similar to the amino acid already present at the substitutable position to produce conservative substitutions, c) an amino acid that is present at the same position in a related antibody to produce a directed substitution, or d) an amino acid that is present at the same position in a similar human antibody to produce a humanizing substitution. A substitution may be made at any part of an antibody variable region, including any framework region or CDR. In certain embodiments, a single substitutable amino acid may be substituted. However, in other embodiments, a plurality of substitutable amino acids (e.g., up to about 5 or 10 or more) may be substituted. In particular embodiments, the type of substitution that can be made at each substitutable position may be indicated by the types of amino acids present at that position in the related antibodies. For example, if unrelated amino acids (e.g., Ala, Gly, Cys, Glu and Thr) are present at a certain position of a group of related antibodies, then any amino acid could be substituted at that position without significantly reducing binding activity of the antibody. Exemplary amino acids substitutions of an anti-KLK5 antibody described herein are set forth in Table 1b:

TABLE 1b
Exemplary Anti-KLK5 Antibody Amino Acid Substitutions

| KLK5 Antibody | | Sequences | SEQ ID NO |
|---|---|---|---|
| KLK5-L1- | HC CDR1 | FTFX₁X₂YX₃MN | 253 |
| Ab0 | | X₁ is S, G, E, or N | |
| | | X₂ is S, H, or D | |
| | | X₃ is S or A | |
| | HC CDR2 | YISSSSSX₄IX₅YADSVKG | 254 |
| | | X₄ is T, F, or Y | |
| | | X₅ is Y or D | |
| | HC CDR3 | ARDLTX₆X₇QSESYYYYGMDV | 255 |
| | | X₆ is S or D | |
| | | X₇ is M or R | |
| | LC CDR1 | RASQGISSWLA | 4 |
| | LC CDR2 | AASSLQS | 5 |
| | LC CDR3 | QQVPIFPYT | 6 |
| KLK5-L2- | HC CDR1 | GSIX₁SSSYYWX₂ | 256 |
| Ab0 | | X₁ is S or P | |
| | | X₂ is G, A, or S | |
| | HC CDR2 | X₃IX₄YSGSTYYNPSLKS | 257 |
| | | X₃ is S or N | |
| | | X₄ is S or Y | |
| | HC CDR3 | ARGEYDRSX₅X₆X₇X₈GX₉DV | 258 |
| | | X₅ is Y or A | |
| | | X₆ is Y or L | |
| | | X₇ is Y or F | |
| | | X₈ is Y or W | |
| | | X₉ is M or L | |
| | LC CDR1 | RASQSVSSSYLA | 22 |
| | LC CDR2 | GASSRAT | 23 |
| | LC CDR3 | QQAFSSPX₉ | 259 |
| | | X₉ is T or S | |
| KLK5-L3- | HC CDR1 | X₁TFX₂X₃YX₄X₅X₆ | 260 |
| Ab0 | | X₁ is Y or G | |
| | | X₂ is T, S, or N | |
| | | X₃ is G or N | |
| | | X₄ is Y, S, H, or V | |
| | | X₅ is M or I | |
| | | X₆ is H or S | |
| | HC CDR2 | X₇IX₈PX₉X₁₀GX₁₁AX₁₂YAQKFQG | 261 |
| | | X₇ is G or L | |
| | | X₈ is I or F | |
| | | X₉ is I, P or T | |
| | | X₁₀ is S or F | |
| | | X₁₁ is T or Y | |
| | | X₁₂ is N, H or V | |
| | HC CDR3 | ARX₁₃X₁₄ASGYYDRSYYX₁₅X₁₆GX₁₇DV | 262 |
| | | X₁₃ is G, Q or A | |
| | | X₁₄ is S, or E | |
| | | X₁₅ is Y or R | |
| | | X₁₆ is Y or D | |
| | | X₁₇ is M, or L | |
| | LC CDR1 | RASQGISSWLA | 4 |
| | LC CDR2 | AASSLQS | 5 |
| | LC CDR3 | QQVX₁₈SFPX₁₉T | 263 |
| | | X₁₈ is S or I | |
| | | X₁₉ is Y or Q | |

In some embodiments, an antibody of the present disclosure comprises a HC CDR1 comprising the amino acid sequence of FTFX₁X₂YX₃MN, in which X₁ is S, G, E, or N, X₂ is S, H, or D, or X₃ is S or A; a HC CDR2 comprising the amino acid sequence of YISSSSSX₄IX₅YADSVKG, in which X₄ is T, F, or Y, X₅ is Y or D; a HC CDR3 comprising the amino acid sequence of ARDLTX₆X₇QSESYYYYGMDV, in which X₆ is S or D, or X₇ is M or R; a LC CDR1 comprising the amino acid sequence of SEQ ID NO: 4; a LC CDR2 comprising the amino acid sequence of SEQ ID NO: 5; and/or a LC CDR3 comprising the amino acid sequence of SEQ ID NO: 6.

In some embodiments, an antibody of the present disclosure comprises a HC CDR1 comprising the amino acid sequence of GSIX₁SSSYYWX₂, in which X₁ is S or P, or X₂ is G, A, or S; a HC CDR2 comprising the amino acid sequence of X₃IX₄YSGSTYYNPSLKS, X₃ is S or N, or X₄ is S or Y; a HC CDR3 comprising the amino acid sequence of ARGEYDRSX₅X₆X₇X₈GX₉DV, in which X₅ is Y or A, X₆ is Y or L, X₇ is Y or F, X₈ is Y or W, or X₉ is M or L; a LC CDR1 comprising the amino acid sequence of SEQ ID NO: 22; a LC CDR2 comprising the amino acid sequence of SEQ ID NO: 23; and/or a LC CDR3 comprising the amino acid sequence of QQAFSSPX₉, in which X₉ is T or S.

In some embodiments, an antibody of the present disclosure comprises a HC CDR1 comprising the amino acid sequence of X₁TFX₂X₃YX₄X₅X₆, in which X₁ is Y or G, X₂ is T, S, or N, X₃ is G or N, X₄ is Y, S, H, or V, X₅ is M or I, or X₆ is H or S; a HC CDR2 comprising the amino acid sequence of X₇IX₈PX₉X₁₀GX₁₁AX₁₂YAQKFQG, in which X₇ is G or L, X₈ is I or F, X₉ is I, P or T, X₁₀ is S or F, X₁₁ is T or Y, or X₁₂ is N, H or V; a HC CDR3 comprising the amino acid sequence of ARX₁₃X₁₄ASGYYDRSYYX₁₅X₁₆GX₁₇DV, in which X₁₃ is G, Q or A, X₁₄ is S or E, X₁₅ is Y or R, X₁₆ is Y or D, or X₁₇ is M or L; a LC CDR1 comprising the amino acid sequence of SEQ ID NO: 4; a LC CDR2 comprising the amino acid sequence of SEQ ID NO: 5; and/or a LC CDR3 comprising the amino acid sequence of QQVX₁₈SFPX₁₉T, in which X₁₈ is S or I, or X₁₉ is Y or Q.

In some embodiments, an antibody of the present disclosure comprises one or more of the HC CDRs (e.g., HC CDR1, HC CDR2, or HC CDR3) amino acid sequences from any one of the anti-KLK5 antibodies selected from Tables 1a and 1b. In some embodiments, an antibody of the present disclosure comprises the HC CDR3 amino acid sequences from any one of the anti-KLK5 antibodies selected from Tables 1a and 1b. In some embodiments, an antibody of the present disclosure comprise the HC CDR1, HC CDR2, and HC CDR3 as provided for any one of the antibodies elected from Tables 1a and 1b. In some embodiments, an antibody of the present disclosure comprises the LC CDR3 amino acid sequences from any one of the anti-KLK5 antibodies selected from Tables 1a and 1b. In some embodiments, an antibody of the present disclosure comprises one or more of the LC CDRs (e.g., LC CDR1, LC CDR2, or LC CDR3) amino acid sequences from any one of the anti-KLK5 antibodies selected from Tables 1a and 1b. In some embodiments, an antibody of the present disclosure comprise the LC CDR1, LC CDR2, and LC CDR3 s provided for any one of the anti-KLK5 antibodies selected from Tables 1a and 1b.

In some embodiments, an antibody of the present disclosure comprises the HC CDR1, HC CDR2, HC CDR3, LC CDR1, LC CDR2, and LC CDR3 as provided for any one of the anti-KLK5 antibodies selected from Tables 1a and 1b. In some embodiments, antibody heavy and/or light chain CDR3 domains may play a particularly important role in the binding specificity/affinity of an antibody for an antigen. Accordingly, an antibody of the disclosure may include at least the heavy and/or light chain CDR3s of any one of the anti-KLK5 antibodies selected from Tables 1a and 1b.

Also within the scope of the present disclosure are variants of any of the exemplary anti-KLK5 antibodies as disclosed herein. A variant may contain one or more amino acid residue variations in the VH and/or VL, or in one or more of the HC CDRs and/or one or more of the LC CDRs as relative to the reference antibody, while retaining substantially similar binding and biological activities (e.g., substantially similar binding affinity, binding specificity, protease activity inhibitory activity, anti-inflammatory activity, or a combination thereof) as the reference antibody.

In some embodiments, an antibody of the disclosure have one or more CDRs (e.g., HC CDR or LC CDR) sequences substantially similar to any of the HC CDR1, HC CDR2, HC CDR3, LC CDR1, LC CDR2, and/or LC CDR3 sequences from one of the anti-KLK5 antibodies selected from Tables 1a and 1b. In some embodiments, the position of one or more CDRs along the VH (e.g., HC CDR1, HC CDR2, or HC CDR3) and/or VL (e.g., LC CDR1, LC CDR2, or LC CDR3) region of an antibody described herein can vary by one, two, three, four, five, or six amino acid positions so long as specific binding to KLK5 (e.g., human or mouse KLK5) is maintained (e.g., substantially maintained, for example, at least 50%, at least 60%, at least 70%, at least 80%, at least 90%, at least 95% of the binding of the original antibody from which it is derived). For example, in some embodiments, the position defining a CDR of any antibody described herein can vary by shifting the N-terminal and/or C-terminal boundary of the CDR by one, two, three, four, five, or six amino acids, relative to the CDR position of any one of the antibodies described herein, so long as specific binding to KLK5 (e.g., human or mouse KLK5) is maintained (e.g., substantially maintained, for example, at least 50%, at least 60%, at least 70%, at least 80%, at least 90%, at least 95% of the binding of the original antibody from which it is derived). In another embodiment, the length of one or more CDRs along the VH (e.g., HC CDR1, HC CDR2, or HC CDR3) and/or VL (e.g., LC CDR1, LC CDR2, or LC CDR3) region of an antibody described herein can vary (e.g., be shorter or longer) by one, two, three, four, five, or more amino acids, so long as immunospecific binding to KLK5 (e.g., human or mouse KLK5) is maintained (e.g., substantially maintained, for example, at least 50%, at least 60%, at least 70%, at least 80%, at least 90%, at least 95% of the binding of the original antibody from which it is derived).

Accordingly, in some embodiments, a HC CDR1, HC CDR2, HC CDR3, LC CDR1, LC CDR2, and/or LC CDR3 described herein may be one, two, three, four, five or more amino acids shorter than one or more of the CDRs described herein (e.g., CDRS from any of the anti-KLK5 antibodies selected from Tables 1a and 1b) so long as specific binding to KLK5 (e.g., human or mouse KLK5) is maintained (e.g., substantially maintained, for example, at least 50%, at least 60%, at least 70%, at least 80%, at least 90%, at least 95% relative to the binding of the original antibody from which it is derived). In some embodiments, a HC CDR1, HC CDR2, HC CDR3, LC CDR1, LC CDR2, and/or LC CDR3 described herein may be one, two, three, four, five or more amino acids longer than one or more of the CDRs described herein (e.g., CDRS from any of the anti-KLK7 antibodies selected from Tables 1a and 1b) so long as specific binding to KLK5 (e.g., human or mouse KLK5) is maintained (e.g., substantially maintained, for example, at least 50%, at least 60%, at least 70%, at least 80%, at least 90%, at least 95% relative to the binding of the original antibody from which it is derived). In some embodiments, the amino portion of a HC CDR1, HC CDR2, HC CDR3, LC CDR1, LC CDR2, and/or LC CDR3 described herein can be extended by one, two, three, four, five or more amino acids compared to one or more of the CDRs described herein (e.g., CDRS from any of the anti-KLK5 antibodies selected from Tables 1a and 1b) so long as specific binding to KLK5 (e.g., human or mouse KLK5) is maintained (e.g., substantially maintained, for example, at least 50%, at least 60%, at least 70%, at least 80%, at least 90%, at least 95% relative to the binding of the original antibody from which it is derived). In some embodiments, the carboxy portion of a HC CDR1, HC CDR2, HC CDR3, LC CDR1, LC CDR2, and/or LC CDR3 described herein can be extended by one, two, three, four, five or more amino acids compared to one or more of the CDRs described herein (e.g., CDRS from any of the anti-KLK5 antibodies selected from Tables 1a and 1b) so long as specific binding to KLK5 (e.g., human or mouse KLK5) is maintained (e.g., substantially maintained, for example, at least 50%, at least 60%, at least 70%, at least 80%, at least 90%, at least 95% relative to the binding of the original antibody from which it is derived). In some embodiments, the amino portion of a HC CDR1, HC CDR2, HC CDR3, LC CDR1, LC CDR2, and/or LC CDR3 described herein can be shortened by one, two, three, four, five or more amino acids compared to one or more of the CDRs described herein (e.g., CDRS from any of the anti-KLK5 antibodies selected from Tables 1a and 1b) so long as specific binding to KLK5 (e.g., human or mouse KLK5) is maintained (e.g., substantially maintained, for example, at least 50%, at least 60%, at least 70%, at least 80%, at least 90%, at least 95% relative to the binding of the original antibody from which it is derived). In some embodiments, the carboxy portion of a HC CDR1, HC CDR2, HC CDR3, LC CDR1, LC CDR2, and/or LC CDR3 described herein can be shortened by one, two, three, four, five or more amino acids compared to one or more of the CDRs described herein (e.g., CDRS from any of the anti-KLK5 antibodies selected from Tables 1a and 1b) so long as specific binding to KLK5 (e.g., human or mouse KLK5) is maintained (e.g., substantially maintained, for example, at least 50%, at least 60%, at least 70%, at least 80%, at least 90%, at least 95% relative to the binding of the original antibody from which it is derived). Any method can be used to ascertain whether immunospecific binding to KLK5 (e.g., human or mouse KLK5) is maintained, for example, using binding assays and conditions described in the art.

In some examples, an antibody of the disclosure have one or more CDR (e.g., HC CDR or LC CDR) sequences substantially similar to any one of the anti-KLK5 antibodies selected from Tables 1a and 1b. For example, an antibody described herein may include one or more CDR sequence(s) from any of the anti-KLK5 antibodies selected from Tables 1a and 1b containing up to 5, 4, 3, 2, or 1 amino acid residue variations as compared to the corresponding CDR region in any one of the CDRs provided herein (e.g., CDRs from any of the anti-KLK5 antibodies selected from Tables 1a and 1b) so long as specific binding to KLK5 (e.g., human or mouse KLK5) is maintained (e.g., substantially maintained, for example, at least 50%, at least 60%, at least 70%, at least 80%, at least 90%, at least 95% relative to the binding of the original antibody from which it is derived). In some embodiments, any of the amino acid variations in any of the CDRs provided herein may be conservative variations. Conservative variations can be introduced into the CDRs at positions where the residues are not likely to be involved in interacting with a KLK5 (e.g., human or mouse KLK5), for example, as determined based on a crystal structure. Some aspects of the disclosure provide antibodies that comprise one or more of the heavy chain variable (VH) and/or light chain variable (VL) domains provided herein. In some embodiments, any of the VH domains provided herein include one or more of the HC CDR sequences (e.g., HC CDR1, HC CDR2, and HC CDR3) provided herein, for example, any of the HC CDR sequences provided in any one of the anti-KLK5 selected from Tables 1a and 1b. In some embodiments, any of the VL domains provided herein include one or more of the LC CDR sequences (e.g., LC CDR1, LC CDR2, and LC CDR3) provided herein, for example, any of the LC CDR sequences provided in any one of the anti-KLK5 antibodies selected from Tables 1a and 1b.

In some embodiments, an antibody of the disclosure include any antibody that includes a heavy chain variable domain and/or a light chain variable domain of any one of the anti-KLK5 antibodies selected from Tables 1a and 1b, and variants thereof. In some embodiments, an antibody of the disclosure include any antibody that includes the heavy chain variable and light chain variable pairs of any anti-KLK5 antibodies selected from Tables 1a and 1b.

Aspects of the disclosure provide antibodies having a heavy chain variable (VH) and/or a light chain variable (VL) domain amino acid sequence homologous to any of those described herein. In some embodiments, an antibody comprises a heavy chain variable sequence or a light chain variable sequence that is at least 75% (e.g., 80%, 85%, 90%, 95%, 98%, or 99%) identical to the heavy chain variable sequence and/or any light chain variable sequence of any one of the anti-KLK5 antibodies selected from Tables 1a and 1b. In some embodiments, the homologous heavy chain variable and/or a light chain variable amino acid sequences do not vary within any of the CDR sequences provided herein. For example, in some embodiments, the degree of sequence variation (e.g., 75%, 80%, 85%, 90%, 95%, 98%, or 99%) may occur within a heavy chain variable and/or a light chain variable sequence excluding any of the CDR sequences provided herein. In some embodiments, an antibody provided herein comprise a heavy chain variable sequence and a light chain variable sequence that comprises a framework sequence that is at least 75%, 80%, 85%, 90%, 95%, 98%, or 99% identical to the framework sequence of any anti-KLK5 antibodies selected from Tables 1a and 1b.

In some embodiments, an antibody of the present disclosure is a humanized antibody (e.g., a humanized variant containing one or more CDRs of Tables 1a and 1b). In some embodiments, an antibody of the present disclosure comprises a HC CDR1, a HC CDR2, a HC CDR3, a LC CDR1, a LC CDR2, and a LC CDR3 that are the same as the HC CDR1, HC CDR2, HC CDR3, LC CDR1, LC CDR2, and LC CDR3 shown in Tables 1a and 1b, and comprises a humanized heavy chain variable region and/or a humanized light chain variable region.

In some embodiments, an antibody of the present disclosure is a humanized antibody comprising a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH of any of the anti-KLK5 antibodies listed in Tables 1a and 1b. Alternatively or in addition, the antibody of the present disclosure is a humanized antibody comprising a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL of any one of the anti-KLK5 antibodies listed in Tables 1a and 1b.

In some embodiments, an anti-KLK5 antibody of the present disclosure comprises a HC CDR1, HC CDR2 and HC CDR3 of a heavy chain variable domain having the amino acid sequence of SEQ ID NO: 3. Alternatively or in addition, the anti-KLK5 antibody of the present disclosure comprises a LC CDR1, LC CDR2 and LC CDR3 of a light chain variable domain having the amino acid sequence of SEQ ID NO: 11.

In some embodiments, an antibody of the present disclosure comprises a HC CDR1 having the amino acid sequence of SEQ ID NO: 1, a HC CDR2 having the amino acid sequence of SEQ ID NO: 2, a HC CDR3 having the amino acid sequence of SEQ ID NO: 9, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 6.

In some embodiments, an anti-KLK5 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 1, HC CDR2 having the amino acid sequence of SEQ ID NO: 2, and HC CDR3 having the amino acid sequence of SEQ ID NO: 9. “Collectively,” as used anywhere in the present disclosure, means that the total number of amino acid variations in all of the three heavy chain CDRs is within the defined range. Alternatively or in addition, the anti-KLK5 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 4, LC CDR2 having the amino acid sequence of SEQ ID NO: 5, and LC CDR3 having the amino acid sequence of SEQ ID NO: 6.

In some embodiments, an anti-KLK5 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 1, HC CDR2 having the amino acid sequence of SEQ ID NO: 2, and HC CDR3 having the amino acid sequence of SEQ ID NO: 9. Alternatively or in addition, the anti-KLK5 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 4, LC CDR2 having the amino acid sequence of SEQ ID NO: 5, and LC CDR3 having the amino acid sequence of SEQ ID NO: 6.

In some embodiments, an anti-KLK5 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 1; a HC CDR2 having no more than 3 amino acid variations (e.g., no more than 3, 2, or 1 amino acid variation) as compared with the HC CDR2 having the amino acid sequence of SEQ ID NO: 2; and/or a HC CDR3 having no more than 3 amino acid variations (e.g., no more than 3, 2, or 1 amino acid variation) as compared with the HC CDR3 having the amino acid sequence of SEQ ID NO: 9. Alternatively or in addition, the anti-KLK5 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of SEQ ID NO: 5; and/or a LC CDR3 having no more than 3 amino acid variations (e.g., no more than 3, 2, or 1 amino acid variation) as compared with the LC CDR3 having the amino acid sequence of SEQ ID NO: 6.

In some embodiments, an anti-KLK5 antibody of the present disclosure comprises a VH comprising the amino acid sequence of SEQ ID NO: 3. Alternatively or in addition, the anti-KLK5 antibody of the present disclosure comprises a VL comprising the amino acid sequence of SEQ ID NO: 11.

In some embodiments, an anti-KLK5 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 3. Alternatively or in addition, the anti-KLK5 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 11.

In some embodiments, an anti-KLK5 antibody of the present disclosure comprises a VH comprising an amino acid sequence that is at least 80% (e.g., 80%, 85%, 90%, 95%, 98%, or 99%) identical to the VH as set forth in SEQ ID NO: 3. Alternatively or in addition, the anti-KLK5 antibody of the present disclosure comprises a VL comprising an amino acid sequence that is at least 80% (e.g., 80%, 85%, 90%, 95%, 98%, or 99%) identical to the VL as set forth in SEQ ID NO: 11.

In some embodiments, an anti-KLK5 antibody of the present disclosure comprises a HC CDR1, HC CDR2 and HC CDR3 of a heavy chain variable domain having the amino acid sequence of SEQ ID NO: 10. Alternatively or in addition, the anti-KLK5 antibody of the present disclosure comprises a LC CDR1, LC CDR2 and LC CDR3 of a light chain variable domain having the amino acid sequence of SEQ ID NO: 11.

In some embodiments, an antibody of the present disclosure comprises a HC CDR1 having the amino acid sequence of SEQ ID NO: 7, a HC CDR2 having the amino acid sequence of SEQ ID NO: 8, a HC CDR3 having the amino acid sequence of SEQ ID NO: 9, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 6.

In some embodiments, an anti-KLK5 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 7, HC CDR2 having the amino acid sequence of SEQ ID NO: 8, and HC CDR3 having the amino acid sequence of SEQ ID NO: 9. “Collectively,” as used anywhere in the present disclosure, means that the total number of amino acid variations in all of the three heavy chain CDRs is within the defined range. Alternatively or in addition, the anti-KLK5 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 4, LC CDR2 having the amino acid sequence of SEQ ID NO: 5, and LC CDR3 having the amino acid sequence of SEQ ID NO: 6.

In some embodiments, an anti-KLK5 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 7, HC CDR2 having the amino acid sequence of SEQ ID NO: 8, and HC CDR3 having the amino acid sequence of SEQ ID NO: 9. Alternatively or in addition, the anti-KLK5 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 4, LC CDR2 having the amino acid sequence of SEQ ID NO: 5, and LC CDR3 having the amino acid sequence of SEQ ID NO: 6.

In some embodiments, an anti-KLK5 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 7; a HC CDR2 having no more than 3 amino acid variations (e.g., no more than 3, 2, or 1 amino acid variation) as compared with the HC CDR2 having the amino acid sequence of SEQ ID NO: 8; and/or a HC CDR3 having no more than 3 amino acid variations (e.g., no more than 3, 2, or 1 amino acid variation) as compared with the HC CDR3 having the amino acid sequence of SEQ ID NO: 9. Alternatively or in addition, the anti-KLK5 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of SEQ ID NO: 5; and/or a LC CDR3 having no more than 3 amino acid variations (e.g., no more than 3, 2, or 1 amino acid variation) as compared with the LC CDR3 having the amino acid sequence of SEQ ID NO: 6.

In some embodiments, an anti-KLK5 antibody of the present disclosure comprises a VH comprising the amino acid sequence of SEQ ID NO: 10. Alternatively or in addition, the anti-KLK5 antibody of the present disclosure comprises a VL comprising the amino acid sequence of SEQ ID NO: 11.

In some embodiments, an anti-KLK5 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 10. Alternatively or in addition, the anti-KLK5 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 11.

In some embodiments, an anti-KLK5 antibody of the present disclosure comprises a VH comprising an amino acid sequence that is at least 80% (e.g., 80%, 85%, 90%, 95%, 98%, or 99%) identical to the VH as set forth in SEQ ID NO: 10. Alternatively or in addition, the anti-KLK5 antibody of the present disclosure comprises a VL comprising an amino acid sequence that is at least 80% (e.g., 80%, 85%, 90%, 95%, 98%, or 99%) identical to the VL as set forth in SEQ ID NO: 11.

In some embodiments, an anti-KLK5 antibody of the present disclosure comprises a HC CDR1, HC CDR2 and HC CDR3 of a heavy chain variable domain having the amino acid sequence of SEQ ID NO: 14. Alternatively or in addition, the anti-KLK5 antibody of the present disclosure comprises a LC CDR1, LC CDR2 and LC CDR3 of a light chain variable domain having the amino acid sequence of SEQ ID NO: 11.

In some embodiments, an antibody of the present disclosure comprises a HC CDR1 having the amino acid sequence of SEQ ID NO: 12, a HC CDR2 having the amino acid sequence of SEQ ID NO: 13, a HC CDR3 having the amino acid sequence of SEQ ID NO: 9, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 6.

In some embodiments, an anti-KLK5 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 12, HC CDR2 having the amino acid sequence of SEQ ID NO: 13, and HC CDR3 having the amino acid sequence of SEQ ID NO: 9. “Collectively,” as used anywhere in the present disclosure, means that the total number of amino acid variations in all of the three heavy chain CDRs is within the defined range. Alternatively or in addition, the anti-KLK5 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 4, LC CDR2 having the amino acid sequence of SEQ ID NO: 5, and LC CDR3 having the amino acid sequence of SEQ ID NO: 6.

In some embodiments, an anti-KLK5 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 12, HC CDR2 having the amino acid sequence of SEQ ID NO: 13, and HC CDR3 having the amino acid sequence of SEQ ID NO: 9. Alternatively or in addition, the anti-KLK5 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 4, LC CDR2 having the amino acid sequence of SEQ ID NO: 5, and LC CDR3 having the amino acid sequence of SEQ ID NO: 6.

In some embodiments, an anti-KLK5 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 12; a HC CDR2 having no more than 3 amino acid variations (e.g., no more than 3, 2, or 1 amino acid variation) as compared with the HC CDR2 having the amino acid sequence of SEQ ID NO: 13; and/or a HC CDR3 having no more than 3 amino acid variations (e.g., no more than 3, 2, or 1 amino acid variation) as compared with the HC CDR3 having the amino acid sequence of SEQ ID NO: 9. Alternatively or in addition, the anti-KLK5 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of SEQ ID NO: 5; and/or a LC CDR3 having no more than 3 amino acid variations (e.g., no more than 3, 2, or 1 amino acid variation) as compared with the LC CDR3 having the amino acid sequence of SEQ ID NO: 6.

In some embodiments, an anti-KLK5 antibody of the present disclosure comprises a VH comprising the amino acid sequence of SEQ ID NO: 14. Alternatively or in addition, the anti-KLK5 antibody of the present disclosure comprises a VL comprising the amino acid sequence of SEQ ID NO: 11.

In some embodiments, an anti-KLK5 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 14. Alternatively or in addition, the anti-KLK5 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 11.

In some embodiments, an anti-KLK5 antibody of the present disclosure comprises a VH comprising an amino acid sequence that is at least 80% (e.g., 80%, 85%, 90%, 95%, 98%, or 99%) identical to the VH as set forth in SEQ ID NO: 14. Alternatively or in addition, the anti-KLK5 antibody of the present disclosure comprises a VL comprising an amino acid sequence that is at least 80% (e.g., 80%, 85%, 90%, 95%, 98%, or 99%) identical to the VL as set forth in SEQ ID NO: 11.

In some embodiments, an anti-KLK5 antibody of the present disclosure comprises a HC CDR1, HC CDR2 and HC CDR3 of a heavy chain variable domain having the amino acid sequence of SEQ ID NO: 16. Alternatively or in addition, the anti-KLK5 antibody of the present disclosure comprises a LC CDR1, LC CDR2 and LC CDR3 of a light chain variable domain having the amino acid sequence of SEQ ID NO: 11.

In some embodiments, an antibody of the present disclosure comprises a HC CDR1 having the amino acid sequence of SEQ ID NO: 15, a HC CDR2 having the amino acid sequence of SEQ ID NO: 13, a HC CDR3 having the amino acid sequence of SEQ ID NO: 9, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 6.

In some embodiments, an anti-KLK5 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 15, HC CDR2 having the amino acid sequence of SEQ ID NO: 13, and HC CDR3 having the amino acid sequence of SEQ ID NO: 9. “Collectively,” as used anywhere in the present disclosure, means that the total number of amino acid variations in all of the three heavy chain CDRs is within the defined range. Alternatively or in addition, the anti-KLK5 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 4, LC CDR2 having the amino acid sequence of SEQ ID NO: 5, and LC CDR3 having the amino acid sequence of SEQ ID NO: 6.

In some embodiments, an anti-KLK5 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 15, HC CDR2 having the amino acid sequence of SEQ ID NO: 13, and HC CDR3 having the amino acid sequence of SEQ ID NO: 9. Alternatively or in addition, the anti-KLK5 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 4, LC CDR2 having the amino acid sequence of SEQ ID NO: 5, and LC CDR3 having the amino acid sequence of SEQ ID NO: 6.

In some embodiments, an anti-KLK5 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 15; a HC CDR2 having no more than 3 amino acid variations (e.g., no more than 3, 2, or 1 amino acid variation) as compared with the HC CDR2 having the amino acid sequence of SEQ ID NO: 13; and/or a HC CDR3 having no more than 3 amino acid variations (e.g., no more than 3, 2, or 1 amino acid variation) as compared with the HC CDR3 having the amino acid sequence of SEQ ID NO: 9. Alternatively or in addition, the anti-KLK5 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of SEQ ID NO: 5; and/or a LC CDR3 having no more than 3 amino acid variations (e.g., no more than 3, 2, or 1 amino acid variation) as compared with the LC CDR3 having the amino acid sequence of SEQ ID NO: 6.

In some embodiments, an anti-KLK5 antibody of the present disclosure comprises a VH comprising the amino acid sequence of SEQ ID NO: 16. Alternatively or in addition, the anti-KLK5 antibody of the present disclosure comprises a VL comprising the amino acid sequence of SEQ ID NO: 11.

In some embodiments, an anti-KLK5 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 16. Alternatively or in addition, the anti-KLK5 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 11.

In some embodiments, an anti-KLK5 antibody of the present disclosure comprises a VH comprising an amino acid sequence that is at least 80% (e.g., 80%, 85%, 90%, 95%, 98%, or 99%) identical to the VH as set forth in SEQ ID NO: 16. Alternatively or in addition, the anti-KLK5 antibody of the present disclosure comprises a VL comprising an amino acid sequence that is at least 80% (e.g., 80%, 85%, 90%, 95%, 98%, or 99%) identical to the VL as set forth in SEQ ID NO: 11.

In some embodiments, an anti-KLK5 antibody of the present disclosure comprises a HC CDR1, HC CDR2 and HC CDR3 of a heavy chain variable domain having the amino acid sequence of SEQ ID NO: 18. Alternatively or in addition, the anti-KLK5 antibody of the present disclosure comprises a LC CDR1, LC CDR2 and LC CDR3 of a light chain variable domain having the amino acid sequence of SEQ ID NO: 11.

In some embodiments, an antibody of the present disclosure comprises a HC CDR1 having the amino acid sequence of SEQ ID NO: 12, a HC CDR2 having the amino acid sequence of SEQ ID NO: 13, a HC CDR3 having the amino acid sequence of SEQ ID NO: 17, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 6.

In some embodiments, an anti-KLK5 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 12, HC CDR2 having the amino acid sequence of SEQ ID NO: 13, and HC CDR3 having the amino acid sequence of SEQ ID NO: 17. “Collectively,” as used anywhere in the present disclosure, means that the total number of amino acid variations in all of the three heavy chain CDRs is within the defined range. Alternatively or in addition, the anti-KLK5 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 4, LC CDR2 having the amino acid sequence of SEQ ID NO: 5, and LC CDR3 having the amino acid sequence of SEQ ID NO: 6.

In some embodiments, an anti-KLK5 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 12, HC CDR2 having the amino acid sequence of SEQ ID NO: 13, and HC CDR3 having the amino acid sequence of SEQ ID NO: 17. Alternatively or in addition, the anti-KLK5 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 4, LC CDR2 having the amino acid sequence of SEQ ID NO: 5, and LC CDR3 having the amino acid sequence of SEQ ID NO: 6.

In some embodiments, an anti-KLK5 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 12; a HC CDR2 having no more than 3 amino acid variations (e.g., no more than 3, 2, or 1 amino acid variation) as compared with the HC CDR2 having the amino acid sequence of SEQ ID NO: 13; and/or a HC CDR3 having no more than 3 amino acid variations (e.g., no more than 3, 2, or 1 amino acid variation) as compared with the HC CDR3 having the amino acid sequence of SEQ ID NO: 17. Alternatively or in addition, the anti-KLK5 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of SEQ ID NO: 5; and/or a LC CDR3 having no more than 3 amino acid variations (e.g., no more than 3, 2, or 1 amino acid variation) as compared with the LC CDR3 having the amino acid sequence of SEQ ID NO: 6.

In some embodiments, an anti-KLK5 antibody of the present disclosure comprises a VH comprising the amino acid sequence of SEQ ID NO: 18. Alternatively or in addition, the anti-KLK5 antibody of the present disclosure comprises a VL comprising the amino acid sequence of SEQ ID NO: 11.

In some embodiments, an anti-KLK5 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 18. Alternatively or in addition, the anti-KLK5 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 11.

In some embodiments, an anti-KLK5 antibody of the present disclosure comprises a VH comprising an amino acid sequence that is at least 80% (e.g., 80%, 85%, 90%, 95%, 98%, or 99%) identical to the VH as set forth in SEQ ID NO: 18. Alternatively or in addition, the anti-KLK5 antibody of the present disclosure comprises a VL comprising an amino acid sequence that is at least 80% (e.g., 80%, 85%, 90%, 95%, 98%, or 99%) identical to the VL as set forth in SEQ ID NO: 11.

In some embodiments, an anti-KLK5 antibody of the present disclosure comprises a HC CDR1, HC CDR2 and HC CDR3 of a heavy chain variable domain having the amino acid sequence of SEQ ID NO: 24. Alternatively or in addition, the anti-KLK5 antibody of the present disclosure comprises a LC CDR1, LC CDR2 and LC CDR3 of a light chain variable domain having the amino acid sequence of SEQ ID NO: 29.

In some embodiments, an antibody of the present disclosure comprises a HC CDR1 having the amino acid sequence of SEQ ID NO: 19, a HC CDR2 having the amino acid sequence of SEQ ID NO: 20, a HC CDR3 having the amino acid sequence of SEQ ID NO: 21, a LC CDR1 having the amino acid sequence of SEQ ID NO: 22, a LC CDR2 having the amino acid sequence of SEQ ID NO: 23, and a LC CDR3 having the amino acid sequence of SEQ ID NO: 34.

In some embodiments, an anti-KLK5 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 19, HC CDR2 having the amino acid sequence of SEQ ID NO: 20, and HC CDR3 having the amino acid sequence of SEQ ID NO: 21. “Collectively,” as used anywhere in the present disclosure, means that the total number of amino acid variations in all of the three heavy chain CDRs is within the defined range. Alternatively or in addition, the anti-KLK5 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 22, LC CDR2 having the amino acid sequence of SEQ ID NO: 23, and LC CDR3 having the amino acid sequence of SEQ ID NO: 34.

In some embodiments, an anti-KLK5 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 19, HC CDR2 having the amino acid sequence of SEQ ID NO: 20, and HC CDR3 having the amino acid sequence of SEQ ID NO: 21. Alternatively or in addition, the anti-KLK5 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO:22, LC CDR2 having the amino acid sequence of SEQ ID NO: 23, and LC CDR3 having the amino acid sequence of SEQ ID NO: 34.

In some embodiments, an anti-KLK5 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 19; a HC CDR2 having no more than 3 amino acid variations (e.g., no more than 3, 2, or 1 amino acid variation) as compared with the HC CDR2 having the amino acid sequence of SEQ ID NO: 20; and/or a HC CDR3 having no more than 3 amino acid variations (e.g., no more than 3, 2, or 1 amino acid variation) as compared with the HC CDR3 having the amino acid sequence of SEQ ID NO: 21. Alternatively or in addition, the anti-KLK5 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 22; a LC CDR2 having no more than 3 amino acid variations (e.g., no more than 3, 2, or 1 amino acid variation) as compared with the LC CDR2 having the amino acid sequence of SEQ ID NO: 23; and/or a LC CDR3 having no more than 3 amino acid variations (e.g., no more than 3, 2, or 1 amino acid variation) as compared with the LC CDR3 having the amino acid sequence of SEQ ID NO: 34.

In some embodiments, an anti-KLK5 antibody of the present disclosure comprises a VH comprising the amino acid sequence of SEQ ID NO: 24. Alternatively or in addition, the anti-KLK5 antibody of the present disclosure comprises a VL comprising the amino acid sequence of SEQ ID NO: 29.

In some embodiments, an anti-KLK5 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 24. Alternatively or in addition, the anti-KLK5 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 29.

In some embodiments, an anti-KLK5 antibody of the present disclosure comprises a VH comprising an amino acid sequence that is at least 80% (e.g., 80%, 85%, 90%, 95%, 98%, or 99%) identical to the VH as set forth in SEQ ID NO: 24. Alternatively or in addition, the anti-KLK5 antibody of the present disclosure comprises a VL comprising an amino acid sequence that is at least 80% (e.g., 80%, 85%, 90%, 95%, 98%, or 99%) identical to the VL as set forth in SEQ ID NO: 29.

In some embodiments, an anti-KLK5 antibody of the present disclosure comprises a HC CDR1, HC CDR2 and HC CDR3 of a heavy chain variable domain having the amino acid sequence of SEQ ID NO: 28. Alternatively or in addition, the anti-KLK5 antibody of the present disclosure comprises a LC CDR1, LC CDR2 and LC CDR3 of a light chain variable domain having the amino acid sequence of SEQ ID NO: 29.

In some embodiments, an antibody of the present disclosure comprises a HC CDR1 having the amino acid sequence of SEQ ID NO: 25, a HC CDR2 having the amino acid sequence of SEQ ID NO: 26, a HC CDR3 having the amino acid sequence of SEQ ID NO: 27, a LC CDR1 having the amino acid sequence of SEQ ID NO: 22, a LC CDR2 having the amino acid sequence of SEQ ID NO: 23, and a LC CDR3 having the amino acid sequence of SEQ ID NO: 34.

In some embodiments, an anti-KLK5 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 25, HC CDR2 having the amino acid sequence of SEQ ID NO: 26, and HC CDR3 having the amino acid sequence of SEQ ID NO: 27. “Collectively,” as used anywhere in the present disclosure, means that the total number of amino acid variations in all of the three heavy chain CDRs is within the defined range. Alternatively or in addition, the anti-KLK5 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 22, LC CDR2 having the amino acid sequence of SEQ ID NO: 23, and LC CDR3 having the amino acid sequence of SEQ ID NO: 34.

In some embodiments, an anti-KLK5 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 25, HC CDR2 having the amino acid sequence of SEQ ID NO: 26, and HC CDR3 having the amino acid sequence of SEQ ID NO: 27. Alternatively or in addition, the anti-KLK5 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 22, LC CDR2 having the amino acid sequence of SEQ ID NO: 23, and LC CDR3 having the amino acid sequence of SEQ ID NO: 34.

In some embodiments, an anti-KLK5 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 25; a HC CDR2 having no more than 3 amino acid variations (e.g., no more than 3, 2, or 1 amino acid variation) as compared with the HC CDR2 having the amino acid sequence of SEQ ID NO: 26; and/or a HC CDR3 having no more than 3 amino acid variations (e.g., no more than 3, 2, or 1 amino acid variation) as compared with the HC CDR3 having the amino acid sequence of SEQ ID NO: 27. Alternatively or in addition, the anti-KLK5 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 22; a LC CDR2 having no more than 3 amino acid variations (e.g., no more than 3, 2, or 1 amino acid variation) as compared with the LC CDR2 having the amino acid sequence of SEQ ID NO: 23; and/or a LC CDR3 having no more than 3 amino acid variations (e.g., no more than 3, 2, or 1 amino acid variation) as compared with the LC CDR3 having the amino acid sequence of SEQ ID NO: 34.

In some embodiments, an anti-KLK5 antibody of the present disclosure comprises a VH comprising the amino acid sequence of SEQ ID NO: 28. Alternatively or in addition, the anti-KLK5 antibody of the present disclosure comprises a VL comprising the amino acid sequence of SEQ ID NO: 29.

In some embodiments, an anti-KLK5 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 28. Alternatively or in addition, the anti-KLK5 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 29.

In some embodiments, an anti-KLK5 antibody of the present disclosure comprises a VH comprising an amino acid sequence that is at least 80% (e.g., 80%, 85%, 90%, 95%, 98%, or 99%) identical to the VH as set forth in SEQ ID NO: 28. Alternatively or in addition, the anti-KLK5 antibody of the present disclosure comprises a VL comprising an amino acid sequence that is at least 80% (e.g., 80%, 85%, 90%, 95%, 98%, or 99%) identical to the VL as set forth in SEQ ID NO: 29.

In some embodiments, an anti-KLK5 antibody of the present disclosure comprises a HC CDR1, HC CDR2 and HC CDR3 of a heavy chain variable domain having the amino acid sequence of SEQ ID NO: 32. Alternatively or in addition, the anti-KLK5 antibody of the present disclosure comprises a LC CDR1, LC CDR2 and LC CDR3 of a light chain variable domain having the amino acid sequence of SEQ ID NO: 37.

In some embodiments, an antibody of the present disclosure comprises a HC CDR1 having the amino acid sequence of SEQ ID NO: 30, a HC CDR2 having the amino acid sequence of SEQ ID NO: 26, a HC CDR3 having the amino acid sequence of SEQ ID NO: 31, a LC CDR1 having the amino acid sequence of SEQ ID NO: 22, a LC CDR2 having the amino acid sequence of SEQ ID NO: 23, and a LC CDR3 having the amino acid sequence of SEQ ID NO: 35.

In some embodiments, an anti-KLK5 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 30, HC CDR2 having the amino acid sequence of SEQ ID NO: 26, and HC CDR3 having the amino acid sequence of SEQ ID NO: 31. “Collectively,” as used anywhere in the present disclosure, means that the total number of amino acid variations in all of the three heavy chain CDRs is within the defined range. Alternatively or in addition, the anti-KLK5 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 22, LC CDR2 having the amino acid sequence of SEQ ID NO: 23, and LC CDR3 having the amino acid sequence of SEQ ID NO: 35.

In some embodiments, an anti-KLK5 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 30, HC CDR2 having the amino acid sequence of SEQ ID NO: 26, and HC CDR3 having the amino acid sequence of SEQ ID NO: 31. Alternatively or in addition, the anti-KLK5 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 22, LC CDR2 having the amino acid sequence of SEQ ID NO: 23, and LC CDR3 having the amino acid sequence of SEQ ID NO: 35.

In some embodiments, an anti-KLK5 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 30; a HC CDR2 having no more than 3 amino acid variations (e.g., no more than 3, 2, or 1 amino acid variation) as compared with the HC CDR2 having the amino acid sequence of SEQ ID NO: 26; and/or a HC CDR3 having no more than 3 amino acid variations (e.g., no more than 3, 2, or 1 amino acid variation) as compared with the HC CDR3 having the amino acid sequence of SEQ ID NO: 31. Alternatively or in addition, the anti-KLK5 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 22; a LC CDR2 having no more than 3 amino acid variations (e.g., no more than 3, 2, or 1 amino acid variation) as compared with the LC CDR2 having the amino acid sequence of SEQ ID NO: 23; and/or a LC CDR3 having no more than 3 amino acid variations (e.g., no more than 3, 2, or 1 amino acid variation) as compared with the LC CDR3 having the amino acid sequence of SEQ ID NO: 35.

In some embodiments, an anti-KLK5 antibody of the present disclosure comprises a VH comprising the amino acid sequence of SEQ ID NO: 32. Alternatively or in addition, the anti-KLK5 antibody of the present disclosure comprises a VL comprising the amino acid sequence of SEQ ID NO: 37.

In some embodiments, an anti-KLK5 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 32. Alternatively or in addition, the anti-KLK5 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 37.

In some embodiments, an anti-KLK5 antibody of the present disclosure comprises a VH comprising an amino acid sequence that is at least 80% (e.g., 80%, 85%, 90%, 95%, 98%, or 99%) identical to the VH as set forth in SEQ ID NO: 32. Alternatively or in addition, the anti-KLK5 antibody of the present disclosure comprises a VL comprising an amino acid sequence that is at least 80% (e.g., 80%, 85%, 90%, 95%, 98%, or 99%) identical to the VL as set forth in SEQ ID NO: 37.

In some embodiments, an anti-KLK5 antibody of the present disclosure comprises a HC CDR1, HC CDR2 and HC CDR3 of a heavy chain variable domain having the amino acid sequence of SEQ ID NO: 36. Alternatively or in addition, the anti-KLK5 antibody of the present disclosure comprises a LC CDR1, LC CDR2 and LC CDR3 of a light chain variable domain having the amino acid sequence of SEQ ID NO: 29.

In some embodiments, an antibody of the present disclosure comprises a HC CDR1 having the amino acid sequence of SEQ ID NO: 30, a HC CDR2 having the amino acid sequence of SEQ ID NO: 26, a HC CDR3 having the amino acid sequence of SEQ ID NO: 33, a LC CDR1 having the amino acid sequence of SEQ ID NO: 22, a LC CDR2 having the amino acid sequence of SEQ ID NO: 23, and a LC CDR3 having the amino acid sequence of SEQ ID NO: 34.

In some embodiments, an anti-KLK5 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 30, HC CDR2 having the amino acid sequence of SEQ ID NO: 26, and HC CDR3 having the amino acid sequence of SEQ ID NO: 33. “Collectively,” as used anywhere in the present disclosure, means that the total number of amino acid variations in all of the three heavy chain CDRs is within the defined range. Alternatively or in addition, the anti-KLK5 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 22, LC CDR2 having the amino acid sequence of SEQ ID NO: 23, and LC CDR3 having the amino acid sequence of SEQ ID NO: 34.

In some embodiments, an anti-KLK5 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 30, HC CDR2 having the amino acid sequence of SEQ ID NO: 26, and HC CDR3 having the amino acid sequence of SEQ ID NO: 33. Alternatively or in addition, the anti-KLK5 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 22, LC CDR2 having the amino acid sequence of SEQ ID NO: 23, and LC CDR3 having the amino acid sequence of SEQ ID NO: 34.

In some embodiments, an anti-KLK5 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 30; a HC CDR2 having no more than 3 amino acid variations (e.g., no more than 3, 2, or 1 amino acid variation) as compared with the HC CDR2 having the amino acid sequence of SEQ ID NO: 26; and/or a HC CDR3 having no more than 3 amino acid variations (e.g., no more than 3, 2, or 1 amino acid variation) as compared with the HC CDR3 having the amino acid sequence of SEQ ID NO: 33. Alternatively or in addition, the anti-KLK5 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 22; a LC CDR2 having no more than 3 amino acid variations (e.g., no more than 3, 2, or 1 amino acid variation) as compared with the LC CDR2 having the amino acid sequence of SEQ ID NO: 23; and/or a LC CDR3 having no more than 3 amino acid variations (e.g., no more than 3, 2, or 1 amino acid variation) as compared with the LC CDR3 having the amino acid sequence of SEQ ID NO: 34.

In some embodiments, an anti-KLK5 antibody of the present disclosure comprises a VH comprising the amino acid sequence of SEQ ID NO: 36. Alternatively or in addition, the anti-KLK5 antibody of the present disclosure comprises a VL comprising the amino acid sequence of SEQ ID NO: 29.

In some embodiments, an anti-KLK5 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 36. Alternatively or in addition, the anti-KLK5 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 29.

In some embodiments, an anti-KLK5 antibody of the present disclosure comprises a VH comprising an amino acid sequence that is at least 80% (e.g., 80%, 85%, 90%, 95%, 98%, or 99%) identical to the VH as set forth in SEQ ID NO: 36. Alternatively or in addition, the anti-KLK5 antibody of the present disclosure comprises a VL comprising an amino acid sequence that is at least 80% (e.g., 80%, 85%, 90%, 95%, 98%, or 99%) identical to the VL as set forth in SEQ ID NO: 29.

In some embodiments, an anti-KLK5 antibody of the present disclosure comprises a HC CDR1, HC CDR2 and HC CDR3 of a heavy chain variable domain having the amino acid sequence of SEQ ID NO: 42. Alternatively or in addition, the anti-KLK5 antibody of the present disclosure comprises a LC CDR1, LC CDR2 and LC CDR3 of a light chain variable domain having the amino acid sequence of SEQ ID NO: 43.

In some embodiments, an antibody of the present disclosure comprises a HC CDR1 having the amino acid sequence of SEQ ID NO: 38, a HC CDR2 having the amino acid sequence of SEQ ID NO: 39, a HC CDR3 having the amino acid sequence of SEQ ID NO: 40, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 41.

In some embodiments, an anti-KLK5 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 38, HC CDR2 having the amino acid sequence of SEQ ID NO: 39, and HC CDR3 having the amino acid sequence of SEQ ID NO: 40. “Collectively,” as used anywhere in the present disclosure, means that the total number of amino acid variations in all of the three heavy chain CDRs is within the defined range. Alternatively or in addition, the anti-KLK5 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 4, LC CDR2 having the amino acid sequence of SEQ ID NO: 5, and LC CDR3 having the amino acid sequence of SEQ ID NO: 41.

In some embodiments, an anti-KLK5 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 38, HC CDR2 having the amino acid sequence of SEQ ID NO: 39, and HC CDR3 having the amino acid sequence of SEQ ID NO: 40. Alternatively or in addition, the anti-KLK5 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 4, LC CDR2 having the amino acid sequence of SEQ ID NO: 5, and LC CDR3 having the amino acid sequence of SEQ ID NO: 41.

In some embodiments, an anti-KLK5 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 38; a HC CDR2 having no more than 3 amino acid variations (e.g., no more than 3, 2, or 1 amino acid variation) as compared with the HC CDR2 having the amino acid sequence of SEQ ID NO: 39; and/or a HC CDR3 having no more than 3 amino acid variations (e.g., no more than 3, 2, or 1 amino acid variation) as compared with the HC CDR3 having the amino acid sequence of SEQ ID NO: 40. Alternatively or in addition, the anti-KLK5 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of SEQ ID NO: 5; and/or a LC CDR3 having no more than 3 amino acid variations (e.g., no more than 3, 2, or 1 amino acid variation) as compared with the LC CDR3 having the amino acid sequence of SEQ ID NO: 41.

In some embodiments, an anti-KLK5 antibody of the present disclosure comprises a VH comprising the amino acid sequence of SEQ ID NO: 42. Alternatively or in addition, the anti-KLK5 antibody of the present disclosure comprises a VL comprising the amino acid sequence of SEQ ID NO: 43.

In some embodiments, an anti-KLK5 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 42. Alternatively or in addition, the anti-KLK5 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 43.

In some embodiments, an anti-KLK5 antibody of the present disclosure comprises a VH comprising an amino acid sequence that is at least 80% (e.g., 80%, 85%, 90%, 95%, 98%, or 99%) identical to the VH as set forth in SEQ ID NO: 42. Alternatively or in addition, the anti-KLK5 antibody of the present disclosure comprises a VL comprising an amino acid sequence that is at least 80% (e.g., 80%, 85%, 90%, 95%, 98%, or 99%) identical to the VL as set forth in SEQ ID NO: 43.

In some embodiments, an anti-KLK5 antibody of the present disclosure comprises a HC CDR1, HC CDR2 and HC CDR3 of a heavy chain variable domain having the amino acid sequence of SEQ ID NO: 46. Alternatively or in addition, the anti-KLK5 antibody of the present disclosure comprises a LC CDR1, LC CDR2 and LC CDR3 of a light chain variable domain having the amino acid sequence of SEQ ID NO: 43.

In some embodiments, an antibody of the present disclosure comprises a HC CDR1 having the amino acid sequence of SEQ ID NO: 44, a HC CDR2 having the amino acid sequence of SEQ ID NO: 45, a HC CDR3 having the amino acid sequence of SEQ ID NO: 40, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 41.

In some embodiments, an anti-KLK5 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 44, HC CDR2 having the amino acid sequence of SEQ ID NO: 45, and HC CDR3 having the amino acid sequence of SEQ ID NO: 40. “Collectively,” as used anywhere in the present disclosure, means that the total number of amino acid variations in all of the three heavy chain CDRs is within the defined range. Alternatively or in addition, the anti-KLK5 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 4, LC CDR2 having the amino acid sequence of SEQ ID NO: 5, and LC CDR3 having the amino acid sequence of SEQ ID NO: 41.

In some embodiments, an anti-KLK5 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 44, HC CDR2 having the amino acid sequence of SEQ ID NO: 45, and HC CDR3 having the amino acid sequence of SEQ ID NO: 40. Alternatively or in addition, the anti-KLK5 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 4, LC CDR2 having the amino acid sequence of SEQ ID NO: 5, and LC CDR3 having the amino acid sequence of SEQ ID NO: 41.

In some embodiments, an anti-KLK5 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 44; a HC CDR2 having no more than 3 amino acid variations (e.g., no more than 3, 2, or 1 amino acid variation) as compared with the HC CDR2 having the amino acid sequence of SEQ ID NO: 45; and/or a HC CDR3 having no more than 3 amino acid variations (e.g., no more than 3, 2, or 1 amino acid variation) as compared with the HC CDR3 having the amino acid sequence of SEQ ID NO: 40. Alternatively or in addition, the anti-KLK5 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of SEQ ID NO: 5; and/or a LC CDR3 having no more than 3 amino acid variations (e.g., no more than 3, 2, or 1 amino acid variation) as compared with the LC CDR3 having the amino acid sequence of SEQ ID NO: 41.

In some embodiments, an anti-KLK5 antibody of the present disclosure comprises a VH comprising the amino acid sequence of SEQ ID NO: 46. Alternatively or in addition, the anti-KLK5 antibody of the present disclosure comprises a VL comprising the amino acid sequence of SEQ ID NO: 43.

In some embodiments, an anti-KLK5 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 46. Alternatively or in addition, the anti-KLK5 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 43.

In some embodiments, an anti-KLK5 antibody of the present disclosure comprises a VH comprising an amino acid sequence that is at least 80% (e.g., 80%, 85%, 90%, 95%, 98%, or 99%) identical to the VH as set forth in SEQ ID NO: 46. Alternatively or in addition, the anti-KLK5 antibody of the present disclosure comprises a VL comprising an amino acid sequence that is at least 80% (e.g., 80%, 85%, 90%, 95%, 98%, or 99%) identical to the VL as set forth in SEQ ID NO: 43.

In some embodiments, an anti-KLK5 antibody of the present disclosure comprises a HC CDR1, HC CDR2 and HC CDR3 of a heavy chain variable domain having the amino acid sequence of SEQ ID NO: 49. Alternatively or in addition, the anti-KLK5 antibody of the present disclosure comprises a LC CDR1, LC CDR2 and LC CDR3 of a light chain variable domain having the amino acid sequence of SEQ ID NO: 43.

In some embodiments, an antibody of the present disclosure comprises a HC CDR1 having the amino acid sequence of SEQ ID NO: 47, a HC CDR2 having the amino acid sequence of SEQ ID NO: 48, a HC CDR3 having the amino acid sequence of SEQ ID NO: 40, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 41.

In some embodiments, an anti-KLK5 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 47, HC CDR2 having the amino acid sequence of SEQ ID NO: 48, and HC CDR3 having the amino acid sequence of SEQ ID NO: 40. “Collectively,” as used anywhere in the present disclosure, means that the total number of amino acid variations in all of the three heavy chain CDRs is within the defined range. Alternatively or in addition, the anti-KLK5 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 4, LC CDR2 having the amino acid sequence of SEQ ID NO: 5, and LC CDR3 having the amino acid sequence of SEQ ID NO: 41.

In some embodiments, an anti-KLK5 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 47, HC CDR2 having the amino acid sequence of SEQ ID NO: 48, and HC CDR3 having the amino acid sequence of SEQ ID NO: 40. Alternatively or in addition, the anti-KLK5 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 4, LC CDR2 having the amino acid sequence of SEQ ID NO: 5, and LC CDR3 having the amino acid sequence of SEQ ID NO: 41.

In some embodiments, an anti-KLK5 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 47; a HC CDR2 having no more than 3 amino acid variations (e.g., no more than 3, 2, or 1 amino acid variation) as compared with the HC CDR2 having the amino acid sequence of SEQ ID NO: 48; and/or a HC CDR3 having no more than 3 amino acid variations (e.g., no more than 3, 2, or 1 amino acid variation) as compared with the HC CDR3 having the amino acid sequence of SEQ ID NO: 40. Alternatively or in addition, the anti-KLK5 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of SEQ ID NO: 5; and/or a LC CDR3 having no more than 3 amino acid variations (e.g., no more than 3, 2, or 1 amino acid variation) as compared with the LC CDR3 having the amino acid sequence of SEQ ID NO: 41.

In some embodiments, an anti-KLK5 antibody of the present disclosure comprises a VH comprising the amino acid sequence of SEQ ID NO: 49. Alternatively or in addition, the anti-KLK5 antibody of the present disclosure comprises a VL comprising the amino acid sequence of SEQ ID NO: 43.

In some embodiments, an anti-KLK5 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 49. Alternatively or in addition, the anti-KLK5 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 43.

In some embodiments, an anti-KLK5 antibody of the present disclosure comprises a VH comprising an amino acid sequence that is at least 80% (e.g., 80%, 85%, 90%, 95%, 98%, or 99%) identical to the VH as set forth in SEQ ID NO: 49. Alternatively or in addition, the anti-KLK5 antibody of the present disclosure comprises a VL comprising an amino acid sequence that is at least 80% (e.g., 80%, 85%, 90%, 95%, 98%, or 99%) identical to the VL as set forth in SEQ ID NO: 43.

In some embodiments, an anti-KLK5 antibody of the present disclosure comprises a HC CDR1, HC CDR2 and HC CDR3 of a heavy chain variable domain having the amino acid sequence of SEQ ID NO: 51. Alternatively or in addition, the anti-KLK5 antibody of the present disclosure comprises a LC CDR1, LC CDR2 and LC CDR3 of a light chain variable domain having the amino acid sequence of SEQ ID NO: 43.

In some embodiments, an antibody of the present disclosure comprises a HC CDR1 having the amino acid sequence of SEQ ID NO: 47, a HC CDR2 having the amino acid sequence of SEQ ID NO: 48, a HC CDR3 having the amino acid sequence of SEQ ID NO: 50, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 41.

In some embodiments, an anti-KLK5 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 47, HC CDR2 having the amino acid sequence of SEQ ID NO: 48, and HC CDR3 having the amino acid sequence of SEQ ID NO: 50. “Collectively,” as used anywhere in the present disclosure, means that the total number of amino acid variations in all of the three heavy chain CDRs is within the defined range. Alternatively or in addition, the anti-KLK5 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 4, LC CDR2 having the amino acid sequence of SEQ ID NO: 5, and LC CDR3 having the amino acid sequence of SEQ ID NO: 41.

In some embodiments, an anti-KLK5 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 47, HC CDR2 having the amino acid sequence of SEQ ID NO: 48, and HC CDR3 having the amino acid sequence of SEQ ID NO: 50. Alternatively or in addition, the anti-KLK5 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 4, LC CDR2 having the amino acid sequence of SEQ ID NO: 5, and LC CDR3 having the amino acid sequence of SEQ ID NO: 41.

In some embodiments, an anti-KLK5 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 47; a HC CDR2 having no more than 3 amino acid variations (e.g., no more than 3, 2, or 1 amino acid variation) as compared with the HC CDR2 having the amino acid sequence of SEQ ID NO: 48; and/or a HC CDR3 having no more than 3 amino acid variations (e.g., no more than 3, 2, or 1 amino acid variation) as compared with the HC CDR3 having the amino acid sequence of SEQ ID NO: 50. Alternatively or in addition, the anti-KLK5 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of SEQ ID NO: 5; and/or a LC CDR3 having no more than 3 amino acid variations (e.g., no more than 3, 2, or 1 amino acid variation) as compared with the LC CDR3 having the amino acid sequence of SEQ ID NO: 41.

In some embodiments, an anti-KLK5 antibody of the present disclosure comprises a VH comprising the amino acid sequence of SEQ ID NO: 51. Alternatively or in addition, the anti-KLK5 antibody of the present disclosure comprises a VL comprising the amino acid sequence of SEQ ID NO: 43.

In some embodiments, an anti-KLK5 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 51. Alternatively or in addition, the anti-KLK5 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 43.

In some embodiments, an anti-KLK5 antibody of the present disclosure comprises a VH comprising an amino acid sequence that is at least 80% (e.g., 80%, 85%, 90%, 95%, 98%, or 99%) identical to the VH as set forth in SEQ ID NO: 51. Alternatively or in addition, the anti-KLK5 antibody of the present disclosure comprises a VL comprising an amino acid sequence that is at least 80% (e.g., 80%, 85%, 90%, 95%, 98%, or 99%) identical to the VL as set forth in SEQ ID NO: 43.

In some embodiments, an anti-KLK5 antibody of the present disclosure comprises a HC CDR1, HC CDR2 and HC CDR3 of a heavy chain variable domain having the amino acid sequence of SEQ ID NO: 53. Alternatively or in addition, the anti-KLK5 antibody of the present disclosure comprises a LC CDR1, LC CDR2 and LC CDR3 of a light chain variable domain having the amino acid sequence of SEQ ID NO: 43.

In some embodiments, an antibody of the present disclosure comprises a HC CDR1 having the amino acid sequence of SEQ ID NO: 44, a HC CDR2 having the amino acid sequence of SEQ ID NO: 45, a HC CDR3 having the amino acid sequence of SEQ ID NO: 52, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 41.

In some embodiments, an anti-KLK5 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 44, HC CDR2 having the amino acid sequence of SEQ ID NO: 45, and HC CDR3 having the amino acid sequence of SEQ ID NO: 52. “Collectively,” as used anywhere in the present disclosure, means that the total number of amino acid variations in all of the three heavy chain CDRs is within the defined range. Alternatively or in addition, the anti-KLK5 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 4, LC CDR2 having the amino acid sequence of SEQ ID NO: 5, and LC CDR3 having the amino acid sequence of SEQ ID NO: 41.

In some embodiments, an anti-KLK5 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 44, HC CDR2 having the amino acid sequence of SEQ ID NO: 45, and HC CDR3 having the amino acid sequence of SEQ ID NO: 52. Alternatively or in addition, the anti-KLK5 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 4, LC CDR2 having the amino acid sequence of SEQ ID NO: 5, and LC CDR3 having the amino acid sequence of SEQ ID NO: 41.

In some embodiments, an anti-KLK5 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 44; a HC CDR2 having no more than 3 amino acid variations (e.g., no more than 3, 2, or 1 amino acid variation) as compared with the HC CDR2 having the amino acid sequence of SEQ ID NO: 45; and/or a HC CDR3 having no more than 3 amino acid variations (e.g., no more than 3, 2, or 1 amino acid variation) as compared with the HC CDR3 having the amino acid sequence of SEQ ID NO: 52. Alternatively or in addition, the anti-KLK5 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of SEQ ID NO: 5; and/or a LC CDR3 having no more than 3 amino acid variations (e.g., no more than 3, 2, or 1 amino acid variation) as compared with the LC CDR3 having the amino acid sequence of SEQ ID NO: 41.

In some embodiments, an anti-KLK5 antibody of the present disclosure comprises a VH comprising the amino acid sequence of SEQ ID NO: 53. Alternatively or in addition, the anti-KLK5 antibody of the present disclosure comprises a VL comprising the amino acid sequence of SEQ ID NO: 43.

In some embodiments, an anti-KLK5 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 53. Alternatively or in addition, the anti-KLK5 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 43.

In some embodiments, an anti-KLK5 antibody of the present disclosure comprises a VH comprising an amino acid sequence that is at least 80% (e.g., 80%, 85%, 90%, 95%, 98%, or 99%) identical to the VH as set forth in SEQ ID NO: 53. Alternatively or in addition, the anti-KLK5 antibody of the present disclosure comprises a VL comprising an amino acid sequence that is at least 80% (e.g., 80%, 85%, 90%, 95%, 98%, or 99%) identical to the VL as set forth in SEQ ID NO: 43.

In some embodiments, an anti-KLK5 antibody of the present disclosure comprises a HC CDR1, HC CDR2 and HC CDR3 of a heavy chain variable domain having the amino acid sequence of SEQ ID NO: 58. Alternatively or in addition, the anti-KLK5 antibody of the present disclosure comprises a LC CDR1, LC CDR2 and LC CDR3 of a light chain variable domain having the amino acid sequence of SEQ ID NO: 59.

In some embodiments, an antibody of the present disclosure comprises a HC CDR1 having the amino acid sequence of SEQ ID NO: 54, a HC CDR2 having the amino acid sequence of SEQ ID NO: 55, a HC CDR3 having the amino acid sequence of SEQ ID NO: 56, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 57.

In some embodiments, an anti-KLK5 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 54, HC CDR2 having the amino acid sequence of SEQ ID NO: 55, and HC CDR3 having the amino acid sequence of SEQ ID NO: 56. “Collectively,” as used anywhere in the present disclosure, means that the total number of amino acid variations in all of the three heavy chain CDRs is within the defined range. Alternatively or in addition, the anti-KLK5 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 4, LC CDR2 having the amino acid sequence of SEQ ID NO: 5, and LC CDR3 having the amino acid sequence of SEQ ID NO: 57.

In some embodiments, an anti-KLK5 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 54, HC CDR2 having the amino acid sequence of SEQ ID NO: 55, and HC CDR3 having the amino acid sequence of SEQ ID NO: 56. Alternatively or in addition, the anti-KLK5 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 4, LC CDR2 having the amino acid sequence of SEQ ID NO: 5, and LC CDR3 having the amino acid sequence of SEQ ID NO: 57.

In some embodiments, an anti-KLK5 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 54; a HC CDR2 having no more than 3 amino acid variations (e.g., no more than 3, 2, or 1 amino acid variation) as compared with the HC CDR2 having the amino acid sequence of SEQ ID NO: 55; and/or a HC CDR3 having no more than 3 amino acid variations (e.g., no more than 3, 2, or 1 amino acid variation) as compared with the HC CDR3 having the amino acid sequence of SEQ ID NO: 56. Alternatively or in addition, the anti-KLK5 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of SEQ ID NO: 5; and/or a LC CDR3 having no more than 3 amino acid variations (e.g., no more than 3, 2, or 1 amino acid variation) as compared with the LC CDR3 having the amino acid sequence of SEQ ID NO: 57.

In some embodiments, an anti-KLK5 antibody of the present disclosure comprises a VH comprising the amino acid sequence of SEQ ID NO: 58. Alternatively or in addition, the anti-KLK5 antibody of the present disclosure comprises a VL comprising the amino acid sequence of SEQ ID NO: 59.

In some embodiments, an anti-KLK5 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 58. Alternatively or in addition, the anti-KLK5 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 59.

In some embodiments, an anti-KLK5 antibody of the present disclosure comprises a VH comprising an amino acid sequence that is at least 80% (e.g., 80%, 85%, 90%, 95%, 98%, or 99%) identical to the VH as set forth in SEQ ID NO: 58. Alternatively or in addition, the anti-KLK5 antibody of the present disclosure comprises a VL comprising an amino acid sequence that is at least 80% (e.g., 80%, 85%, 90%, 95%, 98%, or 99%) identical to the VL as set forth in SEQ ID NO: 59.

In some embodiments, an anti-KLK5 antibody of the present disclosure comprises a HC CDR1, HC CDR2 and HC CDR3 of a heavy chain variable domain having the amino acid sequence of SEQ ID NO: 64. Alternatively or in addition, the anti-KLK5 antibody of the present disclosure comprises a LC CDR1, LC CDR2 and LC CDR3 of a light chain variable domain having the amino acid sequence of SEQ ID NO: 65.

In some embodiments, an antibody of the present disclosure comprises a HC CDR1 having the amino acid sequence of SEQ ID NO: 60, a HC CDR2 having the amino acid sequence of SEQ ID NO: 61, a HC CDR3 having the amino acid sequence of SEQ ID NO: 62, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 63.

In some embodiments, an anti-KLK5 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 60, HC CDR2 having the amino acid sequence of SEQ ID NO: 61, and HC CDR3 having the amino acid sequence of SEQ ID NO: 62. “Collectively,” as used anywhere in the present disclosure, means that the total number of amino acid variations in all of the three heavy chain CDRs is within the defined range. Alternatively or in addition, the anti-KLK5 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 4, LC CDR2 having the amino acid sequence of SEQ ID NO: 5, and LC CDR3 having the amino acid sequence of SEQ ID NO: 63.

In some embodiments, an anti-KLK5 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 60, HC CDR2 having the amino acid sequence of SEQ ID NO: 61, and HC CDR3 having the amino acid sequence of SEQ ID NO: 62. Alternatively or in addition, the anti-KLK5 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 4, LC CDR2 having the amino acid sequence of SEQ ID NO: 5, and LC CDR3 having the amino acid sequence of SEQ ID NO: 63.

In some embodiments, an anti-KLK5 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 60; a HC CDR2 having no more than 3 amino acid variations (e.g., no more than 3, 2, or 1 amino acid variation) as compared with the HC CDR2 having the amino acid sequence of SEQ ID NO: 61; and/or a HC CDR3 having no more than 3 amino acid variations (e.g., no more than 3, 2, or 1 amino acid variation) as compared with the HC CDR3 having the amino acid sequence of SEQ ID NO: 62. Alternatively or in addition, the anti-KLK5 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of SEQ ID NO: 5; and/or a LC CDR3 having no more than 3 amino acid variations (e.g., no more than 3, 2, or 1 amino acid variation) as compared with the LC CDR3 having the amino acid sequence of SEQ ID NO: 63.

In some embodiments, an anti-KLK5 antibody of the present disclosure comprises a VH comprising the amino acid sequence of SEQ ID NO: 64. Alternatively or in addition, the anti-KLK5 antibody of the present disclosure comprises a VL comprising the amino acid sequence of SEQ ID NO: 65.

In some embodiments, an anti-KLK5 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 64. Alternatively or in addition, the anti-KLK5 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 65.

In some embodiments, an anti-KLK5 antibody of the present disclosure comprises a VH comprising an amino acid sequence that is at least 80% (e.g., 80%, 85%, 90%, 95%, 98%, or 99%) identical to the VH as set forth in SEQ ID NO: 64. Alternatively or in addition, the anti-KLK5 antibody of the present disclosure comprises a VL comprising an amino acid sequence that is at least 80% (e.g., 80%, 85%, 90%, 95%, 98%, or 99%) identical to the VL as set forth in SEQ ID NO: 65.

The antibodies described herein can be in any antibody form, including, but not limited to, intact (i.e., full-length) antibodies, antigen-binding fragments thereof (such as Fab, F(ab′), F(ab′)2, Fv), single chain antibodies, bi-specific antibodies, or nanobodies. In some embodiments, the anti-KLK5 antibody described herein is a scFv. In some embodiments, the anti-KLK5 antibody described herein is a scFv-Fab (e.g., scFv fused to a portion of a constant region).

In some embodiments, an anti-KLK5 antibody of the present disclosure is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

In some embodiments, an antibody of the present disclosure comprises a VL domain and/or VH domain of any one of the anti-KLK5 antibodies selected from Tables 1a and 1b, and comprises a constant region comprising the amino acid sequences of the constant regions of an IgG, IgE, IgM, IgD, IgA or IgY immunoglobulin molecule, any class (e.g., IgG1, IgG2, IgG3, IgG4, IgA1 and IgA2), or any subclass (e.g., IgG2a and IgG2b) of immunoglobulin molecule. Non-limiting examples of human constant regions are described in the art, e.g., see Kabat E A et al., (1991) supra.

In some embodiments, the light chain of any of the anti-KLK5 antibodies described herein may further comprise a light chain constant region (CL), which can be any CL known in the art. In some examples, the CL is a kappa light chain. In other examples, the CL is a lambda light chain. In some embodiments, the CL is a kappa light chain.

Other antibody heavy and light chain constant regions are well known in the art, e.g., those provided in the IMGT database (www.imgt.org) or at www.vbase2.org/vbstat.php., both of which are incorporated by reference herein.

In some embodiments, conservative mutations can be introduced into antibody sequences (e.g., CDRs or framework sequences) at positions where the residues are not likely to be involved in interacting with a target antigen (e.g., human or mouse KLK5), for example, as determined based on a crystal structure. In some embodiments, one, two or more mutations (e.g., amino acid substitutions) are introduced into the Fc region of an anti-KLK5 antibody described herein (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to alter one or more functional properties of the antibody, such as serum half-life, complement fixation, Fc receptor binding and/or antigen-dependent cellular cytotoxicity.

In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more mutations (e.g., amino acid substitutions) are introduced into the Fc region of an antibody described herein (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to decrease the half-life of the anti-KLK5 antibody in vivo. In some embodiments, one, two or more amino acid mutations (i.e., substitutions, insertions or deletions) are introduced into an IgG constant domain, or FcRn-binding fragment thereof (preferably an Fc or hinge-Fc domain fragment) to increase the half-life of the antibody in vivo. In some embodiments, the antibodies can have one or more amino acid mutations (e.g., substitutions) in the second constant (CH2) domain (residues 231-340 of human IgG1) and/or the third constant (CH3) domain (residues 341-447 of human IgG1), with numbering according to the EU index in Kabat (Kabat E A et al., (1991) supra). In some embodiments, the constant region of the IgG1 of an antibody described herein comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall'Acqua W F et al., (2006) J Biol Chem 281: 23514-24). In some embodiments, an antibody comprises an IgG constant domain comprising one, two, three or more amino acid substitutions of amino acid residues at positions 251-257, 285-290, 308-314, 385-389, and 428-436, numbered according to the EU index as in Kabat.

In some embodiments, an antibody comprises an Fc region that has been engineered for half-life extension purposes, e.g., by introducing M428L and/or N434A substitutions. Non-limiting examples of such Fc variants effecting half-life in circulation are provided in Saunders K O, Conceptual Approaches to Modulating Antibody Effector Functions and Circulation Half-Life, Front Immunol. 2019; 10: 1296, the contents of which are incorporated herein by reference.

In some embodiments, one, two or more amino acid substitutions are introduced into an IgG constant domain Fc region to alter the effector function(s) of the anti-KLK5 antibody, e.g., by introducing Leu234Ala and Leu235Ala mutations (commonly called LALA mutations). The effector ligand to which affinity is altered can be, for example, an Fc receptor or the C1 component of complement. This approach is described in further detail in U.S. Pat. Nos. 5,624,821 and 5,648,260. In some embodiments, the deletion or inactivation (through point mutations or other means) of a constant region domain can reduce Fc receptor binding of the circulating antibody thereby increasing tumor localization. See, e.g., U.S. Pat. Nos. 5,585,097 and 8,591,886 for a description of mutations that delete or inactivate the constant domain and thereby increase tumor localization. In some embodiments, one or more amino acid substitutions may be introduced into the Fc region of an antibody described herein to remove potential glycosylation sites on Fc region, which may reduce Fc receptor binding (see, e.g., Shields R L et al., (2001) J Biol Chem 276: 6591-604).

In some embodiments, one or more amino in the constant region of an anti-KLK5 antibody described herein can be replaced with a different amino acid residue such that the antibody has altered Clq binding and/or reduced or abolished complement dependent cytotoxicity (CDC). This approach is described in further detail in U.S. Pat. No. 6,194,551 (Idusogie et al). In some embodiments, one or more amino acid residues in the N-terminal region of the CH2 domain of an antibody described herein are altered to thereby alter the ability of the antibody to fix complement. This approach is described further in International Publication No. WO 94/29351. In some embodiments, the Fc region of an antibody described herein is modified to increase the ability of the antibody to mediate antibody dependent cellular cytotoxicity (ADCC) and/or to increase the affinity of the antibody for an Fcγ receptor. This approach is described further in International Publication No. WO 00/42072.

In some embodiments, an antibody comprises an Fc variant comprising amino acid substitutions L234A, L235E, and P329G, wherein numbering is according to the EU index. In some embodiments, the antibody comprising the Fc variant exhibits reduced affinity to one or more or each of FcγRJ, FcγRIIA, FcγRIIIA, and Clq as compared to an antibody comprising the wild-type human Fc region. Examples of such Fc variants are provided in International Patent Application Publication No.: WO 2021/055669 entitled, FC VARIANTS WITH REDUCED EFFECTOR FUNCTION, published on Mar. 25, 2021; and US Patent Application Publication No.: US 2021-0087271 entitled, FC VARIANTS WITH REDUCED EFFECTOR FUNCTION, published on Mar. 25, 2021, the contents of which are incorporated herein by reference.

In some embodiments, the heavy and/or light chain variable domain(s) sequence(s) of the antibodies provided herein can be used to generate, for example, CDR-grafted, chimeric, humanized, or composite human antibodies or antigen-binding fragments, as described elsewhere herein. As understood by one of ordinary skill in the art, any variant, CDR-grafted, chimeric, humanized, or composite antibodies derived from any of the antibodies provided herein may be useful in the compositions and methods described herein and will maintain the ability to specifically bind KLK5, such that the variant, CDR-grafted, chimeric, humanized, or composite antibody has at least 50%, at least 60%, at least 70%, at least 80%, at least 90%, at least 95% or more binding to KLK5 relative to the original antibody from which it is derived.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal S., et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like hinge sequence. Accordingly, any of the antibodies may include a stabilizing ‘Adair’ mutation.

In some embodiments, an antibody is modified, e.g., modified via glycosylation, phosphorylation, sumoylation, and/or methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or phosphoglycosylation. In some embodiments, the one or more sugar or carbohydrate molecules are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, there are about 1-10, about 1-5, about 5-10, about 1-4, about 1-3, or about 2 sugar molecules. In some embodiments, a glycosylated antibody is fully or partially glycosylated. In some embodiments, an antibody is glycosylated by chemical reactions or by enzymatic means. In some embodiments, an antibody is glycosylated in vitro or inside a cell, which may optionally be deficient in an enzyme in the N- or O-glycosylation pathway, e.g. a glycosyltransferase. In some embodiments, an antibody is functionalized with sugar or carbohydrate molecules as described in International Patent Application Publication WO2014065661, published on May 1, 2014, entitled, “Modified antibody, antibody-conjugate and process for the preparation thereof”.

In some embodiments, any one of the anti-KLK5 antibodies described herein may comprise a signal peptide in the heavy and/or light chain sequence (e.g., a N-terminal signal peptide). In some embodiments, the anti-KLK5 antibody described herein comprises any one of the VH and VL sequences, any one of the IgG heavy chain and light chain sequences, or any one of the F(ab′) heavy chain and light chain sequences described herein, and further comprises a signal peptide (e.g., a N-terminal signal peptide).

### (b) Anti-KLK7 Antibodies

In some embodiments, an antibody targeting KLK7 (referred to as an anti-KLK7 antibody) is an antibody specific for Kallikrein-5 (KLK7). Provided herein, in some aspects, are antibodies that bind to KLK7 (e.g., human KLK7, or mouse KLK7) with high specificity and affinity. In some embodiments, an antibody described herein specifically binds to an epitope of KLK7 that is exposed or becomes exposed to an antibody. In some embodiments, anti-KLK7 antibodies provided herein bind specifically to KLK7 from human, non-human primates, mouse, rat, etc. In some embodiments, anti-KLK7 antibodies provided herein specifically bind to human KLK7. In some embodiments, anti-KLK7 antibodies provided herein specifically bind to mouse KLK7.

Kallikrein-7 is a serine protease that in humans is encoded by the KLK7 gene. KLK7 is characterized as stratum corneum chymotryptic enzyme (SCCE). [It is the seventh member of the human kallikrein family, which includes fifteen homologous serine proteases located on chromosome 19.

KLK7 is secreted as an inactive zymogen (e.g., in the stratum granulosum layer of the epidermis), requiring proteolytic cleavage to be activated. In some embodiments, KLK5 or matriptase activates KLK7. Once active, KLK7 is able to cleave proteins which form the stratum corneum and/or stratum granulosum (e.g., Corneodesmosin (CDSN), desmoglein 1 (DSG1), and desmocollin 1 (DSC1), etc) (see, e.g., Caubet et al. (May 2004). Degradation of corneodesmosome proteins by two serine proteases of the kallikrein family, SCTE/KLK5/hK5 and SCCE/KLK7/hK7. The Journal of Investigative Dermatology. 122 (5): 1235-1244). These proteins constitute the extracellular component of corneodesmosomes, intercellular cohesive structures which link the intermediate filaments of adjacent cells in the stratum corneum. In some embodiments, proteolysis of corneodesmosomes leads to desquamation of the epidermis (i.e., the shedding of corneocytes from the outer layer of the epidermis). In some embodiments, the combined role of KLK5 and KLK7 suggests that KLK skin cascade is responsible for coordinating desquamation. KLK7 is a chymotrypsin-like serine protease, which cleave proteins at the residues tyrosine, phenylalanine or leucine. In some embodiments, dysregulation of KLK7 has been linked to several skin disorders including atopic dermatitis, psoriasis and Netherton syndrome. These diseases are characterized by excessively dry, scaly and inflamed skin, due to a disruption of skin homeostasis and correct barrier function.

In some embodiments, an anti-KLK7 antibody described herein specifically binds to an epitope on human KLK7. Exemplary amino acid sequences of human KLK7 are set forth in NCBI Accession Numbers NP_001193982.1, NP_001230055.1, NP_005037.1, NP_644806.1, and UniProt Accession Numbers: M0QYU8, Q6DTY1, X2J289, X2J4X7, A0A024R4H6, P49862, A0A2H4GDB2, and A0A2H4GDB6, the entire sequences of which are incorporated herein by reference.

In some embodiments, an anti-KLK7 antibody described herein specifically binds to an epitope on mouse KLK7. Exemplary amino acid sequences of mouse KLK7 are set forth in NCBI Accession Numbers NP_036002.1, and UniProt Accession Numbers Q91VE3, the entire sequences of which are incorporated herein by reference.

In some embodiments, an anti-KLK7 antibody described herein specifically binds to an epitope on KLK7 (e.g., the catalytic domain/pocket of human KLK7 or mouse KLK7). In some embodiments, an anti-KLK7 antibody described herein prevents KLK7 (e.g., human or mouse KLK7) from cleaving its substrates. In some embodiments, an anti-KLK7 antibody described herein bind to a fragment of a KLK7 (e.g., human or mouse KLK7). The fragment of KLK7 (e.g., human or mouse) may be between about 5 and about 425 amino acids, between about 10 and about 400 amino acids, between about 50 and about 350 amino acids, between about 100 and about 300 amino acids, between about 150 and about 250 amino acids, between about 200 and about 300 amino acids, between about 75 and about 150 amino acids, between about 25 and about 100 amino acids, between about 10 and about 30 amino acids in length. Not wishing to be bound to any particular theory, and in some embodiments, a heavy chain (HC) complementarity-determining region 3 (CDR3) of any one of the anti-KLK7 antibodies described herein inhibits KLK7 (e.g., human or mouse KLK7) by binding to the catalytic domain/pocket of KLK7.

In some embodiments, an anti-KLK7 antibody described herein inhibits KLK7 protease activity. In some embodiments the anti-KLK7 antibody inhibits KLK7 cleavage of KHLF-AMC with an IC50 of less than 6 nM, less than 5 nM, less than 4 nM, less than 3 nM, less than 2.5 nM, less than 2 nM, or less than 1.5 nM, less than 1 nM, less than 0.5 nM, less than 0.4 nM, less than 0.3 nM, less than 0.2 nM, less than 0.16 nM, less than 0.1 nM or less than 0.05 nM.

In some embodiments, an anti-KLK7 antibody described herein binds specifically to the active form of KLK7. In some embodiments, the anti-KLK7 antibody described herein does not bind to the inactive form of KLK7. In some embodiments, an anti-KLK7 antibody described herein binds specifically to the active site of KLK7. The active site of KLK7 is the site at which the KLK7 substrate molecules bind to undergo cleavage. The active site may also be known as the catalytic domain, or catalytic triad. In some embodiments, the active site (i.e., catalytic domain or catalytic triad) of KLK7 consists of amino acids Ser195, His57, and Asp102 of KLK7 (see, e.g., Goettig et al., Natural and synthetic inhibitors of kallikrein-related peptidases (KLKs), Biochimie. 2010 November; 92(11): 1546-1567).

In some embodiments, antibodies described herein are optimized versions (e.g., affinity matured) of the parental antibody. In some embodiments, an antibody described herein specifically binds a KLK7 (e.g., a human or mouse KLK7) with binding affinity (e.g., as indicated by K_{D}) of at least about 10⁻⁴ M, 10⁻¹ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, an antibody described herein specifically binds a KLK7 (e.g., a human or mouse KLK7) with binding affinity (e.g., as indicated by K_{D}) of between 1×10⁻¹⁰ M and 5×10⁻⁹ M, between 1×10⁻¹⁰ M and 1×10⁻⁹ M, between 5×10⁻¹⁰ and 1×10⁻⁹ M, between 5×10⁻¹¹ and 1×10⁻¹⁰ M, between 1×10⁻¹¹ and 5×10⁻¹⁰ M, or between 5×10⁻¹³ and 1×10⁻¹² M. For example, an antibody of the present disclosure can bind to a KLK7 protein (e.g., human or mouse KLK7) with an affinity between 1 pM and 500 nM, e.g., between 50 pM and 100 nM, between 500 pM and 50 nM, between 1 pM and 100 pM, between 10 pM and 100 pM, between 50 pM and 100 pM, between 100 pM and 500 pM, between 500 pM and 1 nM, between 1 nM and 5 nM, between 1 nM and 10 nM, between 5 nM and 25 nM, between 10 nM and 50 nM between 50 nM and 100 nM, between 100 nM and 500 nM. The disclosure also includes antibodies that compete with any of the antibodies described herein for binding to a KLK7 protein (e.g., human or mouse KLK7) and that have an affinity of 100 nM or lower (e.g., 80 nM or lower, 50 nM or lower, 20 nM or lower, 10 nM or lower, 1 nM or lower, 500 pM or lower, 50 pM or lower, or 5 pM or lower). The affinity and binding kinetics of an antibody can be tested using any suitable method including but not limited to biosensor technology (e.g., OCTET or BIACORE). In some embodiments, antibodies described herein binds to KLK7 with a K_{D} of sub-nanomolar range.

Binding affinity (or binding specificity) can be determined by a variety of methods including equilibrium dialysis, equilibrium binding, gel filtration, ELISA, surface plasmon resonance (SPR), florescent activated cell sorting (FACS) or spectroscopy (e.g., using a fluorescence assay). Exemplary conditions for evaluating binding affinity are in HBS-P buffer (10 mM HEPES pH7.4, 150 mM NaCl, 0.005% (v/v) surfactant P20) and PBS buffer (10 mM P04-3, 137 mM NaCl, and 2.7 mM KCl). These techniques can be used to measure the concentration of bound proteins as a function of target protein concentration. The concentration of bound protein ([[Bound]]) is generally related to the concentration of free target protein ([[Free]]) by the following equation:

$$[ [ Bound ] ] = [ [ Free ] ] / ( Kd + [ [ Free ] ] )$$

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

Exemplary anti-KLK7 antibody sequences (e.g., the heavy chain (HC) and light chain (LC) sequences, heavy chain variable domain (VH) and light chain variable domain (VL), CDR sequences are provided in Table 2a.

TABLE 2a
Examples of anti-KLK7 antibodies

| KLK7 Antibody | | Sequences | SEQ ID NO |
|---|---|---|---|
| KLK7-L1- | HC CDR1 | GTFSNYAIS | 66 |
| Ab1 | HC CDR2 | WISAYSGDTNYAQKLQG | 67 |
| | HC CDR3 | ARAAGYGARHWYFDL | 68 |
| | LC CDR1 | RASQSISSYLN | 69 |
| | LC CDR2 | GASSLQS | 70 |
| | LC CDR3 | DQSYSTPPIT | 71 |
| | VH | QVQLVQSGAEVKKPGSSVKVSCKASGGTFSNYAISWVRQAPGQ | 72 |
| | | GLEWMGWISAYSGDTNYAQKLQGRVTMTTDTSTSTAYMELRSL | |
| | | RSDDTAVYYCARAAGYGARHWYFDLWGQGTMVTVSS | |
| | VL | DIQMTQSPSSLSASVGDRVTITCRASQSISSYLNWYQQKPGKA | 73 |
| | | PKLLIYGASSLQSGVPSRFSGSGSGTDFTLTISSLQPEDFATY | |
| | | YCDQSYSTPPITFGGGTKVEIK | |
| KLK7-L1- | HC CDR1 | YTFGPYGIS | 74 |
| Ab2 | HC CDR2 | WISAYSGDTNYAQKLQG | 67 |
| | HC CDR3 | ARAAGYGARWWYFDL | 75 |
| | LC CDR1 | RASQSISSYLN | 69 |
| | LC CDR2 | GASSLQS | 70 |
| | LC CDR3 | DGSYSTPPIT | 198 |
| | VH | QVQLVQSGAEVKKPGASVKVSCKASGYTFGPYGISWVRQAPGQ | 199 |
| | | GLEWMGWISAYSGDTNYAQKLQGRVTMTTDTSTSTAYMELRSL | |
| | | RSDDTAVYYCARAAGYGARWWYFDLWGQGTMVTVSS | |
| | VL | DIQMTQSPSSLSASVGDRVTITCRASQSISSYLNWYQQKPGKA | 200 |
| | | PKLLIYGASSLQSGVPSRFSGSGSGTDFTLTISSLQPEDFATY | |
| | | YCDGSYSTPPITFGGGTKVEIK | |
| KLK7-L1- | HC CDR1 | YTFTSEGIH | 284 |
| Ab3 | HC CDR2 | SISASSGDTNYAQELQG | 201 |
| | HC CDR3 | ARAAGYGARHWYFDL | 68 |
| | LC CDR1 | RASQSISSYLN | 69 |
| | LC CDR2 | GASSLQS | 70 |
| | LC CDR3 | DQFYSTPPIT | 76 |
| | VH | QVQLVQSGAEVKKPGASVKVSCKASGYTFTSEGIHWVRQAPGQ | 77 |
| | | GLEWMGSISASSGDTNYAQELQGRVTMTTDTSTSTAYMELRSL | |
| | | RSDDTAVYYCARAAGYGARHWYFDLWGQGTMVTVSS | |
| | VL | DIQMTQSPSSLSASVGDRVTITCRASQSISSYLNWYQQKPGKA | 78 |
| | | PKLLIYGASSLQSGVPSRFSGSGSGTDFTLTISSLQPEDFATY | |
| | | YCDQFYSTPPITFGGGTKVEIK | |
| KLK7-L1- | HC CDR1 | YTFTSEGIH | 284 |
| Ab4 | HC CDR2 | SISASSGDTNYAQKLQG | 79 |
| | HC CDR3 | ARAAGYGARHWYFDL | 68 |
| | LC CDR1 | RASQSISSYLN | 69 |
| | LC CDR2 | GASSLQS | 70 |
| | LC CDR3 | DQFYSTPPIT | 76 |
| | VH | QVQLVQSGAEVKKPGASVKVSCKASGYTFTSEGIHWVRQAPGQ | 80 |
| | | GLEWMGSISASSGDTNYAQKLQGRVTMTTDTSTSTAYMELRSL | |
| | | RSDDTAVYYCARAAGYGARHWYFDLWGQGTMVTVSS | |
| | VL | DIQMTQSPSSLSASVGDRVTITCRASQSISSYLNWYQQKPGKA | 78 |
| | | PKLLIYGASSLQSGVPSRFSGSGSGTDFTLTISSLQPEDFATY | |
| | | YCDQFYSTPPITFGGGTKVEIK | |
| KLK7-L1- | HC CDR1 | YTFTSEGIH | 284 |
| Ab5 | HC CDR2 | SISASSGDTNYAQKLQG | 79 |
| | HC CDR3 | ARAAGYGARHWYFDL | 68 |
| | LC CDR1 | RASQSISSYLN | 69 |
| | LC CDR2 | GASSLQS | 70 |
| | LC CDR3 | DQWYSTPPIT | 81 |
| | VH | QVQLVQSGAEVKKPGASVKVSCKASGYTFTSEGIHWVRQAPGQ | 80 |
| | | GLEWMGSISASSGDTNYAQKLQGRVTMTTDTSTSTAYMELRSL | |
| | | RSDDTAVYYCARAAGYGARHWYFDLWGQGTMVTVSS | |
| | VL | DIQMTQSPSSLSASVGDRVTITCRASQSISSYLNWYQQKPGKA | 82 |
| | | PKLLIYGASSLQSGVPSRFSGSGSGTDFTLTISSLQPEDFATY | |
| | | YCDQWYSTPPITFGGGTKVEIK | |
| KLK7-L1- | HC CDR1 | YTFTSEGIH | 284 |
| Ab6 | HC CDR2 | SISASSGDTNYAQKLQG | 79 |
| | HC CDR3 | ARAAGYGARSWYFDL | 83 |
| | LC CDR1 | RASQSISSYLN | 69 |
| | LC CDR2 | GASSLQS | 70 |
| | LC CDR3 | DQFYSTPPIT | 76 |
| | VH | QVQLVQSGAEVKKPGASVKVSCKASGYTFTSEGIHWVRQAPGQ | 84 |
| | | GLEWMGSISASSGDTNYAQKLQGRVTMTTDTSTSTAYMELRSL | |
| | | RSDDTAVYYCARAAGYGARSWYFDLWGQGTMVTVSS | |
| | VL | DIQMTQSPSSLSASVGDRVTITCRASQSISSYLNWYQQKPGKA | 78 |
| | | PKLLIYGASSLQSGVPSRFSGSGSGTDFTLTISSLQPEDFATY | |
| | | YCDQFYSTPPITFGGGTKVEIK | |
| KLK7-L1- | HC CDR1 | YTFTSEGIH | 284 |
| Ab7 | HC CDR2 | SISASSGDTNYAQKLQG | 79 |
| | HC CDR3 | ARGAGYGARDWYFDL | 85 |
| | LC CDR1 | RASQSISSYLN | 69 |
| | LC CDR2 | GASSLQS | 70 |
| | LC CDR3 | DQRYSTPPIT | 86 |
| | VH | QVQLVQSGAEVKKPGASVKVSCKASGYTFTSEGIHWVRQAPGQ | 87 |
| | | GLEWMGSISASSGDTNYAQKLQGRVTMTTDTSTSTAYMELRSL | |
| | | RSDDTAVYYCARGAGYGARDWYFDLWGQGTMVTVSS | |
| | VL | DIQMTQSPSSLSASVGDRVTITCRASQSISSYLNWYQQKPGKA | 88 |
| | | PKLLIYGASSLQSGVPSRFSGSGSGTDFTLTISSLQPEDFATY | |
| | | YCDQRYSTPPITFGGGTKVEIK | |
| KLK7-L1- | HC CDR1 | YTFTSEGIH | 284 |
| Ab8 | HC CDR2 | SISASSGDTNYAQKLQG | 79 |
| | HC CDR3 | ARSAGYGARHWYFDL | 89 |
| | LC CDR1 | RASQSISSYLN | 69 |
| | LC CDR2 | GASSLQS | 70 |
| | LC CDR3 | DQWYSTPPIT | 81 |
| | VH | QVQLVQSGAEVKKPGASVKVSCKASGYTFTSEGIHWVRQAPGQ | 285 |
| | | GLEWMGSISASSGDTNYAQKLQGRVTMTTDTSTSTAYMELRSL | |
| | | RSDDTAVYYCARSAGYGARHWYFDLWGQGTMVTVSS | |
| | VL | DIQMTQSPSSLSASVGDRVTITCRASQSISSYLNWYQQKPGKA | 82 |
| | | PKLLIYGASSLQSGVPSRFSGSGSGTDFTLTISSLQPEDFATY | |
| | | YCDQWYSTPPITFGGGTKVEIK | |
| KLK7-L2- | HC CDR1 | GSISSGGYYWS | 90 |
| Ab1 | HC CDR2 | SIYYSGSTYYNPSLKS | 91 |
| | HC CDR3 | ARAKQLTTHRYFDY | 92 |
| | LC CDR1 | QASQDIANYLN | 93 |
| | LC CDR2 | DASNLET | 94 |
| | LC CDR3 | QQASDLPFT | 95 |
| | VH | QVQLQESGPGLVKPSQTLSLTCTVSGGSISSGGYYWSWIRQHP | 98 |
| | | GKGLEWIGSIYYSGSTYYNPSLKSRVTISVDTSKNQFSLKLSS | |
| | | VTAADTAVYYCARAKQLTTHRYFDYWGQGTLVTVSS | |
| | VL | DIQMTQSPSSLSASVGDRVTITCQASQDIANYLNWYQQKPGKA | 97 |
| | | PKLLIYDASNLETGVPSRFSGSGSGTDFTFTISSLQPEDIATY | |
| | | YCQQASDLPFTFGGGTKVEIK | |
| KLK7-L2- | HC CDR1 | GSISSGGYYWH | 96 |
| Ab2 | HC CDR2 | YIYRSGYTYYNPSLKS | 109 |
| | HC CDR3 | ARAKQLTTHRYFDY | 92 |
| | LC CDR1 | QASQDIANYLN | 93 |
| | LC CDR2 | DASNLET | 94 |
| | LC CDR3 | QQASDLPFT | 95 |
| | VH | QVQLQESGPGLVKPSQTLSLTCTVSGGSISSGGYYWHWIRQHP | 153 |
| | | GKGLEWIGYIYRSGYTYYNPSLKSRVTISVDTSKNQFSLKLSS | |
| | | VTAADTAVYYCARAKQLTTHRYFDYWGQGTLVTVSS | |
| | VL | DIQMTQSPSSLSASVGDRVTITCQASQDIANYLNWYQQKPGKA | 97 |
| | | PKLLIYDASNLETGVPSRFSGSGSGTDFTFTISSLQPEDIATY | |
| | | YCQQASDLPFTFGGGTKVEIK | |
| KLK7-L2- | HC CDR1 | GSISSGGYSWN | 99 |
| Ab3 | HC CDR2 | SIYRSGSTYYNPSLKS | 100 |
| | HC CDR3 | ARAKALTTHRYVDY | 101 |
| | LC CDR1 | QASQDIANYLN | 93 |
| | LC CDR2 | DASNLET | 94 |
| | LC CDR3 | QQARDLPFT | 102 |
| | VH | QVQLQESGPGLVKPSQTLSLTCTVSGGSISSGGYSWNWIRQHP | 103 |
| | | GKGLEWIGSIYRSGSTYYNPSLKSRVTISVDTSKNQFSLKLSS | |
| | | VTAADTAVYYCARAKALTTHRYVDYWGQGTLVTVSS | |
| | VL | DIQMTQSPSSLSASVGDRVTITCQASQDIANYLNWYQQKPGKA | 104 |
| | | PKLLIYDASNLETGVPSRFSGSGSGTDFTFTISSLQPEDIATY | |
| | | YCQQARDLPFTFGGGTKVEIK | |
| KLK7-L2- | HC CDR1 | GSISSGGYSWN | 99 |
| Ab4 | HC CDR2 | SIYRSGSTYYNPSLKS | 100 |
| | HC CDR3 | ARAKQLTLTRYFDY | 105 |
| | LC CDR1 | QASQDIANYLN | 93 |
| | LC CDR2 | DASNLET | 94 |
| | LC CDR3 | QQASQLPFT | 106 |
| | VH | QVQLQESGPGLVKPSQTLSLTCTVSGGSISSGGYSWNWIRQHP | 107 |
| | | GKGLEWIGSIYRSGSTYYNPSLKSRVTISVDTSKNQFSLKLSS | |
| | | VTAADTAVYYCARAKQLTLTRYFDYWGQGTLVTVSS | |
| | VL | DIQMTQSPSSLSASVGDRVTITCQASQDIANYLNWYQQKPGKA | 108 |
| | | PKLLIYDASNLETGVPSRFSGSGSGTDFTFTISSLQPEDIATY | |
| | | YCQQASQLPFTFGGGTKVEIK | |
| KLK7-L2- | HC CDR1 | GSISSGGYYWH | 96 |
| Ab5 | HC CDR2 | YIYRSGYTYYNPSLKS | 109 |
| | HC CDR3 | ARAKQLTTSRYFDY | 110 |
| | LC CDR1 | QASQDIANYLN | 93 |
| | LC CDR2 | DASNLET | 94 |
| | LC CDR3 | QQASDLPFT | 95 |
| | VH | QVQLQESGPGLVKPSQTLSLTCTVSGGSISSGGYYWHWIRQHP | 111 |
| | | GKGLEWIGYIYRSGYTYYNPSLKSRVTISVDTSKNQFSLKLSS | |
| | | VTAADTAVYYCARAKQLTTSRYFDYWGQGTLVTVSS | |
| | VL | DIQMTQSPSSLSASVGDRVTITCQASQDIANYLNWYQQKPGKA | 97 |
| | | PKLLIYDASNLETGVPSRFSGSGSGTDFTFTISSLQPEDIATY | |
| | | YCQQASDLPFTFGGGTKVEIK | |
| KLK7-L3- | HC CDR1 | GTFSNYAIS | 66 |
| Ab1 | HC CDR2 | GIIPIFGTANYAQKFQG | 112 |
| | HC CDR3 | ARGAPNYYGSSSSRGIAFDI | 113 |
| | LC CDR1 | RASQGISSWLA | 4 |
| | LC CDR2 | AASSLQS | 5 |
| | LC CDR3 | VQANAFPYT | 114 |
| | VH | QVQLVQSGAEVKKPGSSVKVSCKASGGTFSNYAISWVRQAPGQ | 115 |
| | | GLEWMGGIIPIFGTANYAQKFQGRVTITADESTSTAYMELSSL | |
| | | RSEDTAVYYCARGAPNYYGSSSSRGIAFDIWGQGTMVTVSS | |
| | VL | DIQMTQSPSSVSASVGDRVTITCRASQGISSWLAWYQQKPGKA | 116 |
| | | PKLLIYAASSLQSGVPSRFSGSGSGTDFTLTISSLQPEDFATY | |
| | | YCVQANAFPYTFGGGTKVEIK | |
| KLK7-L3- | HC CDR1 | GTENNYAIS | 117 |
| Ab2 | HC CDR2 | AIIPIFGVANYAQKFQG | 118 |
| | HC CDR3 | ARGAPNYYGAQSSRGIAFDI | 119 |
| | LC CDR1 | RASQGISSWLA | 4 |
| | LC CDR2 | AASSLQS | 5 |
| | LC CDR3 | VQANAFPYK | 120 |
| | VH | QVQLVQSGAEVKKPGSSVKVSCKASGGTFNNYAISWVRQAPGQ | 121 |
| | | GLEWMGAIIPIFGVANYAQKFQGRVTITADESTSTAYMELSSL | |
| | | RSEDTAVYYCARGAPNYYGAQSSRGIAFDIWGQGTMVTVSS | |
| | VL | DIQMTQSPSSVSASVGDRVTITCRASQGISSWLAWYQQKPGKA | 122 |
| | | PKLLIYAASSLQSGVPSRFSGSGSGTDFTLTISSLQPEDFATY | |
| | | YCVQANAFPYKFGGGTKVEIK | |
| KLK7-L3- | HC CDR1 | GTFSSHAIT | 123 |
| Ab3 | HC CDR2 | GIIPIFGFANYAQKFQG | 124 |
| | HC CDR3 | ARGAPNYYGSASSRGIAFDI | 125 |
| | LC CDR1 | RASQGISSWLA | 4 |
| | LC CDR2 | AASSLQS | 5 |
| | LC CDR3 | VQANAFPYS | 126 |
| | VH | QVQLVQSGAEVKKPGSSVKVSCKASGGTFSSHAITWVRQAPGQ | 127 |
| | | GLEWMGGIIPIFGFANYAQKFQGRVTITADESTSTAYMELSSL | |
| | | RSEDTAVYYCARGAPNYYGSASSRGIAFDIWGQGTMVTVSS | |
| | VL | DIQMTQSPSSVSASVGDRVTITCRASQGISSWLAWYQQKPGKA | 128 |
| | | PKLLIYAASSLQSGVPSRFSGSGSGTDFTLTISSLQPEDFATY | |
| | | YCVQANAFPYSFGGGTKVEIK | |
| KLK7-L4- | HC CDR1 | GSISSSSYYWG | 19 |
| Ab1 | HC CDR2 | SISYSGSTYYNPSLKS | 20 |
| | HC CDR3 | ARGAPTYFGAASHYYYYMDV | 129 |
| | LC CDR1 | RASQSISSWLA | 130 |
| | LC CDR2 | KASSLES | 131 |
| | LC CDR3 | EQYGSYSPLT | 132 |
| | VH | QLQLQESGPGLVKPSETLSLTCTVSGGSISSSSYYWGWIRQPP | 133 |
| | | GKGLEWIGSISYSGSTYYNPSLKSRVTISVDTSKNQFSLKLSS | |
| | | VTAADTAVYYCARGAPTYFGAASHYYYYMDVWGKGTTVTVSS | |
| | VL | DIQMTQSPSTLSASVGDRVTITCRASQSISSWLAWYQQKPGKA | 134 |
| | | PKLLIYKASSLESGVPSRFSGSGSGTEFTLTISSLQPDDFATY | |
| | | YCEQYGSYSPLTFGGGTKVEIK | |
| KLK7-L4- | HC CDR1 | GSISSSPYNWV | 135 |
| Ab2 | HC CDR2 | SILHSGSTYYNPSLKS | 136 |
| | HC CDR3 | ARGVPTYWGAASHYYYYMDV | 137 |
| | LC CDR1 | RASQSISSWLA | 130 |
| | LC CDR2 | KASSLES | 131 |
| | LC CDR3 | EQYGSESPLT | 138 |
| | VH | QLQLQESGPGLVKPSETLSLTCTVSGGSISSSPYNWVWIRQPP | 139 |
| | | GKGLEWIGSILHSGSTYYNPSLKSRVTISVDTSKNQFSLKLSS | |
| | | VTAADTAVYYCARGVPTYWGAASHYYYYMDVWGKGTTVTVSS | |
| | VL | DIQMTQSPSTLSASVGDRVTITCRASQSISSWLAWYQQKPGKA | 140 |
| | | PKLLIYKASSLESGVPSRFSGSGSGTEFTLTISSLQPDDFATY | |
| | | YCEQYGSESPLTFGGGTKVEIK | |
| KLK7-L5- | HC CDR1 | YTFTGYYMH | 38 |
| Ab1 | HC CDR2 | SINPSSGGTNYAQKFQG | 141 |
| | HC CDR3 | ARGGPSTWYGGSAYYYYGMDV | 142 |
| | LC CDR1 | RASQSVSSDYLA | 143 |
| | LC CDR2 | GASSRAT | 23 |
| | LC CDR3 | QQYANLWT | 144 |
| | VH | QVQLVQSGAEVKKPGASVKVSCKASGYTFTGYYMHWVRQAPGQ | 145 |
| | | GLEWMGSINPSSGGTNYAQKFQGRVTMTRDTSISTAYMELSRL | |
| | | RSDDTAVYYCARGGPSTWYGGSAYYYYGMDVWGQGTTVTVSS | |
| | VL | EIVLTQSPGTLSLSPGERATLSCRASQSVSSDYLAWYQQKPGQ | 146 |
| | | APRLLIYGASSRATGIPDRFSGSGSGTDFTLTISRLEPEDFAV | |
| | | YYCQQYANLWTFGGGTKVEIK | |
| KLK7-L5- | HC CDR1 | YTFAGYDMH | 147 |
| Ab2 | HC CDR2 | SINPTSGRTNYAQKFQG | 148 |
| | HC CDR3 | ARGGPSTWYGGSAYTYYGADV | 149 |
| | LC CDR1 | RASQSVSSDYLA | 143 |
| | LC CDR2 | GASSRAT | 23 |
| | LC CDR3 | QQYAPLWT | 150 |
| | VH | QVQLVQSGAEVKKPGASVKVSCKASGYTFAGYDMHWVRQAPGQ | 151 |
| | | GLEWMGSINPTSGRTNYAQKFQGRVTMTRDTSISTAYMELSRL | |
| | | RSDDTAVYYCARGGPSTWYGGSAYTYYGADVWGQGTTVTVSS | |
| | VL | EIVLTQSPGTLSLSPGERATLSCRASQSVSSDYLAWYQQKPGQ | 152 |
| | | APRLLIYGASSRATGIPDRFSGSGSGTDFTLTISRLEPEDFAV | |
| | | YYCQQYAPLWTFGGGTKVEIK | |

In some embodiments, certain amino acid positions in an antibody described herein (e.g., amino acids in the VH/VL regions and/or CDR regions) are substitutable and the substitution results in an antibody with substantially similar binding and biological activities (e.g., substantially similar binding affinity, binding specificity, protease activity inhibitory activity, anti-inflammatory activity, or a combination thereof) as the reference antibody. In order to identify a substitutable position of an antibody, the amino acid sequence of that antibody is compared to the sequences of other antibodies belonging to the same group as that antibody. If the identity of that amino acid varies between the different related antibodies of a group at any particular position, that position is a substitutable position of the antibody. In other words, a substitutable position is a position in which the identity of the amino acid varies between the related antibodies. Positions that contain a constant amino acid are not substitutable positions.

In some embodiments, the above method may be employed to provide a consensus antibody sequence. In such a consensus sequence, a non-substitutable position is indicated by the amino acid present at that position, and a substitutable position is indicated as an “X”.

Depending on how the antibodies are to be employed, X may be a) any amino acid, b) any amino acid present at that position in any of the related antibodies in the group or a conservatively substituted variant thereof or c) any amino acid present at that position in any of the related antibodies in the group. Any antibody having a sequence that is encompassed by the consensus should bind to the same antigen as any of the related antibodies.

In some embodiments, the method described above may be employed in methods of designing and making a variant of a parental antibody that at least maintains (i.e. maintains or increases) the antigen binding activity of the parental antibody. Because antibodies containing substitutions at substitutable positions have already been produced and tested, substitutions at those positions can be made in the knowledge that they should not significantly decrease the binding activity of the antibody. In general, an antibody variant of a parental antibody has an antigen binding affinity that is at least 10%, at least 20%, at least 30%, at least 40%, at least 50%, at least 60%, at least 70%, at least 80%, at least 90% or at least 100% (e.g., at least 150%, at least 200%, at least 500%, at least 1000%, usually up to at least 10,000%) of the binding affinity of the parental antibody to a particular antigen.

In some embodiments, a substitutable position of a parental antibody may be substituted by a) any of the 20 naturally occurring amino acids to produce random substitutions, b) an amino acid having biochemical properties similar to the amino acid already present at the substitutable position to produce conservative substitutions, c) an amino acid that is present at the same position in a related antibody to produce a directed substitution, or d) an amino acid that is present at the same position in a similar human antibody to produce a humanizing substitution. A substitution may be made at any part of an antibody variable region, including any framework region or CDR. In certain embodiments, a single substitutable amino acid may be substituted. However, in other embodiments, a plurality of substitutable amino acids (e.g., up to about 5 or 10 or more) may be substituted. In particular embodiments, the type of substitution that can be made at each substitutable position may be indicated by the types of amino acids present at that position in the related antibodies. For example, if unrelated amino acids (e.g., Ala, Gly, Cys, Glu and Thr) are present at a certain position of a group of related antibodies, then any amino acid could be substituted at that position without significantly reducing binding activity of the antibody. Exemplary amino acids substations of an anti-KLK7 antibody described herein are set forth in Table 1b:

TABLE 2b
Exemplary Amino Acids Substitutions in anti-KLK7 antibodies

| KLK7 Antibody | | Sequences | SEQ ID NO |
|---|---|---|---|
| KLK7-L1- | HC CDR1 | X₁TFX₂X₃X₄X₅IX₆ | 264 |
| Ab0 | | X₁ is G or Y | |
| | | X₂ is S, G, or T | |
| | | X₃ is N, P or S | |
| | | X₄ is Y or E | |
| | | X₅ is A or G | |
| | | X₆ is S or H | |
| | HC CDR2 | X₇ISAX₈SGDTNYAQX₉LQG | 265 |
| | | X₇ is W or S | |
| | | X₈ is Y or S | |
| | | X₉ is K or E | |
| | HC CDR3 | ARX₁₀AGYGARX₁₁WYFDL | 266 |
| | | X₁₀ is A, G, or S | |
| | | X₁₁ is H, W, S, or D | |
| | LC CDR1 | RASQSISSYLN | 69 |
| | LC CDR2 | GASSLQS | 70 |
| | LC CDR3 | DX₁₂X₁₃YSTPPIT | 267 |
| | | X₁₂ is Q or G | |
| | | X₁₃ is S, F, W, or R | |
| KLK7-L2- | HC CDR1 | GSISSGGYX₁WX₂ | 268 |
| Ab0 | | X₁ is Y or S | |
| | | X₂ is S, H or N | |
| | HC CDR2 | X₃IYX₄SGX₅TYYNPSLKS | 269 |
| | | X₃ is S or Y | |
| | | X₄ is Y or R | |
| | | X₅ is S or Y | |
| | HC CDR3 | ARAKX₆LTX₇X₈RYX₉DY | 270 |
| | | X₆ is Q or A | |
| | | X₇ is T or L | |
| | | X₈ is H, T or S | |
| | | X₉ is F or V | |
| | LC CDR1 | QASQDIANYLN | 93 |
| | LC CDR2 | DASNLET | 94 |
| | LC CDR3 | QQAX₉X₁₀LPFT | 271 |
| | | X₉ is S or R | |
| | | X₁₀ is D or Q | |
| KLK7-L3- | HC CDR1 | GTFX₁X₂X₃AIX₄ | 272 |
| Ab0 | | X₁ is S or N | |
| | | X₂ is N or S | |
| | | X₃ is Y or H | |
| | | X₄ is S or T | |
| | HC CDR2 | X₅IIPIFGX6ANYAQKFQG | 273 |
| | | X₅ is G or A | |
| | | X₆ is T, V or F | |
| | HC CDR3 | ARGAPNYYGX₇X₈SSRGIAFDI | 274 |
| | | X₇ is S or A | |
| | | X₈ is S, Q or A | |
| | LC CDR1 | RASQGISSWLA | 4 |
| | LC CDR2 | AASSLQS | 5 |
| | LC CDR3 | VQANAFPYX₉ | 275 |
| | | X₉ is T, K or S | |
| KLK7-L4- | HC CDR1 | GSISSSX₁YX₂WX₃ | 276 |
| Ab0 | | X₁ is S or P | |
| | | X₂ is Y or N | |
| | | X₃ is G or V | |
| | HC CDR2 | SIX₄X₅SGSTYYNPSLKS | 277 |
| | | X₄ is S or L | |
| | | X₅ is Y or H | |
| | HC CDR3 | ARGX₆PTYX₇GAASHYYYYMDV | 278 |
| | | X₆ is A or V | |
| | | X₇ is F or W | |
| | LC CDR1 | RASQSISSWLA | 130 |
| | LC CDR2 | KASSLES | 131 |
| | LC CDR3 | EQYGSX₈SPLT | 279 |
| | | X₈ is Y or E | |
| KLK7-L5- | HC CDR1 | YTFX₁GYX₂MH | 280 |
| Ab0 | | X₁ is T or A | |
| | | X₂ is Y or D | |
| | HC CDR2 | SINPX₃SGX₄TNYAQKFQG | 281 |
| | | X₃ is S or T | |
| | | X₄ is G or R | |
| | HC CDR3 | ARGGPSTWYGGSAYX₅YYGX₆DV | 282 |
| | | X₅ is Y or T | |
| | | X₆ is M or A | |
| | LC CDR1 | RASQSVSSDYLA | 143 |
| | LC CDR2 | GASSRAT | 23 |
| | LC CDR3 | QQYAX₇LWT | 283 |
| | | X₇ is N or P | |

In some embodiments, an antibody of the present disclosure comprises a HC CDR1 comprising the amino acid sequence of X₁TFX₂X₃X₄X₅IX₆, in which X₁ is G or Y, X₂ is S, G, or T, X₃ is N, P or S, X₄ is Y or E, X₅ is A or G, or X₆ is S or H; a HC CDR2 comprising the amino acid sequence of X₇ISAX₈SGDTNYAQX₉LQG, in which X₇ is W or 5, X₈ is Y or 5, or X₉ is K or E; a HC CDR3 comprising the ARX₁₀AGYGARX₁₁WYFDL, in which X₁₀ is A, G, or 5, or X₁₁ is H, W, 5, or D; a LC CDR1 comprising the amino acid sequence of SEQ ID NO: 69; a LC CDR2 comprising the amino acid sequence of SEQ ID NO: 70; and/or a LC CDR3 comprising the amino acid sequence of DX₁₂X₁₃YSTPPIT, in which X₁₂ is Q or G, or X₁₃ is 5, F, W, or R.

In some embodiments, an antibody of the present disclosure comprises a HC CDR1 comprising the amino acid sequence of GSISSGGYX₁WX₂, X₁ is Y or S, or X₂ is S, H or N; a HC CDR2 comprising the amino acid sequence of X₃IYX₄SGX₅TYYNPSLKS, in which X₃ is S or Y, X₄ is Y or R, or X₅ is S or Y; a HC CDR3 comprising the amino acid sequence of ARAKX₆LTX₇X₈RYX₉DY, in which X₆ is Q or A, X₇ is T or L, X₈ is H, T or S, or X₉ is F or V; a LC CDR1 comprising the amino acid sequence of SEQ ID NO: 93; a LC CDR2 comprising the amino acid sequence of SEQ ID NO: 94; and/or a LC CDR3 comprising the amino acid sequence of QQAX₉X₁₀LPFT, in which X₉ is S or R, or X₁₀ is D or Q.

In some embodiments, an antibody of the present disclosure comprises a HC CDR1 comprising the amino acid sequence of GTFX₁X₂X₃AIX₄, in which X₁ is S or N, X₂ is N or S, X₃ is Y or H, or X₄ is S or T; a HC CDR2 comprising the amino acid sequence of X₅IIPIFGX₆ANYAQKFQG, in which X₅ is G or A, or X₆ is T, V or F; a HC CDR3 comprising the amino acid sequence of ARGAPNYYGX₇X₈SSRGIAFDI, in which X₇ is S or A, or X₈ is S, Q or A; a LC CDR1 comprising the amino acid sequence of SEQ ID NO: 4; a LC CDR2 comprising the amino acid sequence of SEQ ID NO: 5; and/or a LC CDR3 comprising the amino acid sequence of VQANAFPYX₉, in which X₉ is T, K or S.

In some embodiments, an antibody of the present disclosure comprises a HC CDR1 comprising the amino acid sequence of GSISSSX₁YX₂WX₃, in which X₁ is S or P, X₂ is Y or N, or X₃ is G or V; a HC CDR2 comprising the amino acid sequence of SIX₄X₅SGSTYYNPSLKS, in which X₄ is S or L, or X₅ is Y or H; a HC CDR3 comprising the amino acid sequence of ARGX₆PTYX₇GAASHYYYYMDV, in which X₆ is A or V, X₇ is F or W; a LC CDR1 comprising the amino acid sequence of SEQ ID NO: 130; a LC CDR2 comprising the amino acid sequence of SEQ ID NO: 131; and/or a LC CDR3 comprising the amino acid sequence of EQYGSX₈SPLT, in which X₈ is Y or E.

In some embodiments, an antibody of the present disclosure comprises a HC CDR1 comprising the amino acid sequence of YTFX₁GYX₂MH, in which X₁ is T or A, or X₂ is Y or D; a HC CDR2 comprising the amino acid sequence of SINPX₃SGX₄TNYAQKFQG, in which X₃ is S or T, or X₄ is G or R; a HC CDR3 comprising the amino acid sequence of ARGGPSTWYGGSAYX₅YYGX₆DV, in which X₅ is Y or T, or X₆ is M or A; a LC CDR1 comprising the amino acid sequence of SEQ ID NO: 143; a LC CDR2 comprising the amino acid sequence of SEQ ID NO: 23; and/or a LC CDR3 comprising the amino acid sequence of QQYAX₇LWT, in which X₇ is N or P.

In some embodiments, an antibody of the present disclosure comprises one or more of the HC CDRs (e.g., HC CDR1, HC CDR2, or HC CDR3) amino acid sequences from any one of the anti-KLK7 antibodies selected from Tables 2a and 2b. In some embodiments, an antibody of the present disclosure comprises the HC CDR3 amino acid sequences from any one of the anti-KLK7 antibodies selected from Tables 2a and 2b. In some embodiments, an antibody of the present disclosure comprise the HC CDR1, HC CDR2, and HC CDR3 as provided for any one of the antibodies elected from Tables 2a and 2b. In some embodiments, an antibody of the present disclosure comprises the LC CDR3 amino acid sequences from any one of the anti-KLK7 antibodies selected from Tables 2a and 2b. In some embodiments, an antibody of the present disclosure comprises one or more of the LC CDRs (e.g., LC CDR1, LC CDR2, or LC CDR3) amino acid sequences from any one of the anti-KLK7 antibodies selected from Tables 2a and 2b. In some embodiments, an antibody of the present disclosure comprise the LC CDR1, LC CDR2, and LC CDR3 s provided for any one of the anti-KLK7 antibodies selected from Tables 2a and 2b.

In some embodiments, an antibody of the present disclosure comprises the HC CDR1, HC CDR2, HC CDR3, LC CDR1, LC CDR2, and LC CDR3 as provided for any one of the anti-KLK7 antibodies selected from Tables 2a and 2b. In some embodiments, antibody heavy and/or light chain CDR3 domains may play a particularly important role in the binding specificity/affinity of an antibody for an antigen. Accordingly, an antibody of the disclosure may include at least the heavy and/or light chain CDR3s of any one of the anti-KLK7 antibodies selected from Tables 2a and 2b.

Also within the scope of the present disclosure are variants of any of the exemplary anti-KLK7 antibodies as disclosed herein. A variant may contain one or more amino acid residue variations in the VH and/or VL, or in one or more of the HC CDRs and/or one or more of the LC CDRs as relative to the reference antibody, while retaining substantially similar binding and biological activities (e.g., substantially similar binding affinity, binding specificity, protease activity inhibitory activity, anti-inflammatory activity, or a combination thereof) as the reference antibody.

In some embodiments, an antibody of the disclosure have one or more CDRs (e.g., HC CDR or LC CDR) sequences substantially similar to any of the HC CDR1, HC CDR2, HC CDR3, LC CDR1, LC CDR2, and/or LC CDR3 sequences from one of the anti-KLK7 antibodies selected from Tables 2a and 2b. In some embodiments, the position of one or more CDRs along the VH (e.g., HC CDR1, HC CDR2, or HC CDR3) and/or VL (e.g., LC CDR1, LC CDR2, or LC CDR3) region of an antibody described herein can vary by one, two, three, four, five, or six amino acid positions so long as specific binding to KLK7 (e.g., human or mouse KLK7) is maintained (e.g., substantially maintained, for example, at least 50%, at least 60%, at least 70%, at least 80%, at least 90%, at least 95% of the binding of the original antibody from which it is derived). For example, in some embodiments, the position defining a CDR of any antibody described herein can vary by shifting the N-terminal and/or C-terminal boundary of the CDR by one, two, three, four, five, or six amino acids, relative to the CDR position of any one of the antibodies described herein, so long as specific binding to KLK7 (e.g., human or mouse KLK7) is maintained (e.g., substantially maintained, for example, at least 50%, at least 60%, at least 70%, at least 80%, at least 90%, at least 95% of the binding of the original antibody from which it is derived). In another embodiment, the length of one or more CDRs along the VH (e.g., HC CDR1, HC CDR2, or HC CDR3) and/or VL (e.g., LC CDR1, LC CDR2, or LC CDR3) region of an antibody described herein can vary (e.g., be shorter or longer) by one, two, three, four, five, or more amino acids, so long as immunospecific binding to KLK7 (e.g., human or mouse KLK7) is maintained (e.g., substantially maintained, for example, at least 50%, at least 60%, at least 70%, at least 80%, at least 90%, at least 95% of the binding of the original antibody from which it is derived).

Accordingly, in some embodiments, a HC CDR1, HC CDR2, HC CDR3, LC CDR1, LC CDR2, and/or LC CDR3 described herein may be one, two, three, four, five or more amino acids shorter than one or more of the CDRs described herein (e.g., CDRS from any of the anti-KLK7 antibodies selected from Tables 2a and 2b) so long as specific binding to KLK7 (e.g., human or mouse KLK7) is maintained (e.g., substantially maintained, for example, at least 50%, at least 60%, at least 70%, at least 80%, at least 90%, at least 95% relative to the binding of the original antibody from which it is derived). In some embodiments, a HC CDR1, HC CDR2, HC CDR3, LC CDR1, LC CDR2, and/or LC CDR3 described herein may be one, two, three, four, five or more amino acids longer than one or more of the CDRs described herein (e.g., CDRS from any of the anti-KLK7 antibodies selected from Tables 2a and 2b) so long as specific binding to KLK7 (e.g., human or mouse KLK7) is maintained (e.g., substantially maintained, for example, at least 50%, at least 60%, at least 70%, at least 80%, at least 90%, at least 95% relative to the binding of the original antibody from which it is derived). In some embodiments, the amino portion of a HC CDR1, HC CDR2, HC CDR3, LC CDR1, LC CDR2, and/or LC CDR3 described herein can be extended by one, two, three, four, five or more amino acids compared to one or more of the CDRs described herein (e.g., CDRS from any of the anti-KLK7 antibodies selected from Tables 2a and 2b) so long as specific binding to KLK7 (e.g., human or mouse KLK7) is maintained (e.g., substantially maintained, for example, at least 50%, at least 60%, at least 70%, at least 80%, at least 90%, at least 95% relative to the binding of the original antibody from which it is derived). In some embodiments, the carboxy portion of a HC CDR1, HC CDR2, HC CDR3, LC CDR1, LC CDR2, and/or LC CDR3 described herein can be extended by one, two, three, four, five or more amino acids compared to one or more of the CDRs described herein (e.g., CDRS from any of the anti-KLK7 antibodies selected from Tables 2a and 2b) so long as specific binding to KLK7 (e.g., human or mouse KLK7) is maintained (e.g., substantially maintained, for example, at least 50%, at least 60%, at least 70%, at least 80%, at least 90%, at least 95% relative to the binding of the original antibody from which it is derived). In some embodiments, the amino portion of a HC CDR1, HC CDR2, HC CDR3, LC CDR1, LC CDR2, and/or LC CDR3 described herein can be shortened by one, two, three, four, five or more amino acids compared to one or more of the CDRs described herein (e.g., CDRS from any of the anti-KLK7 antibodies selected from Tables 2a and 2b) so long as specific binding to KLK7 (e.g., human or mouse KLK7) is maintained (e.g., substantially maintained, for example, at least 50%, at least 60%, at least 70%, at least 80%, at least 90%, at least 95% relative to the binding of the original antibody from which it is derived). In some embodiments, the carboxy portion of a HC CDR1, HC CDR2, HC CDR3, LC CDR1, LC CDR2, and/or LC CDR3 described herein can be shortened by one, two, three, four, five or more amino acids compared to one or more of the CDRs described herein (e.g., CDRS from any of the anti-KLK7 antibodies selected from Tables 2a and 2b) so long as specific binding to KLK7 (e.g., human or mouse KLK7) is maintained (e.g., substantially maintained, for example, at least 50%, at least 60%, at least 70%, at least 80%, at least 90%, at least 95% relative to the binding of the original antibody from which it is derived). Any method can be used to ascertain whether immunospecific binding to KLK7 (e.g., human or mouse KLK7) is maintained, for example, using binding assays and conditions described in the art.

In some examples, an antibody of the disclosure have one or more CDR (e.g., HC CDR or LC CDR) sequences substantially similar to any one of the anti-KLK7 antibodies selected from Tables 2a and 2b. For example, an antibody described herein may include one or more CDR sequence(s) from any of the anti-KLK7 antibodies selected from Tables 2a and 2b containing up to 5, 4, 3, 2, or 1 amino acid residue variations as compared to the corresponding CDR region in any one of the CDRs provided herein (e.g., CDRs from any of the anti-KLK7 antibodies selected from Tables 2a and 2b) so long as specific binding to KLK7 (e.g., human or mouse KLK7) is maintained (e.g., substantially maintained, for example, at least 50%, at least 60%, at least 70%, at least 80%, at least 90%, at least 95% relative to the binding of the original antibody from which it is derived). In some embodiments, any of the amino acid variations in any of the CDRs provided herein may be conservative variations. Conservative variations can be introduced into the CDRs at positions where the residues are not likely to be involved in interacting with a KLK7 (e.g., human or mouse KLK7), for example, as determined based on a crystal structure. Some aspects of the disclosure provide antibodies that comprise one or more of the heavy chain variable (VH) and/or light chain variable (VL) domains provided herein. In some embodiments, any of the VH domains provided herein include one or more of the HC CDR sequences (e.g., HC CDR1, HC CDR2, and HC CDR3) provided herein, for example, any of the HC CDR sequences provided in any one of the anti-KLK7 selected from Tables 2a and 2b. In some embodiments, any of the VL domains provided herein include one or more of the LC CDR sequences (e.g., LC CDR1, LC CDR2, and LC CDR3) provided herein, for example, any of the LC CDR sequences provided in any one of the anti-KLK7 antibodies selected from Tables 2a and 2b.

In some embodiments, an antibody of the disclosure include any antibody that includes a heavy chain variable domain and/or a light chain variable domain of any one of the anti-KLK7 antibodies selected from Tables 2a and 2b, and variants thereof. In some embodiments, an antibody of the disclosure include any antibody that includes the heavy chain variable and light chain variable pairs of any anti-KLK7 antibodies selected from Tables 2a and 2b.

Aspects of the disclosure provide antibodies having a heavy chain variable (VH) and/or a light chain variable (VL) domain amino acid sequence homologous to any of those described herein. In some embodiments, an antibody comprises a heavy chain variable sequence or a light chain variable sequence that is at least 75% (e.g., 80%, 85%, 90%, 95%, 98%, or 99%) identical to the heavy chain variable sequence and/or any light chain variable sequence of any one of the anti-KLK7 antibodies selected from Tables 2a and 2b. In some embodiments, the homologous heavy chain variable and/or a light chain variable amino acid sequences do not vary within any of the CDR sequences provided herein. For example, in some embodiments, the degree of sequence variation (e.g., 75%, 80%, 85%, 90%, 95%, 98%, or 99%) may occur within a heavy chain variable and/or a light chain variable sequence excluding any of the CDR sequences provided herein. In some embodiments, an antibody provided herein comprise a heavy chain variable sequence and a light chain variable sequence that comprises a framework sequence that is at least 75%, 80%, 85%, 90%, 95%, 98%, or 99% identical to the framework sequence of any anti-KLK7 antibodies selected from Tables 2a and 2b.

In some embodiments, an antibody of the present disclosure is a humanized antibody (e.g., a humanized variant containing one or more CDRs of Tables 2a and 2b). In some embodiments, an antibody of the present disclosure comprises a HC CDR1, a HC CDR2, a HC CDR3, a LC CDR1, a LC CDR2, and a LC CDR3 that are the same as the HC CDR1, HC CDR2, HC CDR3, LC CDR1, LC CDR2, and LC CDR3 shown in Tables 2a and 2b, and comprises a humanized heavy chain variable region and/or a humanized light chain variable region.

In some embodiments, an antibody of the present disclosure is a humanized antibody comprising a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH of any of the anti-KLK7 antibodies listed in Tables 2a and 2b. Alternatively or in addition, the antibody of the present disclosure is a humanized antibody comprising a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL of any one of the anti-KLK7 antibodies listed in Tables 2a and 2b.

In some embodiments, an anti-KLK7 antibody of the present disclosure comprises a HC CDR1, HC CDR2 and HC CDR3 of a heavy chain variable domain having the amino acid sequence of SEQ ID NO: 72. Alternatively or in addition, the anti-KLK7 antibody of the present disclosure comprises a LC CDR1, LC CDR2 and LC CDR3 of a light chain variable domain having the amino acid sequence of SEQ ID NO: 73.

In some embodiments, an antibody of the present disclosure comprises a HC CDR1 having the amino acid sequence of SEQ ID NO: 66, a HC CDR2 having the amino acid sequence of SEQ ID NO: 67, a HC CDR3 having the amino acid sequence of SEQ ID NO: 68, a LC CDR1 having the amino acid sequence of SEQ ID NO: 69, a LC CDR2 having the amino acid sequence of SEQ ID NO: 70, and a LC CDR3 having the amino acid sequence of SEQ ID NO: 71.

In some embodiments, an anti-KLK7 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 66, HC CDR2 having the amino acid sequence of SEQ ID NO: 67, and HC CDR3 having the amino acid sequence of SEQ ID NO: 68. “Collectively,” as used anywhere in the present disclosure, means that the total number of amino acid variations in all of the three heavy chain CDRs is within the defined range. Alternatively or in addition, the anti-KLK7 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 69, LC CDR2 having the amino acid sequence of SEQ ID NO: 70, and LC CDR3 having the amino acid sequence of SEQ ID NO: 71.

In some embodiments, an anti-KLK7 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 66, HC CDR2 having the amino acid sequence of SEQ ID NO: 67, and HC CDR3 having the amino acid sequence of SEQ ID NO: 68. Alternatively or in addition, the anti-KLK7 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 69, LC CDR2 having the amino acid sequence of SEQ ID NO: 70, and LC CDR3 having the amino acid sequence of SEQ ID NO: 71.

In some embodiments, an anti-KLK7 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 66; a HC CDR2 having no more than 3 amino acid variations (e.g., no more than 3, 2, or 1 amino acid variation) as compared with the HC CDR2 having the amino acid sequence of SEQ ID NO: 67; and/or a HC CDR3 having no more than 3 amino acid variations (e.g., no more than 3, 2, or 1 amino acid variation) as compared with the HC CDR3 having the amino acid sequence of SEQ ID NO: 68. Alternatively or in addition, the anti-KLK7 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 69; a LC CDR2 having no more than 3 amino acid variations (e.g., no more than 3, 2, or 1 amino acid variation) as compared with the LC CDR2 having the amino acid sequence of SEQ ID NO: 70; and/or a LC CDR3 having no more than 3 amino acid variations (e.g., no more than 3, 2, or 1 amino acid variation) as compared with the LC CDR3 having the amino acid sequence of SEQ ID NO: 71.

In some embodiments, an anti-KLK7 antibody of the present disclosure comprises a VH comprising the amino acid sequence of SEQ ID NO: 72. Alternatively or in addition, the anti-KLK7 antibody of the present disclosure comprises a VL comprising the amino acid sequence of SEQ ID NO: 73.

In some embodiments, an anti-KLK7 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 72. Alternatively or in addition, the anti-KLK7 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 73.

In some embodiments, an anti-KLK7 antibody of the present disclosure comprises a VH comprising an amino acid sequence that is at least 80% (e.g., 80%, 85%, 90%, 95%, 98%, or 99%) identical to the VH as set forth in SEQ ID NO: 72. Alternatively or in addition, the anti-KLK7 antibody of the present disclosure comprises a VL comprising an amino acid sequence that is at least 80% (e.g., 80%, 85%, 90%, 95%, 98%, or 99%) identical to the VL as set forth in SEQ ID NO: 73.

In some embodiments, an anti-KLK7 antibody of the present disclosure comprises a HC CDR1, HC CDR2 and HC CDR3 of a heavy chain variable domain having the amino acid sequence of SEQ ID NO: 199. Alternatively or in addition, the anti-KLK7 antibody of the present disclosure comprises a LC CDR1, LC CDR2 and LC CDR3 of a light chain variable domain having the amino acid sequence of SEQ ID NO: 200.

In some embodiments, an antibody of the present disclosure comprises a HC CDR1 having the amino acid sequence of SEQ ID NO: 74, a HC CDR2 having the amino acid sequence of SEQ ID NO: 67, a HC CDR3 having the amino acid sequence of SEQ ID NO: 75, a LC CDR1 having the amino acid sequence of SEQ ID NO: 69, a LC CDR2 having the amino acid sequence of SEQ ID NO: 70, and a LC CDR3 having the amino acid sequence of SEQ ID NO: 198.

In some embodiments, an anti-KLK7 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 74, HC CDR2 having the amino acid sequence of SEQ ID NO: 67, and HC CDR3 having the amino acid sequence of SEQ ID NO: 75. “Collectively,” as used anywhere in the present disclosure, means that the total number of amino acid variations in all of the three heavy chain CDRs is within the defined range. Alternatively or in addition, the anti-KLK7 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 69, LC CDR2 having the amino acid sequence of SEQ ID NO: 70, and LC CDR3 having the amino acid sequence of SEQ ID NO: 198.

In some embodiments, an anti-KLK7 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 74, HC CDR2 having the amino acid sequence of SEQ ID NO: 67, and HC CDR3 having the amino acid sequence of SEQ ID NO: 75. Alternatively or in addition, the anti-KLK7 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 69, LC CDR2 having the amino acid sequence of SEQ ID NO: 70, and LC CDR3 having the amino acid sequence of SEQ ID NO: 198.

In some embodiments, an anti-KLK7 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 74; a HC CDR2 having no more than 3 amino acid variations (e.g., no more than 3, 2, or 1 amino acid variation) as compared with the HC CDR2 having the amino acid sequence of SEQ ID NO: 67; and/or a HC CDR3 having no more than 3 amino acid variations (e.g., no more than 3, 2, or 1 amino acid variation) as compared with the HC CDR3 having the amino acid sequence of SEQ ID NO: 75. Alternatively or in addition, the anti-KLK7 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 69; a LC CDR2 having no more than 3 amino acid variations (e.g., no more than 3, 2, or 1 amino acid variation) as compared with the LC CDR2 having the amino acid sequence of SEQ ID NO: 70; and/or a LC CDR3 having no more than 3 amino acid variations (e.g., no more than 3, 2, or 1 amino acid variation) as compared with the LC CDR3 having the amino acid sequence of SEQ ID NO: 198.

In some embodiments, an anti-KLK7 antibody of the present disclosure comprises a VH comprising the amino acid sequence of SEQ ID NO: 199. Alternatively or in addition, the anti-KLK7 antibody of the present disclosure comprises a VL comprising the amino acid sequence of SEQ ID NO: 200.

In some embodiments, an anti-KLK7 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 199. Alternatively or in addition, the anti-KLK7 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 200.

In some embodiments, an anti-KLK7 antibody of the present disclosure comprises a VH comprising an amino acid sequence that is at least 80% (e.g., 80%, 85%, 90%, 95%, 98%, or 99%) identical to the VH as set forth in SEQ ID NO: 199. Alternatively or in addition, the anti-KLK7 antibody of the present disclosure comprises a VL comprising an amino acid sequence that is at least 80% (e.g., 80%, 85%, 90%, 95%, 98%, or 99%) identical to the VL as set forth in SEQ ID NO: 200.

In some embodiments, an anti-KLK7 antibody of the present disclosure comprises a HC CDR1, HC CDR2 and HC CDR3 of a heavy chain variable domain having the amino acid sequence of SEQ ID NO: 77. Alternatively or in addition, the anti-KLK7 antibody of the present disclosure comprises a LC CDR1, LC CDR2 and LC CDR3 of a light chain variable domain having the amino acid sequence of SEQ ID NO: 78.

In some embodiments, an antibody of the present disclosure comprises a HC CDR1 having the amino acid sequence of SEQ ID NO: 284, a HC CDR2 having the amino acid sequence of SEQ ID NO: 201, a HC CDR3 having the amino acid sequence of SEQ ID NO: 68, a LC CDR1 having the amino acid sequence of SEQ ID NO: 69, a LC CDR2 having the amino acid sequence of SEQ ID NO: 70, and a LC CDR3 having the amino acid sequence of SEQ ID NO: 76.

In some embodiments, an anti-KLK7 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 284, HC CDR2 having the amino acid sequence of SEQ ID NO: 201, and HC CDR3 having the amino acid sequence of SEQ ID NO: 68. “Collectively,” as used anywhere in the present disclosure, means that the total number of amino acid variations in all of the three heavy chain CDRs is within the defined range. Alternatively or in addition, the anti-KLK7 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 69, LC CDR2 having the amino acid sequence of SEQ ID NO: 70, and LC CDR3 having the amino acid sequence of SEQ ID NO: 76.

In some embodiments, an anti-KLK7 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 284, HC CDR2 having the amino acid sequence of SEQ ID NO: 201, and HC CDR3 having the amino acid sequence of SEQ ID NO: 68. Alternatively or in addition, the anti-KLK7 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 69, LC CDR2 having the amino acid sequence of SEQ ID NO: 70, and LC CDR3 having the amino acid sequence of SEQ ID NO: 76.

In some embodiments, an anti-KLK7 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 284; a HC CDR2 having no more than 3 amino acid variations (e.g., no more than 3, 2, or 1 amino acid variation) as compared with the HC CDR2 having the amino acid sequence of SEQ ID NO: 201; and/or a HC CDR3 having no more than 3 amino acid variations (e.g., no more than 3, 2, or 1 amino acid variation) as compared with the HC CDR3 having the amino acid sequence of SEQ ID NO: 68. Alternatively or in addition, the anti-KLK7 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 69; a LC CDR2 having no more than 3 amino acid variations (e.g., no more than 3, 2, or 1 amino acid variation) as compared with the LC CDR2 having the amino acid sequence of SEQ ID NO: 70; and/or a LC CDR3 having no more than 3 amino acid variations (e.g., no more than 3, 2, or 1 amino acid variation) as compared with the LC CDR3 having the amino acid sequence of SEQ ID NO: 76.

In some embodiments, an anti-KLK7 antibody of the present disclosure comprises a VH comprising the amino acid sequence of SEQ ID NO: 77. Alternatively or in addition, the anti-KLK7 antibody of the present disclosure comprises a VL comprising the amino acid sequence of SEQ ID NO: 78.

In some embodiments, an anti-KLK7 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 77. Alternatively or in addition, the anti-KLK7 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 78.

In some embodiments, an anti-KLK7 antibody of the present disclosure comprises a VH comprising an amino acid sequence that is at least 80% (e.g., 80%, 85%, 90%, 95%, 98%, or 99%) identical to the VH as set forth in SEQ ID NO: 77. Alternatively or in addition, the anti-KLK7 antibody of the present disclosure comprises a VL comprising an amino acid sequence that is at least 80% (e.g., 80%, 85%, 90%, 95%, 98%, or 99%) identical to the VL as set forth in SEQ ID NO: 78.

In some embodiments, an anti-KLK7 antibody of the present disclosure comprises a HC CDR1, HC CDR2 and HC CDR3 of a heavy chain variable domain having the amino acid sequence of SEQ ID NO: 80. Alternatively or in addition, the anti-KLK7 antibody of the present disclosure comprises a LC CDR1, LC CDR2 and LC CDR3 of a light chain variable domain having the amino acid sequence of SEQ ID NO: 78.

In some embodiments, an antibody of the present disclosure comprises a HC CDR1 having the amino acid sequence of SEQ ID NO: 284, a HC CDR2 having the amino acid sequence of SEQ ID NO: 79, a HC CDR3 having the amino acid sequence of SEQ ID NO: 68, a LC CDR1 having the amino acid sequence of SEQ ID NO: 69, a LC CDR2 having the amino acid sequence of SEQ ID NO: 70, and a LC CDR3 having the amino acid sequence of SEQ ID NO: 76.

In some embodiments, an anti-KLK7 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 284, HC CDR2 having the amino acid sequence of SEQ ID NO: 79, and HC CDR3 having the amino acid sequence of SEQ ID NO: 68. “Collectively,” as used anywhere in the present disclosure, means that the total number of amino acid variations in all of the three heavy chain CDRs is within the defined range. Alternatively or in addition, the anti-KLK7 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 69, LC CDR2 having the amino acid sequence of SEQ ID NO: 70, and LC CDR3 having the amino acid sequence of SEQ ID NO: 76.

In some embodiments, an anti-KLK7 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 284, HC CDR2 having the amino acid sequence of SEQ ID NO: 79, and HC CDR3 having the amino acid sequence of SEQ ID NO: 68. Alternatively or in addition, the anti-KLK7 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 69, LC CDR2 having the amino acid sequence of SEQ ID NO: 70, and LC CDR3 having the amino acid sequence of SEQ ID NO: 76.

In some embodiments, an anti-KLK7 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 284; a HC CDR2 having no more than 3 amino acid variations (e.g., no more than 3, 2, or 1 amino acid variation) as compared with the HC CDR2 having the amino acid sequence of SEQ ID NO: 79; and/or a HC CDR3 having no more than 3 amino acid variations (e.g., no more than 3, 2, or 1 amino acid variation) as compared with the HC CDR3 having the amino acid sequence of SEQ ID NO: 68. Alternatively or in addition, the anti-KLK7 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 69; a LC CDR2 having no more than 3 amino acid variations (e.g., no more than 3, 2, or 1 amino acid variation) as compared with the LC CDR2 having the amino acid sequence of SEQ ID NO: 70; and/or a LC CDR3 having no more than 3 amino acid variations (e.g., no more than 3, 2, or 1 amino acid variation) as compared with the LC CDR3 having the amino acid sequence of SEQ ID NO: 76.

In some embodiments, an anti-KLK7 antibody of the present disclosure comprises a VH comprising the amino acid sequence of SEQ ID NO: 80. Alternatively or in addition, the anti-KLK7 antibody of the present disclosure comprises a VL comprising the amino acid sequence of SEQ ID NO: 78.

In some embodiments, an anti-KLK7 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 80. Alternatively or in addition, the anti-KLK7 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 78.

In some embodiments, an anti-KLK7 antibody of the present disclosure comprises a VH comprising an amino acid sequence that is at least 80% (e.g., 80%, 85%, 90%, 95%, 98%, or 99%) identical to the VH as set forth in SEQ ID NO: 80. Alternatively or in addition, the anti-KLK7 antibody of the present disclosure comprises a VL comprising an amino acid sequence that is at least 80% (e.g., 80%, 85%, 90%, 95%, 98%, or 99%) identical to the VL as set forth in SEQ ID NO: 78.

In some embodiments, an anti-KLK7 antibody of the present disclosure comprises a HC CDR1, HC CDR2 and HC CDR3 of a heavy chain variable domain having the amino acid sequence of SEQ ID NO: 80. Alternatively or in addition, the anti-KLK7 antibody of the present disclosure comprises a LC CDR1, LC CDR2 and LC CDR3 of a light chain variable domain having the amino acid sequence of SEQ ID NO: 82.

In some embodiments, an antibody of the present disclosure comprises a HC CDR1 having the amino acid sequence of SEQ ID NO: 284, a HC CDR2 having the amino acid sequence of SEQ ID NO: 79, a HC CDR3 having the amino acid sequence of SEQ ID NO: 68, a LC CDR1 having the amino acid sequence of SEQ ID NO: 69, a LC CDR2 having the amino acid sequence of SEQ ID NO: 70, and a LC CDR3 having the amino acid sequence of SEQ ID NO: 81.

In some embodiments, an anti-KLK7 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 284, HC CDR2 having the amino acid sequence of SEQ ID NO: 79, and HC CDR3 having the amino acid sequence of SEQ ID NO: 68. “Collectively,” as used anywhere in the present disclosure, means that the total number of amino acid variations in all of the three heavy chain CDRs is within the defined range. Alternatively or in addition, the anti-KLK7 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 69, LC CDR2 having the amino acid sequence of SEQ ID NO: 70, and LC CDR3 having the amino acid sequence of SEQ ID NO: 81.

In some embodiments, an anti-KLK7 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 284, HC CDR2 having the amino acid sequence of SEQ ID NO: 79, and HC CDR3 having the amino acid sequence of SEQ ID NO: 68. Alternatively or in addition, the anti-KLK7 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 69, LC CDR2 having the amino acid sequence of SEQ ID NO: 70, and LC CDR3 having the amino acid sequence of SEQ ID NO: 81.

In some embodiments, an anti-KLK7 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 284; a HC CDR2 having no more than 3 amino acid variations (e.g., no more than 3, 2, or 1 amino acid variation) as compared with the HC CDR2 having the amino acid sequence of SEQ ID NO: 79; and/or a HC CDR3 having no more than 3 amino acid variations (e.g., no more than 3, 2, or 1 amino acid variation) as compared with the HC CDR3 having the amino acid sequence of SEQ ID NO: 68. Alternatively or in addition, the anti-KLK7 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 69; a LC CDR2 having no more than 3 amino acid variations (e.g., no more than 3, 2, or 1 amino acid variation) as compared with the LC CDR2 having the amino acid sequence of SEQ ID NO: 70; and/or a LC CDR3 having no more than 3 amino acid variations (e.g., no more than 3, 2, or 1 amino acid variation) as compared with the LC CDR3 having the amino acid sequence of SEQ ID NO: 81.

In some embodiments, an anti-KLK7 antibody of the present disclosure comprises a VH comprising the amino acid sequence of SEQ ID NO: 80. Alternatively or in addition, the anti-KLK7 antibody of the present disclosure comprises a VL comprising the amino acid sequence of SEQ ID NO: 82.

In some embodiments, an anti-KLK7 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 80. Alternatively or in addition, the anti-KLK7 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 82.

In some embodiments, an anti-KLK7 antibody of the present disclosure comprises a VH comprising an amino acid sequence that is at least 80% (e.g., 80%, 85%, 90%, 95%, 98%, or 99%) identical to the VH as set forth in SEQ ID NO: 80. Alternatively or in addition, the anti-KLK7 antibody of the present disclosure comprises a VL comprising an amino acid sequence that is at least 80% (e.g., 80%, 85%, 90%, 95%, 98%, or 99%) identical to the VL as set forth in SEQ ID NO: 82.

In some embodiments, an anti-KLK7 antibody of the present disclosure comprises a HC CDR1, HC CDR2 and HC CDR3 of a heavy chain variable domain having the amino acid sequence of SEQ ID NO: 84. Alternatively or in addition, the anti-KLK7 antibody of the present disclosure comprises a LC CDR1, LC CDR2 and LC CDR3 of a light chain variable domain having the amino acid sequence of SEQ ID NO: 78.

In some embodiments, an antibody of the present disclosure comprises a HC CDR1 having the amino acid sequence of SEQ ID NO: 284, a HC CDR2 having the amino acid sequence of SEQ ID NO: 79, a HC CDR3 having the amino acid sequence of SEQ ID NO: 83, a LC CDR1 having the amino acid sequence of SEQ ID NO: 69, a LC CDR2 having the amino acid sequence of SEQ ID NO: 70, and a LC CDR3 having the amino acid sequence of SEQ ID NO: 76.

In some embodiments, an anti-KLK7 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 284, HC CDR2 having the amino acid sequence of SEQ ID NO: 79, and HC CDR3 having the amino acid sequence of SEQ ID NO: 83. “Collectively,” as used anywhere in the present disclosure, means that the total number of amino acid variations in all of the three heavy chain CDRs is within the defined range. Alternatively or in addition, the anti-KLK7 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 69, LC CDR2 having the amino acid sequence of SEQ ID NO: 70, and LC CDR3 having the amino acid sequence of SEQ ID NO: 76.

In some embodiments, an anti-KLK7 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 284, HC CDR2 having the amino acid sequence of SEQ ID NO: 79, and HC CDR3 having the amino acid sequence of SEQ ID NO: 83. Alternatively or in addition, the anti-KLK7 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 69, LC CDR2 having the amino acid sequence of SEQ ID NO: 70, and LC CDR3 having the amino acid sequence of SEQ ID NO: 76.

In some embodiments, an anti-KLK7 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 284; a HC CDR2 having no more than 3 amino acid variations (e.g., no more than 3, 2, or 1 amino acid variation) as compared with the HC CDR2 having the amino acid sequence of SEQ ID NO: 79; and/or a HC CDR3 having no more than 3 amino acid variations (e.g., no more than 3, 2, or 1 amino acid variation) as compared with the HC CDR3 having the amino acid sequence of SEQ ID NO: 83. Alternatively or in addition, the anti-KLK7 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 69; a LC CDR2 having no more than 3 amino acid variations (e.g., no more than 3, 2, or 1 amino acid variation) as compared with the LC CDR2 having the amino acid sequence of SEQ ID NO: 70; and/or a LC CDR3 having no more than 3 amino acid variations (e.g., no more than 3, 2, or 1 amino acid variation) as compared with the LC CDR3 having the amino acid sequence of SEQ ID NO: 76.

In some embodiments, an anti-KLK7 antibody of the present disclosure comprises a VH comprising the amino acid sequence of SEQ ID NO: 84. Alternatively or in addition, the anti-KLK7 antibody of the present disclosure comprises a VL comprising the amino acid sequence of SEQ ID NO: 78.

In some embodiments, an anti-KLK7 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 84. Alternatively or in addition, the anti-KLK7 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 78.

In some embodiments, an anti-KLK7 antibody of the present disclosure comprises a VH comprising an amino acid sequence that is at least 80% (e.g., 80%, 85%, 90%, 95%, 98%, or 99%) identical to the VH as set forth in SEQ ID NO: 84. Alternatively or in addition, the anti-KLK7 antibody of the present disclosure comprises a VL comprising an amino acid sequence that is at least 80% (e.g., 80%, 85%, 90%, 95%, 98%, or 99%) identical to the VL as set forth in SEQ ID NO: 78.

In some embodiments, an anti-KLK7 antibody of the present disclosure comprises a HC CDR1, HC CDR2 and HC CDR3 of a heavy chain variable domain having the amino acid sequence of SEQ ID NO: 87. Alternatively or in addition, the anti-KLK7 antibody of the present disclosure comprises a LC CDR1, LC CDR2 and LC CDR3 of a light chain variable domain having the amino acid sequence of SEQ ID NO: 88.

In some embodiments, an antibody of the present disclosure comprises a HC CDR1 having the amino acid sequence of SEQ ID NO: 284, a HC CDR2 having the amino acid sequence of SEQ ID NO: 79, a HC CDR3 having the amino acid sequence of SEQ ID NO: 85, a LC CDR1 having the amino acid sequence of SEQ ID NO: 69, a LC CDR2 having the amino acid sequence of SEQ ID NO: 70, and a LC CDR3 having the amino acid sequence of SEQ ID NO: 86.

In some embodiments, an anti-KLK7 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 284, HC CDR2 having the amino acid sequence of SEQ ID NO: 79, and HC CDR3 having the amino acid sequence of SEQ ID NO: 85. “Collectively,” as used anywhere in the present disclosure, means that the total number of amino acid variations in all of the three heavy chain CDRs is within the defined range. Alternatively or in addition, the anti-KLK7 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 69, LC CDR2 having the amino acid sequence of SEQ ID NO: 70, and LC CDR3 having the amino acid sequence of SEQ ID NO: 86.

In some embodiments, an anti-KLK7 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 284, HC CDR2 having the amino acid sequence of SEQ ID NO: 79, and HC CDR3 having the amino acid sequence of SEQ ID NO: 85. Alternatively or in addition, the anti-KLK7 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 69, LC CDR2 having the amino acid sequence of SEQ ID NO: 70, and LC CDR3 having the amino acid sequence of SEQ ID NO: 86.

In some embodiments, an anti-KLK7 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 284; a HC CDR2 having no more than 3 amino acid variations (e.g., no more than 3, 2, or 1 amino acid variation) as compared with the HC CDR2 having the amino acid sequence of SEQ ID NO: 79; and/or a HC CDR3 having no more than 3 amino acid variations (e.g., no more than 3, 2, or 1 amino acid variation) as compared with the HC CDR3 having the amino acid sequence of SEQ ID NO: 85. Alternatively or in addition, the anti-KLK7 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 69; a LC CDR2 having no more than 3 amino acid variations (e.g., no more than 3, 2, or 1 amino acid variation) as compared with the LC CDR2 having the amino acid sequence of SEQ ID NO: 70; and/or a LC CDR3 having no more than 3 amino acid variations (e.g., no more than 3, 2, or 1 amino acid variation) as compared with the LC CDR3 having the amino acid sequence of SEQ ID NO: 86.

In some embodiments, an anti-KLK7 antibody of the present disclosure comprises a VH comprising the amino acid sequence of SEQ ID NO: 87. Alternatively or in addition, the anti-KLK7 antibody of the present disclosure comprises a VL comprising the amino acid sequence of SEQ ID NO: 88.

In some embodiments, an anti-KLK7 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 87. Alternatively or in addition, the anti-KLK7 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 88.

In some embodiments, an anti-KLK7 antibody of the present disclosure comprises a VH comprising an amino acid sequence that is at least 80% (e.g., 80%, 85%, 90%, 95%, 98%, or 99%) identical to the VH as set forth in SEQ ID NO: 87. Alternatively or in addition, the anti-KLK7 antibody of the present disclosure comprises a VL comprising an amino acid sequence that is at least 80% (e.g., 80%, 85%, 90%, 95%, 98%, or 99%) identical to the VL as set forth in SEQ ID NO: 88.

In some embodiments, an anti-KLK7 antibody of the present disclosure comprises a HC CDR1, HC CDR2 and HC CDR3 of a heavy chain variable domain having the amino acid sequence of SEQ ID NO: 285. Alternatively or in addition, the anti-KLK7 antibody of the present disclosure comprises a LC CDR1, LC CDR2 and LC CDR3 of a light chain variable domain having the amino acid sequence of SEQ ID NO: 82.

In some embodiments, an antibody of the present disclosure comprises a HC CDR1 having the amino acid sequence of SEQ ID NO: 284, a HC CDR2 having the amino acid sequence of SEQ ID NO: 79, a HC CDR3 having the amino acid sequence of SEQ ID NO: 89, a LC CDR1 having the amino acid sequence of SEQ ID NO: 69, a LC CDR2 having the amino acid sequence of SEQ ID NO: 70, and a LC CDR3 having the amino acid sequence of SEQ ID NO: 81.

In some embodiments, an anti-KLK7 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 284, HC CDR2 having the amino acid sequence of SEQ ID NO: 79, and HC CDR3 having the amino acid sequence of SEQ ID NO: 89. “Collectively,” as used anywhere in the present disclosure, means that the total number of amino acid variations in all of the three heavy chain CDRs is within the defined range. Alternatively or in addition, the anti-KLK7 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 69, LC CDR2 having the amino acid sequence of SEQ ID NO: 70, and LC CDR3 having the amino acid sequence of SEQ ID NO: 81.

In some embodiments, an anti-KLK7 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 284, HC CDR2 having the amino acid sequence of SEQ ID NO: 79, and HC CDR3 having the amino acid sequence of SEQ ID NO: 89. Alternatively or in addition, the anti-KLK7 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 69, LC CDR2 having the amino acid sequence of SEQ ID NO: 70, and LC CDR3 having the amino acid sequence of SEQ ID NO: 81.

In some embodiments, an anti-KLK7 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 284; a HC CDR2 having no more than 3 amino acid variations (e.g., no more than 3, 2, or 1 amino acid variation) as compared with the HC CDR2 having the amino acid sequence of SEQ ID NO: 79; and/or a HC CDR3 having no more than 3 amino acid variations (e.g., no more than 3, 2, or 1 amino acid variation) as compared with the HC CDR3 having the amino acid sequence of SEQ ID NO: 89. Alternatively or in addition, the anti-KLK7 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 69; a LC CDR2 having no more than 3 amino acid variations (e.g., no more than 3, 2, or 1 amino acid variation) as compared with the LC CDR2 having the amino acid sequence of SEQ ID NO: 70; and/or a LC CDR3 having no more than 3 amino acid variations (e.g., no more than 3, 2, or 1 amino acid variation) as compared with the LC CDR3 having the amino acid sequence of SEQ ID NO: 81.

In some embodiments, an anti-KLK7 antibody of the present disclosure comprises a VH comprising the amino acid sequence of SEQ ID NO: 285. Alternatively or in addition, the anti-KLK7 antibody of the present disclosure comprises a VL comprising the amino acid sequence of SEQ ID NO: 82.

In some embodiments, an anti-KLK7 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 285. Alternatively or in addition, the anti-KLK7 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 82.

In some embodiments, an anti-KLK7 antibody of the present disclosure comprises a VH comprising an amino acid sequence that is at least 80% (e.g., 80%, 85%, 90%, 95%, 98%, or 99%) identical to the VH as set forth in SEQ ID NO: 285. Alternatively or in addition, the anti-KLK7 antibody of the present disclosure comprises a VL comprising an amino acid sequence that is at least 80% (e.g., 80%, 85%, 90%, 95%, 98%, or 99%) identical to the VL as set forth in SEQ ID NO: 82.

In some embodiments, an anti-KLK7 antibody of the present disclosure comprises a HC CDR1, HC CDR2 and HC CDR3 of a heavy chain variable domain having the amino acid sequence of SEQ ID NO: 98. Alternatively or in addition, the anti-KLK7 antibody of the present disclosure comprises a LC CDR1, LC CDR2 and LC CDR3 of a light chain variable domain having the amino acid sequence of SEQ ID NO: 97.

In some embodiments, an antibody of the present disclosure comprises a HC CDR1 having the amino acid sequence of SEQ ID NO: 90, a HC CDR2 having the amino acid sequence of SEQ ID NO: 91, a HC CDR3 having the amino acid sequence of SEQ ID NO: 92, a LC CDR1 having the amino acid sequence of SEQ ID NO: 93, a LC CDR2 having the amino acid sequence of SEQ ID NO: 94, and a LC CDR3 having the amino acid sequence of SEQ ID NO: 95.

In some embodiments, an anti-KLK7 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 90, HC CDR2 having the amino acid sequence of SEQ ID NO: 91, and HC CDR3 having the amino acid sequence of SEQ ID NO: 92. “Collectively,” as used anywhere in the present disclosure, means that the total number of amino acid variations in all of the three heavy chain CDRs is within the defined range. Alternatively or in addition, the anti-KLK7 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 93, LC CDR2 having the amino acid sequence of SEQ ID NO: 94, and LC CDR3 having the amino acid sequence of SEQ ID NO: 95.

In some embodiments, an anti-KLK7 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 90, HC CDR2 having the amino acid sequence of SEQ ID NO: 91, and HC CDR3 having the amino acid sequence of SEQ ID NO: 92. Alternatively or in addition, the anti-KLK7 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 93, LC CDR2 having the amino acid sequence of SEQ ID NO: 94, and LC CDR3 having the amino acid sequence of SEQ ID NO: 95.

In some embodiments, an anti-KLK7 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 90; a HC CDR2 having no more than 3 amino acid variations (e.g., no more than 3, 2, or 1 amino acid variation) as compared with the HC CDR2 having the amino acid sequence of SEQ ID NO: 91; and/or a HC CDR3 having no more than 3 amino acid variations (e.g., no more than 3, 2, or 1 amino acid variation) as compared with the HC CDR3 having the amino acid sequence of SEQ ID NO: 92. Alternatively or in addition, the anti-KLK7 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 93; a LC CDR2 having no more than 3 amino acid variations (e.g., no more than 3, 2, or 1 amino acid variation) as compared with the LC CDR2 having the amino acid sequence of SEQ ID NO: 94; and/or a LC CDR3 having no more than 3 amino acid variations (e.g., no more than 3, 2, or 1 amino acid variation) as compared with the LC CDR3 having the amino acid sequence of SEQ ID NO: 95.

In some embodiments, an anti-KLK7 antibody of the present disclosure comprises a VH comprising the amino acid sequence of SEQ ID NO: 98. Alternatively or in addition, the anti-KLK7 antibody of the present disclosure comprises a VL comprising the amino acid sequence of SEQ ID NO: 97.

In some embodiments, an anti-KLK7 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 98. Alternatively or in addition, the anti-KLK7 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 97.

In some embodiments, an anti-KLK7 antibody of the present disclosure comprises a VH comprising an amino acid sequence that is at least 80% (e.g., 80%, 85%, 90%, 95%, 98%, or 99%) identical to the VH as set forth in SEQ ID NO: 98. Alternatively or in addition, the anti-KLK7 antibody of the present disclosure comprises a VL comprising an amino acid sequence that is at least 80% (e.g., 80%, 85%, 90%, 95%, 98%, or 99%) identical to the VL as set forth in SEQ ID NO: 97.

In some embodiments, an anti-KLK7 antibody of the present disclosure comprises a HC CDR1, HC CDR2 and HC CDR3 of a heavy chain variable domain having the amino acid sequence of SEQ ID NO: 153. Alternatively or in addition, the anti-KLK7 antibody of the present disclosure comprises a LC CDR1, LC CDR2 and LC CDR3 of a light chain variable domain having the amino acid sequence of SEQ ID NO: 97.

In some embodiments, an antibody of the present disclosure comprises a HC CDR1 having the amino acid sequence of SEQ ID NO: 96, a HC CDR2 having the amino acid sequence of SEQ ID NO: 109, a HC CDR3 having the amino acid sequence of SEQ ID NO: 92, a LC CDR1 having the amino acid sequence of SEQ ID NO: 93, a LC CDR2 having the amino acid sequence of SEQ ID NO: 94, and a LC CDR3 having the amino acid sequence of SEQ ID NO: 95.

In some embodiments, an anti-KLK7 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 96, HC CDR2 having the amino acid sequence of SEQ ID NO: 109, and HC CDR3 having the amino acid sequence of SEQ ID NO: 92. “Collectively,” as used anywhere in the present disclosure, means that the total number of amino acid variations in all of the three heavy chain CDRs is within the defined range. Alternatively or in addition, the anti-KLK7 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 93, LC CDR2 having the amino acid sequence of SEQ ID NO: 94, and LC CDR3 having the amino acid sequence of SEQ ID NO: 95.

In some embodiments, an anti-KLK7 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 96, HC CDR2 having the amino acid sequence of SEQ ID NO: 109, and HC CDR3 having the amino acid sequence of SEQ ID NO: 92. Alternatively or in addition, the anti-KLK7 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 93, LC CDR2 having the amino acid sequence of SEQ ID NO: 94, and LC CDR3 having the amino acid sequence of SEQ ID NO: 95.

In some embodiments, an anti-KLK7 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 96; a HC CDR2 having no more than 3 amino acid variations (e.g., no more than 3, 2, or 1 amino acid variation) as compared with the HC CDR2 having the amino acid sequence of SEQ ID NO: 109; and/or a HC CDR3 having no more than 3 amino acid variations (e.g., no more than 3, 2, or 1 amino acid variation) as compared with the HC CDR3 having the amino acid sequence of SEQ ID NO: 92. Alternatively or in addition, the anti-KLK7 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 93; a LC CDR2 having no more than 3 amino acid variations (e.g., no more than 3, 2, or 1 amino acid variation) as compared with the LC CDR2 having the amino acid sequence of SEQ ID NO: 94; and/or a LC CDR3 having no more than 3 amino acid variations (e.g., no more than 3, 2, or 1 amino acid variation) as compared with the LC CDR3 having the amino acid sequence of SEQ ID NO: 95.

In some embodiments, an anti-KLK7 antibody of the present disclosure comprises a VH comprising the amino acid sequence of SEQ ID NO: 153. Alternatively or in addition, the anti-KLK7 antibody of the present disclosure comprises a VL comprising the amino acid sequence of SEQ ID NO: 97.

In some embodiments, an anti-KLK7 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 153. Alternatively or in addition, the anti-KLK7 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 97.

In some embodiments, an anti-KLK7 antibody of the present disclosure comprises a VH comprising an amino acid sequence that is at least 80% (e.g., 80%, 85%, 90%, 95%, 98%, or 99%) identical to the VH as set forth in SEQ ID NO: 153. Alternatively or in addition, the anti-KLK7 antibody of the present disclosure comprises a VL comprising an amino acid sequence that is at least 80% (e.g., 80%, 85%, 90%, 95%, 98%, or 99%) identical to the VL as set forth in SEQ ID NO: 97.

In some embodiments, an anti-KLK7 antibody of the present disclosure comprises a HC CDR1, HC CDR2 and HC CDR3 of a heavy chain variable domain having the amino acid sequence of SEQ ID NO: 103. Alternatively or in addition, the anti-KLK7 antibody of the present disclosure comprises a LC CDR1, LC CDR2 and LC CDR3 of a light chain variable domain having the amino acid sequence of SEQ ID NO: 104.

In some embodiments, an antibody of the present disclosure comprises a HC CDR1 having the amino acid sequence of SEQ ID NO: 99, a HC CDR2 having the amino acid sequence of SEQ ID NO: 100, a HC CDR3 having the amino acid sequence of SEQ ID NO: 101, a LC CDR1 having the amino acid sequence of SEQ ID NO: 93, a LC CDR2 having the amino acid sequence of SEQ ID NO: 94, and a LC CDR3 having the amino acid sequence of SEQ ID NO: 102.

In some embodiments, an anti-KLK7 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 99, HC CDR2 having the amino acid sequence of SEQ ID NO: 100, and HC CDR3 having the amino acid sequence of SEQ ID NO: 101. “Collectively,” as used anywhere in the present disclosure, means that the total number of amino acid variations in all of the three heavy chain CDRs is within the defined range. Alternatively or in addition, the anti-KLK7 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 93, LC CDR2 having the amino acid sequence of SEQ ID NO: 94, and LC CDR3 having the amino acid sequence of SEQ ID NO: 102.

In some embodiments, an anti-KLK7 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 99, HC CDR2 having the amino acid sequence of SEQ ID NO: 100, and HC CDR3 having the amino acid sequence of SEQ ID NO: 101. Alternatively or in addition, the anti-KLK7 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 93, LC CDR2 having the amino acid sequence of SEQ ID NO: 94, and LC CDR3 having the amino acid sequence of SEQ ID NO: 102.

In some embodiments, an anti-KLK7 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 99; a HC CDR2 having no more than 3 amino acid variations (e.g., no more than 3, 2, or 1 amino acid variation) as compared with the HC CDR2 having the amino acid sequence of SEQ ID NO: 100; and/or a HC CDR3 having no more than 3 amino acid variations (e.g., no more than 3, 2, or 1 amino acid variation) as compared with the HC CDR3 having the amino acid sequence of SEQ ID NO: 101. Alternatively or in addition, the anti-KLK7 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 93; a LC CDR2 having no more than 3 amino acid variations (e.g., no more than 3, 2, or 1 amino acid variation) as compared with the LC CDR2 having the amino acid sequence of SEQ ID NO: 94; and/or a LC CDR3 having no more than 3 amino acid variations (e.g., no more than 3, 2, or 1 amino acid variation) as compared with the LC CDR3 having the amino acid sequence of SEQ ID NO: 102.

In some embodiments, an anti-KLK7 antibody of the present disclosure comprises a VH comprising the amino acid sequence of SEQ ID NO: 103. Alternatively or in addition, the anti-KLK7 antibody of the present disclosure comprises a VL comprising the amino acid sequence of SEQ ID NO: 104.

In some embodiments, an anti-KLK7 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 103. Alternatively or in addition, the anti-KLK7 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 104.

In some embodiments, an anti-KLK7 antibody of the present disclosure comprises a VH comprising an amino acid sequence that is at least 80% (e.g., 80%, 85%, 90%, 95%, 98%, or 99%) identical to the VH as set forth in SEQ ID NO: 103. Alternatively or in addition, the anti-KLK7 antibody of the present disclosure comprises a VL comprising an amino acid sequence that is at least 80% (e.g., 80%, 85%, 90%, 95%, 98%, or 99%) identical to the VL as set forth in SEQ ID NO: 104.

In some embodiments, an anti-KLK7 antibody of the present disclosure comprises a HC CDR1, HC CDR2 and HC CDR3 of a heavy chain variable domain having the amino acid sequence of SEQ ID NO: 107. Alternatively or in addition, the anti-KLK7 antibody of the present disclosure comprises a LC CDR1, LC CDR2 and LC CDR3 of a light chain variable domain having the amino acid sequence of SEQ ID NO: 108.

In some embodiments, an antibody of the present disclosure comprises a HC CDR1 having the amino acid sequence of SEQ ID NO: 99, a HC CDR2 having the amino acid sequence of SEQ ID NO: 100, a HC CDR3 having the amino acid sequence of SEQ ID NO: 105, a LC CDR1 having the amino acid sequence of SEQ ID NO: 93, a LC CDR2 having the amino acid sequence of SEQ ID NO: 94, and a LC CDR3 having the amino acid sequence of SEQ ID NO: 106.

In some embodiments, an anti-KLK7 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 99, HC CDR2 having the amino acid sequence of SEQ ID NO: 100, and HC CDR3 having the amino acid sequence of SEQ ID NO: 105. “Collectively,” as used anywhere in the present disclosure, means that the total number of amino acid variations in all of the three heavy chain CDRs is within the defined range. Alternatively or in addition, the anti-KLK7 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 93, LC CDR2 having the amino acid sequence of SEQ ID NO: 94, and LC CDR3 having the amino acid sequence of SEQ ID NO: 106.

In some embodiments, an anti-KLK7 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 99, HC CDR2 having the amino acid sequence of SEQ ID NO: 100, and HC CDR3 having the amino acid sequence of SEQ ID NO: 105. Alternatively or in addition, the anti-KLK7 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 93, LC CDR2 having the amino acid sequence of SEQ ID NO: 94, and LC CDR3 having the amino acid sequence of SEQ ID NO: 106.

In some embodiments, an anti-KLK7 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 99; a HC CDR2 having no more than 3 amino acid variations (e.g., no more than 3, 2, or 1 amino acid variation) as compared with the HC CDR2 having the amino acid sequence of SEQ ID NO: 100; and/or a HC CDR3 having no more than 3 amino acid variations (e.g., no more than 3, 2, or 1 amino acid variation) as compared with the HC CDR3 having the amino acid sequence of SEQ ID NO: 105. Alternatively or in addition, the anti-KLK7 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 93; a LC CDR2 having no more than 3 amino acid variations (e.g., no more than 3, 2, or 1 amino acid variation) as compared with the LC CDR2 having the amino acid sequence of SEQ ID NO: 94; and/or a LC CDR3 having no more than 3 amino acid variations (e.g., no more than 3, 2, or 1 amino acid variation) as compared with the LC CDR3 having the amino acid sequence of SEQ ID NO: 106.

In some embodiments, an anti-KLK7 antibody of the present disclosure comprises a VH comprising the amino acid sequence of SEQ ID NO: 107. Alternatively or in addition, the anti-KLK7 antibody of the present disclosure comprises a VL comprising the amino acid sequence of SEQ ID NO: 108.

In some embodiments, an anti-KLK7 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 107. Alternatively or in addition, the anti-KLK7 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 108.

In some embodiments, an anti-KLK7 antibody of the present disclosure comprises a VH comprising an amino acid sequence that is at least 80% (e.g., 80%, 85%, 90%, 95%, 98%, or 99%) identical to the VH as set forth in SEQ ID NO: 107. Alternatively or in addition, the anti-KLK7 antibody of the present disclosure comprises a VL comprising an amino acid sequence that is at least 80% (e.g., 80%, 85%, 90%, 95%, 98%, or 99%) identical to the VL as set forth in SEQ ID NO: 108.

In some embodiments, an anti-KLK7 antibody of the present disclosure comprises a HC CDR1, HC CDR2 and HC CDR3 of a heavy chain variable domain having the amino acid sequence of SEQ ID NO: 111. Alternatively or in addition, the anti-KLK7 antibody of the present disclosure comprises a LC CDR1, LC CDR2 and LC CDR3 of a light chain variable domain having the amino acid sequence of SEQ ID NO: 97.

In some embodiments, an antibody of the present disclosure comprises a HC CDR1 having the amino acid sequence of SEQ ID NO: 96, a HC CDR2 having the amino acid sequence of SEQ ID NO: 109, a HC CDR3 having the amino acid sequence of SEQ ID NO: 110, a LC CDR1 having the amino acid sequence of SEQ ID NO: 93, a LC CDR2 having the amino acid sequence of SEQ ID NO: 94, and a LC CDR3 having the amino acid sequence of SEQ ID NO: 95.

In some embodiments, an anti-KLK7 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 96, HC CDR2 having the amino acid sequence of SEQ ID NO: 109, and HC CDR3 having the amino acid sequence of SEQ ID NO: 110. “Collectively,” as used anywhere in the present disclosure, means that the total number of amino acid variations in all of the three heavy chain CDRs is within the defined range. Alternatively or in addition, the anti-KLK7 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 93, LC CDR2 having the amino acid sequence of SEQ ID NO: 94, and LC CDR3 having the amino acid sequence of SEQ ID NO: 95.

In some embodiments, an anti-KLK7 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 96, HC CDR2 having the amino acid sequence of SEQ ID NO: 109, and HC CDR3 having the amino acid sequence of SEQ ID NO: 110. Alternatively or in addition, the anti-KLK7 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 93, LC CDR2 having the amino acid sequence of SEQ ID NO: 94, and LC CDR3 having the amino acid sequence of SEQ ID NO: 95.

In some embodiments, an anti-KLK7 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 96; a HC CDR2 having no more than 3 amino acid variations (e.g., no more than 3, 2, or 1 amino acid variation) as compared with the HC CDR2 having the amino acid sequence of SEQ ID NO: 109; and/or a HC CDR3 having no more than 3 amino acid variations (e.g., no more than 3, 2, or 1 amino acid variation) as compared with the HC CDR3 having the amino acid sequence of SEQ ID NO: 110. Alternatively or in addition, the anti-KLK7 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 93; a LC CDR2 having no more than 3 amino acid variations (e.g., no more than 3, 2, or 1 amino acid variation) as compared with the LC CDR2 having the amino acid sequence of SEQ ID NO: 94; and/or a LC CDR3 having no more than 3 amino acid variations (e.g., no more than 3, 2, or 1 amino acid variation) as compared with the LC CDR3 having the amino acid sequence of SEQ ID NO: 95.

In some embodiments, an anti-KLK7 antibody of the present disclosure comprises a VH comprising the amino acid sequence of SEQ ID NO: 111. Alternatively or in addition, the anti-KLK7 antibody of the present disclosure comprises a VL comprising the amino acid sequence of SEQ ID NO: 97.

In some embodiments, an anti-KLK7 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 111. Alternatively or in addition, the anti-KLK7 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 97.

In some embodiments, an anti-KLK7 antibody of the present disclosure comprises a VH comprising an amino acid sequence that is at least 80% (e.g., 80%, 85%, 90%, 95%, 98%, or 99%) identical to the VH as set forth in SEQ ID NO: 111. Alternatively or in addition, the anti-KLK7 antibody of the present disclosure comprises a VL comprising an amino acid sequence that is at least 80% (e.g., 80%, 85%, 90%, 95%, 98%, or 99%) identical to the VL as set forth in SEQ ID NO: 97.

In some embodiments, an anti-KLK7 antibody of the present disclosure comprises a HC CDR1, HC CDR2 and HC CDR3 of a heavy chain variable domain having the amino acid sequence of SEQ ID NO: 115. Alternatively or in addition, the anti-KLK7 antibody of the present disclosure comprises a LC CDR1, LC CDR2 and LC CDR3 of a light chain variable domain having the amino acid sequence of SEQ ID NO: 116.

In some embodiments, an antibody of the present disclosure comprises a HC CDR1 having the amino acid sequence of SEQ ID NO: 66, a HC CDR2 having the amino acid sequence of SEQ ID NO: 112, a HC CDR3 having the amino acid sequence of SEQ ID NO: 113, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 114.

In some embodiments, an anti-KLK7 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 66, HC CDR2 having the amino acid sequence of SEQ ID NO: 112, and HC CDR3 having the amino acid sequence of SEQ ID NO: 113. “Collectively,” as used anywhere in the present disclosure, means that the total number of amino acid variations in all of the three heavy chain CDRs is within the defined range. Alternatively or in addition, the anti-KLK7 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 4, LC CDR2 having the amino acid sequence of SEQ ID NO: 5, and LC CDR3 having the amino acid sequence of SEQ ID NO: 114.

In some embodiments, an anti-KLK7 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 66, HC CDR2 having the amino acid sequence of SEQ ID NO: 112, and HC CDR3 having the amino acid sequence of SEQ ID NO: 113. Alternatively or in addition, the anti-KLK7 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 4, LC CDR2 having the amino acid sequence of SEQ ID NO: 5, and LC CDR3 having the amino acid sequence of SEQ ID NO: 114.

In some embodiments, an anti-KLK7 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 66; a HC CDR2 having no more than 3 amino acid variations (e.g., no more than 3, 2, or 1 amino acid variation) as compared with the HC CDR2 having the amino acid sequence of SEQ ID NO: 112; and/or a HC CDR3 having no more than 3 amino acid variations (e.g., no more than 3, 2, or 1 amino acid variation) as compared with the HC CDR3 having the amino acid sequence of SEQ ID NO: 113. Alternatively or in addition, the anti-KLK7 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of SEQ ID NO: 5; and/or a LC CDR3 having no more than 3 amino acid variations (e.g., no more than 3, 2, or 1 amino acid variation) as compared with the LC CDR3 having the amino acid sequence of SEQ ID NO: 114.

In some embodiments, an anti-KLK7 antibody of the present disclosure comprises a VH comprising the amino acid sequence of SEQ ID NO: 115. Alternatively or in addition, the anti-KLK7 antibody of the present disclosure comprises a VL comprising the amino acid sequence of SEQ ID NO: 116.

In some embodiments, an anti-KLK7 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 115. Alternatively or in addition, the anti-KLK7 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 116.

In some embodiments, an anti-KLK7 antibody of the present disclosure comprises a VH comprising an amino acid sequence that is at least 80% (e.g., 80%, 85%, 90%, 95%, 98%, or 99%) identical to the VH as set forth in SEQ ID NO: 115. Alternatively or in addition, the anti-KLK7 antibody of the present disclosure comprises a VL comprising an amino acid sequence that is at least 80% (e.g., 80%, 85%, 90%, 95%, 98%, or 99%) identical to the VL as set forth in SEQ ID NO: 116.

In some embodiments, an anti-KLK7 antibody of the present disclosure comprises a HC CDR1, HC CDR2 and HC CDR3 of a heavy chain variable domain having the amino acid sequence of SEQ ID NO: 121. Alternatively or in addition, the anti-KLK7 antibody of the present disclosure comprises a LC CDR1, LC CDR2 and LC CDR3 of a light chain variable domain having the amino acid sequence of SEQ ID NO: 122.

In some embodiments, an antibody of the present disclosure comprises a HC CDR1 having the amino acid sequence of SEQ ID NO: 117, a HC CDR2 having the amino acid sequence of SEQ ID NO: 118, a HC CDR3 having the amino acid sequence of SEQ ID NO: 119, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 120.

In some embodiments, an anti-KLK7 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 117, HC CDR2 having the amino acid sequence of SEQ ID NO: 118, and HC CDR3 having the amino acid sequence of SEQ ID NO: 119. “Collectively,” as used anywhere in the present disclosure, means that the total number of amino acid variations in all of the three heavy chain CDRs is within the defined range. Alternatively or in addition, the anti-KLK7 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 4, LC CDR2 having the amino acid sequence of SEQ ID NO: 5, and LC CDR3 having the amino acid sequence of SEQ ID NO: 120.

In some embodiments, an anti-KLK7 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 117, HC CDR2 having the amino acid sequence of SEQ ID NO: 118, and HC CDR3 having the amino acid sequence of SEQ ID NO: 119. Alternatively or in addition, the anti-KLK7 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 4, LC CDR2 having the amino acid sequence of SEQ ID NO: 5, and LC CDR3 having the amino acid sequence of SEQ ID NO: 120.

In some embodiments, an anti-KLK7 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 117; a HC CDR2 having no more than 3 amino acid variations (e.g., no more than 3, 2, or 1 amino acid variation) as compared with the HC CDR2 having the amino acid sequence of SEQ ID NO: 118; and/or a HC CDR3 having no more than 3 amino acid variations (e.g., no more than 3, 2, or 1 amino acid variation) as compared with the HC CDR3 having the amino acid sequence of SEQ ID NO: 119. Alternatively or in addition, the anti-KLK7 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of SEQ ID NO: 5; and/or a LC CDR3 having no more than 3 amino acid variations (e.g., no more than 3, 2, or 1 amino acid variation) as compared with the LC CDR3 having the amino acid sequence of SEQ ID NO: 120.

In some embodiments, an anti-KLK7 antibody of the present disclosure comprises a VH comprising the amino acid sequence of SEQ ID NO: 121. Alternatively or in addition, the anti-KLK7 antibody of the present disclosure comprises a VL comprising the amino acid sequence of SEQ ID NO: 122.

In some embodiments, an anti-KLK7 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 121. Alternatively or in addition, the anti-KLK7 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 122.

In some embodiments, an anti-KLK7 antibody of the present disclosure comprises a VH comprising an amino acid sequence that is at least 80% (e.g., 80%, 85%, 90%, 95%, 98%, or 99%) identical to the VH as set forth in SEQ ID NO: 121. Alternatively or in addition, the anti-KLK7 antibody of the present disclosure comprises a VL comprising an amino acid sequence that is at least 80% (e.g., 80%, 85%, 90%, 95%, 98%, or 99%) identical to the VL as set forth in SEQ ID NO: 122.

In some embodiments, an anti-KLK7 antibody of the present disclosure comprises a HC CDR1, HC CDR2 and HC CDR3 of a heavy chain variable domain having the amino acid sequence of SEQ ID NO: 127. Alternatively or in addition, the anti-KLK7 antibody of the present disclosure comprises a LC CDR1, LC CDR2 and LC CDR3 of a light chain variable domain having the amino acid sequence of SEQ ID NO: 128.

In some embodiments, an antibody of the present disclosure comprises a HC CDR1 having the amino acid sequence of SEQ ID NO: 123, a HC CDR2 having the amino acid sequence of SEQ ID NO: 124, a HC CDR3 having the amino acid sequence of SEQ ID NO: 125, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 126.

In some embodiments, an anti-KLK7 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 123, HC CDR2 having the amino acid sequence of SEQ ID NO: 124, and HC CDR3 having the amino acid sequence of SEQ ID NO: 125. “Collectively,” as used anywhere in the present disclosure, means that the total number of amino acid variations in all of the three heavy chain CDRs is within the defined range. Alternatively or in addition, the anti-KLK7 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 4, LC CDR2 having the amino acid sequence of SEQ ID NO: 5, and LC CDR3 having the amino acid sequence of SEQ ID NO: 126.

In some embodiments, an anti-KLK7 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 123, HC CDR2 having the amino acid sequence of SEQ ID NO: 124, and HC CDR3 having the amino acid sequence of SEQ ID NO: 125. Alternatively or in addition, the anti-KLK7 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 4, LC CDR2 having the amino acid sequence of SEQ ID NO: 5, and LC CDR3 having the amino acid sequence of SEQ ID NO: 126.

In some embodiments, an anti-KLK7 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 123; a HC CDR2 having no more than 3 amino acid variations (e.g., no more than 3, 2, or 1 amino acid variation) as compared with the HC CDR2 having the amino acid sequence of SEQ ID NO: 124; and/or a HC CDR3 having no more than 3 amino acid variations (e.g., no more than 3, 2, or 1 amino acid variation) as compared with the HC CDR3 having the amino acid sequence of SEQ ID NO: 125. Alternatively or in addition, the anti-KLK7 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of SEQ ID NO: 5; and/or a LC CDR3 having no more than 3 amino acid variations (e.g., no more than 3, 2, or 1 amino acid variation) as compared with the LC CDR3 having the amino acid sequence of SEQ ID NO: 126.

In some embodiments, an anti-KLK7 antibody of the present disclosure comprises a VH comprising the amino acid sequence of SEQ ID NO: 127. Alternatively or in addition, the anti-KLK7 antibody of the present disclosure comprises a VL comprising the amino acid sequence of SEQ ID NO: 128.

In some embodiments, an anti-KLK7 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 127. Alternatively or in addition, the anti-KLK7 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 128.

In some embodiments, an anti-KLK7 antibody of the present disclosure comprises a VH comprising an amino acid sequence that is at least 80% (e.g., 80%, 85%, 90%, 95%, 98%, or 99%) identical to the VH as set forth in SEQ ID NO: 127. Alternatively or in addition, the anti-KLK7 antibody of the present disclosure comprises a VL comprising an amino acid sequence that is at least 80% (e.g., 80%, 85%, 90%, 95%, 98%, or 99%) identical to the VL as set forth in SEQ ID NO: 128.

In some embodiments, an anti-KLK7 antibody of the present disclosure comprises a HC CDR1, HC CDR2 and HC CDR3 of a heavy chain variable domain having the amino acid sequence of SEQ ID NO: 133. Alternatively or in addition, the anti-KLK7 antibody of the present disclosure comprises a LC CDR1, LC CDR2 and LC CDR3 of a light chain variable domain having the amino acid sequence of SEQ ID NO: 134.

In some embodiments, an antibody of the present disclosure comprises a HC CDR1 having the amino acid sequence of SEQ ID NO: 19, a HC CDR2 having the amino acid sequence of SEQ ID NO: 20, a HC CDR3 having the amino acid sequence of SEQ ID NO: 129, a LC CDR1 having the amino acid sequence of SEQ ID NO: 130, a LC CDR2 having the amino acid sequence of SEQ ID NO: 131, and a LC CDR3 having the amino acid sequence of SEQ ID NO: 132.

In some embodiments, an anti-KLK7 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 19, HC CDR2 having the amino acid sequence of SEQ ID NO: 20, and HC CDR3 having the amino acid sequence of SEQ ID NO: 129. “Collectively,” as used anywhere in the present disclosure, means that the total number of amino acid variations in all of the three heavy chain CDRs is within the defined range. Alternatively or in addition, the anti-KLK7 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 130, LC CDR2 having the amino acid sequence of SEQ ID NO: 131, and LC CDR3 having the amino acid sequence of SEQ ID NO: 132.

In some embodiments, an anti-KLK7 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 19, HC CDR2 having the amino acid sequence of SEQ ID NO: 20, and HC CDR3 having the amino acid sequence of SEQ ID NO: 129. Alternatively or in addition, the anti-KLK7 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 130, LC CDR2 having the amino acid sequence of SEQ ID NO: 131, and LC CDR3 having the amino acid sequence of SEQ ID NO: 132.

In some embodiments, an anti-KLK7 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 19; a HC CDR2 having no more than 3 amino acid variations (e.g., no more than 3, 2, or 1 amino acid variation) as compared with the HC CDR2 having the amino acid sequence of SEQ ID NO: 20; and/or a HC CDR3 having no more than 3 amino acid variations (e.g., no more than 3, 2, or 1 amino acid variation) as compared with the HC CDR3 having the amino acid sequence of SEQ ID NO: 129. Alternatively or in addition, the anti-KLK7 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 130; a LC CDR2 having no more than 3 amino acid variations (e.g., no more than 3, 2, or 1 amino acid variation) as compared with the LC CDR2 having the amino acid sequence of SEQ ID NO: 131; and/or a LC CDR3 having no more than 3 amino acid variations (e.g., no more than 3, 2, or 1 amino acid variation) as compared with the LC CDR3 having the amino acid sequence of SEQ ID NO: 132.

In some embodiments, an anti-KLK7 antibody of the present disclosure comprises a VH comprising the amino acid sequence of SEQ ID NO: 133. Alternatively or in addition, the anti-KLK7 antibody of the present disclosure comprises a VL comprising the amino acid sequence of SEQ ID NO: 134.

In some embodiments, an anti-KLK7 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 133. Alternatively or in addition, the anti-KLK7 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 134.

In some embodiments, an anti-KLK7 antibody of the present disclosure comprises a VH comprising an amino acid sequence that is at least 80% (e.g., 80%, 85%, 90%, 95%, 98%, or 99%) identical to the VH as set forth in SEQ ID NO: 133. Alternatively or in addition, the anti-KLK7 antibody of the present disclosure comprises a VL comprising an amino acid sequence that is at least 80% (e.g., 80%, 85%, 90%, 95%, 98%, or 99%) identical to the VL as set forth in SEQ ID NO: 134.

In some embodiments, an anti-KLK7 antibody of the present disclosure comprises a HC CDR1, HC CDR2 and HC CDR3 of a heavy chain variable domain having the amino acid sequence of SEQ ID NO: 139. Alternatively or in addition, the anti-KLK7 antibody of the present disclosure comprises a LC CDR1, LC CDR2 and LC CDR3 of a light chain variable domain having the amino acid sequence of SEQ ID NO: 140.

In some embodiments, an antibody of the present disclosure comprises a HC CDR1 having the amino acid sequence of SEQ ID NO: 135, a HC CDR2 having the amino acid sequence of SEQ ID NO: 136, a HC CDR3 having the amino acid sequence of SEQ ID NO: 137, a LC CDR1 having the amino acid sequence of SEQ ID NO: 130, a LC CDR2 having the amino acid sequence of SEQ ID NO: 131, and a LC CDR3 having the amino acid sequence of SEQ ID NO: 138.

In some embodiments, an anti-KLK7 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 135, HC CDR2 having the amino acid sequence of SEQ ID NO: 136, and HC CDR3 having the amino acid sequence of SEQ ID NO: 137. “Collectively,” as used anywhere in the present disclosure, means that the total number of amino acid variations in all of the three heavy chain CDRs is within the defined range. Alternatively or in addition, the anti-KLK7 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 130, LC CDR2 having the amino acid sequence of SEQ ID NO: 131, and LC CDR3 having the amino acid sequence of SEQ ID NO: 138.

In some embodiments, an anti-KLK7 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 135, HC CDR2 having the amino acid sequence of SEQ ID NO: 136, and HC CDR3 having the amino acid sequence of SEQ ID NO: 137. Alternatively or in addition, the anti-KLK7 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 130, LC CDR2 having the amino acid sequence of SEQ ID NO: 131, and LC CDR3 having the amino acid sequence of SEQ ID NO: 138.

In some embodiments, an anti-KLK7 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 135; a HC CDR2 having no more than 3 amino acid variations (e.g., no more than 3, 2, or 1 amino acid variation) as compared with the HC CDR2 having the amino acid sequence of SEQ ID NO: 136; and/or a HC CDR3 having no more than 3 amino acid variations (e.g., no more than 3, 2, or 1 amino acid variation) as compared with the HC CDR3 having the amino acid sequence of SEQ ID NO: 137. Alternatively or in addition, the anti-KLK7 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 130; a LC CDR2 having no more than 3 amino acid variations (e.g., no more than 3, 2, or 1 amino acid variation) as compared with the LC CDR2 having the amino acid sequence of SEQ ID NO: 131; and/or a LC CDR3 having no more than 3 amino acid variations (e.g., no more than 3, 2, or 1 amino acid variation) as compared with the LC CDR3 having the amino acid sequence of SEQ ID NO: 138.

In some embodiments, an anti-KLK7 antibody of the present disclosure comprises a VH comprising the amino acid sequence of SEQ ID NO: 139. Alternatively or in addition, the anti-KLK7 antibody of the present disclosure comprises a VL comprising the amino acid sequence of SEQ ID NO: 140.

In some embodiments, an anti-KLK7 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 139. Alternatively or in addition, the anti-KLK7 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 140.

In some embodiments, an anti-KLK7 antibody of the present disclosure comprises a VH comprising an amino acid sequence that is at least 80% (e.g., 80%, 85%, 90%, 95%, 98%, or 99%) identical to the VH as set forth in SEQ ID NO: 139. Alternatively or in addition, the anti-KLK7 antibody of the present disclosure comprises a VL comprising an amino acid sequence that is at least 80% (e.g., 80%, 85%, 90%, 95%, 98%, or 99%) identical to the VL as set forth in SEQ ID NO: 140.

In some embodiments, an anti-KLK7 antibody of the present disclosure comprises a HC CDR1, HC CDR2 and HC CDR3 of a heavy chain variable domain having the amino acid sequence of SEQ ID NO: 145. Alternatively or in addition, the anti-KLK7 antibody of the present disclosure comprises a LC CDR1, LC CDR2 and LC CDR3 of a light chain variable domain having the amino acid sequence of SEQ ID NO: 146.

In some embodiments, an antibody of the present disclosure comprises a HC CDR1 having the amino acid sequence of SEQ ID NO: 38, a HC CDR2 having the amino acid sequence of SEQ ID NO: 141, a HC CDR3 having the amino acid sequence of SEQ ID NO: 142, a LC CDR1 having the amino acid sequence of SEQ ID NO: 143, a LC CDR2 having the amino acid sequence of SEQ ID NO: 23, and a LC CDR3 having the amino acid sequence of SEQ ID NO: 144.

In some embodiments, an anti-KLK7 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 38, HC CDR2 having the amino acid sequence of SEQ ID NO: 141, and HC CDR3 having the amino acid sequence of SEQ ID NO: 142. “Collectively,” as used anywhere in the present disclosure, means that the total number of amino acid variations in all of the three heavy chain CDRs is within the defined range. Alternatively or in addition, the anti-KLK7 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 143, LC CDR2 having the amino acid sequence of SEQ ID NO: 23, and LC CDR3 having the amino acid sequence of SEQ ID NO: 144.

In some embodiments, an anti-KLK7 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 38, HC CDR2 having the amino acid sequence of SEQ ID NO: 141, and HC CDR3 having the amino acid sequence of SEQ ID NO: 142. Alternatively or in addition, the anti-KLK7 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 143, LC CDR2 having the amino acid sequence of SEQ ID NO: 23, and LC CDR3 having the amino acid sequence of SEQ ID NO: 144.

In some embodiments, an anti-KLK7 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 38; a HC CDR2 having no more than 3 amino acid variations (e.g., no more than 3, 2, or 1 amino acid variation) as compared with the HC CDR2 having the amino acid sequence of SEQ ID NO: 141; and/or a HC CDR3 having no more than 3 amino acid variations (e.g., no more than 3, 2, or 1 amino acid variation) as compared with the HC CDR3 having the amino acid sequence of SEQ ID NO: 142. Alternatively or in addition, the anti-KLK7 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 143; a LC CDR2 having no more than 3 amino acid variations (e.g., no more than 3, 2, or 1 amino acid variation) as compared with the LC CDR2 having the amino acid sequence of SEQ ID NO: 23; and/or a LC CDR3 having no more than 3 amino acid variations (e.g., no more than 3, 2, or 1 amino acid variation) as compared with the LC CDR3 having the amino acid sequence of SEQ ID NO: 144.

In some embodiments, an anti-KLK7 antibody of the present disclosure comprises a VH comprising the amino acid sequence of SEQ ID NO: 145. Alternatively or in addition, the anti-KLK7 antibody of the present disclosure comprises a VL comprising the amino acid sequence of SEQ ID NO: 146.

In some embodiments, an anti-KLK7 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 145. Alternatively or in addition, the anti-KLK7 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 146.

In some embodiments, an anti-KLK7 antibody of the present disclosure comprises a VH comprising an amino acid sequence that is at least 80% (e.g., 80%, 85%, 90%, 95%, 98%, or 99%) identical to the VH as set forth in SEQ ID NO: 145. Alternatively or in addition, the anti-KLK7 antibody of the present disclosure comprises a VL comprising an amino acid sequence that is at least 80% (e.g., 80%, 85%, 90%, 95%, 98%, or 99%) identical to the VL as set forth in SEQ ID NO: 146.

In some embodiments, an anti-KLK7 antibody of the present disclosure comprises a HC CDR1, HC CDR2 and HC CDR3 of a heavy chain variable domain having the amino acid sequence of SEQ ID NO: 151. Alternatively or in addition, the anti-KLK7 antibody of the present disclosure comprises a LC CDR1, LC CDR2 and LC CDR3 of a light chain variable domain having the amino acid sequence of SEQ ID NO: 152.

In some embodiments, an antibody of the present disclosure comprises a HC CDR1 having the amino acid sequence of SEQ ID NO: 147, a HC CDR2 having the amino acid sequence of SEQ ID NO: 148, a HC CDR3 having the amino acid sequence of SEQ ID NO: 149, a LC CDR1 having the amino acid sequence of SEQ ID NO: 143, a LC CDR2 having the amino acid sequence of SEQ ID NO: 23, and a LC CDR3 having the amino acid sequence of SEQ ID NO: 150.

In some embodiments, an anti-KLK7 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 147, HC CDR2 having the amino acid sequence of SEQ ID NO: 148, and HC CDR3 having the amino acid sequence of SEQ ID NO: 149. “Collectively,” as used anywhere in the present disclosure, means that the total number of amino acid variations in all of the three heavy chain CDRs is within the defined range. Alternatively or in addition, the anti-KLK7 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 143, LC CDR2 having the amino acid sequence of SEQ ID NO: 23, and LC CDR3 having the amino acid sequence of SEQ ID NO: 150.

In some embodiments, an anti-KLK7 antibody of the present disclosure comprises a HC CDR1, a HC CDR2, and a HC CDR3 that collectively are at least 80% (e.g., 80%, 85%, 90%, 95%, 98%, or 99%) identical to the HC CDR1 having the amino acid sequence of SEQ ID NO: 147, HC CDR2 having the amino acid sequence of SEQ ID NO: 148, and HC CDR3 having the amino acid sequence of SEQ ID NO: 149. Alternatively or in addition, the anti-KLK7 antibody of the present disclosure comprises a LC CDR1, a LC CDR2, and a LC CDR3 that collectively are at least 80% (e.g., 80%, 85%, 90%, 95%, 98%, or 99%) identical to the to the LC CDR1 having the amino acid sequence of SEQ ID NO: 143, LC CDR2 having the amino acid sequence of SEQ ID NO: 23, and LC CDR3 having the amino acid sequence of SEQ ID NO: 150.

In some embodiments, an anti-KLK7 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 147; a HC CDR2 having no more than 3 amino acid variations (e.g., no more than 3, 2, or 1 amino acid variation) as compared with the HC CDR2 having the amino acid sequence of SEQ ID NO: 148; and/or a HC CDR3 having no more than 3 amino acid variations (e.g., no more than 3, 2, or 1 amino acid variation) as compared with the HC CDR3 having the amino acid sequence of SEQ ID NO: 149. Alternatively or in addition, the anti-KLK7 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 143; a LC CDR2 having no more than 3 amino acid variations (e.g., no more than 3, 2, or 1 amino acid variation) as compared with the LC CDR2 having the amino acid sequence of SEQ ID NO: 23; and/or a LC CDR3 having no more than 3 amino acid variations (e.g., no more than 3, 2, or 1 amino acid variation) as compared with the LC CDR3 having the amino acid sequence of SEQ ID NO: 150.

In some embodiments, an anti-KLK7 antibody of the present disclosure comprises a VH comprising the amino acid sequence of SEQ ID NO: 151. Alternatively or in addition, the anti-KLK7 antibody of the present disclosure comprises a VL comprising the amino acid sequence of SEQ ID NO: 152.

In some embodiments, an anti-KLK7 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 151. Alternatively or in addition, the anti-KLK7 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 152.

In some embodiments, an anti-KLK7 antibody of the present disclosure comprises a VH comprising an amino acid sequence that is at least 80% (e.g., 80%, 85%, 90%, 95%, 98%, or 99%) identical to the VH as set forth in SEQ ID NO: 151. Alternatively or in addition, the anti-KLK7 antibody of the present disclosure comprises a VL comprising an amino acid sequence that is at least 80% (e.g., 80%, 85%, 90%, 95%, 98%, or 99%) identical to the VL as set forth in SEQ ID NO: 152.

The antibodies described herein can be in any antibody form, including, but not limited to, intact (i.e., full-length) antibodies, antigen-binding fragments thereof (such as Fab, F(ab′), F(ab′)2, Fv), single chain antibodies, bi-specific antibodies, or nanobodies. In some embodiments, the anti-KLK7 antibody described herein is a scFv. In some embodiments, the anti-KLK7 antibody described herein is a scFv-Fab (e.g., scFv fused to a portion of a constant region).

In some embodiments, an anti-KLK7 antibody of the present disclosure is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

In some embodiments, an antibody of the present disclosure comprises a VL domain and/or VH domain of any one of the anti-KLK7 antibodies selected from Tables 2a and 2b, and comprises a constant region comprising the amino acid sequences of the constant regions of an IgG, IgE, IgM, IgD, IgA or IgY immunoglobulin molecule, any class (e.g., IgG1, IgG2, IgG3, IgG4, IgA1 and IgA2), or any subclass (e.g., IgG2a and IgG2b) of immunoglobulin molecule. Non-limiting examples of human constant regions are described in the art, e.g., see Kabat E A et al., (1991) supra.

In some embodiments, the light chain of any of the anti-KLK7 antibodies described herein may further comprise a light chain constant region (CL), which can be any CL known in the art. In some examples, the CL is a kappa light chain. In other examples, the CL is a lambda light chain. In some embodiments, the CL is a kappa light chain.

Other antibody heavy and light chain constant regions are well known in the art, e.g., those provided in the IMGT database (www.imgt.org) or at www.vbase2.org/vbstat.php., both of which are incorporated by reference herein.

Examples of heavy chain and light chain amino acid sequences of the anti-KLK7 antibodies described are provided in Tables 2a and 2b above.

In certain embodiments, the CDRs of an antibody may have different amino acid sequences when different definition systems are used (e.g., the IMGT definition, the Kabat definition, or the Chothia definition). A definition system annotates each amino acid in a given antibody sequence (e.g., VH or VL sequence) with a number, and numbers corresponding to the heavy chain and light chain CDRs are provided in Table 3.

CDRs are listed in Tables 1a, 1b, 2a, and 2b. One skilled in the art is able to derive the CDR sequences using the different numbering systems for the antibodies provided in Tables 1a and 2a.

TABLE 3
CDR Definitions

| | IMGT¹ | Kabat² | Chothia³ |
|---|---|---|---|
| HC CDR1 | 27-38 | 31-35 | 26-32 |
| HC CDR2 | 56-65 | 50-65 | 53-55 |
| HC CDR3 | 105-116/117 | 95-102 | 96-101 |
| LC CDR1 | 27-38 | 24-34 | 26-32 |
| LC CDR2 | 56-65 | 50-56 | 50-52 |
| LC CDR3 | 105-116/117 | 89-97 | 91-96 |

¹IMGT ®, the international ImMunoGeneTics information system ®, imgt.org, Lefranc, M.-P. et al., Nucleic Acids Res., 27: 209-212 (1999)
²Kabat et al. (1991) Sequences of Proteins of Immunological Interest, Fifth Edition, U.S. Department of Health and Human Services, NIH Publication No. 91-3242
³Chothia et al., J. Mol. Biol. 196: 901-917 (1987))

In some embodiments, conservative mutations can be introduced into antibody sequences (e.g., CDRs or framework sequences) at positions where the residues are not likely to be involved in interacting with a target antigen (e.g., human or mouse KLK7), for example, as determined based on a crystal structure. In some embodiments, one, two or more mutations (e.g., amino acid substitutions) are introduced into the Fc region of an anti-KLK7 antibody described herein (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to alter one or more functional properties of the antibody, such as serum half-life, complement fixation, Fc receptor binding and/or antigen-dependent cellular cytotoxicity.

In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more mutations (e.g., amino acid substitutions) are introduced into the Fc region of an antibody described herein (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to decrease the half-life of the anti-KLK7 antibody in vivo. In some embodiments, one, two or more amino acid mutations (i.e., substitutions, insertions or deletions) are introduced into an IgG constant domain, or FcRn-binding fragment thereof (preferably an Fc or hinge-Fc domain fragment) to increase the half-life of the antibody in vivo. In some embodiments, the antibodies can have one or more amino acid mutations (e.g., substitutions) in the second constant (CH2) domain (residues 231-340 of human IgG1) and/or the third constant (CH3) domain (residues 341-447 of human IgG1), with numbering according to the EU index in Kabat (Kabat E A et al., (1991) supra). In some embodiments, the constant region of the IgG1 of an antibody described herein comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall'Acqua W F et al., (2006) J Biol Chem 281: 23514-24). In some embodiments, an antibody comprises an IgG constant domain comprising one, two, three or more amino acid substitutions of amino acid residues at positions 251-257, 285-290, 308-314, 385-389, and 428-436, numbered according to the EU index as in Kabat.

In some embodiments, an antibody comprises an Fc region that has been engineered for half-life extension purposes, e.g., by introducing M428L and/or N434A substitutions. Non-limiting examples of such Fc variants effecting half-life in circulation are provided in Saunders K O, Conceptual Approaches to Modulating Antibody Effector Functions and Circulation Half-Life, Front Immunol. 2019; 10: 1296, the contents of which are incorporated herein by reference.

In some embodiments, one, two or more amino acid substitutions are introduced into an IgG constant domain Fc region to alter the effector function(s) of the anti-KLK7 antibody, e.g., by introducing Leu234Ala and Leu235Ala mutations (commonly called LALA mutations). The effector ligand to which affinity is altered can be, for example, an Fc receptor or the C1 component of complement. This approach is described in further detail in U.S. Pat. Nos. 5,624,821 and 5,648,260. In some embodiments, the deletion or inactivation (through point mutations or other means) of a constant region domain can reduce Fc receptor binding of the circulating antibody thereby increasing tumor localization. See, e.g., U.S. Pat. Nos. 5,585,097 and 8,591,886 for a description of mutations that delete or inactivate the constant domain and thereby increase tumor localization. In some embodiments, one or more amino acid substitutions may be introduced into the Fc region of an antibody described herein to remove potential glycosylation sites on Fc region, which may reduce Fc receptor binding (see, e.g., Shields R L et al., (2001) J Biol Chem 276: 6591-604).

In some embodiments, one or more amino in the constant region of an anti-KLK7 antibody described herein can be replaced with a different amino acid residue such that the antibody has altered Clq binding and/or reduced or abolished complement dependent cytotoxicity (CDC). This approach is described in further detail in U.S. Pat. No. 6,194,551 (Idusogie et al). In some embodiments, one or more amino acid residues in the N-terminal region of the CH2 domain of an antibody described herein are altered to thereby alter the ability of the antibody to fix complement. This approach is described further in International Publication No. WO 94/29351. In some embodiments, the Fc region of an antibody described herein is modified to increase the ability of the antibody to mediate antibody dependent cellular cytotoxicity (ADCC) and/or to increase the affinity of the antibody for an Fcγ receptor. This approach is described further in International Publication No. WO 00/42072.

In some embodiments, an antibody comprises an Fc variant comprising amino acid substitutions L234A, L235E, and P329G, wherein numbering is according to the EU index. In some embodiments, the antibody comprising the Fc variant exhibits reduced affinity to one or more or each of FcγRJ, FcγRIIA, FcγRIIIA, and Clq as compared to an antibody comprising the wild-type human Fc region. Examples of such Fc variants are provided in International Patent Application Publication No.: WO 2021/055669 entitled, FC VARIANTS WITH REDUCED EFFECTOR FUNCTION, published on Mar. 25, 2021; and US Patent Application Publication No.: US 2021-0087271 entitled, FC VARIANTS WITH REDUCED EFFECTOR FUNCTION, published on Mar. 25, 2021, the contents of which are incorporated herein by reference.

In some embodiments, the heavy and/or light chain variable domain(s) sequence(s) of the antibodies provided herein can be used to generate, for example, CDR-grafted, chimeric, humanized, or composite human antibodies or antigen-binding fragments, as described elsewhere herein. As understood by one of ordinary skill in the art, any variant, CDR-grafted, chimeric, humanized, or composite antibodies derived from any of the antibodies provided herein may be useful in the compositions and methods described herein and will maintain the ability to specifically bind KLK7, such that the variant, CDR-grafted, chimeric, humanized, or composite antibody has at least 50%, at least 60%, at least 70%, at least 80%, at least 90%, at least 95% or more binding to KLK7 relative to the original antibody from which it is derived.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal S., et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like hinge sequence. Accordingly, any of the antibodies may include a stabilizing ‘Adair’ mutation.

In some embodiments, an antibody is modified, e.g., modified via glycosylation, phosphorylation, sumoylation, and/or methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or phosphoglycosylation. In some embodiments, the one or more sugar or carbohydrate molecules are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, there are about 1-10, about 1-5, about 5-10, about 1-4, about 1-3, or about 2 sugar molecules. In some embodiments, a glycosylated antibody is fully or partially glycosylated. In some embodiments, an antibody is glycosylated by chemical reactions or by enzymatic means. In some embodiments, an antibody is glycosylated in vitro or inside a cell, which may optionally be deficient in an enzyme in the N- or O-glycosylation pathway, e.g. a glycosyltransferase. In some embodiments, an antibody is functionalized with sugar or carbohydrate molecules as described in International Patent Application Publication WO2014065661, published on May 1, 2014, entitled, “Modified antibody, antibody-conjugate and process for the preparation thereof”.

In some embodiments, any one of the anti-KLK7 antibodies described herein may comprise a signal peptide in the heavy and/or light chain sequence (e.g., a N-terminal signal peptide). In some embodiments, the anti-KLK7 antibody described herein comprises any one of the VH and VL sequences, any one of the IgG heavy chain and light chain sequences, or any one of the F(ab′) heavy chain and light chain sequences described herein, and further comprises a signal peptide (e.g., a N-terminal signal peptide).

### (c) Multispecific Antibodies Targeting KLK5 and KLK7

In some embodiments, an antibody provided here is a multi-specific antibody (e.g., a bispecific antibody). For example, in some embodiments, one or more anti-KLK5 antibodies may be combined with one or more anti-KLK7 antibody to produce a multi-specific or a bispecific anti-KLK5/KLK7 antibody. For example, one or more anti-KLK5 antibody as described herein (Table 1a and Table 1b), can be combined with one or more anti-KLK7 antibody described herein (Table 2a and Table 2b) to produce a multi-specific antibody.

In some embodiments, an anti-KLK5/KLK7 multispecific antibody described herein may be characterized by reference to certain functional properties. In some embodiments, an anti-KLK5/KLK7 multispecific antibody described herein has a first arm that binds specifically to KLK5 and a second arm that binds specifically to KLK7. In some embodiments, an anti-KLK5/KLK7 multispecific antibody has a first arm that binds specifically to the active form of KLK5 and a second arm that binds specifically to the active form of KLK7. In some embodiments, an anti-KLK5/KLK7 multispecific antibody does not bind the inactive form (sometimes referred to as the proform or pro-form) of KLK5 and KLK7. In some embodiments, an anti-KLK5/KLK7 multispecific antibody inhibits KLK5 and KLK7 protease activity. In some embodiments, an anti-KLK5/KLK7 multispecific antibody is not cleaved by KLK5 or KLK7 when bound to KLK5 or KLK7. In some embodiments, an anti-KLK5/KLK7 multispecific antibody competes with SPINK5 and/or leupeptin for binding of the active site of KLK5 and KLK7. In some embodiments, an anti-KLK5/KLK7 multispecific antibody reduces hyperkeratosis and desquamation. In some embodiments, an anti-KLK5/KLK7 multispecific antibody reduces stratum corneum thickness. In some embodiments, an anti-KLK5/KLK7 multispecific antibody reduces inflammation and epidermal effects.

In some embodiments, a bispecific antibody is provided that comprises an antigen specific binding site from an anti-KLK5 antibody selected from (Table 1a and Table 1b) and comprises an antigen specific binding site from an anti-KLK7 antibody selected from (Table 2a and Table 2b), examples of such pairs of anti-KLK5 antibodies and anti-KLK7 antibodies are listed in Table 4. In some embodiments, a bispecific antibody is provided that comprises HC CDR1, a HC CDR2, and a HC CDR3 of an anti-KLK5 antibody selected from (Table 1a and Table 1b) and comprises HC CDR1, a HC CDR2, and a HC CDR3 of an anti-KLK7 antibody selected from (Table 2a and Table 2b), examples of such pairs of anti-KLK5 antibodies and anti-KLK7 antibodies are listed in Table 4. In some embodiments, a bispecific antibody is provided that comprises LC CDR1, a LC CDR2, and a LC CDR3 of an anti-KLK5 antibody selected from (Table 1a and Table 1b) and comprises LC CDR1, a LC CDR2, and a LC CDR3 of an anti-KLK7 antibody selected from (Table 2a and Table 2b), examples of such pairs of anti-KLK5 antibodies and anti-KLK7 antibodies are listed in Table 4.

In some embodiments, a bispecific antibody is provided that comprises a VH domain of an anti-KLK5 antibody selected from (Table 1a and Table 1b) and comprises VH domain of an anti-KLK7 antibody selected from (Table 2a and Table 2b), examples of such pairs of anti-KLK5 antibodies and anti-KLK7 antibodies are listed in Table 4. In some embodiments, a bispecific antibody is provided that comprises VL domain of an anti-KLK5 antibody selected from (Table 1a and Table 1b) and comprises VL domain of an anti-KLK7 antibody selected from (Table 2a and Table 2b), examples of such pairs of anti-KLK5 antibodies and anti-KLK7 antibodies are listed in Table 4.

In some embodiments, a bispecific antibody is provided that comprises a heavy chain of an anti-KLK5 antibody selected from (Table 1a and Table 1b) and comprises heavy chain of an anti-KLK7 antibody selected from (Table 2a and Table 2b), examples of such pairs of anti-KLK5 antibodies and anti-KLK7 antibodies are listed in Table 4. In some embodiments, a bispecific antibody is provided that comprises light chain of an anti-KLK5 antibody selected from (Table 1a and Table 1b) and comprises light chain of an anti-KLK7 antibody selected from (Table 2a and Table 2b), examples of such pairs of anti-KLK5 antibodies and anti-KLK7 antibodies are listed in Table 4.

TABLE 4
KLK5 / KLK7 Bispecific Antibody Combinations

| Anti-KLK5 | Anti-KLK7 |
|---|---|
| Antibody | Antibody |
| KLK5-L1-Ab0 | KLK7-L1-Ab0 |
| KLK5-L1-Ab0 | KLK7-L1-Ab1 |
| KLK5-L1-Ab0 | KLK7-L1-Ab2 |
| KLK5-L1-Ab0 | KLK7-L1-Ab3 |
| KLK5-L1-Ab0 | KLK7-L1-Ab4 |
| KLK5-L1-Ab0 | KLK7-L1-Ab5 |
| KLK5-L1-Ab0 | KLK7-L1-Ab6 |
| KLK5-L1-Ab0 | KLK7-L1-Ab7 |
| KLK5-L1-Ab0 | KLK7-L1-Ab8 |
| KLK5-L1-Ab0 | KLK7-L2-Ab0 |
| KLK5-L1-Ab0 | KLK7-L2-Ab1 |
| KLK5-L1-Ab0 | KLK7-L2-Ab2 |
| KLK5-L1-Ab0 | KLK7-L2-Ab3 |
| KLK5-L1-Ab0 | KLK7-L2-Ab4 |
| KLK5-L1-Ab0 | KLK7-L2-Ab5 |
| KLK5-L1-Ab0 | KLK7-L3-Ab0 |
| KLK5-L1-Ab0 | KLK7-L3-Ab1 |
| KLK5-L1-Ab0 | KLK7-L3-Ab2 |
| KLK5-L1-Ab0 | KLK7-L3-Ab3 |
| KLK5-L1-Ab0 | KLK7-L4-Ab0 |
| KLK5-L1-Ab1 | KLK7-L1-Ab0 |
| KLK5-L1-Ab1 | KLK7-L1-Ab1 |
| KLK5-L1-Ab1 | KLK7-L1-Ab2 |
| KLK5-L1-Ab1 | KLK7-L1-Ab3 |
| KLK5-L1-Ab1 | KLK7-L1-Ab4 |
| KLK5-L1-Ab1 | KLK7-L1-Ab5 |
| KLK5-L1-Ab1 | KLK7-L1-Ab6 |
| KLK5-L1-Ab1 | KLK7-L1-Ab7 |
| KLK5-L1-Ab1 | KLK7-L1-Ab8 |
| KLK5-L1-Ab1 | KLK7-L2-Ab0 |
| KLK5-L1-Ab1 | KLK7-L2-Ab1 |
| KLK5-L1-Ab1 | KLK7-L2-Ab2 |
| KLK5-L1-Ab1 | KLK7-L2-Ab3 |
| KLK5-L1-Ab1 | KLK7-L2-Ab4 |
| KLK5-L1-Ab1 | KLK7-L2-Ab5 |
| KLK5-L1-Ab1 | KLK7-L3-Ab0 |
| KLK5-L1-Ab1 | KLK7-L3-Ab1 |
| KLK5-L1-Ab1 | KLK7-L3-Ab2 |
| KLK5-L1-Ab1 | KLK7-L3-Ab3 |
| KLK5-L1-Ab1 | KLK7-L4-Ab0 |
| KLK5-L1-Ab2 | KLK7-L1-Ab0 |
| KLK5-L1-Ab2 | KLK7-L1-Ab1 |
| KLK5-L1-Ab2 | KLK7-L1-Ab2 |
| KLK5-L1-Ab2 | KLK7-L1-Ab3 |
| KLK5-L1-Ab2 | KLK7-L1-Ab4 |
| KLK5-L1-Ab2 | KLK7-L1-Ab5 |
| KLK5-L1-Ab2 | KLK7-L1-Ab6 |
| KLK5-L1-Ab2 | KLK7-L1-Ab7 |
| KLK5-L1-Ab2 | KLK7-L1-Ab8 |
| KLK5-L1-Ab2 | KLK7-L2-Ab0 |
| KLK5-L1-Ab2 | KLK7-L2-Ab1 |
| KLK5-L1-Ab2 | KLK7-L2-Ab2 |
| KLK5-L1-Ab2 | KLK7-L2-Ab3 |
| KLK5-L1-Ab2 | KLK7-L2-Ab4 |
| KLK5-L1-Ab2 | KLK7-L2-Ab5 |
| KLK5-L1-Ab2 | KLK7-L3-Ab0 |
| KLK5-L1-Ab2 | KLK7-L3-Ab1 |
| KLK5-L1-Ab2 | KLK7-L3-Ab2 |
| KLK5-L1-Ab2 | KLK7-L3-Ab3 |
| KLK5-L1-Ab2 | KLK7-L4-Ab0 |
| KLK5-L1-Ab3 | KLK7-L1-Ab0 |
| KLK5-L1-Ab3 | KLK7-L1-Ab1 |
| KLK5-L1-Ab3 | KLK7-L1-Ab2 |
| KLK5-L1-Ab3 | KLK7-L1-Ab3 |
| KLK5-L1-Ab3 | KLK7-L1-Ab4 |
| KLK5-L1-Ab3 | KLK7-L1-Ab5 |
| KLK5-L1-Ab3 | KLK7-L1-Ab6 |
| KLK5-L1-Ab3 | KLK7-L1-Ab7 |
| KLK5-L1-Ab3 | KLK7-L1-Ab8 |
| KLK5-L1-Ab3 | KLK7-L2-Ab0 |
| KLK5-L1-Ab3 | KLK7-L2-Ab1 |
| KLK5-L1-Ab3 | KLK7-L2-Ab2 |
| KLK5-L1-Ab3 | KLK7-L2-Ab3 |
| KLK5-L1-Ab3 | KLK7-L2-Ab4 |
| KLK5-L1-Ab3 | KLK7-L2-Ab5 |
| KLK5-L1-Ab3 | KLK7-L3-Ab0 |
| KLK5-L1-Ab3 | KLK7-L3-Ab1 |
| KLK5-L1-Ab3 | KLK7-L3-Ab2 |
| KLK5-L1-Ab3 | KLK7-L3-Ab3 |
| KLK5-L1-Ab3 | KLK7-L4-Ab0 |
| KLK5-L1-Ab4 | KLK7-L1-Ab0 |
| KLK5-L1-Ab4 | KLK7-L1-Ab1 |
| KLK5-L1-Ab4 | KLK7-L1-Ab2 |
| KLK5-L1-Ab4 | KLK7-L1-Ab3 |
| KLK5-L1-Ab4 | KLK7-L1-Ab4 |
| KLK5-L1-Ab4 | KLK7-L1-Ab5 |
| KLK5-L1-Ab4 | KLK7-L1-Ab6 |
| KLK5-L1-Ab4 | KLK7-L1-Ab7 |
| KLK5-L1-Ab4 | KLK7-L1-Ab8 |
| KLK5-L1-Ab4 | KLK7-L2-Ab0 |
| KLK5-L1-Ab4 | KLK7-L2-Ab1 |
| KLK5-L1-Ab4 | KLK7-L2-Ab2 |
| KLK5-L1-Ab4 | KLK7-L2-Ab3 |
| KLK5-L1-Ab4 | KLK7-L2-Ab4 |
| KLK5-L1-Ab4 | KLK7-L2-Ab5 |
| KLK5-L1-Ab4 | KLK7-L3-Ab0 |
| KLK5-L1-Ab4 | KLK7-L3-Ab1 |
| KLK5-L1-Ab4 | KLK7-L3-Ab2 |
| KLK5-L1-Ab4 | KLK7-L3-Ab3 |
| KLK5-L1-Ab4 | KLK7-L4-Ab0 |
| KLK5-L1-Ab5 | KLK7-L1-Ab0 |
| KLK5-L1-Ab5 | KLK7-L1-Ab1 |
| KLK5-L1-Ab5 | KLK7-L1-Ab2 |
| KLK5-L1-Ab5 | KLK7-L1-Ab3 |
| KLK5-L1-Ab5 | KLK7-L1-Ab4 |
| KLK5-L1-Ab5 | KLK7-L1-Ab5 |
| KLK5-L1-Ab5 | KLK7-L1-Ab6 |
| KLK5-L1-Ab5 | KLK7-L1-Ab7 |
| KLK5-L1-Ab5 | KLK7-L1-Ab8 |
| KLK5-L1-Ab5 | KLK7-L2-Ab0 |
| KLK5-L1-Ab5 | KLK7-L2-Ab1 |
| KLK5-L1-Ab5 | KLK7-L2-Ab2 |
| KLK5-L1-Ab5 | KLK7-L2-Ab3 |
| KLK5-L1-Ab5 | KLK7-L2-Ab4 |
| KLK5-L1-Ab5 | KLK7-L2-Ab5 |
| KLK5-L1-Ab5 | KLK7-L3-Ab0 |
| KLK5-L1-Ab5 | KLK7-L3-Ab1 |
| KLK5-L1-Ab5 | KLK7-L3-Ab2 |
| KLK5-L1-Ab5 | KLK7-L3-Ab3 |
| KLK5-L1-Ab5 | KLK7-L4-Ab0 |
| KLK5-L2-Ab0 | KLK7-L1-Ab0 |
| KLK5-L2-Ab0 | KLK7-L1-Ab1 |
| KLK5-L2-Ab0 | KLK7-L1-Ab2 |
| KLK5-L2-Ab0 | KLK7-L1-Ab3 |
| KLK5-L2-Ab0 | KLK7-L1-Ab4 |
| KLK5-L2-Ab0 | KLK7-L1-Ab5 |
| KLK5-L2-Ab0 | KLK7-L1-Ab6 |
| KLK5-L2-Ab0 | KLK7-L1-Ab7 |
| KLK5-L2-Ab0 | KLK7-L1-Ab8 |
| KLK5-L2-Ab0 | KLK7-L2-Ab0 |
| KLK5-L2-Ab0 | KLK7-L2-Ab1 |
| KLK5-L2-Ab0 | KLK7-L2-Ab2 |
| KLK5-L2-Ab0 | KLK7-L2-Ab3 |
| KLK5-L2-Ab0 | KLK7-L2-Ab4 |
| KLK5-L2-Ab0 | KLK7-L2-Ab5 |
| KLK5-L2-Ab0 | KLK7-L3-Ab0 |
| KLK5-L2-Ab0 | KLK7-L3-Ab1 |
| KLK5-L2-Ab0 | KLK7-L3-Ab2 |
| KLK5-L2-Ab0 | KLK7-L3-Ab3 |
| KLK5-L2-Ab0 | KLK7-L4-Ab0 |
| KLK5-L2-Ab1 | KLK7-L1-Ab0 |
| KLK5-L2-Ab1 | KLK7-L1-Ab1 |
| KLK5-L2-Ab1 | KLK7-L1-Ab2 |
| KLK5-L2-Ab1 | KLK7-L1-Ab3 |
| KLK5-L2-Ab1 | KLK7-L1-Ab4 |
| KLK5-L2-Ab1 | KLK7-L1-Ab5 |
| KLK5-L2-Ab1 | KLK7-L1-Ab6 |
| KLK5-L2-Ab1 | KLK7-L1-Ab7 |
| KLK5-L2-Ab1 | KLK7-L1-Ab8 |
| KLK5-L2-Ab1 | KLK7-L2-Ab0 |
| KLK5-L2-Ab1 | KLK7-L2-Ab1 |
| KLK5-L2-Ab1 | KLK7-L2-Ab2 |
| KLK5-L2-Ab1 | KLK7-L2-Ab3 |
| KLK5-L2-Ab1 | KLK7-L2-Ab4 |
| KLK5-L2-Ab1 | KLK7-L2-Ab5 |
| KLK5-L2-Ab1 | KLK7-L3-Ab0 |
| KLK5-L2-Ab1 | KLK7-L3-Ab1 |
| KLK5-L2-Ab1 | KLK7-L3-Ab2 |
| KLK5-L2-Ab1 | KLK7-L3-Ab3 |
| KLK5-L2-Ab1 | KLK7-L4-Ab0 |
| KLK5-L2-Ab2 | KLK7-L1-Ab0 |
| KLK5-L2-Ab2 | KLK7-L1-Ab1 |
| KLK5-L2-Ab2 | KLK7-L1-Ab2 |
| KLK5-L2-Ab2 | KLK7-L1-Ab3 |
| KLK5-L2-Ab2 | KLK7-L1-Ab4 |
| KLK5-L2-Ab2 | KLK7-L1-Ab5 |
| KLK5-L2-Ab2 | KLK7-L1-Ab6 |
| KLK5-L2-Ab2 | KLK7-L1-Ab7 |
| KLK5-L2-Ab2 | KLK7-L1-Ab8 |
| KLK5-L2-Ab2 | KLK7-L2-Ab0 |
| KLK5-L2-Ab2 | KLK7-L2-Ab1 |
| KLK5-L2-Ab2 | KLK7-L2-Ab2 |
| KLK5-L2-Ab2 | KLK7-L2-Ab3 |
| KLK5-L2-Ab2 | KLK7-L2-Ab4 |
| KLK5-L2-Ab2 | KLK7-L2-Ab5 |
| KLK5-L2-Ab2 | KLK7-L3-Ab0 |
| KLK5-L2-Ab2 | KLK7-L3-Ab1 |
| KLK5-L2-Ab2 | KLK7-L3-Ab2 |
| KLK5-L2-Ab2 | KLK7-L3-Ab3 |
| KLK5-L2-Ab2 | KLK7-L4-Ab0 |
| KLK5-L2-Ab3 | KLK7-L1-Ab0 |
| KLK5-L2-Ab3 | KLK7-L1-Ab1 |
| KLK5-L2-Ab3 | KLK7-L1-Ab2 |
| KLK5-L2-Ab3 | KLK7-L1-Ab3 |
| KLK5-L2-Ab3 | KLK7-L1-Ab4 |
| KLK5-L2-Ab3 | KLK7-L1-Ab5 |
| KLK5-L2-Ab3 | KLK7-L1-Ab6 |
| KLK5-L2-Ab3 | KLK7-L1-Ab7 |
| KLK5-L2-Ab3 | KLK7-L1-Ab8 |
| KLK5-L2-Ab3 | KLK7-L2-Ab0 |
| KLK5-L2-Ab3 | KLK7-L2-Ab1 |
| KLK5-L2-Ab3 | KLK7-L2-Ab2 |
| KLK5-L2-Ab3 | KLK7-L2-Ab3 |
| KLK5-L2-Ab3 | KLK7-L2-Ab4 |
| KLK5-L2-Ab3 | KLK7-L2-Ab5 |
| KLK5-L2-Ab3 | KLK7-L3-Ab0 |
| KLK5-L2-Ab3 | KLK7-L3-Ab1 |
| KLK5-L2-Ab3 | KLK7-L3-Ab2 |
| KLK5-L2-Ab3 | KLK7-L3-Ab3 |
| KLK5-L2-Ab3 | KLK7-L4-Ab0 |
| KLK5-L2-Ab4 | KLK7-L1-Ab0 |
| KLK5-L2-Ab4 | KLK7-L1-Ab1 |
| KLK5-L2-Ab4 | KLK7-L1-Ab2 |
| KLK5-L2-Ab4 | KLK7-L1-Ab3 |
| KLK5-L2-Ab4 | KLK7-L1-Ab4 |
| KLK5-L2-Ab4 | KLK7-L1-Ab5 |
| KLK5-L2-Ab4 | KLK7-L1-Ab6 |
| KLK5-L2-Ab4 | KLK7-L1-Ab7 |
| KLK5-L2-Ab4 | KLK7-L1-Ab8 |
| KLK5-L2-Ab4 | KLK7-L2-Ab0 |
| KLK5-L2-Ab4 | KLK7-L2-Ab1 |
| KLK5-L2-Ab4 | KLK7-L2-Ab2 |
| KLK5-L2-Ab4 | KLK7-L2-Ab3 |
| KLK5-L2-Ab4 | KLK7-L2-Ab4 |
| KLK5-L2-Ab4 | KLK7-L2-Ab5 |
| KLK5-L2-Ab4 | KLK7-L3-Ab0 |
| KLK5-L2-Ab4 | KLK7-L3-Ab1 |
| KLK5-L2-Ab4 | KLK7-L3-Ab2 |
| KLK5-L2-Ab4 | KLK7-L3-Ab3 |
| KLK5-L2-Ab4 | KLK7-L4-Ab0 |
| KLK5-L3-Ab0 | KLK7-L1-Ab0 |
| KLK5-L3-Ab0 | KLK7-L1-Ab1 |
| KLK5-L3-Ab0 | KLK7-L1-Ab2 |
| KLK5-L3-Ab0 | KLK7-L1-Ab3 |
| KLK5-L3-Ab0 | KLK7-L1-Ab4 |
| KLK5-L3-Ab0 | KLK7-L1-Ab5 |
| KLK5-L3-Ab0 | KLK7-L1-Ab6 |
| KLK5-L3-Ab0 | KLK7-L1-Ab7 |
| KLK5-L3-Ab0 | KLK7-L1-Ab8 |
| KLK5-L3-Ab0 | KLK7-L2-Ab0 |
| KLK5-L3-Ab0 | KLK7-L2-Ab1 |
| KLK5-L3-Ab0 | KLK7-L2-Ab2 |
| KLK5-L3-Ab0 | KLK7-L2-Ab3 |
| KLK5-L3-Ab0 | KLK7-L2-Ab4 |
| KLK5-L3-Ab0 | KLK7-L2-Ab5 |
| KLK5-L3-Ab0 | KLK7-L3-Ab0 |
| KLK5-L3-Ab0 | KLK7-L3-Ab1 |
| KLK5-L3-Ab0 | KLK7-L3-Ab2 |
| KLK5-L3-Ab0 | KLK7-L3-Ab3 |
| KLK5-L3-Ab0 | KLK7-L4-Ab0 |
| KLK5-L3-Ab1 | KLK7-L1-Ab0 |
| KLK5-L3-Ab1 | KLK7-L1-Ab1 |
| KLK5-L3-Ab1 | KLK7-L1-Ab2 |
| KLK5-L3-Ab1 | KLK7-L1-Ab3 |
| KLK5-L3-Ab1 | KLK7-L1-Ab4 |
| KLK5-L3-Ab1 | KLK7-L1-Ab5 |
| KLK5-L3-Ab1 | KLK7-L1-Ab6 |
| KLK5-L3-Ab1 | KLK7-L1-Ab7 |
| KLK5-L3-Ab1 | KLK7-L1-Ab8 |
| KLK5-L3-Ab1 | KLK7-L2-Ab0 |
| KLK5-L3-Ab1 | KLK7-L2-Ab1 |
| KLK5-L3-Ab1 | KLK7-L2-Ab2 |
| KLK5-L3-Ab1 | KLK7-L2-Ab3 |
| KLK5-L3-Ab1 | KLK7-L2-Ab4 |
| KLK5-L3-Ab1 | KLK7-L2-Ab5 |
| KLK5-L3-Ab1 | KLK7-L3-Ab0 |
| KLK5-L3-Ab1 | KLK7-L3-Ab1 |
| KLK5-L3-Ab1 | KLK7-L3-Ab2 |
| KLK5-L3-Ab1 | KLK7-L3-Ab3 |
| KLK5-L3-Ab1 | KLK7-L4-Ab0 |
| KLK5-L3-Ab2 | KLK7-L1-Ab0 |
| KLK5-L3-Ab2 | KLK7-L1-Ab1 |
| KLK5-L3-Ab2 | KLK7-L1-Ab2 |
| KLK5-L3-Ab2 | KLK7-L1-Ab3 |
| KLK5-L3-Ab2 | KLK7-L1-Ab4 |
| KLK5-L3-Ab2 | KLK7-L1-Ab5 |
| KLK5-L3-Ab2 | KLK7-L1-Ab6 |
| KLK5-L3-Ab2 | KLK7-L1-Ab7 |
| KLK5-L3-Ab2 | KLK7-L1-Ab8 |
| KLK5-L3-Ab2 | KLK7-L2-Ab0 |
| KLK5-L3-Ab2 | KLK7-L2-Ab1 |
| KLK5-L3-Ab2 | KLK7-L2-Ab2 |
| KLK5-L3-Ab2 | KLK7-L2-Ab3 |
| KLK5-L3-Ab2 | KLK7-L2-Ab4 |
| KLK5-L3-Ab2 | KLK7-L2-Ab5 |
| KLK5-L3-Ab2 | KLK7-L3-Ab0 |
| KLK5-L3-Ab2 | KLK7-L3-Ab1 |
| KLK5-L3-Ab2 | KLK7-L3-Ab2 |
| KLK5-L3-Ab2 | KLK7-L3-Ab3 |
| KLK5-L3-Ab2 | KLK7-L4-Ab0 |
| KLK5-L3-Ab3 | KLK7-L1-Ab0 |
| KLK5-L3-Ab3 | KLK7-L1-Ab1 |
| KLK5-L3-Ab3 | KLK7-L1-Ab2 |
| KLK5-L3-Ab3 | KLK7-L1-Ab3 |
| KLK5-L3-Ab3 | KLK7-L1-Ab4 |
| KLK5-L3-Ab3 | KLK7-L1-Ab5 |
| KLK5-L3-Ab3 | KLK7-L1-Ab6 |
| KLK5-L3-Ab3 | KLK7-L1-Ab7 |
| KLK5-L3-Ab3 | KLK7-L1-Ab8 |
| KLK5-L3-Ab3 | KLK7-L2-Ab0 |
| KLK5-L3-Ab3 | KLK7-L2-Ab1 |
| KLK5-L3-Ab3 | KLK7-L2-Ab2 |
| KLK5-L3-Ab3 | KLK7-L2-Ab3 |
| KLK5-L3-Ab3 | KLK7-L2-Ab4 |
| KLK5-L3-Ab3 | KLK7-L2-Ab5 |
| KLK5-L3-Ab3 | KLK7-L3-Ab0 |
| KLK5-L3-Ab3 | KLK7-L3-Ab1 |
| KLK5-L3-Ab3 | KLK7-L3-Ab2 |
| KLK5-L3-Ab3 | KLK7-L3-Ab3 |
| KLK5-L3-Ab3 | KLK7-L4-Ab0 |
| KLK5-L3-Ab4 | KLK7-L1-Ab0 |
| KLK5-L3-Ab4 | KLK7-L1-Ab1 |
| KLK5-L3-Ab4 | KLK7-L1-Ab2 |
| KLK5-L3-Ab4 | KLK7-L1-Ab3 |
| KLK5-L3-Ab4 | KLK7-L1-Ab4 |
| KLK5-L3-Ab4 | KLK7-L1-Ab5 |
| KLK5-L3-Ab4 | KLK7-L1-Ab6 |
| KLK5-L3-Ab4 | KLK7-L1-Ab7 |
| KLK5-L3-Ab4 | KLK7-L1-Ab8 |
| KLK5-L3-Ab4 | KLK7-L2-Ab0 |
| KLK5-L3-Ab4 | KLK7-L2-Ab1 |
| KLK5-L3-Ab4 | KLK7-L2-Ab2 |
| KLK5-L3-Ab4 | KLK7-L2-Ab3 |
| KLK5-L3-Ab4 | KLK7-L2-Ab4 |
| KLK5-L3-Ab4 | KLK7-L2-Ab5 |
| KLK5-L3-Ab4 | KLK7-L3-Ab0 |
| KLK5-L3-Ab4 | KLK7-L3-Ab1 |
| KLK5-L3-Ab4 | KLK7-L3-Ab2 |
| KLK5-L3-Ab4 | KLK7-L3-Ab3 |
| KLK5-L3-Ab4 | KLK7-L4-Ab0 |
| KLK5-L3-Ab5 | KLK7-L1-Ab0 |
| KLK5-L3-Ab5 | KLK7-L1-Ab1 |
| KLK5-L3-Ab5 | KLK7-L1-Ab2 |
| KLK5-L3-Ab5 | KLK7-L1-Ab3 |
| KLK5-L3-Ab5 | KLK7-L1-Ab4 |
| KLK5-L3-Ab5 | KLK7-L1-Ab5 |
| KLK5-L3-Ab5 | KLK7-L1-Ab6 |
| KLK5-L3-Ab5 | KLK7-L1-Ab7 |
| KLK5-L3-Ab5 | KLK7-L1-Ab8 |
| KLK5-L3-Ab5 | KLK7-L2-Ab0 |
| KLK5-L3-Ab5 | KLK7-L2-Ab1 |
| KLK5-L3-Ab5 | KLK7-L2-Ab2 |
| KLK5-L3-Ab5 | KLK7-L2-Ab3 |
| KLK5-L3-Ab5 | KLK7-L2-Ab4 |
| KLK5-L3-Ab5 | KLK7-L2-Ab5 |
| KLK5-L3-Ab5 | KLK7-L3-Ab0 |
| KLK5-L3-Ab5 | KLK7-L3-Ab1 |
| KLK5-L3-Ab5 | KLK7-L3-Ab2 |
| KLK5-L3-Ab5 | KLK7-L3-Ab3 |
| KLK5-L3-Ab5 | KLK7-L4-Ab0 |
| KLK5-L3-Ab6 | KLK7-L1-Ab0 |
| KLK5-L3-Ab6 | KLK7-L1-Ab1 |
| KLK5-L3-Ab6 | KLK7-L1-Ab2 |
| KLK5-L3-Ab6 | KLK7-L1-Ab3 |
| KLK5-L3-Ab6 | KLK7-L1-Ab4 |
| KLK5-L3-Ab6 | KLK7-L1-Ab5 |
| KLK5-L3-Ab6 | KLK7-L1-Ab6 |
| KLK5-L3-Ab6 | KLK7-L1-Ab7 |
| KLK5-L3-Ab6 | KLK7-L1-Ab8 |
| KLK5-L3-Ab6 | KLK7-L2-Ab0 |
| KLK5-L3-Ab6 | KLK7-L2-Ab1 |
| KLK5-L3-Ab6 | KLK7-L2-Ab2 |
| KLK5-L3-Ab6 | KLK7-L2-Ab3 |
| KLK5-L3-Ab6 | KLK7-L2-Ab4 |
| KLK5-L3-Ab6 | KLK7-L2-Ab5 |
| KLK5-L3-Ab6 | KLK7-L3-Ab0 |
| KLK5-L3-Ab6 | KLK7-L3-Ab1 |
| KLK5-L3-Ab6 | KLK7-L3-Ab2 |
| KLK5-L3-Ab6 | KLK7-L3-Ab3 |
| KLK5-L3-Ab6 | KLK7-L4-Ab0 |
| KLK5-L3-Ab7 | KLK7-L1-Ab0 |
| KLK5-L3-Ab7 | KLK7-L1-Ab1 |
| KLK5-L3-Ab7 | KLK7-L1-Ab2 |
| KLK5-L3-Ab7 | KLK7-L1-Ab3 |
| KLK5-L3-Ab7 | KLK7-L1-Ab4 |
| KLK5-L3-Ab7 | KLK7-L1-Ab5 |
| KLK5-L3-Ab7 | KLK7-L1-Ab6 |
| KLK5-L3-Ab7 | KLK7-L1-Ab7 |
| KLK5-L3-Ab7 | KLK7-L1-Ab8 |
| KLK5-L3-Ab7 | KLK7-L2-Ab0 |
| KLK5-L3-Ab7 | KLK7-L2-Ab1 |
| KLK5-L3-Ab7 | KLK7-L2-Ab2 |
| KLK5-L3-Ab7 | KLK7-L2-Ab3 |
| KLK5-L3-Ab7 | KLK7-L2-Ab4 |
| KLK5-L3-Ab7 | KLK7-L2-Ab5 |
| KLK5-L3-Ab7 | KLK7-L3-Ab0 |
| KLK5-L3-Ab7 | KLK7-L3-Ab1 |
| KLK5-L3-Ab7 | KLK7-L3-Ab2 |
| KLK5-L3-Ab7 | KLK7-L3-Ab3 |
| KLK5-L3-Ab7 | KLK7-L4-Ab0 |
| KLK5-L1-Ab0 | KLK7-L4-Ab1 |
| KLK5-L1-Ab1 | KLK7-L4-Ab1 |
| KLK5-L1-Ab2 | KLK7-L4-Ab1 |
| KLK5-L1-Ab3 | KLK7-L4-Ab1 |
| KLK5-L1-Ab4 | KLK7-L4-Ab1 |
| KLK5-L1-Ab5 | KLK7-L4-Ab1 |
| KLK5-L2-Ab0 | KLK7-L4-Ab1 |
| KLK5-L2-Ab1 | KLK7-L4-Ab1 |
| KLK5-L2-Ab2 | KLK7-L4-Ab1 |
| KLK5-L2-Ab3 | KLK7-L4-Ab1 |
| KLK5-L2-Ab4 | KLK7-L4-Ab1 |
| KLK5-L3-Ab0 | KLK7-L4-Ab1 |
| KLK5-L3-Ab1 | KLK7-L4-Ab1 |
| KLK5-L3-Ab2 | KLK7-L4-Ab1 |
| KLK5-L3-Ab3 | KLK7-L4-Ab1 |
| KLK5-L3-Ab4 | KLK7-L4-Ab1 |
| KLK5-L3-Ab5 | KLK7-L4-Ab1 |
| KLK5-L3-Ab6 | KLK7-L4-Ab1 |
| KLK5-L3-Ab7 | KLK7-L4-Ab1 |
| KLK5-L1-Ab0 | KLK7-L4-Ab2 |
| KLK5-L1-Ab1 | KLK7-L4-Ab2 |
| KLK5-L1-Ab2 | KLK7-L4-Ab2 |
| KLK5-L1-Ab3 | KLK7-L4-Ab2 |
| KLK5-L1-Ab4 | KLK7-L4-Ab2 |
| KLK5-L1-Ab5 | KLK7-L4-Ab2 |
| KLK5-L2-Ab0 | KLK7-L4-Ab2 |
| KLK5-L2-Ab1 | KLK7-L4-Ab2 |
| KLK5-L2-Ab2 | KLK7-L4-Ab2 |
| KLK5-L2-Ab3 | KLK7-L4-Ab2 |
| KLK5-L2-Ab4 | KLK7-L4-Ab2 |
| KLK5-L3-Ab0 | KLK7-L4-Ab2 |
| KLK5-L3-Ab1 | KLK7-L4-Ab2 |
| KLK5-L3-Ab2 | KLK7-L4-Ab2 |
| KLK5-L3-Ab3 | KLK7-L4-Ab2 |
| KLK5-L3-Ab4 | KLK7-L4-Ab2 |
| KLK5-L3-Ab5 | KLK7-L4-Ab2 |
| KLK5-L3-Ab6 | KLK7-L4-Ab2 |
| KLK5-L3-Ab7 | KLK7-L4-Ab2 |
| KLK5-L1-Ab0 | KLK7-L5-Ab0 |
| KLK5-L1-Ab1 | KLK7-L5-Ab0 |
| KLK5-L1-Ab2 | KLK7-L5-Ab0 |
| KLK5-L1-Ab3 | KLK7-L5-Ab0 |
| KLK5-L1-Ab4 | KLK7-L5-Ab0 |
| KLK5-L1-Ab5 | KLK7-L5-Ab0 |
| KLK5-L2-Ab0 | KLK7-L5-Ab0 |
| KLK5-L2-Ab1 | KLK7-L5-Ab0 |
| KLK5-L2-Ab2 | KLK7-L5-Ab0 |
| KLK5-L2-Ab3 | KLK7-L5-Ab0 |
| KLK5-L2-Ab4 | KLK7-L5-Ab0 |
| KLK5-L3-Ab0 | KLK7-L5-Ab0 |
| KLK5-L3-Ab1 | KLK7-L5-Ab0 |
| KLK5-L3-Ab2 | KLK7-L5-Ab0 |
| KLK5-L3-Ab3 | KLK7-L5-Ab0 |
| KLK5-L3-Ab4 | KLK7-L5-Ab0 |
| KLK5-L3-Ab5 | KLK7-L5-Ab0 |
| KLK5-L3-Ab6 | KLK7-L5-Ab0 |
| KLK5-L3-Ab7 | KLK7-L5-Ab0 |
| KLK5-L1-Ab0 | KLK7-L5-Ab1 |
| KLK5-L1-Ab1 | KLK7-L5-Ab1 |
| KLK5-L1-Ab2 | KLK7-L5-Ab1 |
| KLK5-L1-Ab3 | KLK7-L5-Ab1 |
| KLK5-L1-Ab4 | KLK7-L5-Ab1 |
| KLK5-L1-Ab5 | KLK7-L5-Ab1 |
| KLK5-L2-Ab0 | KLK7-L5-Ab1 |
| KLK5-L2-Ab1 | KLK7-L5-Ab1 |
| KLK5-L2-Ab2 | KLK7-L5-Ab1 |
| KLK5-L2-Ab3 | KLK7-L5-Ab1 |
| KLK5-L2-Ab4 | KLK7-L5-Ab1 |
| KLK5-L3-Ab0 | KLK7-L5-Ab1 |
| KLK5-L3-Ab1 | KLK7-L5-Ab1 |
| KLK5-L3-Ab2 | KLK7-L5-Ab1 |
| KLK5-L3-Ab3 | KLK7-L5-Ab1 |
| KLK5-L3-Ab4 | KLK7-L5-Ab1 |
| KLK5-L3-Ab5 | KLK7-L5-Ab1 |
| KLK5-L3-Ab6 | KLK7-L5-Ab1 |
| KLK5-L3-Ab7 | KLK7-L5-Ab1 |
| KLK5-L1-Ab0 | KLK7-L5-Ab2 |
| KLK5-L1-Ab1 | KLK7-L5-Ab2 |
| KLK5-L1-Ab2 | KLK7-L5-Ab2 |
| KLK5-L1-Ab3 | KLK7-L5-Ab2 |
| KLK5-L1-Ab4 | KLK7-L5-Ab2 |
| KLK5-L1-Ab5 | KLK7-L5-Ab2 |
| KLK5-L2-Ab0 | KLK7-L5-Ab2 |
| KLK5-L2-Ab1 | KLK7-L5-Ab2 |
| KLK5-L2-Ab2 | KLK7-L5-Ab2 |
| KLK5-L2-Ab3 | KLK7-L5-Ab2 |
| KLK5-L2-Ab4 | KLK7-L5-Ab2 |
| KLK5-L3-Ab0 | KLK7-L5-Ab2 |
| KLK5-L3-Ab1 | KLK7-L5-Ab2 |
| KLK5-L3-Ab2 | KLK7-L5-Ab2 |
| KLK5-L3-Ab3 | KLK7-L5-Ab2 |
| KLK5-L3-Ab4 | KLK7-L5-Ab2 |
| KLK5-L3-Ab5 | KLK7-L5-Ab2 |
| KLK5-L3-Ab6 | KLK7-L5-Ab2 |
| KLK5-L3-Ab7 | KLK7-L5-Ab2 |

In some embodiments, one or more anti-KLK5 antibodies may be combined with any other appropriate anti-KLK7 antibody to produce a multi-specific or a bispecific anti-KLK5/KLK7 antibody. For example, an anti-KLK5 antibody as described herein (Table 1a and Table 1b), can be combined with other any appropriate anti-KLK7 antibody to produce a bispecific antibody (referred to as an anti-KLK5xKLK7 bispecific antibody). In some embodiments, an anti-KLK5xKLK7 bispecific antibody comprises (i) a first arm comprising an antigen-specific binding site of KLK5-L2-Ab2; and (ii) a second arm comprising an antigen-specific binding site of KLK7-L1-Ab8. In some embodiments, an anti-KLK5xKLK7 bispecific antibody comprises (1) a first arm comprising the HC CDR1, HC CDR2, and HC CDR3 of a heavy chain variable domain (VH) comprising the amino acid sequence of SEQ ID NO: 28 and/or a LC CDR1, LC CDR2, and LC CDR3 of a light chain variable domain (VL) comprising the amino acid sequence of SEQ ID NO: 29; and (2) a second arm comprising the HC CDR1, HC CDR2, and HC CDR3 of a VH comprising the amino acid sequence of SEQ ID NO: 285 and/or a LC CDR1, LC CDR2, and LC CDR3 of a VL comprising the amino acid sequence of SEQ ID NO: 82. In some embodiments, an anti-KLK5xKLK7 bispecific antibody comprises (1) a first arm comprising a HC CDR1 comprising the amino acid sequence of SEQ ID NO: 25, a HC CDR2 comprising the amino acid sequence of SEQ ID NO: 26, a HC CDR1 comprising the amino acid sequence of SEQ ID NO: 27, a LC CDR1 comprising the amino acid sequence of SEQ ID NO: 22, a LC CDR2 comprising the amino acid sequence of SEQ ID NO: 23, and a LC CDR3 comprising the amino acid sequence of SEQ ID NO: 34; and (2) a second arm comprising a HC CDR1 comprising the amino acid sequence of SEQ ID NO: 284, a HC CDR2 comprising the amino acid sequence of SEQ ID NO: 79, a HC CDR3 comprising the amino acid sequence of SEQ ID NO: 89, a LC CDR1 comprising the amino acid sequence of SEQ ID NO: 69, a LC CDR2 comprising the amino acid sequence of SEQ ID NO: 70, and a LC CDR3 comprising the amino acid sequence of SEQ ID NO: 81. In some embodiments, an anti-KLK5xKLK7 bispecific antibody can comprise (1) a first arm comprising a VH having the amino acid sequence of SEQ ID NO: 28 and/or a VL comprising the amino acid sequence of SEQ ID NO: 29; and (2) a second arm comprising a VH having the amino acid sequence of SEQ ID NO: 285 and/or a VL comprising the amino acid sequence of SEQ ID NO: 82.

Non-limiting examples of appropriate anti-KLK7 antibodies are provided in US Patent Application Publication No.: 2021-0130492 entitled, “ANTI-KLK7 ANTIBODIES, ANTI-KLK5 ANTIBODIES, MULTISPECIFIC ANTI-KLK5/KLK7 ANTIBODIES, AND METHODS OF USE”, published on May 6, 2021; International Patent Application Publication No.: WO2021226695 entitled, RECOMBINANT HUMAN ANTIBODIES FOR INHIBITING HUMAN TISSUE KALLIKREIN 7 (KLK7) AND USE IN DISEASES RELATED TO THE PROCESS OF SKIN DESQUAMATION”, published Nov. 18, 2021; and International Patent Application Publication No.: WO2005075667 entitled, “DIAGNOSTICS AND THERAPEUTICS FOR DISEASES ASSOCIATED WITH KALLIKREIN 7 (KLK7)”, published on Aug. 18, 2005, the contents of which are incorporated herein by reference.

In some embodiments, one or more anti-KLK7 antibodies may be combined with any appropriate anti-KLK5 antibody to produce a multi-specific or a bispecific anti-KLK5/KLK7 antibody. For example, a KLK7 antibody as described herein (Table 2a and Table 2b), can be combined with any appropriate KLK5 antibody. Non-limiting examples of anti-KLK5 antibodies are provided in U.S. Pat. No. 11,292,828 entitled, “KLK5 INHIBITORY PEPTIDE”, granted on Apr. 5, 2022; US Patent Application Publication No.: 2022-0306725 entitled, “KLK5 INHIBITORY PEPTIDE”, published on Sep. 29, 2022; US Patent Application Publication No.: 2019-0078160 entitled, “USE OF KLK5 ANTAGONISTS FOR TREATMENT OF A DISEASE”, published on Mar. 14, 2019; International Patent Application Publication No.: WO2021156171 entitled, “ANTIBODIES AGAINST KLK5”, published on Aug. 12, 2021; International Patent Application Publication No.: WO2021156170 entitled, “ANTIBODIES AGAINST KLK5”, published on Aug. 12, 2021; US Patent Application Publication No.: 2021-0301032 entitled, “ANTI-KLK5 ANTIBODIES AND METHODS OF USE”, published on Sep. 30, 2021; and US Patent Application Publication No.: 2021-0130492 entitled, “ANTI-KLK7 ANTIBODIES, ANTI-KLK5 ANTIBODIES, MULTISPECIFIC ANTI-KLK5/KLK7 ANTIBODIES, AND METHODS OF USE”, published on May 6, 2021, the contents of which are incorporated herein by reference.

In some embodiments, one or more anti-KLK5 antibodies or one or more anti-KLK7 antibodies described here (Table 1a, Table 1b, Table 2a, and Table 2b) may be combined with any appropriate anti-KLK5 or anti-KLK7 antibody to produce a bispecific, or multi-specific antibody with specificity to KLK5, KLK7, or both (KLK5 and KLK7). Non-limiting examples of anti-KLK5 antibodies are provided in U.S. Pat. No. 11,292,828 entitled, “KLK5 INHIBITORY PEPTIDE”, granted on Apr. 5, 2022; US Patent Application Publication No.: 2022-0306725 entitled, “KLK5 INHIBITORY PEPTIDE”, published on Sep. 29, 2022; US Patent Application Publication No.: 2019-0078160 entitled, “USE OF KLK5 ANTAGONISTS FOR TREATMENT OF A DISEASE”, published on Mar. 14, 2019; International Patent Application Publication No.: WO2021156171 entitled, “ANTIBODIES AGAINST KLK5”, published on Aug. 12, 2021; International Patent Application Publication No.: WO2021156170 entitled, “ANTIBODIES AGAINST KLK5”, published on Aug. 12, 2021; US Patent Application Publication No.: 2021-0301032 entitled, “ANTI-KLK5 ANTIBODIES AND METHODS OF USE”, published on Sep. 30, 2021; and US Patent Application Publication No.: 2021-0130492 entitled, “ANTI-KLK7 ANTIBODIES, ANTI-KLK5 ANTIBODIES, MULTISPECIFIC ANTI-KLK5/KLK7 ANTIBODIES, AND METHODS OF USE”, published on May 6, 2021, the contents of which are incorporated herein by reference. Non-limiting examples of appropriate anti-KLK7 antibodies are provided in US Patent Application Publication No.: 2021-0130492 entitled, “ANTI-KLK7 ANTIBODIES, ANTI-KLK5 ANTIBODIES, MULTISPECIFIC ANTI-KLK5/KLK7 ANTIBODIES, AND METHODS OF USE”, published on May 6, 2021; International Patent Application Publication No.: WO2021226695 entitled, RECOMBINANT HUMAN ANTIBODIES FOR INHIBITING HUMAN TISSUE KALLIKREIN 7 (KLK7) AND USE IN DISEASES RELATED TO THE PROCESS OF SKIN DESQUAMATION”, published Nov. 18, 2021; and International Patent Application Publication No.: WO2005075667 entitled, “DIAGNOSTICS AND THERAPEUTICS FOR DISEASES ASSOCIATED WITH KALLIKREIN 7 (KLK7)”, published on Aug. 18, 2005, the contents of which are incorporated herein by reference.

In some embodiments, one or more anti-KLK5 antibodies may be combined with one or more anti-KLK7 antibody to produce a multi-specific or a bispecific anti-KLK5/KLK7 antibody. For example, an appropriate anti-KLK5 antibody, can be combined with an appropriate anti-KLK7 antibody. Non-limiting examples of anti-KLK5 antibodies are provided in U.S. Pat. No. 11,292,828 entitled, “KLK5 INHIBITORY PEPTIDE”, granted on Apr. 5, 2022; US Patent Application Publication No.: 2022-0306725 entitled, “KLK5 INHIBITORY PEPTIDE”, published on Sep. 29, 2022; US Patent Application Publication No.: 2019-0078160 entitled, “USE OF KLK5 ANTAGONISTS FOR TREATMENT OF A DISEASE”, published on Mar. 14, 2019; International Patent Application Publication No.: WO2021156171 entitled, “ANTIBODIES AGAINST KLK5”, published on Aug. 12, 2021; International Patent Application Publication No.: WO2021156170 entitled, “ANTIBODIES AGAINST KLK5”, published on Aug. 12, 2021; US Patent Application Publication No.: 2021-0301032 entitled, “ANTI-KLK5 ANTIBODIES AND METHODS OF USE”, published on Sep. 30, 2021; and US Patent Application Publication No.: 2021-0130492 entitled, “ANTI-KLK7 ANTIBODIES, ANTI-KLK5 ANTIBODIES, MULTISPECIFIC ANTI-KLK5/KLK7 ANTIBODIES, AND METHODS OF USE”, published on May 6, 2021, the contents of which are incorporated herein by reference. Non-limiting examples of appropriate anti-KLK7 antibodies are provided in US Patent Application Publication No.: 2021-0130492 entitled, “ANTI-KLK7 ANTIBODIES, ANTI-KLK5 ANTIBODIES, MULTISPECIFIC ANTI-KLK5/KLK7 ANTIBODIES, AND METHODS OF USE”, published on May 6, 2021; International Patent Application Publication No.: WO2021226695 entitled, RECOMBINANT HUMAN ANTIBODIES FOR INHIBITING HUMAN TISSUE KALLIKREIN 7 (KLK7) AND USE IN DISEASES RELATED TO THE PROCESS OF SKIN DESQUAMATION”, published Nov. 18, 2021; and International Patent Application Publication No.: WO2005075667 entitled, “DIAGNOSTICS AND THERAPEUTICS FOR DISEASES ASSOCIATED WITH KALLIKREIN 7 (KLK7)”, published on Aug. 18, 2005, the contents of which are incorporated herein by reference.

In some embodiments, a multi-specific antibody comprises three, four, five, six, seven, eight, or more distinct antigen-specific binding sites. In some embodiments, each distinct antigen-specific binding site of a multi-specific antibody targets a different antigen. In some embodiments, each distinct antigen-specific binding site of a multi-specific antibody targets a different region of the same antigen. In some embodiments, a multi-specific antibody comprises distinct antigen-specific binding sites targeting different antigens and/or distinct antigen-specific binding sites targeting different regions of the same antigen. In some embodiments, a multi-specific antibody comprises at least one antigen-specific binding site targeting a first antigen and at least one antigen-specific binding site targeting a second antigen. In some embodiments, a multi-specific antibody comprises two or more antigen-specific binding sites targeting different regions of a first antigen and/or two or more antigen-specific binding sites targeting different regions of a second antigen.

In some embodiments, a multi-specific antibody targets two antigens and contains one antigen-specific binding site for each antigen (1+1). In some embodiments, a multi-specific antibody targets two antigens and contains two antigen-specific binding site for each antigen (2+2). In some embodiments, a multi-specific antibody targets two antigens and contains one antigen-specific binding site for one antigen and two antigen-specific binding sites for another antigen (1+2). In some embodiments, a multi-specific antibody targets two antigens and contains two antigen-specific binding site for one antigen and three antigen-specific binding sites for another antigen (2+3). In some embodiments, a multi-specific antibody targets two antigens and contains three antigen-specific binding site for one antigen and three antigen-specific binding sites for another antigen (3+3).

In some embodiments, a multispecific antibody lacks Fc-mediated effector functions, such as antibody-dependent cell-mediated cytotoxicity (ADCC), antibody-dependent cellular phagocytosis (ADCP), complement fixation, and FcRn-mediated recycling. However, in some embodiments, a multi-specific antibody comprises one or more Fc regions that support Fc-mediated effector functions, such as antibody-dependent cell-mediated cytotoxicity (ADCC), antibody-dependent cellular phagocytosis (ADCP), complement fixation, and FcRn-mediated recycling.

In some embodiments, an antibody provided herein is a bispecific antibody. In some embodiments, a bispecific antibody comprises at least two different Fv regions. In some embodiments, a bispecific antibody comprises two different heavy chains, and two different light chains. In some embodiments, a bispecific antibody comprises one or more IgG molecules. In some embodiments, a bispecific antibody comprises one or more IgG molecules that contain additional antigen-specific binding sites, e.g., IgG molecules that comprise an appended or modified Ig-like structure.

In some embodiments, a bispecific antibody comprises two single-chain variable fragments (scFvs) connected via a linker. In some embodiments, a bispecific antibody comprises two single-domain antibodies, such as VH or VL domains, VHH, VNAR or Nanobodies connected via a linker (e.g., a flexible glycine rich linker, such as a (G4S)3¬ linkers). In some embodiments, a bispecific antibody is in a diabody format, such as described in P Holliger, T Prospero, and G Winter, “Diabodies”: small bivalent and bispecific antibody fragments, Proc Natl Acad Sci USA. 1993 Jul. 15; 90(14): 6444-6448, the entire contents of which are incorporated herein by reference in their entirety. In some embodiments, a bispecific antibody is a Fab fusion protein, such as a Fab-Fab fusion protein, a Fab-scFv fusion protein, or a Fab-Fv fusion protein. In some embodiments, a bispecific antibody comprises an antigen-binding site, e.g., a scFv, that is modified to contain a second and distinct antigen-specific binding site as an integral part of the antibody, e.g., scFv.

In some embodiments, a bispecific antibody is in a fragment-based format, symmetric format, or asymmetric format. In some embodiments, a bispecific antibody in a fragment-based format does not comprise an Fc region. In some embodiments, a bispecific antibody is in a tandem VHHs, a tandem scFvs, a DART, a diabody, a F(ab)2, a scFv-Fab, a tandem VHHs, a (scFv)2-Fab, or a tandem diabodies format. In some embodiments, a bispecific antibody is in an asymmetric format selected from: arat-mouse hybrid IgG, hetero H HL exchange and/or assembly IgG, hetero H forced HL IgG, cH IgG, hetero H CrossMab, scFv-Fab IgG, DART-Fc, LP-DART, CODV-Fab-TL, HLE-BiTE, and F(ab)3CrossMab format. In some embodiments, a bispecific antibody is in a symmetrical format selected from: IgG-(scFv)2, Bs4Ab, DVD-Ig, tetravalent DART-Fc, (scFV)4-Fc, CODV-Ig, two-in-one, mAb2, F(ab)4 CrossMab, and tandem VHH-Fc format.

In some embodiments, a bispecific antibody is engineered to facilitate formation via the knobs-into-holes technique, e.g., to facilitate heterodimerization. The knobs-into-holes technique may be used, in some embodiments, to produce bispecific IgG molecules, trivalent Ig-like antibodies, bispecific Fc and CH3 fusion proteins, and other formats, as discussed in Ridgway J B, et al., ‘Knobs-into-holes’ engineering of antibody CH3 domains for heavy chain heterodimerization. Protein Eng 1996; 9:617-21; Atwell S, et al., Stable heterodimers from remodeling the domain interface of a homodimer using a phage display library, J Mol Biol 1997; 270:26-35; and Merchant A M, et al., An efficient route to human bispecific IgG, Nat Biotechnol 1998; 16:677-681, the entire contents of each of which are incorporated herein by reference in their entireties.

In some embodiments, a bispecific antibody lacks Fc-mediated effector functions, such as antibody-dependent cell-mediated cytotoxicity (ADCC), antibody-dependent cellular phagocytosis (ADCP), complement fixation, and/or FcRn-mediated recycling. However, in some embodiments, a bispecific antibody comprises one or more Fc regions that support Fc-mediated effector functions, such as antibody-dependent cell-mediated cytotoxicity (ADCC), antibody-dependent cellular phagocytosis (ADCP), complement fixation, and FcRn-mediated recycling.

### III. PREPARATION OF THE ANTI-KLK5 ANTIBODIES, ANTI-KLK7 ANTIBODIES, AND ANTI-KLK5/KLK7 BISPECIFIC ANTIBODIES

Antibodies described herein can be made by any method known in the art. See, for example, Harlow and Lane, (1998) Antibodies: A Laboratory Manual, Cold Spring Harbor Laboratory, New York.

In some embodiments, antibodies specific to a target antigen (e.g., KLK5 or KLK7) can be made by the conventional hybridoma technology. The full-length target antigen or a fragment thereof, optionally coupled to a carrier protein such as KLH, can be used to immunize a host animal for generating antibodies binding to that antigen. The route and schedule of immunization of the host animal are generally in keeping with established and conventional techniques for antibody stimulation and production, as further described herein. General techniques for production of mouse, humanized, and human antibodies are known in the art and are described herein. It is contemplated that any mammalian subject including humans or antibody producing cells therefrom can be manipulated to serve as the basis for production of mammalian, including human hybridoma cell lines. Typically, the host animal is inoculated intraperitoneally, intramuscularly, orally, subcutaneously, intraplantar, and/or intradermally with an amount of immunogen, including as described herein.

If desired, an antibody (monoclonal or polyclonal) of interest (e.g., produced by a hybridoma) may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to “humanize” the antibody or to improve the affinity (affinity maturation), or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to the target antigen and greater efficacy. It will be apparent to one of skill in the art that one or more polynucleotide changes can be made to the antibody and still maintain its binding specificity to the target antigen.

In other embodiments, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse® from Amgen, Inc. (Fremont, CA) and HuMAb-Mouse® and TC Mouse™ from Medarex, Inc. (Princeton, NJ) or H2L2 mice from Harbour Antibodies BV (Holland). In another alternative, antibodies may be made recombinantly by phage display or yeast technology. See, for example, U.S. Pat. Nos. 5,565,332; 5,580,717; 5,733,743; and 6,265,150; and Winter et al., (1994) Annu. Rev. Immunol. 12:433-455. Alternatively, the phage display technology (McCafferty et al., (1990) Nature 348:552-553) can be used to produce human antibodies and antibody fragments in vitro, from immunoglobulin variable (V) domain gene repertoires from unimmunized donors.

Antigen-binding fragments of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments. Genetically engineered antibodies, such as humanized antibodies, chimeric antibodies, single-chain antibodies, and bi-specific antibodies, can be produced via, e.g., conventional recombinant technology. In one example, DNA encoding a monoclonal antibodies specific to a target antigen can be readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into one or more expression vectors, which are then transfected into host cells such as E. coli cells, simian COS cells, Chinese hamster ovary (CHO) cells, human HEK293 cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions.

Alternatively, techniques described for the production of single chain antibodies (U.S. Pat. Nos. 4,946,778 and 4,704,692) can be adapted to produce a phage or yeast scFv library and scFv clones specific to KLK5 or KLK7 can be identified from the library following routine procedures. Positive clones can be subjected to further screening to identify those that has high KLK5 or KLK7 binding affinity.

Antibodies obtained following a method known in the art and described herein can be characterized using methods well known in the art. For example, one method is to identify the epitope to which the antigen binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In one example, epitope mapping can be accomplished use H/D-Ex (hydrogen deuterium exchange) coupled with proteolysis and mass spectrometry. In an additional example, epitope mapping can be used to determine the sequence to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). Peptides of varying lengths (e.g., at least 4-6 amino acids long) can be isolated or synthesized (e.g., recombinantly) and used for binding assays with an antibody. In another example, the epitope to which the antibody binds can be determined in a systematic screening by using overlapping peptides derived from the target antigen sequence and determining binding by the antibody. According to the gene fragment expression assays, the open reading frame encoding the target antigen is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis. Certain epitopes can also be identified by using large libraries of random peptide sequences displayed on the surface of phage particles (phage libraries). Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. Alternatively, competition assays can be performed using other antibodies known to bind to the same antigen to determine whether an antibody binds to the same epitope as the other antibodies. Competition assays are well known to those of skill in the art.

In some examples, an antibody described herein is prepared by recombinant technology as exemplified below. Nucleic acids encoding the heavy and light chain of an antibody as described herein can be cloned into one expression vector, each nucleotide sequence being in operable linkage to a suitable promoter. In one example, each of the nucleotide sequences encoding the heavy chain and light chain is in operable linkage to a distinct promoter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV promoter, and the herpes simplex tk virus promoter.

Regulatable promoters can also be used. Such regulatable promoters include those using the lac repressor from E. coli as a transcription modulator to regulate transcription from lac operator bearing mammalian cell promoters [[Brown, M. et al., Cell, 49:603-612 (1987)]], those using the tetracycline repressor (tetR) [[Gossen, M., and Bujard, H., Proc. Natl. Acad. Sci. USA 89:5547-555115 (1992); Yao, F. et al., Human Gene Therapy, 9:1939-1950 (1998); Shockelt, P., et al., Proc. Natl. Acad. Sci. USA, 92:6522-6526 (1995)]]. Other systems include FK506 dimer, VP16 or p65 using astradiol, RU486, diphenol murislerone, or rapamycin. Inducible systems are available from Invitrogen, Clontech and Ariad, among others.

Regulatable promoters that include a repressor with the operon can be used. In one embodiment, the lac repressor from E. coli can function as a transcriptional modulator to regulate transcription from lac operator-bearing mammalian cell promoters [[M. Brown et al., Cell, 49:603-612 (1987)]]; Gossen and Bujard (1992); [[M. Gossen et al., Natl. Acad. Sci. USA, 89:5547-5551(1992)]] combined the tetracycline repressor (tetR) with the transcription activator (VP 16) to create a tetR-mammalian cell transcription activator fusion protein, tTa (tetR-VP 16), with the tetO bearing minimal promoter derived from the human cytomegalovirus (hCMV) promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy). One particular advantage of this tetracycline inducible switch is that it does not require the use of a tetracycline repressor-mammalian cells transactivator or repressor fusion protein, which in some instances can be toxic to cells (Gossen 5 et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art. Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies (e.g., the nucleic acid coding sequence listed in Table 3) may be introduced into suitable host cells for producing the antibodies. Non-limiting examples of the host cells include Chinese hamster ovary (CHO) cells, dhfr-CHO cell, human embryonic kidney (HEK)-293 cells, verda reno (VERO) cells, nonsecreting null (NSO) cells, human embryonic retinal (PER.C6) cells, Sp2/0 cells, baby hamster kidney (BHK) cells, Madin-Darby Canine Kidney (MDCK) cells, Madin-Darby Bovine Kidney (MDBK) cells, and monkey kidney CV1 line transformed by SV40 (COS) cells. In some embodiments, the host cell expressing the antibodies described herein are CHO cells. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody. In some embodiments, the host cell comprises the nucleic acid encoding the heavy chain of the an antibody described herein. In some embodiments, the host cell comprises the nucleic acid encoding the light chain of the antibody described herein. In some embodiments, the host cell comprises the nucleic acid encoding the heavy chain and the nucleic acid encoding the light chain.

In some embodiments, methods for preparing an antibody described herein involve a recombinant expression vector that encodes both the heavy chain and the light chain of an antibody described herein, as also described herein. The recombinant expression vector can be introduced into a suitable host cell (e.g., a dhfr-CHO cell) by a conventional method, e.g., calcium phosphate mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody.

In one example, two recombinant expression vectors are provided, one encoding the heavy chain of the antibody and the other encoding the light chain of the antibody. Both of the two recombinant expression vectors can be introduced into a suitable host cell (e.g., dhfr-CHO cell) by a conventional method, e.g., calcium phosphate-mediated transfection.

Alternatively, each of the expression vectors can be introduced into a suitable host cells. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, the light chain, or both of an antibody as described herein (e.g., as provided in Table 3), vectors (e.g., expression vectors) containing such; and host cells comprising the vectors are within the scope of the present disclosure.

TABLE 5
Nucleic acids Sequences encoding the VH/VL of anti-KLK5 and anti-KLK7
antibodies listed in Tables 1a and 2a

| | | | SEQ ID |
|---|---|---|---|
| Antibody | | Nucleic Acid Sequences | NO |
| KLK5- | VH | GAAGTGCAGCTTGTGGAGTCCGGAGGAGGCCTCGTGCAACCGGGCGGAAG | 202 |
| L1-Ab1 | | CCTGAGGCTGAGCTGCGCAGCCTCAGGGTTCACTTTTTCCTCCTACTCCA | |
| | | TGAATTGGGTCAGACAGGCTCCTGGGAAGGGTCTGGAATGGGTGTCCTAC | |
| | | ATCTCCTCCTCGTCATCTACCATCTACTACGCCGACTCCGTCAAGGGCCG | |
| | | CTTCACGATTTCCCGCGACAACGCCAAGAACTCGCTGTACCTCCAAATGA | |
| | | ACTCCTTGCGGGCCGAGGATACCGCGGTGTACTACTGCGCCCGGGATCTG | |
| | | ACTAGCATGCAGAGCCGGGACTACTACTATTACGGAATGGACGTCTGGGG | |
| | | ACAGGGCACCACCGTGACCGTGTCGAGC | |
| | VL | GATATTCAAATGACCCAGTCCCCGTCGTCCGTGAGCGCCTCAGTGGGCGA | 203 |
| | | CAGAGTCACTATTACCTGTCGCGCTAGCCAGGGCATCTCCTCCTGGCTCG | |
| | | CCTGGTACCAGCAGAAACCCGGAAAGGCACCCAAGCTGCTGATCTACGCG | |
| | | GCCAGCTCGCTTCAGTCCGGGGTGCCATCTCGGTTCTCCGGATCCGGCTC | |
| | | AGGAACCGACTTTACTCTCACCATCTCGAGCCTGCAGCCCGAGGACTTCG | |
| | | CCACCTACTACTGCCAACAAGTGCCGATCTTCCCTTACACTTTCGGGGGT | |
| | | GGAACCAAGGTCGAAATCAAG | |
| KLK5- | VH | GAAGTGCAACTGGTGGAGTCTGGTGGCGGACTCGTGCAGCCCGGCGGCTC | 204 |
| L1-Ab2 | | CCTGCGGCTCTCCTGCGCGGCCAGCGGCTTTACCTTCGGATCCTACGCCA | |
| | | TGAATTGGGTCAGACAGGCCCCGGGAAAAGGGCTTGAATGGGTGTCATAC | |
| | | ATCAGCTCGTCCTCCTCCTTCATCGACTACGCGGATAGCGTGAAGGGTCG | |
| | | CTTCACCATTTCGCGGGACAACGCAAAGAACTCACTGTACTTGCAAATGA | |
| | | ACTCCCTGCGGGCCGAGGACACCGCCGTGTACTACTGTGCTCGCGACCTG | |
| | | ACCAGCATGCAGTCGAGGGACTACTACTATTACGGAATGGATGTCTGGGG | |
| | | ACAGGGGACTACTGTGACCGTGTCCTCC | |
| | VL | GATATTCAAATGACCCAGTCCCCGTCGTCCGTGAGCGCCTCAGTGGGCGA | 203 |
| | | CAGAGTCACTATTACCTGTCGCGCTAGCCAGGGCATCTCCTCCTGGCTCG | |
| | | CCTGGTACCAGCAGAAACCCGGAAAGGCACCCAAGCTGCTGATCTACGCG | |
| | | GCCAGCTCGCTTCAGTCCGGGGTGCCATCTCGGTTCTCCGGATCCGGCTC | |
| | | AGGAACCGACTTTACTCTCACCATCTCGAGCCTGCAGCCCGAGGACTTCG | |
| | | CCACCTACTACTGCCAACAAGTGCCGATCTTCCCTTACACTTTCGGGGGT | |
| | | GGAACCAAGGTCGAAATCAAG | |
| KLK5- | VH | GAAGTGCAGCTCGTGGAGTCGGGTGGCGGACTGGTGCAGCCCGGGGGATC | 205 |
| L1-Ab3 | | CCTCCGGCTGAGCTGCGCCGCCTCTGGATTCACCTTCGAACACTACAGCA | |
| | | TGAACTGGGTCAGACAGGCTCCGGGAAAGGGCCTGGAATGGGTGTCCTAC | |
| | | ATTTCCTCGTCCTCATCGTACATCGACTACGCCGACTCCGTGAAGGGCCG | |
| | | GTTTACCATCTCCCGGGATAATGCCAAGAACTCCCTGTACCTTCAAATGA | |
| | | ACTCCTTGAGGGCCGAGGATACCGCGGTGTACTACTGTGCACGCGACCTG | |
| | | ACTAGCATGCAGTCACGCGACTATTACTACTACGGGATGGACGTCTGGGG | |
| | | CCAGGGTACCACCGTGACTGTGTCCAGC | |
| | VL | GATATTCAAATGACCCAGTCCCCGTCGTCCGTGAGCGCCTCAGTGGGCGA | 203 |
| | | CAGAGTCACTATTACCTGTCGCGCTAGCCAGGGCATCTCCTCCTGGCTCG | |
| | | CCTGGTACCAGCAGAAACCCGGAAAGGCACCCAAGCTGCTGATCTACGCG | |
| | | GCCAGCTCGCTTCAGTCCGGGGTGCCATCTCGGTTCTCCGGATCCGGCTC | |
| | | AGGAACCGACTTTACTCTCACCATCTCGAGCCTGCAGCCCGAGGACTTCG | |
| | | CCACCTACTACTGCCAACAAGTGCCGATCTTCCCTTACACTTTCGGGGGT | |
| | | GGAACCAAGGTCGAAATCAAG | |
| KLK5- | VH | GAAGTGCAGCTCGTGGAGAGCGGCGGCGGATTGGTGCAGCCCGGCGGATC | 206 |
| L1-Ab4 | | ACTGAGACTTTCCTGCGCGGCCAGCGGTTTCACTTTCAACGACTACTCTA | |
| | | TGAACTGGGTCCGCCAAGCTCCGGGAAAGGGTCTGGAATGGGTGTCCTAC | |
| | | ATTAGCTCCTCCTCCTCGTACATCGATTACGCCGATTCCGTGAAGGGCCG | |
| | | CTTTACCATCTCCCGGGACAACGCCAAGAACTCCCTGTACCTCCAAATGA | |
| | | ATTCCCTGAGGGCCGAGGATACCGCAGTGTACTACTGTGCCCGGGACCTG | |
| | | ACTAGCATGCAGTCACGGGACTACTACTATTACGGGATGGACGTCTGGGG | |
| | | ACAGGGGACCACCGTGACCGTGTCGTCG | |
| | VL | GATATTCAAATGACCCAGTCCCCGTCGTCCGTGAGCGCCTCAGTGGGCGA | 203 |
| | | CAGAGTCACTATTACCTGTCGCGCTAGCCAGGGCATCTCCTCCTGGCTCG | |
| | | CCTGGTACCAGCAGAAACCCGGAAAGGCACCCAAGCTGCTGATCTACGCG | |
| | | GCCAGCTCGCTTCAGTCCGGGGTGCCATCTCGGTTCTCCGGATCCGGCTC | |
| | | AGGAACCGACTTTACTCTCACCATCTCGAGCCTGCAGCCCGAGGACTTCG | |
| | | CCACCTACTACTGCCAACAAGTGCCGATCTTCCCTTACACTTTCGGGGGT | |
| | | GGAACCAAGGTCGAAATCAAG | |
| KLK5- | VH | GAAGTGCAGCTTGTGGAGAGCGGTGGCGGACTGGTGCAGCCCGGCGGATC | 207 |
| L1-Ab5 | | CCTGCGGCTCTCCTGCGCCGCGAGCGGTTTCACCTTCGAACACTACTCCA | |
| | | TGAATTGGGTCAGACAGGCCCCGGGAAAGGGCCTGGAATGGGTGTCCTAT | |
| | | ATCTCGAGCTCCTCCTCATACATCGACTACGCTGACTCCGTGAAGGGCCG | |
| | | GTTTACCATTTCGAGGGACAACGCCAAGAACTCTCTGTACCTCCAAATGA | |
| | | ACTCCCTGAGAGCCGAGGATACCGCAGTGTACTACTGTGCCCGCGACTTG | |
| | | ACTGATCGCCAGTCGCGGGATTACTACTACTACGGGATGGACGTCTGGGG | |
| | | ACAGGGGACCACCGTGACTGTGTCAAGC | |
| | VL | GATATTCAAATGACCCAGTCCCCGTCGTCCGTGAGCGCCTCAGTGGGCGA | 203 |
| | | CAGAGTCACTATTACCTGTCGCGCTAGCCAGGGCATCTCCTCCTGGCTCG | |
| | | CCTGGTACCAGCAGAAACCCGGAAAGGCACCCAAGCTGCTGATCTACGCG | |
| | | GCCAGCTCGCTTCAGTCCGGGGTGCCATCTCGGTTCTCCGGATCCGGCTC | |
| | | AGGAACCGACTTTACTCTCACCATCTCGAGCCTGCAGCCCGAGGACTTCG | |
| | | CCACCTACTACTGCCAACAAGTGCCGATCTTCCCTTACACTTTCGGGGGT | |
| | | GGAACCAAGGTCGAAATCAAG | |
| KLK5- | VH | CAGCTGCAACTGCAGGAATCCGGACCAGGCCTCGTGAAGCCCAGCGAAAC | 208 |
| L2-Ab1 | | TTTGTCCCTTACCTGTACCGTCAGCGGAGGTTCAATCTCCTCCTCTTCCT | |
| | | ACTACTGGGGTTGGATTAGGCAGCCTCCGGGAAAGGGGCTCGAGTGGATC | |
| | | GGCAGCATTTCCTACTCGGGCTCGACCTACTACAACCCGTCCCTGAAGTC | |
| | | GCGCGTGACCATCTCGGTGGACACCTCCAAGAACCAGTTCAGCCTGAAAC | |
| | | TGTCCTCAGTGACTGCCGCTGATACGGCGGTGTATTACTGCGCAAGAGGG | |
| | | GAGTACGACCGGTCATACTATTACTACGGAATGGACGTCTGGGGACAGGG | |
| | | CACCACTGTGACCGTGTCCAGC | |
| | VL | GAGATCGTGCTGACCCAGTCCCCCGGAACCCTGAGCCTCTCCCCTGGCGA | 209 |
| | | AAGGGCCACTCTGTCGTGCCGGGCGTCACAGTCAGTGTCCAGCTCCTACC | |
| | | TCGCTTGGTACCAGCAAAAGCCTGGACAAGCCCCACGCCTTCTGATCTAC | |
| | | GGCGCCTCGAGCAGAGCCACTGGAATTCCCGACCGGTTCTCCGGTTCTGG | |
| | | GTCCGGCACCGATTTCACCCTGACCATTTCCCGCTTGGAACCGGAGGACT | |
| | | TTGCCGTGTATTACTGCCAGCAGGCATTCTCGAGCCCGACCTTCGGTGGA | |
| | | GGGACTAAGGTCGAGATCAAG | |
| KLK5- | VH | CAGCTGCAACTCCAAGAATCCGGACCGGGTCTTGTGAAGCCATCTGAGAC | 210 |
| L2-Ab2 | | TCTGTCACTGACTTGCACCGTGTCGGGTGGCAGCATCCCCTCGAGCTCCT | |
| | | ACTACTGGGCCTGGATTAGGCAGCCGCCTGGGAAGGGACTCGAGTGGATT | |
| | | GGGAACATCTACTACTCCGGCTCCACGTACTACAACCCCTCCCTGAAATC | |
| | | CCGGGTCACCATCAGCGTGGACACCTCCAAGAACCAGTTCAGCCTGAAGC | |
| | | TGTCCTCGGTGACCGCAGCCGATACTGCCGTGTACTACTGTGCTCGCGGC | |
| | | GAATATGACAGATCCGCGTTGTACTACGGAATGGACGTCTGGGGACAGGG | |
| | | CACCACCGTGACCGTGTCGTCA | |
| | VL | GAGATCGTGCTGACCCAGTCCCCCGGAACCCTGAGCCTCTCCCCTGGCGA | 209 |
| | | AAGGGCCACTCTGTCGTGCCGGGCGTCACAGTCAGTGTCCAGCTCCTACC | |
| | | TCGCTTGGTACCAGCAAAAGCCTGGACAAGCCCCACGCCTTCTGATCTAC | |
| | | GGCGCCTCGAGCAGAGCCACTGGAATTCCCGACCGGTTCTCCGGTTCTGG | |
| | | GTCCGGCACCGATTTCACCCTGACCATTTCCCGCTTGGAACCGGAGGACT | |
| | | TTGCCGTGTATTACTGCCAGCAGGCATTCTCGAGCCCGACCTTCGGTGGA | |
| | | GGGACTAAGGTCGAGATCAAG | |
| KLK5- | VH | CAGCTGCAACTGCAAGAATCGGGGCCGGGCTTGGTCAAGCCAAGCGAAAC | 211 |
| L2-Ab3 | | CCTCTCTCTGACCTGTACTGTGTCCGGCGGAAGCATCCCCTCCTCCTCCT | |
| | | ATTACTGGTCGTGGATTAGGCAGCCTCCCGGGAAGGGACTGGAGTGGATC | |
| | | GGCAACATCTACTACTCGGGCTCGACTTACTACAACCCGAGCCTGAAGTC | |
| | | CCGCGTGACCATTAGCGTGGACACCTCCAAAAACCAGTTCAGCCTTAAGC | |
| | | TCTCATCAGTGACGGCCGCCGATACCGCTGTGTACTACTGCGCGCGGGGA | |
| | | GAGTACGACAGATCCTACTACTTCTGGGGAATGGACGTCTGGGGACAGGG | |
| | | TACCACTGTGACCGTGTCCTCC | |
| | VL | GAAATCGTGTTGACTCAGTCCCCTGGTACCCTCTCACTGTCCCCGGGAGA | 212 |
| | | AAGGGCCACCCTGTCCTGTCGGGCATCGCAGTCCGTGTCCTCCTCGTACC | |
| | | TTGCCTGGTACCAGCAGAAGCCCGGCCAGGCTCCCCGCCTCCTGATCTAC | |
| | | GGCGCGAGCAGCAGAGCCACTGGTATTCCTGACCGGTTCTCTGGGTCCGG | |
| | | AAGCGGAACCGACTTCACTCTGACCATTTCACGCCTGGAGCCGGAAGATT | |
| | | TCGCCGTGTATTACTGCCAACAAGCCTTCAGCTCGCCATCCTTTGGCGGG | |
| | | GGAACCAAAGTCGAGATCAAG | |
| KLK5- | VH | CAGCTGCAACTGCAAGAATCGGGACCCGGACTCGTGAAGCCTAGCGAGAC | 213 |
| L2-Ab4 | | TCTGAGCCTCACCTGTACTGTGTCTGGCGGATCCATCCCGTCCTCGAGCT | |
| | | ACTACTGGTCCTGGATTCGGCAGCCACCGGGGAAGGGCCTGGAGTGGATT | |
| | | GGGAACATCTACTACTCCGGTTCGACCTACTATAACCCCTCGCTGAAGTC | |
| | | CCGCGTCACCATCAGCGTGGACACCTCCAAGAACCAGTTCTCCCTGAAAT | |
| | | TGTCCAGCGTGACCGCCGCTGATACGGCCGTGTACTACTGCGCGAGGGGA | |
| | | GAATACGACAGATCCTACTACTATTGGGGCCTTGACGTCTGGGGCCAGGG | |
| | | TACTACCGTGACCGTGTCATCA | |
| | VL | GAGATCGTGCTGACCCAGTCCCCCGGAACCCTGAGCCTCTCCCCTGGCGA | 209 |
| | | AAGGGCCACTCTGTCGTGCCGGGCGTCACAGTCAGTGTCCAGCTCCTACC | |
| | | TCGCTTGGTACCAGCAAAAGCCTGGACAAGCCCCACGCCTTCTGATCTAC | |
| | | GGCGCCTCGAGCAGAGCCACTGGAATTCCCGACCGGTTCTCCGGTTCTGG | |
| | | GTCCGGCACCGATTTCACCCTGACCATTTCCCGCTTGGAACCGGAGGACT | |
| | | TTGCCGTGTATTACTGCCAGCAGGCATTCTCGAGCCCGACCTTCGGTGGA | |
| | | GGGACTAAGGTCGAGATCAAG | |
| KLK5- | VH | CAGGTCCAGCTGGTGCAATCGGGGGCCGAAGTCAAGAAGCCTGGAGCGTC | 214 |
| L3-Ab1 | | CGTGAAGGTGTCCTGCAAAGCCTCCGGCTACACCTTCACCGGTTACTACA | |
| | | TGCACTGGGTCAGACAGGCACCCGGACAGGGACTCGAATGGATGGGCGGC | |
| | | ATCATCCCGATCAGCGGAACCGCCAACTATGCCCAAAAGTTCCAGGGTCG | |
| | | CGTGACTATTACGGCTGACGAGTCCACCTCCACTGCCTACATGGAACTGT | |
| | | CCTCCCTGCGGAGCGAGGATACCGCCGTGTACTACTGCGCGAGGGGATCA | |
| | | GCCTCGGGGTATTACGACCGCTCGTACTACTACTACGGGATGGACGTGTG | |
| | | GGGACAGGGCACCACCGTGACTGTGTCAAGC | |
| | VL | GACATTCAGATGACTCAGTCCCCATCCTCCGTGAGCGCCTCAGTGGGGGA | 215 |
| | | TCGGGTCACCATTACCTGTAGAGCCTCCCAGGGCATCTCCAGCTGGCTGG | |
| | | CTTGGTACCAGCAGAAGCCTGGAAAGGCCCCTAAGTTGCTGATCTACGCA | |
| | | GCGAGCAGCCTGCAGTCAGGAGTGCCGTCCCGCTTCTCGGGATCCGGTTC | |
| | | TGGAACCGATTTCACCCTCACGATCTCGTCGCTGCAACCGGAGGACTTCG | |
| | | CCACCTACTACTGCCAACAAGTGTCGTCCTTCCCCTATACTTTTGGCGGC | |
| | | GGGACCAAAGTGGAAATCAAG | |
| KLK5- | VH | CAGGTCCAACTGGTGCAATCCGGCGCCGAAGTGAAGAAGCCGGGATCGAG | 216 |
| L3-Ab2 | | CGTGAAGGTGTCCTGCAAAGCCTCCGGAGGGACCTTTTCCGGCTACTCCA | |
| | | TCTCATGGGTCAGACAGGCACCTGGACAGGGTCTGGAATGGATGGGCCTC | |
| | | ATCTTCCCCATTTCGGGGACCGCCAACTACGCCCAGAAGTTCCAGGGAAG | |
| | | GGTCACTATCACCGCCGACGAGAGCACTTCCACGGCCTACATGGAGCTGA | |
| | | GCTCTCTGCGGAGCGAAGATACCGCGGTGTACTATTGTGCTCGGGGATCA | |
| | | GCGAGCGGTTACTACGACCGCTCCTACTACTACTACGGGATGGACGTCTG | |
| | | GGGACAGGGCACCACTGTGACCGTGTCCTCG | |
| | VL | GACATTCAGATGACTCAGTCCCCATCCTCCGTGAGCGCCTCAGTGGGGGA | 215 |
| | | TCGGGTCACCATTACCTGTAGAGCCTCCCAGGGCATCTCCAGCTGGCTGG | |
| | | CTTGGTACCAGCAGAAGCCTGGAAAGGCCCCTAAGTTGCTGATCTACGCA | |
| | | GCGAGCAGCCTGCAGTCAGGAGTGCCGTCCCGCTTCTCGGGATCCGGTTC | |
| | | TGGAACCGATTTCACCCTCACGATCTCGTCGCTGCAACCGGAGGACTTCG | |
| | | CCACCTACTACTGCCAACAAGTGTCGTCCTTCCCCTATACTTTTGGCGGC | |
| | | GGGACCAAAGTGGAAATCAAG | |
| KLK5- | VH | CAGGTCCAACTGGTGCAGTCAGGCGCAGAAGTCAAGAAGCCCGGAAGCTC | 217 |
| L3-Ab2 | | GGTGAAAGTGTCCTGCAAGGCCTCCGGGGGAACCTTTAACAACTACCACA | |
| | | TTTCGTGGGTCCGCCAGGCCCCGGGACAGGGTCTGGAGTGGATGGGCGGC | |
| | | ATCATCCCTCCATTCGGAACCGCCCATTACGCCCAAAAGTTCCAGGGCAG | |
| | | AGTGACGATCACCGCGGACGAATCGACCTCCACCGCCTACATGGAACTGA | |
| | | GCTCTCTCCGGTCCGAGGATACTGCCGTGTACTACTGTGCGAGGGGTAGC | |
| | | GCTTCCGGGTATTACGACCGGTCCTACTACTACTACGGAATGGACGTCTG | |
| | | GGGCCAGGGAACCACTGTGACTGTGTCATCC | |
| | VL | GACATTCAGATGACTCAGTCCCCATCCTCCGTGAGCGCCTCAGTGGGGGA | 215 |
| | | TCGGGTCACCATTACCTGTAGAGCCTCCCAGGGCATCTCCAGCTGGCTGG | |
| | | CTTGGTACCAGCAGAAGCCTGGAAAGGCCCCTAAGTTGCTGATCTACGCA | |
| | | GCGAGCAGCCTGCAGTCAGGAGTGCCGTCCCGCTTCTCGGGATCCGGTTC | |
| | | TGGAACCGATTTCACCCTCACGATCTCGTCGCTGCAACCGGAGGACTTCG | |
| | | CCACCTACTACTGCCAACAAGTGTCGTCCTTCCCCTATACTTTTGGCGGC | |
| | | GGGACCAAAGTGGAAATCAAG | |
| KLK5- | VH | CAAGTGCAGCTTGTGCAGTCCGGGGCAGAAGTCAAGAAGCCCGGAAGCTC | 218 |
| L3-Ab4 | | GGTGAAAGTGTCCTGCAAGGCCTCCGGTGGAACCTTCAACAACTACCACA | |
| | | TCTCCTGGGTCCGCCAGGCTCCGGGACAGGGTCTGGAGTGGATGGGCGGC | |
| | | ATCATCCCTCCATTTGGGACCGCGCATTATGCCCAAAAGTTCCAGGGCCG | |
| | | CGTGACCATTACTGCCGATGAATCCACGAGCACCGCCTACATGGAACTGA | |
| | | GCTCGCTGAGATCAGAGGACACTGCCGTGTACTACTGTGCGAGGGGAGAG | |
| | | GCCTCCGGCTACTACGACCGGTCCTACTACTACTACGGACTCGACGTCTG | |
| | | GGGACAGGGCACCACTGTGACCGTGTCGTCA | |
| | VL | GACATTCAGATGACTCAGTCCCCATCCTCCGTGAGCGCCTCAGTGGGGGA | 215 |
| | | TCGGGTCACCATTACCTGTAGAGCCTCCCAGGGCATCTCCAGCTGGCTGG | |
| | | CTTGGTACCAGCAGAAGCCTGGAAAGGCCCCTAAGTTGCTGATCTACGCA | |
| | | GCGAGCAGCCTGCAGTCAGGAGTGCCGTCCCGCTTCTCGGGATCCGGTTC | |
| | | TGGAACCGATTTCACCCTCACGATCTCGTCGCTGCAACCGGAGGACTTCG | |
| | | CCACCTACTACTGCCAACAAGTGTCGTCCTTCCCCTATACTTTTGGCGGC | |
| | | GGGACCAAAGTGGAAATCAAG | |
| KLK5- | VH | CAAGTGCAGCTCGTGCAGTCCGGAGCCGAAGTCAAGAAGCCGGGCTCATC | 219 |
| L3-Ab5 | | CGTGAAAGTGTCCTGCAAGGCCTCCGGTGGAACCTTCAGCGGATACTCGA | |
| | | TTTCCTGGGTCCGCCAAGCCCCTGGACAAGGCCTTGAGTGGATGGGACTG | |
| | | ATCTTTCCCATCTCGGGGACCGCAAACTATGCGCAGAAGTTCCAGGGCAG | |
| | | AGTGACCATCACTGCCGACGAAAGCACCAGCACGGCCTACATGGAACTGT | |
| | | CAAGCCTGAGGTCCGAGGATACTGCGGTGTACTACTGCGCCCGGCAGTCG | |
| | | GCTTCCGGGTACTACGACCGGTCTTACTACCGCTACGGAATGGACGTCTG | |
| | | GGGCCAGGGTACCACTGTGACCGTGTCCTCC | |
| | VL | GACATTCAGATGACTCAGTCCCCATCCTCCGTGAGCGCCTCAGTGGGGGA | 215 |
| | | TCGGGTCACCATTACCTGTAGAGCCTCCCAGGGCATCTCCAGCTGGCTGG | |
| | | CTTGGTACCAGCAGAAGCCTGGAAAGGCCCCTAAGTTGCTGATCTACGCA | |
| | | GCGAGCAGCCTGCAGTCAGGAGTGCCGTCCCGCTTCTCGGGATCCGGTTC | |
| | | TGGAACCGATTTCACCCTCACGATCTCGTCGCTGCAACCGGAGGACTTCG | |
| | | CCACCTACTACTGCCAACAAGTGTCGTCCTTCCCCTATACTTTTGGCGGC | |
| | | GGGACCAAAGTGGAAATCAAG | |
| KLK5- | VH | CAACTGGTGCAGTCCGGTGCTGAAGTCAAGAAGCCCGGCTCTAGCGTGAA | 220 |
| L3-Ab6 | | AGTGTCGTGCAAGGCATCCGGGGGAACCTTCTCCGGTTACGTGATTAGCT | |
| | | GGGTCAGACAGGCGCCGGGACAGGGCCTCGAGTGGATGGGCGGGATCTTC | |
| | | CCTATCTCGGGATACGCCGTGTATGCCCAGAAGTTTCAGGGCCGCGTGAC | |
| | | CATCACTGCCGATGAATCCACCTCCACGGCCTACATGGAACTGAGCTCCC | |
| | | TGCGGTCCGAGGACACCGCCGTGTATTACTGTGCCCGGGCCTCAGCGTCG | |
| | | GGATACTACGACCGCTCCTACTACTACTACGGAATGGACGTCTGGGGCCA | |
| | | GGGAACTACTGTGACCGTGTCAAGC | |
| | VL | GACATTCAGATGACCCAGTCCCCGTCCTCCGTGTCGGCTTCCGTGGGAGA | 221 |
| | | CAGAGTCACTATCACTTGTCGGGCCAGCCAGGGAATTTCCTCATGGCTGG | |
| | | CCTGGTATCAGCAGAAGCCTGGAAAGGCCCCAAAGCTTCTCATCTACGCC | |
| | | GCGTCGTCACTGCAATCCGGCGTGCCCAGCCGCTTCTCTGGGTCCGGATC | |
| | | GGGGACTGACTTTACCCTGACCATCAGCAGCCTGCAGCCCGAGGATTTCG | |
| | | CAACGTACTACTGCCAACAAGTGTCCTCCTTCCCGCAAACCTTCGGCGGC | |
| | | GGTACCAAAGTCGAAATCAAG | |
| KLK5- | VH | CAAGTGCAACTGGTGCAGAGCGGAGCCGAAGTCCGCAAGCCCGGCTCGAG | 222 |
| L3-Ab7 | | CGTGAAAGTGTCCTGCAAGGCCTCGGGCGGCACCTTCTCCAACTACCACA | |
| | | TCTCATGGGTCAGACAGGCGCCTGGACAGGGACTCGAGTGGATGGGCGGC | |
| | | ATTTTCCCGACCTTCGGTACCGCCAACTACGCTCAGAAGTTTCAGGGTCG | |
| | | GGTCACTATCACCGCGGACGAAAGCACCTCCACGGCCTACATGGAACTGT | |
| | | CATCGCTGCGGTCCGAGGACACCGCCGTGTACTACTGTGCCCGCGGAAGC | |
| | | GCATCCGGATATTACGATAGGTCCTACTACTACGACGGGATGGATGTCTG | |
| | | GGGACAGGGGACTACCGTGACTGTGTCCTCT | |
| | VL | GATATTCAAATGACCCAGTCACCGTCTAGCGTGTCAGCCTCCGTGGGCGA | 223 |
| | | CCGCGTGACTATCACCTGTCGGGCCAGCCAGGGAATCTCCTCGTGGCTGG | |
| | | CTTGGTATCAGCAGAAACCCGGAAAGGCCCCGAAGCTGCTCATCTACGCC | |
| | | GCGAGCTCGCTGCAGTCCGGCGTGCCATCGAGATTTTCCGGAAGCGGATC | |
| | | CGGGACCGACTTCACGCTTACTATCTCCTCCCTGCAACCCGAGGACTTCG | |
| | | CAACCTACTACTGCCAACAAGTCATTTCCTTCCCTTACACCTTCGGTGGC | |
| | | GGGACTAAGGTCGAAATCAAG | |
| KLK7- | VH | CAAGTGCAGTTGGTGCAGTCAGGCGCCGAAGTCAAGAAGCCGGGTAGCAG | 224 |
| L1-Ab1 | | CGTGAAAGTGTCGTGCAAGGCATCCGGCGGCACTTTCTCGAACTACGCCA | |
| | | TCTCCTGGGTCAGACAGGCGCCTGGACAAGGACTGGAATGGATGGGGTGG | |
| | | ATTTCCGCCTACTCCGGGGACACCAACTACGCCCAGAAGCTGCAGGGACG | |
| | | GGTCACCATGACGACCGACACTTCCACCTCCACCGCCTACATGGAGCTCC | |
| | | GCTCACTGCGGTCCGACGATACTGCCGTGTACTATTGTGCGAGAGCAGCC | |
| | | GGCTACGGAGCTAGGCACTGGTACTTCGATCTGTGGGGACAGGGTACCAT | |
| | | GGTCACTGTGTCCAGC | |
| | VL | GACATTCAGATGACCCAGAGCCCCTCCTCCCTCTCCGCATCCGTGGGCGA | 225 |
| | | CAGAGTCACCATCACTTGTCGGGCTAGCCAGTCGATCTCCTCATATCTTA | |
| | | ACTGGTACCAGCAAAAGCCTGGAAAGGCGCCGAAGCTGCTGATCTACGGA | |
| | | GCCAGCAGCCTGCAGTCCGGAGTGCCATCCCGCTTCTCCGGCTCCGGATC | |
| | | TGGAACCGATTTCACCCTGACTATCTCCTCGCTGCAACCGGAGGACTTTG | |
| | | CCACTTACTACTGCGACCAGTCGTACTCAACGCCCCCTATTACCTTCGGT | |
| | | GGCGGGACCAAAGTGGAAATCAAG | |
| KLK7- | VH | CAGGTCCAACTCGTGCAGTCCGGGGCTGAAGTCAAGAAGCCGGGAGCTAG | 295 |
| L1-Ab2 | | CGTGAAAGTGTCCTGCAAGGCCTCCGGCTACACCTTCGGACCCTACGGAA | |
| | | TTTCATGGGTCAGACAGGCGCCTGGACAGGGTCTGGAGTGGATGGGGTGG | |
| | | ATCTCCGCCTACTCGGGCGACACTAACTACGCGCAGAAGCTGCAAGGACG | |
| | | CGTGACCATGACTACCGACACCTCGACCAGCACCGCCTACATGGAACTTA | |
| | | GGTCCCTGCGGTCCGACGATACGGCCGTGTACTACTGTGCAAGAGCCGCC | |
| | | GGCTATGGAGCCCGGTGGTGGTACTTCGATCTGTGGGGCCAGGGTACTAT | |
| | | GGTCACCGTGTCAAGC | |
| | VL | GATATCCAAATGACCCAGTCCCCGTCATCCCTTTCCGCCTCCGTGGGAGA | 227 |
| | | TAGAGTGACCATCACTTGTCGGGCGTCGCAGTCCATCTCGTCGTACCTGA | |
| | | ACTGGTACCAGCAGAAACCTGGGAAGGCCCCCAAGCTGCTGATCTACGGA | |
| | | GCCAGCAGCCTGCAGTCCGGAGTGCCTTCACGCTTCTCCGGGTCTGGAAG | |
| | | CGGCACCGACTTTACCTTGACTATTTCCTCCCTCCAACCCGAGGACTTCG | |
| | | CTACGTACTATTGCGACGGCTCGTACAGCACCCCGCCAATTACCTTCGGT | |
| | | GGTGGCACTAAGGTCGAAATCAAG | |
| KLK7- | VH | CAAGTGCAGCTGGTGCAGTCCGGAGCGGAAGTCAAGAAGCCGGGAGCTTC | 228 |
| L1-Ab3 | | CGTGAAAGTGTCCTGCAAGGCCTCCGGGTACACTTTCACATCGGAGGGCA | |
| | | TCCACTGGGTCAGACAGGCCCCTGGCCAAGGACTGGAGTGGATGGGGTCA | |
| | | ATTTCGGCCAGCTCCGGAGACACCAACTACGCCCAGGAACTTCAGGGCCG | |
| | | CGTGACTATGACCACCGACACCAGCACCTCCACTGCCTACATGGAACTGC | |
| | | GGTCGCTGAGGAGCGACGATACCGCCGTGTACTACTGTGCAAGAGCGGCT | |
| | | GGTTATGGTGCCCGGCATTGGTACTTCGATCTCTGGGGACAGGGCACCAT | |
| | | GGTCACGGTGTCATCC | |
| | VL | GACATTCAAATGACTCAGTCCCCCTCATCTCTGTCCGCGAGCGTGGGAGA | 229 |
| | | CAGAGTGACCATTACGTGTCGCGCCAGCCAGAGCATCTCGTCATACCTGA | |
| | | ACTGGTACCAGCAGAAGCCTGGCAAAGCCCCGAAGCTTTTGATCTACGGG | |
| | | GCTTCCTCCCTCCAATCGGGAGTGCCTTCCCGGTTCTCCGGAAGCGGATC | |
| | | CGGGACCGATTTCACTCTGACCATCTCCTCGCTGCAACCAGAAGATTTCG | |
| | | CCACCTACTACTGCGACCAGTTCTATTCCACCCCGCCCATCACTTTTGGC | |
| | | GGCGGTACCAAGGTCGAGATCAAG | |
| KLK7- | VH | CAAGTGCAGTTGGTGCAGTCAGGCGCCGAGGTCAAGAAACCCGGAGCCAG | 230 |
| L1-Ab4 | | CGTGAAGGTGTCCTGCAAGGCGTCCGGTTACACCTTCACTAGCGAAGGGA | |
| | | TCCACTGGGTCCGCCAAGCTCCGGGACAGGGACTGGAATGGATGGGGTCG | |
| | | ATTTCCGCCTCGTCGGGAGATACCAACTATGCCCAGAAGCTGCAGGGTCG | |
| | | CGTGACCATGACTACTGACACCTCCACCTCCACGGCCTACATGGAGCTGC | |
| | | GGTCACTGCGGAGCGACGACACCGCCGTGTACTACTGTGCGAGAGCTGCC | |
| | | GGCTACGGAGCAAGGCATTGGTACTTCGATCTCTGGGGCCAGGGCACTAT | |
| | | GGTCACCGTGTCCTCC | |
| | VL | GATATTCAAATGACCCAGTCACCGAGCTCCTTGAGCGCGTCCGTGGGAGA | 286 |
| | | CAGAGTGACCATTACTTGTCGCGCCTCGCAATCGATCTCATCCTATCTCA | |
| | | ACTGGTACCAGCAGAAGCCCGGCAAAGCTCCAAAGCTTCTGATCTACGGC | |
| | | GCAAGCAGCCTGCAGTCTGGAGTGCCTTCGCGGTTCTCCGGTTCCGGATC | |
| | | CGGGACCGACTTCACTCTGACCATCTCCTCCCTGCAACCTGAGGATTTCG | |
| | | CCACCTACTACTGCGACCAGTTTTACTCCACCCCGCCCATCACGTTCGGA | |
| | | GGGGGCACTAAGGTCGAAATCAAG | |
| KLK7- | VH | CAAGTGCAGCTGGTGCAGAGCGGGGCTGAAGTCAAGAAGCCTGGAGCCAG | 231 |
| L1-Ab5 | | CGTGAAAGTGTCCTGCAAGGCCTCAGGCTACACCTTCACATCCGAGGGCA | |
| | | TTCACTGGGTCAGACAGGCCCCGGGACAAGGACTGGAGTGGATGGGTTCG | |
| | | ATCTCGGCCTCATCCGGCGATACTAACTACGCGCAGAAGCTGCAGGGTCG | |
| | | CGTGACCATGACCACTGACACTAGCACCTCGACCGCTTACATGGAACTGA | |
| | | GGTCCCTCCGGTCCGACGATACCGCCGTGTACTACTGTGCGAGAGCCGCA | |
| | | GGATATGGGGCCCGGCATTGGTACTTCGACCTTTGGGGACAGGGCACCAT | |
| | | GGTCACGGTGTCCTCC | |
| | VL | GACATCCAAATGACCCAGTCCCCATCTTCGCTGTCGGCCAGCGTGGGAGA | 232 |
| | | TCGCGTGACTATTACCTGTCGGGCTAGCCAGTCCATCTCGTCCTATCTCA | |
| | | ACTGGTACCAGCAGAAGCCGGGAAAGGCCCCTAAGCTGTTGATCTACGGG | |
| | | GCGTCATCCCTTCAATCCGGGGTGCCTTCAAGATTCTCCGGATCCGGTTC | |
| | | CGGCACCGACTTCACTCTGACCATTAGCAGCCTGCAGCCCGAAGATTTCG | |
| | | CCACCTACTACTGCGACCAGTGGTACTCCACGCCGCCCATCACTTTTGGC | |
| | | GGCGGAACCAAGGTCGAGATCAAA | |
| KLK7- | VH | CAAGTGCAGCTGGTGCAGTCCGGTGCCGAAGTGAAAAAGCCGGGTGCTAG | 233 |
| L1-Ab6 | | CGTGAAGGTGTCCTGCAAGGCTTCCGGGTACACCTTCACATCTGAGGGCA | |
| | | TTCACTGGGTCAGACAGGCCCCTGGACAAGGGCTGGAGTGGATGGGATCG | |
| | | ATCTCAGCGTCCTCCGGCGACACCAACTACGCCCAGAAGCTGCAGGGACG | |
| | | GGTCACTATGACCACTGACACGAGCACTTCCACCGCCTACATGGAACTCC | |
| | | GGTCACTGCGCTCGGATGATACCGCCGTGTACTACTGTGCAAGAGCGGCC | |
| | | GGATACGGAGCCAGGTCGTGGTATTTCGACCTTTGGGGCCAGGGCACCAT | |
| | | GGTCACCGTGTCCAGC | |
| | VL | GACATTCAAATGACCCAGTCCCCATCGTCCCTGAGCGCCTCAGTGGGAGA | 287 |
| | | TAGAGTGACCATCACCTGTCGCGCTAGCCAGTCCATCTCCTCGTACCTGA | |
| | | ACTGGTACCAGCAGAAGCCTGGAAAAGCCCCGAAGCTTCTGATCTACGGT | |
| | | GCCAGCAGCCTGCAGTCTGGCGTGCCTTCCCGGTTCTCGGGATCAGGCTC | |
| | | CGGGACCGACTTCACTTTGACTATCTCCTCCCTCCAACCCGAGGATTTCG | |
| | | CGACCTACTATTGCGACCAGTTCTACTCCACGCCGCCCATTACCTTTGGC | |
| | | GGGGGAACTAAGGTCGAAATCAAG | |
| KLK7- | VH | CAAGTGCAGCTGGTGCAGTCTGGCGCCGAGGTCAAGAAGCCGGGAGCCTC | 234 |
| L1-Ab7 | | AGTGAAAGTGTCCTGCAAGGCCTCCGGATACACCTTCACATCGGAAGGGA | |
| | | TTCACTGGGTCCGCCAAGCTCCCGGTCAAGGACTTGAGTGGATGGGCTCC | |
| | | ATCAGCGCTTCGTCCGGGGACACTAACTACGCCCAGAAGCTGCAGGGACG | |
| | | CGTGACCATGACTACCGACACCTCCACCTCAACCGCCTACATGGAACTGA | |
| | | GAAGCCTGCGGTCGGACGATACTGCCGTGTATTACTGTGCGAGGGGTGCA | |
| | | GGATACGGCGCGAGAGATTGGTACTTCGACCTCTGGGGCCAGGGAACCAT | |
| | | GGTCACGGTGTCCAGC | |
| | VL | GATATCCAAATGACTCAGAGCCCATCCTCCTTGTCGGCCTCAGTGGGAGA | 235 |
| | | CAGAGTGACCATTACGTGTCGCGCGAGCCAGTCGATCTCCTCCTACCTCA | |
| | | ACTGGTACCAGCAGAAGCCCGGCAAAGCTCCGAAGCTTCTGATCTATGGA | |
| | | GCCTCCTCCCTGCAATCGGGCGTGCCGTCAAGGTTCTCTGGTTCCGGATC | |
| | | CGGCACCGACTTTACCCTGACCATTAGCAGCCTGCAGCCCGAGGACTTCG | |
| | | CCACTTACTACTGCGATCAGCGGTACTCCACCCCTCCTATCACCTTCGGG | |
| | | GGGGGAACTAAGGTCGAAATCAAG | |
| KLK7- | VH | CAAGTGCAGCTGGTGCAGAGCGGAGCCGAAGTCAAGAAGCCGGGAGCTTC | 236 |
| L1-Ab8 | | CGTGAAAGTGTCCTGCAAGGCCTCCGGCTACACCTTCACATCGGAGGGTA | |
| | | TTCACTGGGTCCGCCAAGCACCTGGACAGGGCCTGGAGTGGATGGGCTCC | |
| | | ATCTCGGCCTCATCCGGGGACACCAACTACGCCCAGAAGCTGCAGGGACG | |
| | | GGTCACGATGACTACTGACACCTCCACCTCAACCGCCTATATGGAACTTC | |
| | | GCAGCCTGAGGAGCGATGACACTGCCGTGTACTACTGTGCGAGATCTGCG | |
| | | GGATACGGTGCCCGGCATTGGTACTTCGATCTCTGGGGCCAGGGGACTAT | |
| | | GGTCACCGTGTCGTCC | |
| | VL | GATATCCAAATGACCCAGTCCCCGAGCTCCCTGTCCGCCTCCGTGGGAGA | 288 |
| | | TCGCGTGACCATTACGTGTAGAGCCTCCCAGTCGATCAGCAGCTATCTGA | |
| | | ACTGGTACCAGCAGAAGCCGGGCAAAGCTCCTAAGCTCCTTATCTACGGC | |
| | | GCCAGCTCACTGCAGTCCGGAGTGCCATCGCGGTTCTCCGGATCCGGCTC | |
| | | AGGAACCGACTTCACCTTGACTATTTCTTCGCTGCAACCTGAGGACTTTG | |
| | | CGACCTACTACTGCGACCAGTGGTACTCCACCCCCCCCATCACTTTCGGG | |
| | | GGGGGTACTAAGGTCGAAATCAAG | |
| KLK7- | VH | CAAGTGCAACTGCAGGAGTCTGGACCTGGTTTGGTCAAGCCGAGCCAGAC | 237 |
| L2-Ab1 | | GCTTTCCCTCACCTGTACCGTGTCGGGGGGATCCATCTCATCGGGAGGCT | |
| | | ACTACTGGTCCTGGATTAGACAGCATCCCGGAAAGGGGCTGGAATGGATT | |
| | | GGTTCCATCTACTACTCCGGCTCCACTTACTATAACCCAAGCCTCAAGTC | |
| | | AAGGGTCACTATCTCGGTGGATACCTCCAAGAACCAGTTCAGCCTGAAAC | |
| | | TGTCCAGCGTGACCGCTGCCGACACTGCGGTGTACTACTGCGCCCGGGCA | |
| | | AAGCAGCTGACCACCCACCGCTACTTCGACTACTGGGGCCAGGGCACTCT | |
| | | GGTCACCGTGTCGAGC | |
| | VL | GACATCCAAATGACCCAGAGCCCCTCCTCCTTGTCGGCTTCCGTGGGAGA | 238 |
| | | CAGGGTCACTATCACGTGTCAGGCGTCACAGGACATCGCGAATTACCTGA | |
| | | ACTGGTACCAGCAGAAGCCCGGAAAGGCACCTAAGCTCCTGATCTATGAC | |
| | | GCCTCCAACCTGGAAACCGGGGTGCCGTCTAGATTCTCCGGTTCCGGAAG | |
| | | CGGCACCGATTTTACTTTCACCATTAGCAGCCTCCAGCCTGAGGATATTG | |
| | | CCACCTACTACTGCCAACAAGCCTCGGACCTTCCATTCACCTTCGGTGGC | |
| | | GGCACTAAGGTCGAAATCAAA | |
| KLK7- | VH | CAAGTGCAACTGCAGGAGTCAGGGCCTGGTCTGGTCAAGCCAAGCCAGAC | 239 |
| L2-Ab2 | | CCTGTCTCTGACGTGCACTGTGTCGGGAGGATCCATCAGCTCGGGGGGTT | |
| | | ACTACTGGCATTGGATTAGGCAGCACCCCGGAAAGGGCCTTGAATGGATT | |
| | | GGCTACATCTACCGGTCCGGCTACACCTACTACAACCCGTCCCTGAAGTC | |
| | | GAGAGTGACCATCTCCGTCGACACCAGCAAAAACCAGTTCAGCCTGAAGT | |
| | | TGTCATCCGTGACCGCCGCTGACACTGCGGTGTACTATTGTGCACGGGCC | |
| | | AAGCAGCTCACTACCCACCGCTACTTCGATTACTGGGGACAGGGCACTCT | |
| | | CGTGACCGTGTCCTCC | |
| | VL | GACATCCAAATGACCCAGAGCCCCTCCTCCTTGTCGGCTTCCGTGGGAGA | 238 |
| | | CAGGGTCACTATCACGTGTCAGGCGTCACAGGACATCGCGAATTACCTGA | |
| | | ACTGGTACCAGCAGAAGCCCGGAAAGGCACCTAAGCTCCTGATCTATGAC | |
| | | GCCTCCAACCTGGAAACCGGGGTGCCGTCTAGATTCTCCGGTTCCGGAAG | |
| | | CGGCACCGATTTTACTTTCACCATTAGCAGCCTCCAGCCTGAGGATATTG | |
| | | CCACCTACTACTGCCAACAAGCCTCGGACCTTCCATTCACCTTCGGTGGC | |
| | | GGCACTAAGGTCGAAATCAAA | |
| KLK7-L2- | VH | CAGGTCCAACTGCAAGAATCAGGGCCGGGACTCGTGAAGCCATCTCAGAC | 240 |
| Ab3 | | CTTGTCGCTGACCTGTACTGTGTCCGGGGGTTCCATTTCGTCCGGCGGAT | |
| | | ACTCCTGGAACTGGATTCGGCAGCACCCCGGAAAAGGCCTCGAGTGGATC | |
| | | GGCTCCATCTACCGGTCGGGTAGCACTTATTACAACCCTAGCCTGAAGTC | |
| | | GAGAGTGACCATCTCCGTCGACACGTCCAAGAACCAGTTCAGCCTGAAGC | |
| | | TGAGCTCAGTGACCGCTGCCGACACTGCCGTGTACTACTGCGCAAGGGCC | |
| | | AAGGCGCTGACTACCCATCGCTACGTGGATTACTGGGGACAGGGCACCCT | |
| | | TGTGACCGTGTCCTCC | |
| | VL | GACATTCAAATGACCCAGTCCCCCTCCTCCCTGTCGGCTTCCGTGGGAGA | 241 |
| | | CAGAGTGACCATCACTTGCCAAGCCAGCCAGGACATCGCCAACTACCTGA | |
| | | ACTGGTATCAGCAGAAGCCAGGGAAGGCACCGAAGTTGCTTATCTACGAC | |
| | | GCCTCGAACCTGGAAACGGGTGTCCCCTCACGGTTCTCTGGGAGCGGAAG | |
| | | CGGCACTGATTTCACCTTCACCATCTCCTCCCTCCAACCGGAGGACATTG | |
| | | CGACCTACTACTGTCAGCAGGCCCGCGATCTGCCTTTCACTTTTGGCGGA | |
| | | GGCACCAAAGTGGAGATCAAG | |
| KLK7-L2- | VH | CAGGTCCAACTTCAAGAATCCGGCCCTGGACTCGTGAAACCAAGCCAGAC | 242 |
| Ab4 | | TCTGAGCCTGACTTGTACCGTGTCCGGAGGTTCGATCTCCTCCGGCGGCT | |
| | | ACTCATGGAACTGGATTAGACAGCACCCGGGGAAGGGACTGGAGTGGATT | |
| | | GGTTCGATCTACCGGTCCGGCTCAACCTACTACAACCCCTCGCTGAAGTC | |
| | | CCGGGTCACCATCTCGGTGGATACCTCCAAGAACCAGTTCAGCCTGAAGC | |
| | | TGAGCTCCGTGACTGCCGCCGACACTGCTGTGTACTATTGCGCAAGGGCG | |
| | | AAGCAGTTGACCCTGACCCGCTACTTCGACTACTGGGGACAGGGGACGCT | |
| | | CGTGACCGTGTCCTCT | |
| | VL | GACATTCAAATGACTCAGAGCCCGAGCAGCTTGTCAGCATCGGTCGGGGA | 243 |
| | | TCGCGTGACTATCACCTGTCAAGCGTCGCAGGACATCGCTAACTACCTGA | |
| | | ACTGGTACCAGCAGAAGCCGGGAAAGGCCCCTAAGCTGCTGATCTATGAC | |
| | | GCCTCCAACCTGGAAACGGGAGTGCCTTCCCGGTTCTCCGGTTCCGGATC | |
| | | GGGCACCGACTTCACCTTCACCATCTCCTCCCTCCAACCCGAGGATATTG | |
| | | CCACCTACTACTGCCAGCAGGCGTCACAGCTTCCATTCACTTTTGGCGGC | |
| | | GGGACCAAAGTGGAAATCAAG | |
| KLK7-L2- | VH | CAAGTGCAGCTGCAGGAATCCGGCCCGGGTCTTGTGAAGCCCTCACAAAC | 244 |
| Ab5 | | CCTGAGCTTGACCTGTACCGTGTCTGGAGGGTCGATCTCCTCCGGCGGCT | |
| | | ACTACTGGCACTGGATTAGGCAGCATCCTGGGAAGGGTCTGGAGTGGATC | |
| | | GGATACATCTACCGGTCCGGATACACGTACTATAACCCAAGCCTGAAGTC | |
| | | CCGCGTGACCATTAGCGTGGACACCTCCAAGAACCAGTTCTCGCTGAAAC | |
| | | TCAGCTCAGTCACTGCTGCCGATACCGCCGTCTACTACTGCGCAAGAGCG | |
| | | AAGCAGCTGACTACCTCGCGGTACTTCGACTACTGGGGCCAGGGAACTCT | |
| | | CGTGACTGTGTCCTCC | |
| | VL | GACATCCAAATGACCCAGAGCCCCTCCTCCTTGTCGGCTTCCGTGGGAGA | 238 |
| | | CAGGGTCACTATCACGTGTCAGGCGTCACAGGACATCGCGAATTACCTGA | |
| | | ACTGGTACCAGCAGAAGCCCGGAAAGGCACCTAAGCTCCTGATCTATGAC | |
| | | GCCTCCAACCTGGAAACCGGGGTGCCGTCTAGATTCTCCGGTTCCGGAAG | |
| | | CGGCACCGATTTTACTTTCACCATTAGCAGCCTCCAGCCTGAGGATATTG | |
| | | CCACCTACTACTGCCAACAAGCCTCGGACCTTCCATTCACCTTCGGTGGC | |
| | | GGCACTAAGGTCGAAATCAAA | |
| KLK7-L3- | VH | CAGGTCCAGCTCGTGCAGTCAGGCGCCGAAGTCAAGAAGCCGGGTTCCAG | 289 |
| Ab1 | | CGTGAAGGTGTCCTGCAAAGCCAGCGGAGGCACTTTCTCCAACTATGCGA | |
| | | TCAGCTGGGTCAGACAGGCCCCCGGACAAGGACTGGAGTGGATGGGCGGG | |
| | | ATCATTCCAATCTTCGGGACTGCGAACTACGCACAGAAGTTTCAGGGTCG | |
| | | CGTGACCATCACTGCTGACGAGTCGACCTCTACCGCCTACATGGAGCTGT | |
| | | CCTCGCTGCGGTCCGAAGATACCGCCGTGTACTACTGTGCGAGAGGAGCC | |
| | | CCTAATTACTACGGAAGCTCATCCTCGCGGGGGATTGCCTTCGACATCTG | |
| | | GGGACAAGGCACCATGGTCACCGTGTCCTCG | |
| | VL | GACATCCAAATGACCCAGTCGCCATCCTCCGTGAGCGCATCCGTGGGAGA | 290 |
| | | TCGCGTCACCATTACCTGTCGGGCGTCACAGGGCATTAGCTCTTGGCTGG | |
| | | CCTGGTACCAGCAGAAGCCCGGAAAGGCTCCCAAACTTCTGATCTACGCC | |
| | | GCGTCCTCACTGCAAAGCGGCGTGCCTTCGAGATTCAGCGGATCCGGTTC | |
| | | CGGCACTGACTTTACTCTCACCATCTCGTCCCTGCAACCGGAGGACTTCG | |
| | | CCACCTACTACTGCGTGCAGGCCAACGCCTTCCCTTACACCTTCGGAGGG | |
| | | GGGACTAAGGTCGAAATCAAG | |
| KLK7-L3- | VH | CAAGTGCAACTGGTGCAGTCGGGTGCCGAGGTCAAGAAGCCTGGGAGCTC | 291 |
| Ab2 | | CGTGAAAGTGTCCTGCAAGGCCTCCGGAGGAACTTTCAACAACTACGCGA | |
| | | TCAGCTGGGTCAGACAGGCTCCAGGCCAAGGCCTCGAATGGATGGGCGCG | |
| | | ATTATTCCCATCTTCGGAGTGGCCAACTACGCCCAGAAGTTTCAGGGACG | |
| | | CGTGACTATCACCGCCGATGAGTCCACCTCCACTGCCTACATGGAACTGA | |
| | | GCTCACTGCGGTCCGAGGACACCGCAGTGTACTACTGTGCAAGGGGTGCC | |
| | | CCGAATTACTATGGAGCCCAGTCCTCACGGGGGATCGCTTTCGACATCTG | |
| | | GGGACAGGGCACCATGGTCACCGTGTCGTCG | |
| | VL | GACATTCAAATGACCCAGTCGCCTTCGTCCGTGAGCGCATCCGTGGGTGA | 292 |
| | | TCGCGTGACCATCACTTGCCGGGCTTCACAAGGCATTTCCTCCTGGCTGG | |
| | | CCTGGTACCAGCAGAAGCCCGGAAAAGCGCCCAAGCTGCTCATCTACGCC | |
| | | GCCTCCTCTCTTCAATCCGGCGTGCCGTCAAGATTCTCGGGAAGCGGATC | |
| | | CGGCACCGACTTCACGCTGACTATCAGCAGCCTGCAGCCAGAGGACTTCG | |
| | | CGACCTACTACTGTGTGCAGGCCAACGCCTTTCCGTATAAGTTCGGAGGG | |
| | | GGGACCAAGGTCGAAATCAAG | |
| KLK7-L3- | VH | CAAGTGCAGCTCGTGCAGTCGGGAGCCGAGGTCAAGAAGCCTGGCAGCAG | 293 |
| Ab3 | | CGTGAAGGTGTCCTGCAAAGCGTCCGGAGGCACCTTCAGCTCCCACGCCA | |
| | | TCACCTGGGTCAGACAGGCTCCGGGACAGGGTCTGGAGTGGATGGGCGGG | |
| | | ATCATTCCCATCTTCGGGTTCGCCAACTACGCTCAGAAGTTCCAGGGACG | |
| | | CGTGACTATCACCGCGGACGAATCAACCAGCACTGCCTATATGGAACTGT | |
| | | CCTCCCTGCGGTCGGAAGATACCGCCGTGTACTACTGTGCAAGGGGAGCG | |
| | | CCAAACTACTACGGCTCTGCCTCCTCACGGGGAATCGCCTTTGACATTTG | |
| | | GGGTCAAGGCACTATGGTCACGGTGTCCTCG | |
| | VL | GACATCCAAATGACTCAGTCACCGTCCTCCGTGTCGGCCTCCGTGGGAGA | 294 |
| | | TAGAGTGACCATTACTTGTCGCGCAAGCCAGGGCATCAGCAGCTGGCTCG | |
| | | CCTGGTACCAGCAGAAGCCCGGGAAAGCCCCTAAGCTGCTGATCTACGCC | |
| | | GCTTCCTCCCTTCAATCCGGAGTGCCATCCCGGTTCTCTGGTTCGGGATC | |
| | | AGGCACCGACTTTACCCTGACCATTAGCTCGCTGCAGCCCGAGGACTTCG | |
| | | CGACCTACTATTGCGTGCAAGCCAACGCGTTCCCGTACTCCTTCGGGGGC | |
| | | GGAACTAAGGTCGAAATCAAG | |
| KLK7-L4- | VH | CAGCTTCAACTGCAAGAGTCGGGACCCGGACTCGTGAAACCCTCGGAAAC | 245 |
| Ab1 | | TCTGTCGCTGACTTGCACCGTGTCCGGAGGGTCCATTAGCTCCTCCTCAT | |
| | | ACTACTGGGGTTGGATCCGGCAGCCACCTGGAAAGGGCCTCGAATGGATC | |
| | | GGCTCAATTTCCTATTCCGGAAGCACCTACTACAACCCGAGCTTGAAGTC | |
| | | TCGCGTCACCATCTCCGTGGACACCTCGAAGAACCAGTTCAGCCTGAAGC | |
| | | TGTCCAGCGTCACGGCAGCCGATACTGCCGTGTACTACTGCGCCAGAGGT | |
| | | GCTCCTACCTACTTCGGAGCGGCCTCACACTATTACTACTACATGGACGT | |
| | | CTGGGGGAAGGGCACTACCGTGACCGTGTCCTCC | |
| | VL | GACATTCAAATGACTCAGAGCCCGTCCACCCTGTCCGCCTCTGTGGGAGA | 246 |
| | | CAGAGTGACCATCACTTGTCGGGCTAGCCAGTCCATTTCCTCCTGGCTCG | |
| | | CCTGGTACCAGCAGAAACCTGGGAAGGCCCCAAAGCTGCTTATCTACAAG | |
| | | GCATCGTCCCTGGAATCAGGAGTGCCCAGCCGCTTCAGCGGATCCGGATC | |
| | | GGGCACCGAATTCACTCTGACCATCTCCTCACTCCAACCCGACGATTTTG | |
| | | CGACCTACTACTGCGAGCAGTACGGCTCGTATTCGCCGTTGACTTTCGGG | |
| | | GGCGGTACCAAGGTCGAGATCAAG | |
| KLK7-L4- | VH | CAGCTGCAACTGCAGGAATCCGGCCCAGGGCTTGTGAAGCCGTCGGAGAC | 247 |
| Ab2 | | TCTCTCGCTCACTTGCACCGTGTCCGGAGGCTCCATTTCCTCCTCCCCCT | |
| | | ACAACTGGGTCTGGATCCGGCAGCCTCCTGGAAAGGGTCTGGAATGGATC | |
| | | GGCTCAATCCTGCACTCGGGAAGCACCTACTACAACCCGAGCTTGAAGTC | |
| | | CCGCGTGACCATTAGCGTGGACACCTCCAAGAACCAGTTCTCGCTGAAAC | |
| | | TGTCCTCTGTGACCGCGGCAGATACCGCCGTCTACTATTGTGCGAGAGGG | |
| | | GTGCCCACTTACTGGGGTGCCGCTAGCCATTACTACTACTACATGGACGT | |
| | | CTGGGGAAAGGGCACGACTGTGACCGTGTCAAGC | |
| | VL | GACATCCAAATGACCCAGAGCCCCAGCACCTTGTCCGCATCCGTGGGAGA | 248 |
| | | TAGAGTGACCATTACTTGCCGGGCCTCACAGTCCATTTCCTCGTGGCTCG | |
| | | CGTGGTACCAGCAGAAGCCGGGAAAGGCCCCAAAGCTCCTGATCTATAAG | |
| | | GCTTCCTCGCTGGAATCTGGGGTCCCTTCGCGCTTCTCCGGATCCGGCTC | |
| | | AGGGACTGAGTTCACGCTGACTATCTCCTCCCTGCAACCGGACGACTTCG | |
| | | CCACCTACTACTGCGAGCAGTACGGCAGCGAAAGCCCCCTTACCTTTGGT | |
| | | GGAGGCACCAAAGTGGAGATCAAG | |
| KLK7-L5- | VH | CAGGTCCAGCTGGTGCAGTCGGGAGCTGAGGTCAAGAAGCCTGGAGCCTC | 249 |
| Ab1 | | AGTGAAGGTGTCCTGCAAAGCCTCGGGCTACACCTTCACCGGCTACTACA | |
| | | TGCACTGGGTCAGACAGGCCCCCGGACAAGGACTGGAGTGGATGGGGTCC | |
| | | ATCAACCCGAGCTCAGGAGGCACTAACTACGCCCAGAAGTTCCAAGGCCG | |
| | | CGTGACCATGACTCGGGACACCTCCATTTCCACCGCGTACATGGAACTGT | |
| | | CCAGGCTCCGGAGCGACGATACGGCGGTGTACTACTGTGCACGCGGTGGA | |
| | | CCATCCACTTGGTACGGCGGTTCGGCCTACTACTACTATGGGATGGACGT | |
| | | GTGGGGACAGGGGACTACCGTGACCGTCAGCTCC | |
| | VL | GAAATCGTGCTGACCCAGTCGCCTGGCACTCTGTCACTGTCCCCGGGAGA | 250 |
| | | GAGAGCCACCCTGAGCTGTAGGGCGTCCCAGTCCGTGAGCTCGGACTACC | |
| | | TGGCCTGGTATCAGCAGAAGCCAGGACAAGCACCTCGCCTTCTCATCTAC | |
| | | GGAGCCTCATCCCGGGCCACTGGGATCCCCGACCGGTTTTCGGGAAGCGG | |
| | | TTCCGGCACTGATTTCACCCTGACTATCTCCCGCTTGGAACCGGAGGACT | |
| | | TCGCTGTGTACTACTGCCAACAGTACGCGAACCTCTGGACCTTCGGAGGC | |
| | | GGTACCAAGGTCGAGATTAAG | |
| KLK7-L5- | VH | CAGGTCCAGCTCGTGCAGTCTGGGGCCGAAGTGAAGAAGCCAGGAGCGTC | 251 |
| Ab2 | | GGTGAAAGTGTCCTGCAAGGCCTCCGGTTACACCTTCGCCGGCTACGATA | |
| | | TGCACTGGGTCAGACAGGCTCCTGGCCAAGGCCTGGAATGGATGGGGTCG | |
| | | ATTAACCCGACTTCCGGACGGACCAACTACGCCCAGAAGTTCCAAGGCCG | |
| | | CGTGACCATGACTCGGGACACTAGCATCTCAACGGCCTACATGGAGCTGT | |
| | | CCCGCCTGAGGTCCGACGATACCGCCGTGTACTATTGTGCGAGAGGAGGA | |
| | | CCCTCAACTTGGTACGGGGGATCCGCCTACACATACTACGGTGCAGACGT | |
| | | CTGGGGCCAGGGAACCACCGTGACCGTGTCCAGC | |
| | VL | GAAATCGTGCTGACCCAGTCCCCTGGGACTCTGAGCCTTTCCCCTGGCGA | 252 |
| | | AAGGGCCACTCTCTCATGTCGGGCGTCACAGTCGGTGTCCAGCGACTACT | |
| | | TGGCTTGGTACCAGCAGAAGCCTGGACAAGCCCCCAGACTCCTGATCTAT | |
| | | GGGGCCTCCTCGAGAGCAACTGGAATCCCGGACCGGTTCTCCGGATCGGG | |
| | | AAGCGGTACCGACTTCACCCTGACGATTTCCCGCCTGGAACCGGAGGATT | |
| | | TCGCCGTGTACTACTGCCAACAGTACGCCCCACTGTGGACCTTTGGCGGC | |
| | | GGTACCAAGGTCGAGATTAAG | |

In some embodiments, the present disclosure provides isolated nucleic acid comprising any one of the nucleic acid sequence encoding any one of the VH set forth in Table 5. In some embodiments, the present disclosure provides isolated nucleic acid comprising any one of the nucleic acid sequence encoding any one of the VH set forth in Table 5. In some embodiments, the present disclosure provides isolated nucleic acid comprising any one of the nucleic acid sequence encoding any one of the VH/VL pairs set forth in Table 5.

In some embodiments, the present disclosure provides nucleic acid sequences comprising:
(1) a sequence at least 60% identical, at least 70% identical, at least 80% identical, at least 90% identical, at least 95% identical, at least 99% identical or 100% identical of SEQ ID NO: 202 and/or a sequence at least 60% identical, at least 70% identical, at least 80% identical, at least 90% identical, at least 95% identical, at least 99% identical or 100% identical of SEQ ID NO: 203;
(2) a sequence at least 60% identical, at least 70% identical, at least 80% identical, at least 90% identical, at least 95% identical, at least 99% identical or 100% identical of SEQ ID NO: 204 and/or a sequence at least 60% identical, at least 70% identical, at least 80% identical, at least 90% identical, at least 95% identical, at least 99% identical or 100% identical of SEQ ID NO: 203;
(3) a sequence at least 60% identical, at least 70% identical, at least 80% identical, at least 90% identical, at least 95% identical, at least 99% identical or 100% identical of SEQ ID NO: 205 and/or a sequence at least 60% identical, at least 70% identical, at least 80% identical, at least 90% identical, at least 95% identical, at least 99% identical or 100% identical of SEQ ID NO: 203;
(4) a sequence at least 60% identical, at least 70% identical, at least 80% identical, at least 90% identical, at least 95% identical, at least 99% identical or 100% identical of SEQ ID NO: 206 and/or a sequence at least 60% identical, at least 70% identical, at least 80% identical, at least 90% identical, at least 95% identical, at least 99% identical or 100% identical of SEQ ID NO: 203;
(5) a sequence at least 60% identical, at least 70% identical, at least 80% identical, at least 90% identical, at least 95% identical, at least 99% identical or 100% identical of SEQ ID NO: 207 and/or a sequence at least 60% identical, at least 70% identical, at least 80% identical, at least 90% identical, at least 95% identical, at least 99% identical or 100% identical of SEQ ID NO: 203;
(6) a sequence at least 60% identical, at least 70% identical, at least 80% identical, at least 90% identical, at least 95% identical, at least 99% identical or 100% identical of SEQ ID NO: 208 and/or a sequence at least 60% identical, at least 70% identical, at least 80% identical, at least 90% identical, at least 95% identical, at least 99% identical or 100% identical of SEQ ID NO: 209;
(7) a sequence at least 60% identical, at least 70% identical, at least 80% identical, at least 90% identical, at least 95% identical, at least 99% identical or 100% identical of SEQ ID NO: 210 and/or a sequence at least 60% identical, at least 70% identical, at least 80% identical, at least 90% identical, at least 95% identical, at least 99% identical or 100% identical of SEQ ID NO: 209;
(8) a sequence at least 60% identical, at least 70% identical, at least 80% identical, at least 90% identical, at least 95% identical, at least 99% identical or 100% identical of SEQ ID NO: 211 and/or a sequence at least 60% identical, at least 70% identical, at least 80% identical, at least 90% identical, at least 95% identical, at least 99% identical or 100% identical of SEQ ID NO: 212;
(9) a sequence at least 60% identical, at least 70% identical, at least 80% identical, at least 90% identical, at least 95% identical, at least 99% identical or 100% identical of SEQ ID NO: 213 and/or a sequence at least 60% identical, at least 70% identical, at least 80% identical, at least 90% identical, at least 95% identical, at least 99% identical or 100% identical of SEQ ID NO: 209;
(10) a sequence at least 60% identical, at least 70% identical, at least 80% identical, at least 90% identical, at least 95% identical, at least 99% identical or 100% identical of SEQ ID NO: 214 and/or a sequence at least 60% identical, at least 70% identical, at least 80% identical, at least 90% identical, at least 95% identical, at least 99% identical or 100% identical of SEQ ID NO: 215;
(11) a sequence at least 60% identical, at least 70% identical, at least 80% identical, at least 90% identical, at least 95% identical, at least 99% identical or 100% identical of SEQ ID NO: 216 and/or a sequence at least 60% identical, at least 70% identical, at least 80% identical, at least 90% identical, at least 95% identical, at least 99% identical or 100% identical of SEQ ID NO: 216;
(12) a sequence at least 60% identical, at least 70% identical, at least 80% identical, at least 90% identical, at least 95% identical, at least 99% identical or 100% identical of SEQ ID NO: 217 and/or a sequence at least 60% identical, at least 70% identical, at least 80% identical, at least 90% identical, at least 95% identical, at least 99% identical or 100% identical of SEQ ID NO: 215;
(13) a sequence at least 60% identical, at least 70% identical, at least 80% identical, at least 90% identical, at least 95% identical, at least 99% identical or 100% identical of SEQ ID NO: 218 and/or a sequence at least 60% identical, at least 70% identical, at least 80% identical, at least 90% identical, at least 95% identical, at least 99% identical or 100% identical of SEQ ID NO: 215;
(14) a sequence at least 60% identical, at least 70% identical, at least 80% identical, at least 90% identical, at least 95% identical, at least 99% identical or 100% identical of SEQ ID NO: 219 and/or a sequence at least 60% identical, at least 70% identical, at least 80% identical, at least 90% identical, at least 95% identical, at least 99% identical or 100% identical of SEQ ID NO: 215;
(15) a sequence at least 60% identical, at least 70% identical, at least 80% identical, at least 90% identical, at least 95% identical, at least 99% identical or 100% identical of SEQ ID NO: 220 and/or a sequence at least 60% identical, at least 70% identical, at least 80% identical, at least 90% identical, at least 95% identical, at least 99% identical or 100% identical of SEQ ID NO: 221;
(16) a sequence at least 60% identical, at least 70% identical, at least 80% identical, at least 90% identical, at least 95% identical, at least 99% identical or 100% identical of SEQ ID NO: 222 and/or a sequence at least 60% identical, at least 70% identical, at least 80% identical, at least 90% identical, at least 95% identical, at least 99% identical or 100% identical of SEQ ID NO: 223;
(17) a sequence at least 60% identical, at least 70% identical, at least 80% identical, at least 90% identical, at least 95% identical, at least 99% identical or 100% identical of SEQ ID NO: 224 and/or a sequence at least 60% identical, at least 70% identical, at least 80% identical, at least 90% identical, at least 95% identical, at least 99% identical or 100% identical of SEQ ID NO: 225;
(18) a sequence at least 60% identical, at least 70% identical, at least 80% identical, at least 90% identical, at least 95% identical, at least 99% identical or 100% identical of SEQ ID NO: 295 and/or a sequence at least 60% identical, at least 70% identical, at least 80% identical, at least 90% identical, at least 95% identical, at least 99% identical or 100% identical of SEQ ID NO: 227;
(19) a sequence at least 60% identical, at least 70% identical, at least 80% identical, at least 90% identical, at least 95% identical, at least 99% identical or 100% identical of SEQ ID NO: 228 and/or a sequence at least 60% identical, at least 70% identical, at least 80% identical, at least 90% identical, at least 95% identical, at least 99% identical or 100% identical of SEQ ID NO: 229;
(20) a sequence at least 60% identical, at least 70% identical, at least 80% identical, at least 90% identical, at least 95% identical, at least 99% identical or 100% identical of SEQ ID NO: 230 and/or a sequence at least 60% identical, at least 70% identical, at least 80% identical, at least 90% identical, at least 95% identical, at least 99% identical or 100% identical of SEQ ID NO: 286;
(21) a sequence at least 60% identical, at least 70% identical, at least 80% identical, at least 90% identical, at least 95% identical, at least 99% identical or 100% identical of SEQ ID NO: 231 and/or a sequence at least 60% identical, at least 70% identical, at least 80% identical, at least 90% identical, at least 95% identical, at least 99% identical or 100% identical of SEQ ID NO: 232;
(22) a sequence at least 60% identical, at least 70% identical, at least 80% identical, at least 90% identical, at least 95% identical, at least 99% identical or 100% identical of SEQ ID NO: 233 and/or a sequence at least 60% identical, at least 70% identical, at least 80% identical, at least 90% identical, at least 95% identical, at least 99% identical or 100% identical of SEQ ID NO: 287;
(23) a sequence at least 60% identical, at least 70% identical, at least 80% identical, at least 90% identical, at least 95% identical, at least 99% identical or 100% identical of SEQ ID NO: 234 and/or a sequence at least 60% identical, at least 70% identical, at least 80% identical, at least 90% identical, at least 95% identical, at least 99% identical or 100% identical of SEQ ID NO: 234;
(24) a sequence at least 60% identical, at least 70% identical, at least 80% identical, at least 90% identical, at least 95% identical, at least 99% identical or 100% identical of SEQ ID NO: 236 and/or a sequence at least 60% identical, at least 70% identical, at least 80% identical, at least 90% identical, at least 95% identical, at least 99% identical or 100% identical of SEQ ID NO: 288;
(25) a sequence at least 60% identical, at least 70% identical, at least 80% identical, at least 90% identical, at least 95% identical, at least 99% identical or 100% identical of SEQ ID NO: 237 and/or a sequence at least 60% identical, at least 70% identical, at least 80% identical, at least 90% identical, at least 95% identical, at least 99% identical or 100% identical of SEQ ID NO: 238;
(26) a sequence at least 60% identical, at least 70% identical, at least 80% identical, at least 90% identical, at least 95% identical, at least 99% identical or 100% identical of SEQ ID NO: 239 and/or a sequence at least 60% identical, at least 70% identical, at least 80% identical, at least 90% identical, at least 95% identical, at least 99% identical or 100% identical of SEQ ID NO: 238;
(27) a sequence at least 60% identical, at least 70% identical, at least 80% identical, at least 90% identical, at least 95% identical, at least 99% identical or 100% identical of SEQ ID NO: 240 and/or a sequence at least 60% identical, at least 70% identical, at least 80% identical, at least 90% identical, at least 95% identical, at least 99% identical or 100% identical of SEQ ID NO: 241;
(28) a sequence at least 60% identical, at least 70% identical, at least 80% identical, at least 90% identical, at least 95% identical, at least 99% identical or 100% identical of SEQ ID NO: 242 and/or a sequence at least 60% identical, at least 70% identical, at least 80% identical, at least 90% identical, at least 95% identical, at least 99% identical or 100% identical of SEQ ID NO: 243;
(29) a sequence at least 60% identical, at least 70% identical, at least 80% identical, at least 90% identical, at least 95% identical, at least 99% identical or 100% identical of SEQ ID NO: 244 and/or a sequence at least 60% identical, at least 70% identical, at least 80% identical, at least 90% identical, at least 95% identical, at least 99% identical or 100% identical of SEQ ID NO: 238;
(30) a sequence at least 60% identical, at least 70% identical, at least 80% identical, at least 90% identical, at least 95% identical, at least 99% identical or 100% identical of SEQ ID NO: 289 and/or a sequence at least 60% identical, at least 70% identical, at least 80% identical, at least 90% identical, at least 95% identical, at least 99% identical or 100% identical of SEQ ID NO: 290;
(31) a sequence at least 60% identical, at least 70% identical, at least 80% identical, at least 90% identical, at least 95% identical, at least 99% identical or 100% identical of SEQ ID NO: 291 and/or a sequence at least 60% identical, at least 70% identical, at least 80% identical, at least 90% identical, at least 95% identical, at least 99% identical or 100% identical of SEQ ID NO: 292;
(32) a sequence at least 60% identical, at least 70% identical, at least 80% identical, at least 90% identical, at least 95% identical, at least 99% identical or 100% identical of SEQ ID NO: 293 and/or a sequence at least 60% identical, at least 70% identical, at least 80% identical, at least 90% identical, at least 95% identical, at least 99% identical or 100% identical of SEQ ID NO: 294;
(33) a sequence at least 60% identical, at least 70% identical, at least 80% identical, at least 90% identical, at least 95% identical, at least 99% identical or 100% identical of SEQ ID NO: 245 and/or a sequence at least 60% identical, at least 70% identical, at least 80% identical, at least 90% identical, at least 95% identical, at least 99% identical or 100% identical of SEQ ID NO: 246;
(34) a sequence at least 60% identical, at least 70% identical, at least 80% identical, at least 90% identical, at least 95% identical, at least 99% identical or 100% identical of SEQ ID NO: 249 and/or a sequence at least 60% identical, at least 70% identical, at least 80% identical, at least 90% identical, at least 95% identical, at least 99% identical or 100% identical of SEQ ID NO: 250;
(35) a sequence at least 60% identical, at least 70% identical, at least 80% identical, at least 90% identical, at least 95% identical, at least 99% identical or 100% identical of SEQ ID NO: 202 and/or a sequence at least 60% identical, at least 70% identical, at least 80% identical, at least 90% identical, at least 95% identical, at least 99% identical or 100% identical of SEQ ID NO: 203; or
(36) a sequence at least 60% identical, at least 70% identical, at least 80% identical, at least 90% identical, at least 95% identical, at least 99% identical or 100% identical of SEQ ID NO: 251 and/or a sequence at least 60% identical, at least 70% identical, at least 80% identical, at least 90% identical, at least 95% identical, at least 99% identical or 100% identical of SEQ ID NO: 252.

In some embodiments, an antibody described herein is produced by expressing in a host cell an isolated nucleic acid comprising:
(1) a sequence at least 60% identical, at least 70% identical, at least 80% identical, at least 90% identical, at least 95% identical, at least 99% identical or 100% identical of SEQ ID NO: 202 and/or a sequence at least 60% identical, at least 70% identical, at least 80% identical, at least 90% identical, at least 95% identical, at least 99% identical or 100% identical of SEQ ID NO: 203;
(2) a sequence at least 60% identical, at least 70% identical, at least 80% identical, at least 90% identical, at least 95% identical, at least 99% identical or 100% identical of SEQ ID NO: 204 and/or a sequence at least 60% identical, at least 70% identical, at least 80% identical, at least 90% identical, at least 95% identical, at least 99% identical or 100% identical of SEQ ID NO: 203;
(3) a sequence at least 60% identical, at least 70% identical, at least 80% identical, at least 90% identical, at least 95% identical, at least 99% identical or 100% identical of SEQ ID NO: 205 and/or a sequence at least 60% identical, at least 70% identical, at least 80% identical, at least 90% identical, at least 95% identical, at least 99% identical or 100% identical of SEQ ID NO: 203;
(4) a sequence at least 60% identical, at least 70% identical, at least 80% identical, at least 90% identical, at least 95% identical, at least 99% identical or 100% identical of SEQ ID NO: 206 and/or a sequence at least 60% identical, at least 70% identical, at least 80% identical, at least 90% identical, at least 95% identical, at least 99% identical or 100% identical of SEQ ID NO: 203;
(5) a sequence at least 60% identical, at least 70% identical, at least 80% identical, at least 90% identical, at least 95% identical, at least 99% identical or 100% identical of SEQ ID NO: 207 and/or a sequence at least 60% identical, at least 70% identical, at least 80% identical, at least 90% identical, at least 95% identical, at least 99% identical or 100% identical of SEQ ID NO: 203;
(6) a sequence at least 60% identical, at least 70% identical, at least 80% identical, at least 90% identical, at least 95% identical, at least 99% identical or 100% identical of SEQ ID NO: 208 and/or a sequence at least 60% identical, at least 70% identical, at least 80% identical, at least 90% identical, at least 95% identical, at least 99% identical or 100% identical of SEQ ID NO: 209;
(7) a sequence at least 60% identical, at least 70% identical, at least 80% identical, at least 90% identical, at least 95% identical, at least 99% identical or 100% identical of SEQ ID NO: 210 and/or a sequence at least 60% identical, at least 70% identical, at least 80% identical, at least 90% identical, at least 95% identical, at least 99% identical or 100% identical of SEQ ID NO: 209;
(8) a sequence at least 60% identical, at least 70% identical, at least 80% identical, at least 90% identical, at least 95% identical, at least 99% identical or 100% identical of SEQ ID NO: 211 and/or a sequence at least 60% identical, at least 70% identical, at least 80% identical, at least 90% identical, at least 95% identical, at least 99% identical or 100% identical of SEQ ID NO: 212;
(9) a sequence at least 60% identical, at least 70% identical, at least 80% identical, at least 90% identical, at least 95% identical, at least 99% identical or 100% identical of SEQ ID NO: 213 and/or a sequence at least 60% identical, at least 70% identical, at least 80% identical, at least 90% identical, at least 95% identical, at least 99% identical or 100% identical of SEQ ID NO: 209;
(10) a sequence at least 60% identical, at least 70% identical, at least 80% identical, at least 90% identical, at least 95% identical, at least 99% identical or 100% identical of SEQ ID NO: 214 and/or a sequence at least 60% identical, at least 70% identical, at least 80% identical, at least 90% identical, at least 95% identical, at least 99% identical or 100% identical of SEQ ID NO: 215;
(11) a sequence at least 60% identical, at least 70% identical, at least 80% identical, at least 90% identical, at least 95% identical, at least 99% identical or 100% identical of SEQ ID NO: 216 and/or a sequence at least 60% identical, at least 70% identical, at least 80% identical, at least 90% identical, at least 95% identical, at least 99% identical or 100% identical of SEQ ID NO: 216;
(12) a sequence at least 60% identical, at least 70% identical, at least 80% identical, at least 90% identical, at least 95% identical, at least 99% identical or 100% identical of SEQ ID NO: 217 and/or a sequence at least 60% identical, at least 70% identical, at least 80% identical, at least 90% identical, at least 95% identical, at least 99% identical or 100% identical of SEQ ID NO: 215;
(13) a sequence at least 60% identical, at least 70% identical, at least 80% identical, at least 90% identical, at least 95% identical, at least 99% identical or 100% identical of SEQ ID NO: 218 and/or a sequence at least 60% identical, at least 70% identical, at least 80% identical, at least 90% identical, at least 95% identical, at least 99% identical or 100% identical of SEQ ID NO: 215;
(14) a sequence at least 60% identical, at least 70% identical, at least 80% identical, at least 90% identical, at least 95% identical, at least 99% identical or 100% identical of SEQ ID NO: 219 and/or a sequence at least 60% identical, at least 70% identical, at least 80% identical, at least 90% identical, at least 95% identical, at least 99% identical or 100% identical of SEQ ID NO: 215;
(15) a sequence at least 60% identical, at least 70% identical, at least 80% identical, at least 90% identical, at least 95% identical, at least 99% identical or 100% identical of SEQ ID NO: 220 and/or a sequence at least 60% identical, at least 70% identical, at least 80% identical, at least 90% identical, at least 95% identical, at least 99% identical or 100% identical of SEQ ID NO: 221;
(16) a sequence at least 60% identical, at least 70% identical, at least 80% identical, at least 90% identical, at least 95% identical, at least 99% identical or 100% identical of SEQ ID NO: 222 and/or a sequence at least 60% identical, at least 70% identical, at least 80% identical, at least 90% identical, at least 95% identical, at least 99% identical or 100% identical of SEQ ID NO: 223;
(17) a sequence at least 60% identical, at least 70% identical, at least 80% identical, at least 90% identical, at least 95% identical, at least 99% identical or 100% identical of SEQ ID NO: 224 and/or a sequence at least 60% identical, at least 70% identical, at least 80% identical, at least 90% identical, at least 95% identical, at least 99% identical or 100% identical of SEQ ID NO: 225;
(18) a sequence at least 60% identical, at least 70% identical, at least 80% identical, at least 90% identical, at least 95% identical, at least 99% identical or 100% identical of SEQ ID NO: 295 and/or a sequence at least 60% identical, at least 70% identical, at least 80% identical, at least 90% identical, at least 95% identical, at least 99% identical or 100% identical of SEQ ID NO: 227;
(19) a sequence at least 60% identical, at least 70% identical, at least 80% identical, at least 90% identical, at least 95% identical, at least 99% identical or 100% identical of SEQ ID NO: 228 and/or a sequence at least 60% identical, at least 70% identical, at least 80% identical, at least 90% identical, at least 95% identical, at least 99% identical or 100% identical of SEQ ID NO: 229;
(20) a sequence at least 60% identical, at least 70% identical, at least 80% identical, at least 90% identical, at least 95% identical, at least 99% identical or 100% identical of SEQ ID NO: 230 and/or a sequence at least 60% identical, at least 70% identical, at least 80% identical, at least 90% identical, at least 95% identical, at least 99% identical or 100% identical of SEQ ID NO: 286;
(21) a sequence at least 60% identical, at least 70% identical, at least 80% identical, at least 90% identical, at least 95% identical, at least 99% identical or 100% identical of SEQ ID NO: 231 and/or a sequence at least 60% identical, at least 70% identical, at least 80% identical, at least 90% identical, at least 95% identical, at least 99% identical or 100% identical of SEQ ID NO: 232;
(22) a sequence at least 60% identical, at least 70% identical, at least 80% identical, at least 90% identical, at least 95% identical, at least 99% identical or 100% identical of SEQ ID NO: 233 and/or a sequence at least 60% identical, at least 70% identical, at least 80% identical, at least 90% identical, at least 95% identical, at least 99% identical or 100% identical of SEQ ID NO: 287;
(23) a sequence at least 60% identical, at least 70% identical, at least 80% identical, at least 90% identical, at least 95% identical, at least 99% identical or 100% identical of SEQ ID NO: 234 and/or a sequence at least 60% identical, at least 70% identical, at least 80% identical, at least 90% identical, at least 95% identical, at least 99% identical or 100% identical of SEQ ID NO: 234;
(24) a sequence at least 60% identical, at least 70% identical, at least 80% identical, at least 90% identical, at least 95% identical, at least 99% identical or 100% identical of SEQ ID NO: 236 and/or a sequence at least 60% identical, at least 70% identical, at least 80% identical, at least 90% identical, at least 95% identical, at least 99% identical or 100% identical of SEQ ID NO: 288;
(25) a sequence at least 60% identical, at least 70% identical, at least 80% identical, at least 90% identical, at least 95% identical, at least 99% identical or 100% identical of SEQ ID NO: 237 and/or a sequence at least 60% identical, at least 70% identical, at least 80% identical, at least 90% identical, at least 95% identical, at least 99% identical or 100% identical of SEQ ID NO: 238;
(26) a sequence at least 60% identical, at least 70% identical, at least 80% identical, at least 90% identical, at least 95% identical, at least 99% identical or 100% identical of SEQ ID NO: 239 and/or a sequence at least 60% identical, at least 70% identical, at least 80% identical, at least 90% identical, at least 95% identical, at least 99% identical or 100% identical of SEQ ID NO: 238;
(27) a sequence at least 60% identical, at least 70% identical, at least 80% identical, at least 90% identical, at least 95% identical, at least 99% identical or 100% identical of SEQ ID NO: 240 and/or a sequence at least 60% identical, at least 70% identical, at least 80% identical, at least 90% identical, at least 95% identical, at least 99% identical or 100% identical of SEQ ID NO: 241;
(28) a sequence at least 60% identical, at least 70% identical, at least 80% identical, at least 90% identical, at least 95% identical, at least 99% identical or 100% identical of SEQ ID NO: 242 and/or a sequence at least 60% identical, at least 70% identical, at least 80% identical, at least 90% identical, at least 95% identical, at least 99% identical or 100% identical of SEQ ID NO: 243;
(29) a sequence at least 60% identical, at least 70% identical, at least 80% identical, at least 90% identical, at least 95% identical, at least 99% identical or 100% identical of SEQ ID NO: 244 and/or a sequence at least 60% identical, at least 70% identical, at least 80% identical, at least 90% identical, at least 95% identical, at least 99% identical or 100% identical of SEQ ID NO: 238;
(30) a sequence at least 60% identical, at least 70% identical, at least 80% identical, at least 90% identical, at least 95% identical, at least 99% identical or 100% identical of SEQ ID NO: 289 and/or a sequence at least 60% identical, at least 70% identical, at least 80% identical, at least 90% identical, at least 95% identical, at least 99% identical or 100% identical of SEQ ID NO: 290;
(31) a sequence at least 60% identical, at least 70% identical, at least 80% identical, at least 90% identical, at least 95% identical, at least 99% identical or 100% identical of SEQ ID NO: 291 and/or a sequence at least 60% identical, at least 70% identical, at least 80% identical, at least 90% identical, at least 95% identical, at least 99% identical or 100% identical of SEQ ID NO: 292;
(32) a sequence at least 60% identical, at least 70% identical, at least 80% identical, at least 90% identical, at least 95% identical, at least 99% identical or 100% identical of SEQ ID NO: 293 and/or a sequence at least 60% identical, at least 70% identical, at least 80% identical, at least 90% identical, at least 95% identical, at least 99% identical or 100% identical of SEQ ID NO: 294;
(33) a sequence at least 60% identical, at least 70% identical, at least 80% identical, at least 90% identical, at least 95% identical, at least 99% identical or 100% identical of SEQ ID NO: 245 and/or a sequence at least 60% identical, at least 70% identical, at least 80% identical, at least 90% identical, at least 95% identical, at least 99% identical or 100% identical of SEQ ID NO: 246;
(34) a sequence at least 60% identical, at least 70% identical, at least 80% identical, at least 90% identical, at least 95% identical, at least 99% identical or 100% identical of SEQ ID NO: 249 and/or a sequence at least 60% identical, at least 70% identical, at least 80% identical, at least 90% identical, at least 95% identical, at least 99% identical or 100% identical of SEQ ID NO: 250;
(35) a sequence at least 60% identical, at least 70% identical, at least 80% identical, at least 90% identical, at least 95% identical, at least 99% identical or 100% identical of SEQ ID NO: 202 and/or a sequence at least 60% identical, at least 70% identical, at least 80% identical, at least 90% identical, at least 95% identical, at least 99% identical or 100% identical of SEQ ID NO: 203; or
(36) a sequence at least 60% identical, at least 70% identical, at least 80% identical, at least 90% identical, at least 95% identical, at least 99% identical or 100% identical of SEQ ID NO: 251 and/or a sequence at least 60% identical, at least 70% identical, at least 80% identical, at least 90% identical, at least 95% identical, at least 99% identical or 100% identical of SEQ ID NO: 252.

In some embodiments, the antibodies described herein is used to modulate the activity or function of at least one gene, protein, and/or nucleic acid. In some embodiments, the molecular payload is responsible for the modulation of a gene, protein, and/or nucleic acids. A molecular payload may be a small molecule, protein, nucleic acid, oligonucleotide, or any molecular entity capable of modulating the activity or function of a gene, protein, and/or nucleic acid in a cell.

In some embodiments, a multi-specific antibody comprises direct fusion or linking of different antigen-specific binding sites. In some embodiments, a multi-specific antibody comprises immunoglobulin-derived heteromerization domains to generate multi-specific antibodies. In some embodiments, a multi-specific antibody can be formed by co-expression of different heavy chains and two different light chains. In some embodiments, a multi-specific antibody can be formed by co-expression of different heavy chains and a common light chain. In some embodiments, a multi-specific antibody comprises an engineered CH1 domain (first constant Ig domain of the heavy chain) that facilitates proper heavy chain-light chain pairing, e.g., in such co-expression system, such as is disclosed in International Patent Application Publication Number WO2021067404, “CH1 DOMAIN VARIANTS ENGINEERED FOR PREFERENTIAL LIGHT CHAIN PAIRING AND MULTISPECIFIC ANTIBODIES COMPRISING THE SAME”, published on Apr. 8, 2021, the contents of which are incorporated herein by reference.

In some embodiments, a multi-specific antibody comprises variant CH1 domain that pairs (e.g., preferentially pairs) with a particular variant CL domain. For example, in some embodiments, a multi-specific antibody comprises a heavy chain comprising a variant CH1 domain that preferentially pairs with a variant CLK or CLλ domain. Not limiting examples of such multi-specific antibody configurations are provided in International Patent Application Publication Number WO2022150787, “VARIANT CH1 DOMAINS AND VARIANT CL DOMAINS ENGINEERED FOR PREFERENTIAL CHAIN PAIRING AND MULTI-SPECIFIC ANTIBODIES COMPRISING THE SAME”, published on Jul. 14, 2022, the contents of which are incorporated herein by reference in their entirety.

In some embodiments, a multi-specific antibody comprises variant CH3 domains that preferentially form CH3-CH3 heterodimers over CH3-CH3 homodimers. Incorporation of such variant CH3 domains facilitates heterodimerization, e.g., of different antibodies to form multi-specific antibodies. Not limiting examples of such multi-specific antibody configurations are provided in International Patent Application Publication Number WO2022150785, “VARIANT CH3 DOMAINS ENGINEERED FOR PREFERENTIAL CH3 HETERODIMERIZATION, MULTI-SPECIFIC ANTIBODIES COMPRISING THE SAME, AND METHODS OF MAKING THEREOF”; published on Jul. 14, 2022, the contents of which are incorporated herein by reference in their entirety.

In some embodiments, a bispecific antibody comprises direct fusion or linking of different antigen-specific binding sites. In some embodiments, a bispecific antibody comprises immunoglobulin-derived heterodimerization domains to generate bispecific antibodies. For example, in some embodiments, a bispecific antibody can be formed by co-expression of two different heavy chains and two different light chains. In some embodiments, a bispecific antibody can be formed by co-expression of two different heavy chains and a common light chain. In some embodiments, fusion of two antibody-producing cell lines allows the combination of the heavy and light chains of two different antibodies, such that resulting bispecific antibodies comprise the heavy and light chain of the first antibody and the heavy and light chain of the second antibody. In some embodiments, heavy and light chain constant regions are of the same isotype. In some embodiments, heavy and light chain constant regions are of different isotype.

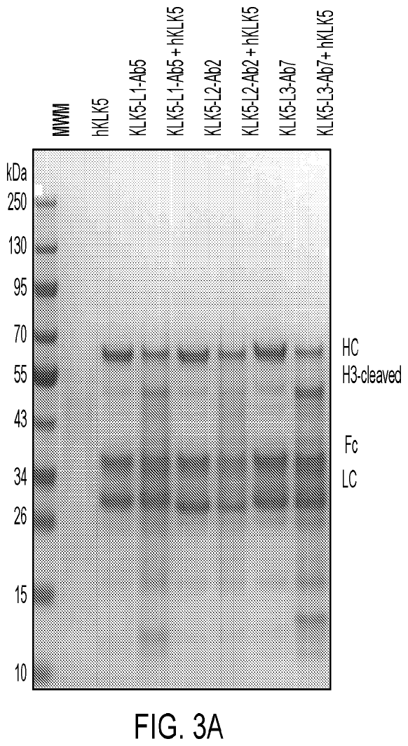

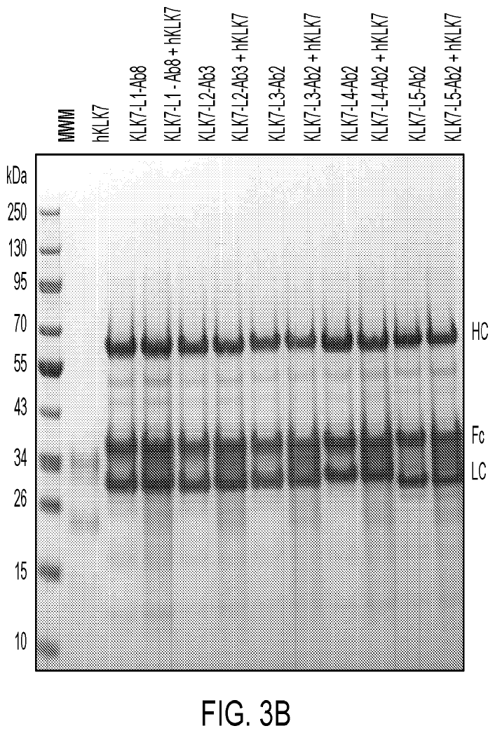

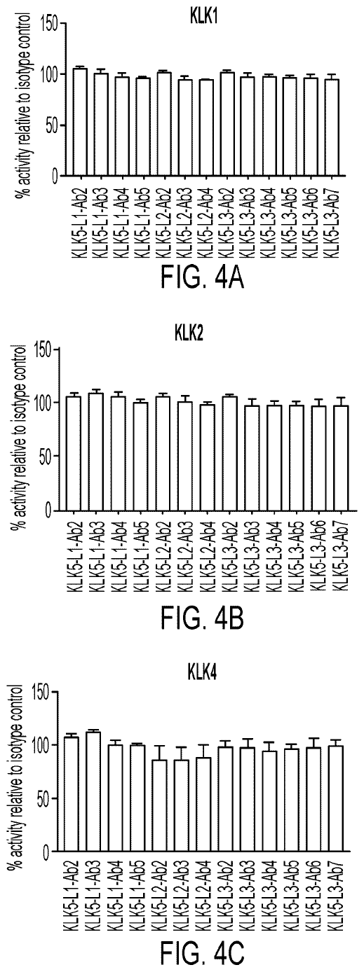

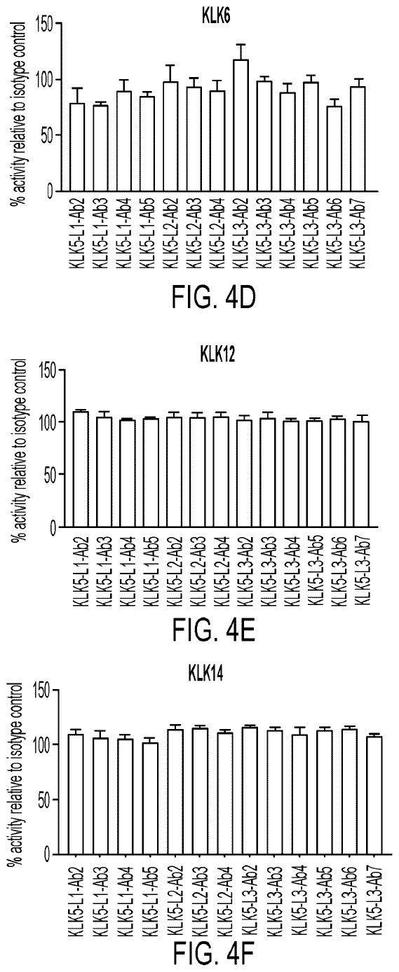

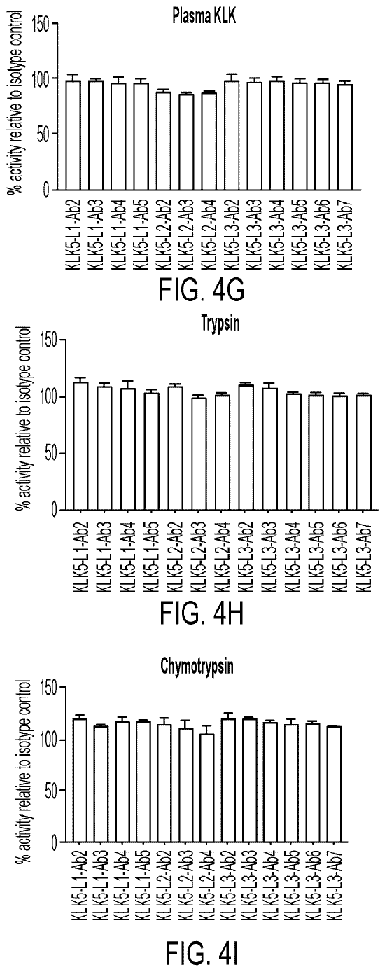

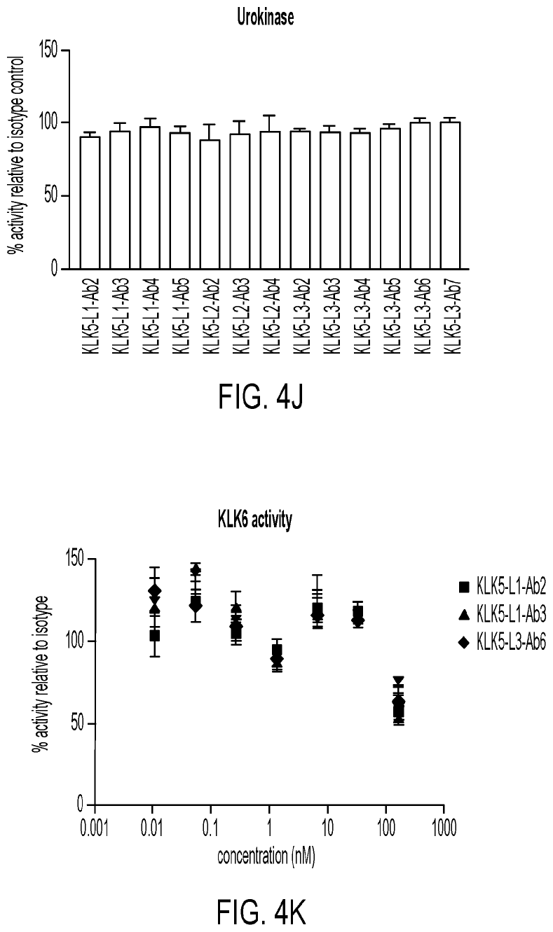

In some embodiments, a bispecific antibody comprises variant heavy chains and/or light chains that force correct assembly between the two heavy chains and cognate heavy and light chains, or to facilitate purification of correctly assembled bispecific antibodies (See, e.g., FIGS. 3 and 4 and Table 1 in Brinkmann U and Kontermann E E, The making of bispecific antibodies, MAbs. 2017 February/March; 9(2):182-212). In some embodiments, a bispecific antibody is formed using the knobs-into-holes technique, e.g., to facilitate heterodimerization. The knobs-into-holes technique may be used, in some embodiments, to produce bispecific IgG molecules, trivalent Ig-like antibodies, bispecific Fc and CH3 fusion proteins, and other formats, as discussed in Ridgway J B, et al., ‘Knobs-into-holes’ engineering of antibody CH3 domains for heavy chain heterodimerization. Protein Eng 1996; 9:617-21; Atwell S, et al., Stable heterodimers from remodeling the domain interface of a homodimer using a phage display library, J Mol Biol 1997; 270:26-35; and Merchant A M, et al., An efficient route to human bispecific IgG, Nat Biotechnol 1998; 16:677-681, the entire contents of each of which are incorporated herein by reference in their entireties.

In some embodiments, a bispecific antibody comprises an engineered CH1 domain (first constant Ig domain of the heavy chain) that facilitates proper heavy chain-light chain pairing, e.g., in such co-expression system, such as is disclosed in International Patent Application Publication No.: WO2021067404, “CH1 DOMAIN VARIANTS ENGINEERED FOR PREFERENTIAL LIGHT CHAIN PAIRING AND MULTISPECIFIC ANTIBODIES COMPRISING THE SAME”, published on Apr. 8, 2021, the contents of which are incorporated herein by reference.

In some embodiments, a bispecific antibody comprises variant CH1 domain that pairs (e.g., preferentially pairs) with a particular variant CL domain. For example, in some embodiments, a bispecific antibody comprises a heavy chain comprising a variant CH1 domain that preferentially pairs with a variant CLK or CLλ domain. Not limiting examples of such bispecific antibody configurations are provided in International Patent Application Publication No.: WO2022150787, “VARIANT CH1 DOMAINS AND VARIANT CL DOMAINS ENGINEERED FOR PREFERENTIAL CHAIN PAIRING AND MULTI-SPECIFIC ANTIBODIES COMPRISING THE SAME”, published on Jul. 14, 2022, the contents of which are incorporated herein by reference in their entirety.

In some embodiments, a bispecific antibody comprises variant CH3 domains that preferentially form CH3-CH3 heterodimers over CH3-CH3 homodimers. Incorporation of such variant CH3 domains facilitates heterodimerization, e.g., of different antibodies to form bispecific antibodies. Not limiting examples of such bispecific antibody configurations are provided in International Patent Application Publication No.: WO2022150785, “VARIANT CH3 DOMAINS ENGINEERED FOR PREFERENTIAL CH3 HETERODIMERIZATION, MULTI-SPECIFIC ANTIBODIES COMPRISING THE SAME, AND METHODS OF MAKING THEREOF”; published on Jul. 14, 2022, the contents of which are incorporated herein by reference in their entirety.

In some embodiments, a bispecific antibody may be formed using non-immunoglobulin heterodimerization modules to combine different antigen-specific binding sites in a non-covalent or covalent manner. For example, in some embodiments, a bispecific antibody is formed via a dock-and-lock method (DNL) utilizing heterodimeric assembly of the regulatory subunit of cAMP-dependent protein kinase (PKA) and the anchoring domains (AD) of A kinase anchor proteins (AKAPs). In some embodiments, a bispecific antibody may be formed using non-immunoglobulin heterodimerization modules to combine different antigen-specific binding sites, such as the barnase-barstar system, adapter/docking tag modules based on mutated RNase I fragments, and SNARE modules based on interaction of the three proteins syntaxin, synaptobrevin and SNAP25.

### IV. PHARMACEUTICAL COMPOSITION

The antibodies, as well as the encoding nucleic acids or nucleic acid sets, vectors comprising such, or host cells comprising the vectors, as described herein can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The anti-KLK5 and/or anti-KLK7 antibody containing pharmaceutical composition disclosed herein may further comprise a suitable buffer agent. A buffer agent is a weak acid or base used to maintain the pH of a solution near a chosen value after the addition of another acid or base. In some examples, the buffer agent disclosed herein can be a buffer agent capable of maintaining physiological pH despite changes in carbon dioxide concentration (produced by cellular respiration). Exemplary buffer agents include, but are not limited to a HEPES (4-(2-hydroxyethyl)-1-piperazineethanesulfonic acid) buffer, Dulbecco's phosphate-buffered saline (DPBS) buffer, or Phosphate-buffered Saline (PBS) buffer. Such buffers may comprise disodium hydrogen phosphate and sodium chloride, or potassium dihydrogen phosphate and potassium chloride.

The pharmaceutical composition described herein comprises one or more suitable salts. A salt is an ionic compound that can be formed by the neutralization reaction of an acid and a base. (Skoog, D. A; West, D. M.; Holler, J. F.; Crouch, S. R. (2004). “chapters 14-16”. Fundamentals of Analytical Chemistry (8th ed.)). Salts are composed of related numbers of cations (positively charged ions) and anions (negative ions) so that the product is electrically neutral (without a net charge).

In some embodiments, the pharmaceutical compositions can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). In some embodiments, the pharmaceutical composition can be formulated for intravenous injection. In some embodiments, the pharmaceutical composition can be formulated for subcutaneous injection.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous or subcutaneous solution bag or vial having a stopper pierceable by a hypodermic injection needle.

### V. METHODS OF USE

In certain aspects, the disclosure provides methods and related compositions for treating conditions associated with KLK5 and KLK7 related dysregulation, including, for example, Netherton Syndrome, atopic dermatitis (with and without filaggrin mutations), eosinophilic esophagitis, prurigo nodularis, chronic pruritis of unknown origin (CPUO), dry skin, asthma (e.g., KLK5 related asthma specifically), ichthyosis vulgaris, and skin itch.

Aspects of the disclosure relate to methods and compositions (e.g., anti-KLK5 antibodies, anti-KLK7 antibodies, and/or anti-KLK5/KLK7 antibodies) useful promoting proper barrier function (e.g., epidermal barrier function). Hyperactive kallikrein 5/7 causes both genetic and spontaneous disruption of epidermal barrier function and is associated with related disorders, such as Netherton syndrome, eosinophilic esophagitis, atopic dermatitis. Accordingly, in some embodiments, methods provided herein comprise administering an effective amount of one or more anti-KLK5 antibodies, anti-KLK7 antibodies, or anti-KLK5/KLK7 antibodies provided herein to a subject for purposes of restoring the epithelial barrier in a subject in need thereof. In other aspects, methods are provided for addressing one or more aspects of altered barrier function. For example, in some embodiments, methods provided herein comprise administering an effective amount of one or more anti-KLK5 antibodies, anti-KLK7 antibodies, or anti-KLK5/KLK7 antibodies provided herein to a subject for purposes of reducing dermal infiltrates in a subject in need thereof. In some embodiments, methods provided herein comprise administering an effective amount of one or more anti-KLK5 antibodies, anti-KLK7 antibodies, or anti-KLK5/KLK7 antibodies provided herein to a subject for purposes of reducing epithelium inflammation in a subject in need thereof. In some embodiments, methods provided herein comprise administering an effective amount of one or more anti-KLK5 antibodies, anti-KLK7 antibodies, or anti-KLK5/KLK7 antibodies provided herein to a subject for purposes of reducing epithelium permeability in a subject in need thereof. In some embodiments, methods provided herein comprise administering an effective amount of one or more anti-KLK5 antibodies, anti-KLK7 antibodies, or anti-KLK5/KLK7 antibodies provided herein to a subject for purposes of reducing parakeratosis in a subject in need thereof. In some embodiments, methods provided herein comprise administering an effective amount of one or more anti-KLK5 antibodies, anti-KLK7 antibodies, or anti-KLK5/KLK7 antibodies provided herein to a subject for purposes of reducing skin inflammatory cytokines in a subject in need thereof. In some embodiments, methods provided herein comprise administering an effective amount of one or more anti-KLK5 antibodies, anti-KLK7 antibodies, or anti-KLK5/KLK7 antibodies provided herein to a subject for purposes of reducing transepidermal water loss in a subject in need thereof.

Further aspects of the disclosure relate to methods and compositions (e.g., anti-KLK5 antibodies, anti-KLK7 antibodies, and/or anti-KLK5/KLK7 antibodies) useful for treating atopic dermatitis. Atopic dermatitis (AD), also known as eczema, is a common chronic pruritic inflammatory skin disease. In some embodiments, atopic dermatitis begins in a subject in infancy or early childhood (e.g., at or about 2 years of age). According, in some embodiments, methods provided herein are useful for treating subjects having atopic dermatitis who are 2 year of age or older. Atopic dermatitis may be associated with elevations of the total serum IgE concentration. According, in some embodiments, methods provided herein are useful for treating subjects having atopic dermatitis with elevated levels of total serum IgE concentrations (e.g., compared with normal IgE levels in subjects who do not have atopic dermatitis or related conditions). In some embodiments, atopic dermatitis is associated with a chronic relapsing form of skin inflammation, a disturbance of epidermal barrier function (e.g., that culminates in dry skin), and/or IgE-mediated sensitization to allergens, such as food and environmental allergens. Accordingly, in some embodiments, methods of treating a subject having atopic dermatitis are provided herein that comprise administering an effective amount of one or more anti-KLK5 antibodies, anti-KLK7 antibodies, or anti-KLK5/KLK7 antibodies to the subject. Further, in some embodiments, subjects to be treated exhibit a chronic relapsing form of skin inflammation, a disturbance of epidermal barrier function, and/or IgE-mediated sensitization to allergens.

Further aspects of the disclosure relate to methods and compositions (e.g., anti-KLK5 antibodies, anti-KLK7 antibodies, and/or anti-KLK5/KLK7 antibodies) useful for treating subjects having Netherton syndrome. In some embodiments, methods of treating a subject having Netherton syndrome are provided herein that comprise administering an effective amount of one or more anti-KLK5 antibodies, anti-KLK7 antibodies, or anti-KLK5/KLK7 antibodies to the subject. Netherton syndrome is a rare and severe autosomal recessive skin disorder. In some embodiments, Netherton syndrome is associated with congenital erythroderma, a specific hair-shaft abnormality, and/or atopic manifestations with high IgE levels (e.g., compared with normal IgE levels in subjects who do not have Netherton Syndrome or related conditions). In some embodiments, subjects having Netherton syndrome exhibit atopic manifestations include eczema-like rashes, atopic dermatitis, pruritus, hay fever, angioedema, urticaria, high levels of IgE in the serum, and/or hypereosinophilia. In some embodiments, Netherton syndrome is caused by mutations in the serine protease inhibitor Kazal-type 5 (SPINK5) gene, which encodes the protease inhibitor lymphoepithelial Kazal-type-related inhibitor. In some embodiments, absence of this protease inhibitor causes stratum corneum detachment secondary to epidermal proteases hyperactivity. Accordingly, in some embodiments, methods provided herein comprise administering an effective amount of one or more anti-KLK5 antibodies, anti-KLK7 antibodies, or anti-KLK5/KLK7 antibodies provided herein to a subject for purposes of ameliorating one or more aspects or symptoms (e.g., atopic manifestations, e.g., skin rash scaling, stratum corneum detachment) associated with Netherton Syndrome.

Further aspects of the disclosure relate to methods and compositions (e.g., anti-KLK5 antibodies, anti-KLK7 antibodies, and/or anti-KLK5/KLK7 antibodies) useful for treating eosinophilic esophagitis. In some embodiments, symptoms of eosinophilic esophagitis include difficulty feeding, failure to thrive, vomiting, epigastric or chest pain, dysphagia, and food impaction. In some embodiments, subjects having eosinophilic esophagitis are young males with a relatively high predisposition to atopic disease. In some embodiments, subjects having eosinophilic esophagitis are diagnosed by endoscopy and/or biopsy findings of isolated eosinophils in the esophagus. In some embodiments, eosinophilic esophagitis is defined histologically by the presence of proliferative changes, which, in some embodiments, includes thickening of the basal epithelial layer and/or elongation of papillae, a minimum of 24 eosinophils per high-power field in the distal esophagus, and/or the absence of eosinophilia in any other evaluated intestinal segment. In some embodiments, subjects having eosinophilic esophagitis exhibit low levels or lack of certain serine protease inhibitors belonging to the lympho-epithelial Kazal-type inhibitor protein family, such as SPINK7, e.g., in esophageal biopsies. In some embodiments, eosinophilic esophagitis is differentiated from reflux esophagitis on the basis of the magnitude of mucosal eosinophilia and lack of response to acid suppression. In some embodiments, methods of treating a subject having eosinophilic esophagitis are provided herein that comprise administering an effective amount of one or more anti-KLK5 antibodies, anti-KLK7 antibodies, or anti-KLK5/KLK7 antibodies to the subject.

Further aspects of the disclosure relate to methods and compositions (e.g., anti-KLK5 antibodies, anti-KLK7 antibodies, and/or anti-KLK5/KLK7 antibodies) useful for treating prurigo nodularis. Prurigo nodularis is a chronic inflammatory skin disease where an extremely itchy, symmetrically distributed rash appears most commonly on the arms, legs, the upper back and/or the abdomen. In some embodiments, prurigo nodularis appears on its own. However, in some embodiments, prurigo nodularis is associated with other skin diseases or medical conditions, such as cancer, diabetes, chronic kidney disease or AIDS. In some embodiments, altered function of the immune system and nerves in the skin is believed to be associated with heightened sensations of itchiness (pruritus) that leads to frequent scratching; whereby such frequent scratching and picking of the skin contributes to further lesion thickening and formation. Accordingly, in some embodiments, methods of treating a subject having prurigo nodularis are provided herein that comprise administering an effective amount of one or more anti-KLK5 antibodies, anti-KLK7 antibodies, or anti-KLK5/KLK7 antibodies to the subject.

Further aspects of the disclosure relate to methods and compositions (e.g., anti-KLK5 antibodies, anti-KLK7 antibodies, and/or anti-KLK5/KLK7 antibodies) useful for treating chronic pruritis. In some embodiments, chronic pruritus is associated with an itch lasting greater than 6 weeks (e.g., up 3 months, up 6 months, up to 1 year, or more). In some embodiments, chronic pruritus arises in association with potentially unrelated diseases, including chronic kidney disease, hepatobiliary disease, and neuropathic entities such as brachioradial pruritus and notalgia paresthetica. In some embodiments, chronic pruritus of unknown origin (CPUO) is established when no underlying origin for pruritus can be determined. In some embodiments, chronic pruritus is associated with severe itch and significant scratch lesions. in some embodiments, methods of treating a subject having chronic pruritis, including CPUO, are provided herein that comprise administering an effective amount of one or more anti-KLK5 antibodies, anti-KLK7 antibodies, or anti-KLK5/KLK7 antibodies to the subject.

Further aspects of the disclosure relate to methods and compositions (e.g., anti-KLK5 antibodies, anti-KLK7 antibodies, and/or anti-KLK5/KLK7 antibodies) useful for treating ichthyosis vulgaris. In some embodiments, ichthyosis vulgaris is caused by heterozygous mutation in the filaggrin gene. In some embodiments, subjects with homozygous or compound heterozygous mutations in this gene have a more severe phenotype. In some embodiments, ichthyosis vulgaris is characterized histologically by absent or reduced keratohyalin granules in the epidermis and mild hyperkeratosis. Keratohyalin contains a histidine-rich protein which is the precursor form (profilaggrin) of filaggrin, a keratin filament-aggregating protein. In some embodiments, profilaggrin and filaggrin are reduced or absent in subjects having ichthyosis vulgaris. In some embodiments, ichthyosis vulgaris comprises palmar hyperlinearity, keratosis pilaris, and a fine scale that is most prominent over the lower abdomen, arms, and legs. In some embodiments, a subject may exhibit prominent scaling. In some embodiments, a subject may exhibit palmar hyperlinearity, keratosis pilaris, and, in some cases, fine scaling. Accordingly, in some embodiments, methods of treating a subject having ichthyosis vulgaris or one or more symptoms or phenotypic characteristics thereof are provided herein that comprise administering an effective amount of one or more anti-KLK5 antibodies, anti-KLK7 antibodies, or anti-KLK5/KLK7 antibodies to the subject.

Further aspects of the disclosure relate to methods and compositions (e.g., anti-KLK5 antibodies, anti-KLK7 antibodies, and/or anti-KLK5/KLK7 antibodies) useful for treating psoriasis. Psoriasis (or psoriasis vulgaris) is a chronic inflammatory dermatosis. In some embodiments, psoriasis is characterized by red, scaly skin patches that may be found on the scalp, elbows, and/or knees of a subject. In some embodiments, psoriasis is associated with severe arthritis in a subject. In some embodiments, psoriasis associated lesions are caused by abnormal keratinocyte proliferation and infiltration of inflammatory cells into the dermis and epidermis. In some embodiments, subject's experience onset of psoriasis between 15 and 30 years of age. In some embodiments, psoriatic lesions are characterized by skin induration, scaling, and/or erythema, which may be accompanied by histologic evidence of inflammation, abnormal keratinocyte proliferation/terminal differentiation, and/or dermal angiogenesis. In some embodiments, psoriatic inflammatory infiltrates, which may be pronounced at the dermal-epidermal junction, comprise activated T cells and antigen-presenting cells (APCs). In some embodiments, presence of activated T cells and APCs in such infiltrates precede development of epidermal hyperproliferation. In some embodiments, increased levels of inflammatory cytokines are detectable in lesional psoriatic epidermis, which may result in the potentiation of T-cell activation as well as hyperproliferation and accelerated differentiation of keratinocytes. In some embodiments, methods of treating a subject having psoriasis are provided herein that comprise administering an effective amount of one or more anti-KLK5 antibodies, anti-KLK7 antibodies, or anti-KLK5/KLK7 antibodies to the subject. In some embodiments, subjects are treated prior to development of epidermal hyperproliferation. However, in some embodiments, subjects are treated after development of epidermal hyperproliferation and accelerated differentiation of keratinocytes.

Further aspects of the disclosure relate to methods and compositions (e.g., anti-KLK5 antibodies, anti-KLK7 antibodies, and/or anti-KLK5/KLK7 antibodies) useful for treating rosacea. Rosacea is an inflammatory disease characterized by erythema, papulopustules, and/or telangiectasia. In some embodiments, subjects having rosacea express abnormally high levels of cathelicidin in their facial skin. In some embodiments, proteolytically processed forms of cathelicidin peptides found in rosacea are different from those present in normal subjects. In some embodiments, methods of treating a subject having rosacea are provided herein that comprise administering an effective amount of one or more anti-KLK5 antibodies, anti-KLK7 antibodies, or anti-KLK5/KLK7 antibodies to the subject.

Further aspects of the disclosure relate to methods of treating a subject having asthma are provided herein that comprise administering an effective amount of one or more antibodies disclosed herein (e.g., anti-KLK5 antibodies, anti-KLK5/KLK7 antibodies) to the subject. In some embodiments, methods of treating a subject having asthma are provided herein that comprise administering an effective amount of one or more anti-KLK5 antibodies. In some embodiments, the subject has asthma (e.g., a subject to be treated with a KLK5 targeting antibody provided herein). In some embodiments, the asthma is selected from: allergic asthma, aspirin sensitive/exacerbated asthma, asthma due to smoking, asthma uncontrolled on corticosteroids or other chronic asthma controller medications, atopic asthma, bronchial obstruction associated asthma, pathogenesis-related asthma, chronic asthma, corticosteroid naïve asthma, corticosteroid refractory asthma, corticosteroid resistant asthma, eosinophil-high asthma, eosinophilic asthma, eosinophil-low asthma, exercise-induced asthma, mild asthma, moderate to severe asthma, Netherton Syndrome asthma, newly diagnosed and/or untreated asthma, non-allergic asthma, non-Th2-driven asthma, periostin-high asthma, periostin-low asthma, Th2-low asthma, Type 2 (T2)-driven asthma, and Type 2 low inflammation asthma. In some embodiments, the subject has atopic asthma or allergic asthma. In some embodiments, the subject has aspirin-sensitive or aspirin-exacerbated asthma. In some embodiments, the subject has asthma associated with a non-steroidial anti-inflammatory drug (NSAID). Accordingly, in some embodiments, the subject has asthma that was triggered by aspirin or similar NSAID (e.g., recently ingested aspirin or similar NSAID). In some embodiments, the subject has bronchial spasms that may or may not be characterized as asthma. For example, in some embodiments, the subject has exercise-induce bronchial spasms.

In some embodiments, the subject has eosinophilic asthma. In some embodiments, the subject has an eosinophilic inflammation positive (EIP) asthma. In some embodiments, the subject has eosinophilic inflammation negative (EIN) asthma. In some embodiments, the subject has eosinophil-high asthma (e.g., at least about any of 150, 200, 250, 300, 350 or 400 eosinophil counts/ml blood). In some embodiments, the subject has eosinophil-low asthma (e.g., less than about 150 eosinophil counts/μl blood or less than about 100 eosinophil counts/μl blood). These and further examples of asthma related conditions treatable using compositions provided herein (e.g., anti-KLK5 antibodies) are disclosed in WO2015/061441, METHODS OF DIAGNOSING AND TREATING EOSINOPHILIC DISORDERS, published on Apr. 30, 2015, the relevant contents of which are incorporated herein by reference.

In some embodiments, the subject has exercise-induced asthma, intermittent or exercise-induced, mild asthma, mild or corticosteroid naive asthma, moderate to severe asthma, Netherton Syndrome asthma, newly diagnosed asthma untreated asthma, or severe asthma. In some embodiments, the subject has asthma not previously requiring or involving use (e.g., chronic use) of inhaled topical or systemic steroids to control or manage symptoms (e.g., symptoms such as cough, wheezing, shortness of breath/breathlessness, and chest pain)

In some embodiments, the subject has periostin-low asthma (e.g. having periostin level less than about 20 ng/mL serum). In some embodiments, the subject has periostin-high asthma (e.g. having periostin level at least about any of 20 ng/mL, 25 ng/mL, or 50 ng/mL serum). In some embodiments, the subject has non-allergic asthma (e.g., which may or may not be triggered by infection, e.g., with a respiratory virus (e.g., influenza, coronavirus, parainfluenza, rhinovirus, human metapneumovirus, and respiratory syncytial virus) or inhaled irritant (air pollutant, smog, combustion particles (e.g., diesel particles), volatile chemicals, gases indoors or outdoors) or by relatively cold dry air. In some embodiments, the subject has asthma due to acute or chronic primary or second-hand exposure to smoke (cigarettes, cigars, pipes, or other combustion products) or as a result of inhaling or vaping (nicotine, cannabis or other similar substances). In some embodiments, the subject has persistent chronic severe asthma with acute events of worsening symptoms (exacerbations or flares) that can be life threatening.

In some embodiments, the subject has a T helper lymphocyte type 2 (Th2) or type 2 (Th2) high asthmatic condition. In some embodiments, the subject has Th2 induced asthma. For example, in some embodiments, Th2 cells and/or their secreted effector molecules mediate immune response to allergens and are triggered by exposure to specific allergens leading to allergic asthma in a subject. In some embodiments, a subject has activated Th2 cell-mediated asthma, which may be caused in part by the secretion of interleukins, e.g., IL-4, IL-5 and IL-13. Accordingly, in some embodiments, KLK5 antibodies (or other antibodies) may be combine separately or in a multi-specific antibody format with one or more antibodies targeting cytokines such as IL-13, IL-17, IL-5, and IL-4 as well as targets associated with allergy such as IgE. Examples of such antibodies for the treatment of asthma include, but are not limited to, omalizumab (XOLAIR®) (targeting soluble IgE); lebrikizumab (targeting IL-13); mepoiizumab (targeting IL-5); and quilizumab (targeting membrane-bound IgE).

Still, in other embodiments, antibodies as described herein are used to treat, but are not limited to the following, inflammatory disorders, infectious diseases, allergic diseases, and autoimmune disorders. In some embodiments, the inflammatory disorders are selected from but not limited to, rosacea, prurigo nodularis, Crohn's disease, ankylosing spondylitis, ulcerative colitis, hidradenitis suppurativa, and uveitis or one or more barrier function related symptoms thereof. In some embodiments, the allergic diseases are selected from but not limited to, eczema, atopic dermatitis, asthma, sinusitis, and eosinophilic esophagitis. In some embodiments, the autoimmune diseases are selected from but not limited to, rheumatoid arthritis, psoriatic arthritis, juvenile idiopathic arthritis, Behçet's disease, and plaque psoriasis or one or more barrier function related symptoms thereof.

Determination of whether an amount of the antibody (e.g., anti-KLK5 antibody, anti-KLK7 antibody, anti-KLK5/7 antibody) achieved the therapeutic effect would be evident to one of skill in the art based on the teachings provided herein. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. The particular dosage regimen, i.e., dose, timing and repetition, used in the method described herein will depend on the particular subject and that subject's medical history, as discussed herein.

Empirical considerations, such as time to maximum effect, the half-life, and/or time above a specific concentration generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Other reasons for dose-adjusting include differences in pharmacokinetics or pharmacodynamic response driven by sex, age, individual response, polymorphisms on the antibody target and/or receptors involved in antibody clearance. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

Dosing frequencies may vary in accordance with the claimed methods. In some embodiments, a composition may be administered once. In some embodiments, a composition will be administered on multiple occasions. In some embodiments, dosing frequency is every week, every 2 weeks, every 3 weeks, every 4 weeks, every 5 weeks, every 6 weeks, every 7 weeks, every 8 weeks, every 9 weeks, or every 10 weeks; or once every month, every 2 months, or every 3 months, or longer. In some embodiments, a composition will be administered daily, biweekly, weekly, bimonthly, monthly, or at any time interval that provide suitable (e.g., maximal) efficacy while minimizing safety risks to the subject. Generally, the efficacy and the treatment and safety risks may be monitored throughout the course of treatment.

In some embodiments, a subject may be administered a composition provided herein (e.g., an anti-KLK5 antibody and/or an anti-KLK7 antibody) at one or more intervals during a set period of time. In some cases, periods of time during which a subject is administered a composition at one or more intervals may be separated by periods of time in which the subject is not administered the composition. In some embodiments, the relative durations of respective periods of time may depend on the subject's response to treatment or severity of disease or both and/or may be determined based on the judgment of a treating physician.

In some embodiments, an antibody can be administered parenterally. For example, a parenterally administered composition may be administered topically, transmucosally, by subcutaneous, intracutaneous, intravenous, intraperitoneal, intratumor, intramuscular, intraarticular, intraarterial, or infusion techniques.

In some embodiments, an antibody (e.g., an anti-KLK5 antibody and/or anti-KLK7 antibody) is administered intravenously. In some embodiments, an antibody (e.g., an anti-KLK5 antibody and/or anti-KLK7 antibody) is administered subcutaneously or topically.

For intravenous injection, water soluble antibodies can be administered by the drip method, whereby a pharmaceutical formulation containing the antibody and a physiologically acceptable excipient is infused. Physiologically acceptable excipients may include, for example, 5% dextrose, 0.9% saline. Ringer's solution or other suitable excipients. Other injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). In some cases, preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, an antibody is administered via site-specific or targeted local delivery techniques. Examples of site-specific or targeted local delivery techniques include various implantable, transdermal, or transmucosal depot sources of the antibody or local delivery systems.

An anti-KLK5 antibody and/or anti-KLK7 antibody and treatment methods involving such as described in the present disclosure may be utilized in combination with other types of therapy for the target disease or disorder disclosed herein. In this context, an antibody composition and a therapeutic agent may be given either simultaneously or sequentially. Such therapies can be administered simultaneously or sequentially (in any order) with the treatment according to the present disclosure.

Accordingly, aspects of the disclosure related to methods and compositions (e.g., anti-KLK5 antibodies, anti-KLK7 antibodies, and/or anti-KLK5/KLK7 antibodies). In some embodiments, antibodies as described herein, can be administered as a combination therapy (concomitantly or sequentially, e.g., over time). In some embodiments, the combination therapy comprises administering one or more of the antibodies as described herein (e.g., anti-KLK5 antibodies, anti-KLK7 antibodies, and/or anti-KLK5/KLK7 antibodies) and at least one additional therapeutic agent (e.g., one, two, three, four, five, six, or seven therapeutic agents). In some embodiments, the antibodies are monospecific. In some embodiments, the antibodies are bispecific, e.g., a KLK5/KLK7 antibody. In some embodiments, the antibodies are multi-specific. In some embodiments, one or more of the antibodies as described herein and at least one additional therapeutic agent (e.g., one, two, three, four, five, six, or seven therapeutic agents) are administered together. In some embodiments, one or more of the antibodies as described herein and at least one additional therapeutic agent (e.g., one, two, three, four, five, six, or seven therapeutic agents) are administered separately.

In some embodiments, the additional therapeutic agent is an anti-inflammatory agent. In some embodiments, the anti-inflammatory agent is selected from but is not limited to, low-dose antibiotics, steroids, corticosteroids, tacrolimus, anti-IL4R antibodies (e.g., dupilumab), anti-IL-13 antibodies, TNF inhibitors (e.g., anti-TNF), IL-12/23 inhibitors, IL-17 inhibitors, and IL-4 receptor inhibitors, doxycycline, methotrexate, prednisone, cyclosporine, mycophenolate mofetil, dupilumab, certolizumab pegol, etanercept, adalimumab, infliximab, golimumab, ustekinumab, secukinumab, ixekizumab, brodalumab, abatacept, tidrakizumab-asmn, risankisumab-rzaa, and guselkumab. In some embodiments, the anti-inflammatory agent is administered orally. In some embodiments, the anti-inflammatory agent is administered topically. In some embodiments, the anti-inflammatory agent is administered via injection (e.g., intravenous, subcutaneous, or intramuscular).

In some embodiments the therapeutic combination comprises one or more of the antibodies as described herein (e.g., anti-KLK5 antibodies, anti-KLK7 antibodies, and/or anti-KLK5/KLK7 antibodies) delivered with one or more additional antibodies or fragments thereof (e.g., one, two, three, four, five, six, or seven antibodies). In some embodiments, the therapeutic combination comprising one or more of the antibodies as described herein and one or more additional antibodies or fragments thereof (e.g., one, two, three, four, five, six, or seven antibodies) are delivered separately. In some embodiments, the therapeutic combination comprising one or more of the antibodies as described herein and one or more additional antibodies or fragments thereof (e.g., one, two, three, four, five, six, or seven antibodies) are delivered together.

In some embodiments, the therapeutic combination comprising one or more of the antibodies as described herein and one or more additional antibodies or fragment thereof (e.g., one, two, three, four, five, six, or seven antibodies) is as a multi-specific antibody combination. In some embodiments, the multi-specific antibody combination, comprises a KLK5 and a KLK7 distinct antigen binding site, and one or more additional distinct antigen binding sites from one or more additional antibodies. In some embodiments, a multi-specific antibody comprises direct fusion or linking of different antigen-specific binding sites. In some embodiments, the additional antibodies or fragments thereof are selected from but not limited to, an anti-IL4R antibody, an anti-IL-13 antibodies, an anti-TNF antibody, an anti-IL-12/23 antibody, an anti-IL-17 antibody, doxycycline, dupilumab, certolizumab pegol, etanercept, adalimumab, infliximab, golimumab, ustekinumab, secukinumab, ixekizumab, brodalumab, abatacept, tildrakizumab-asmn, risankizumab-rzaa, and/or guselkumab.

In some embodiments, in a non-limiting example, in the treatment of rosacea, additional therapeutics could be selected from a topically administered steroid; oral administration of methotrexate; oral administration of cyclosporine; and/or administration of a TNF inhibitor. In some embodiments, in a non-limiting example, in the treatment of atopic dermatitis, additional therapeutics could be selected from subcutaneous administration of dupilumab, and/or topical administration of a steroid.

Any of the anti-KLK5 antibodies disclosed herein can also be used for detecting presence of KLK5 in vitro or in vivo. Results obtained from such detection methods can be used for diagnostic purposes (e.g., diagnosing diseases associated with KLK5) or for scientific research purposes (e.g., identifying new KLK5 secreting cell types, studying bioactivity and/or regulation of secreted KLK5). For assay uses such as diagnostic uses, an anti-KLK5 antibody as described herein may be conjugated with a detectable label (e.g., an imaging agent such as a contrast agent) for detecting presence of KLK5, either in vivo or in vitro.

Any of the anti-KLK7 antibodies disclosed herein can also be used for detecting presence of KLK7 in vitro or in vivo. Results obtained from such detection methods can be used for diagnostic purposes (e.g., diagnosing diseases associated with KLK7) or for scientific research purposes (e.g., identifying new KLK7 secreting cell types, studying bioactivity and/or regulation of secreted KLK7). For assay uses such as diagnostic uses, an anti-KLK7 antibody as described herein may be conjugated with a detectable label (e.g., an imaging agent such as a contrast agent) for detecting presence of KLK7, either in vivo or in vitro.

As used herein, “conjugated” or “attached” means two entities are associated, preferably with sufficient affinity that the therapeutic/diagnostic benefit of the association between the two entities is realized. The association between the two entities can be either direct or via a linker, such as a polymer linker. Conjugated or attached can include covalent or noncovalent bonding as well as other forms of association, such as entrapment, e.g., of one entity on or within the other, or of either or both entities on or within a third entity, such as a micelle.

In other embodiments, an anti-KLK5 or an anti-KLK7 antibody as described herein can be attached to a detectable label, which is a compound that is capable of releasing a detectable signal, either directly or indirectly, such that the aptamer can be detected, measured, and/or qualified, in vitro or in vivo. Examples of such “detectable labels” are intended to include, but are not limited to, fluorescent labels, chemiluminescent labels, colorimetric labels, enzymatic markers, radioactive isotopes, and affinity tags such as biotin. Such labels can be conjugated to the aptamer, directly or indirectly, by conventional methods.

The reporting agent can also be a dye, e.g., a fluorophore, which is useful in detecting a disease mediated by KLK5 or KLK7 expressing cells in tissue samples respectively.

To perform a diagnostic assay in vitro, an anti-KLK5 antibody can be brought in contact with a sample suspected of containing KLK5, e.g., KLK5 expressing cells or soluble KLK5 in disease microenvironment. The antibody and the sample may be incubated under suitable conditions for a suitable period to allow for binding of the antibody to the KLK5 antigen. Such an interaction can then be detected via routine methods, e.g., ELISA, histological staining or FACS. To perform a diagnostic assay in vivo, a suitable amount of anti-KLK5 antibodies, conjugated with a label (e.g., an imaging agent or a contrast agent), can be administered to a subject in need of the examination. Presence of the labeled antibody can be detected based on the signal released from the label by routine methods.

To perform a diagnostic assay in vitro, an anti-KLK7 antibody can be brought in contact with a sample suspected of containing KLK7, e.g., KLK7 expressing cells or soluble KLK7 in disease microenvironment. The antibody and the sample may be incubated under suitable conditions for a suitable period to allow for binding of the antibody to the KLK7 antigen. Such an interaction can then be detected via routine methods, e.g., ELISA, histological staining or FACS. To perform a diagnostic assay in vivo, a suitable amount of anti-KLK7 antibodies, conjugated with a label (e.g., an imaging agent or a contrast agent), can be administered to a subject in need of the examination. Presence of the labeled antibody can be detected based on the signal released from the label by routine methods.

To perform scientific research assays, an anti-KLK5 antibody can be used to study bioactivity of KLK5, detect the presence of KLK5 intracellularly, and or regulating the effect of KLK5. For example, a suitable amount of anti-KLK5 can be brought in contact with a sample (e.g. a new cell type that is not previously identified as KLK5 producing cells) suspected of producing KLK5. The cells are permeabilized prior to contacting the anti-KLK5 antibody. The antibody and the sample may be incubated under suitable conditions for a suitable period to allow for binding of the antibody to the KLK5 antigen. Such an interaction can then be detected via routine methods, e.g., ELISA, histological staining or FACS.

To perform scientific research assays, an anti-KLK7 antibody can be used to study bioactivity of KLK7, detect the presence of KLK7 intracellularly, and or regulating the effect of KLK7. For example, a suitable amount of anti-KLK7 can be brought in contact with a sample (e.g. a new cell type that is not previously identified as KLK7 producing cells) suspected of producing KLK7. The cells are permeabilized prior to contacting the anti-KLK7 antibody. The antibody and the sample may be incubated under suitable conditions for a suitable period to allow for binding of the antibody to the KLK7 antigen. Such an interaction can then be detected via routine methods, e.g., ELISA, histological staining or FACS.

### VI. KITS FOR THERAPEUTIC AND DIAGNOSTIC APPLICATIONS

The present disclosure also provides kits for the therapeutic or diagnostic applications as disclosed herein. Such kits can include one or more containers comprising an antibody, e.g., any of those described herein.

In some embodiments, the kit can comprise instructions for use in accordance with any of the methods described herein. The included instructions can comprise a description of administration of the antibody to treat, delay the onset, or alleviate a target disease as those described herein. The kit may further comprise a description of selecting an individual suitable for treatment based on identifying whether that individual has the target disease. In still other embodiments, the instructions comprise a description of administering an antibody to an individual at risk of the target disease.

The instructions relating to the use of an antibody described generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating a disease or disorder. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like.

Also contemplated are packages for use in combination with a specific device, such as an infusion device, such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an antibody as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

Also provided herein are kits for use in detecting the target protein (e.g., KLK5 and/or KLK7) in a sample. Such a kit may comprise any of the antibodies described herein. In some instances, the antibody can be conjugated with a detectable label as those described herein. As used herein, “conjugated” or “attached” means two entities are associated, preferably with sufficient affinity that the therapeutic/diagnostic benefit of the association between the two entities is realized. The association between the two entities can be either direct or via a linker, such as a polymer linker. Conjugated or attached can include covalent or noncovalent bonding as well as other forms of association, such as entrapment, e.g., of one entity on or within the other, or of either or both entities on or within a third entity, such as a micelle.

Alternatively or in addition, the kit may comprise a secondary antibody capable of binding to an antibody described herein. The kit may further comprise instructions for using the antibody for detecting the target protein (e.g., KLK5 and/or KLK7).

### EXAMPLES

### Example 1: Generation and Selection of Anti-KLK5 and Anti-KLK7 Antibodies

### (i) Affinity

Antibody binding kinetic experiments via Biacore were performed to screen for anti-KLK5 and anti-KLK7 antibodies having high affinity to the respective targets. All screening assays were performed at 25 C. The running buffer used was 20 mM HEPES, 300 mM NaCl, 0.01% Tween-20, pH 7.5. Antibodies (1 ug/mL) were captured on Fc2-4 of a Series S ProteinA Sensor Chip (Cytiva) flowing at 10 ul/mL for 30 s. Kinetics measurements were made in single-cycle kinetics mode with a series of 4-5 concentrations with top concentration of 25-100 nM and serial dilution 4-fold. Contact time was typically 300 s and dissociation time ranged from 1800-3600 s. Flow rate of 30 ul/mL was typically used. The sensor chip was regenerated with 10 mM glycine, pH 1.5 with a contact time of 30 s flowing at 50 ul/mL.

A summary of the binding Kinetics for the anti-KLK5 antibodies are provided below in Table 6.

TABLE 6
Anti-KLK5 Antibody Affinity and Potency

| | hKLK5 | hKLK5 | mKLK5 | mKLK5 | mKLK5 | hKLK5 |
|---|---|---|---|---|---|---|
| | IC50 | IC90 | IC50 | IC90 | affinity | affinity |
| Ab | (nM) | (nM) | (nM) | (nM) | (nM) | (nM) |
| KLK5-L1- | 2.26 | 13.34 | 4.43 | 34.6 | 0.41 | * |
| Ab2 | | | | | | |
| KLK5-L1- | 1.71 | 15.04 | 4.97 | 40.1 | 0.32 | * |
| Ab3 | | | | | | |
| KLK5-L1- | 2.49 | 10.64 | 9.26 | 81.24 | 0.66 | * |
| Ab4 | | | | | | |
| KLK5-L1- | 2.24 | 6.707 | 4.1 | 25 | 0.26 | * |
| Ab5 | | | | | | |
| KLK5-L2- | 1.71 | 8.455 | 2.21 | 13.12 | 0.44 | 0.003 |
| Ab2 | | | | | | |
| KLK5-L2- | 2.26 | 10.16 | 1.82 | 29.47 | 0.59 | * |
| Ab3 | | | | | | |
| KLK5-L2- | 2.04 | 9.86 | 1.68 | 15.61 | 0.47 | * |
| Ab4 | | | | | | |
| KLK5-L3- | 4.446 | 23.98 | 15.04 | 91.33 | 3.8 | * |
| Ab2 | | | | | | |
| KLK5-L3- | 2.808 | 11.09 | 14.86 | 87.04 | 7.2 | * |
| Ab3 | | | | | | |
| KLK5-L3- | 1.76 | 9.809 | 8.27 | 83.15 | 8.8 | * |
| Ab4 | | | | | | |
| KLK5-L3- | 2.58 | 10.63 | 4.437 | 28.38 | 3.4 | * |
| Ab5 | | | | | | |
| KLK5-L3- | 2.895 | 12.07 | 12.67 | 80.83 | 12 | * |
| Ab6 | | | | | | |
| KLK5-L3- | 1.446 | 8.763 | 2.168 | 10.69 | 1.4 | * |
| Ab7 | | | | | | |
| KLK5-L1- | 3.45 | 83.13 | 29.39 | 103.13 | 4.44 | 0.783 |
| Ab1 | | | | | | |
| KLK5-L2- | 11.02 | 92.90 | 45.93 | 93.18 | 20.5 | 2.53 |
| Ab1 | | | | | | |
| KLK5-L3- | 12.74 | 111.91 | 81.35 | 129.64 | 16.6 | 2.62 |
| Ab1 | | | | | | |

* Below limit of detection

A summary of the binding Kinetics for the anti-KLK7 antibodies are provided below in Table 7.

TABLE 7
Anti-KLK7 Antibody Affinity and Potency

| | hKLK7 | hKLK7 | mKLK7 | mKLK7 | mKLK7 | hKLK |
|---|---|---|---|---|---|---|
| | IC50 | IC90 | IC50 | IC90 | affinity | affinity |
| Ab | (nM) | (nM) | (nM) | (nM) | (nM) | (nM) |
| KLK7-L1- | 0.93 | 20.68 | 0.50 | 108.47 | 1.62 | 0.134 |
| Ab1 | | | | | | |
| KLK7-L1- | 1.69 | 6.114 | 3.423 | 19.81 | 0.0354 | * |
| Ab2 | | | | | | |
| KLK7-L1- | 1.196 | 5.025 | 1.464 | 6.446 | 0.0402 | * |
| Ab3 | | | | | | |
| KLK7-L1- | 0.9142 | 3.667 | 1.423 | 7.125 | 0.0107 | * |
| Ab4 | | | | | | |
| KLK7-L1- | 1.23 | 4.66 | 1.353 | 7.248 | 0.017 | * |
| Ab5 | | | | | | |
| KLK7-L1- | 0.8397 | 3.748 | 1.255 | 4.583 | 0.0144 | * |
| Ab6 | | | | | | |
| KLK7-L1- | 0.979 | 5.014 | 1.062 | 5.025 | 0.0302 | * |
| Ab7 | | | | | | |
| KLK7-L1- | 0.7671 | 3.949 | 1.1 | 5.303 | 0.0569 | 0.01 |
| Ab8 | | | | | | |
| KLK7-L2- | 3.48 | 83.82 | −0.99 | 5.63 | 0.0796 | 0.202 |
| Ab1 | | | | | | |
| KLK7-L2- | 1.56 | 5.661 | 2.314 | 6.015 | 0.0357 | * |
| Ab2 | | | | | | |
| KLK7-L2- | 0.8334 | 3.391 | 1.493 | 4.881 | * | * |
| Ab3 | | | | | | |
| KLK7-L2- | 0.7955 | 3.175 | 1.634 | 6.509 | 0.0101 | * |
| Ab4 | | | | | | |
| KLK7-L2- | 0.8135 | 4.128 | 1.905 | 6.518 | 0.018 | * |
| Ab5 | | | | | | |
| KLK7-L3- | 0.37 | 10.54 | 10.94 | 212.10 | 9.03 | 0.0205 |
| Ab1 | | | | | | |
| KLK7-L3- | 0.8019 | 4.435 | 2.682 | 11.49 | 0.125 | * |
| Ab2 | | | | | | |
| KLK7-L3- | 1.396 | 8.254 | 3.34 | 11.41 | 0.217 | * |
| Ab3 | | | | | | |
| KLK7-L4- | 0.52 | 14.95 | 16.33 | 117.53 | 6 | 0.219 |
| Ab1 | | | | | | |
| KLK7-L4- | 1.422 | 6.345 | 2.423 | 9.045 | 0.0266 | * |
| Ab2 | | | | | | |
| KLK7-L5- | 0.30 | 4.85 | 14.97 | 67.26 | 4.86 | 0.192 |
| Ab1 | | | | | | |
| KLK7-L5- | 0.3299 | 2.295 | 1.478 | 26.51 | 0.0253 | * |
| Ab2 | | | | | | |

* Below limit of detection

### (ii) Protease Inhibition Assay

To evaluate the anti-KLK5 antibodies and anti-KLK7 antibodies' ability to inhibit KLK5 or KLK7 protease activity respectively, 1.5 nM of human or mouse KLK5 or 0.5 nM of human or mouse KLK7 were prepared in assay buffer (0.1 M NaH2PO4 pH=7.5 for KLK5 and 50 mM Tris, 150 mM NaCl, pH 7.5 for KLK7). Antibodies diluted in PBS to testing concentrations were added. Substrate was then added (50 uM BOC-Val-Pro-Arg-AMC for KLK5 and 30 uM KHLF-AMC for KLK7). Fluorescence signal was then measured every min for 30 min at room temperature.

The results show that the anti-KLK5 antibodies are capable of inhibiting KLK5 protease activity, and the anti-KLK7 antibodies are capable of inhibiting KLK7 protease activity.

### Example 2. Anti-KLK5 and Anti-KLK7 Antibodies Specifically Bind the Active Form of Human KLK5 and Human KLK7

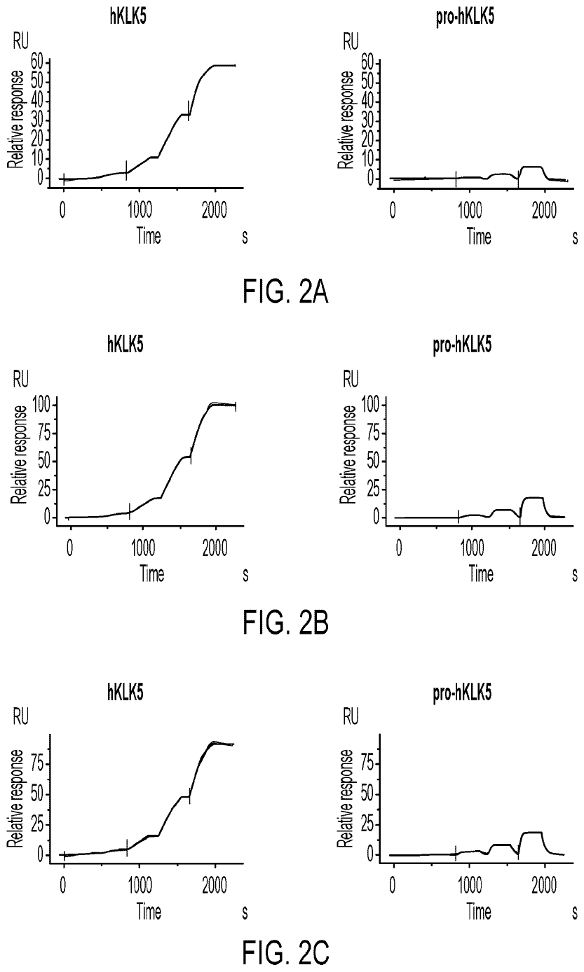

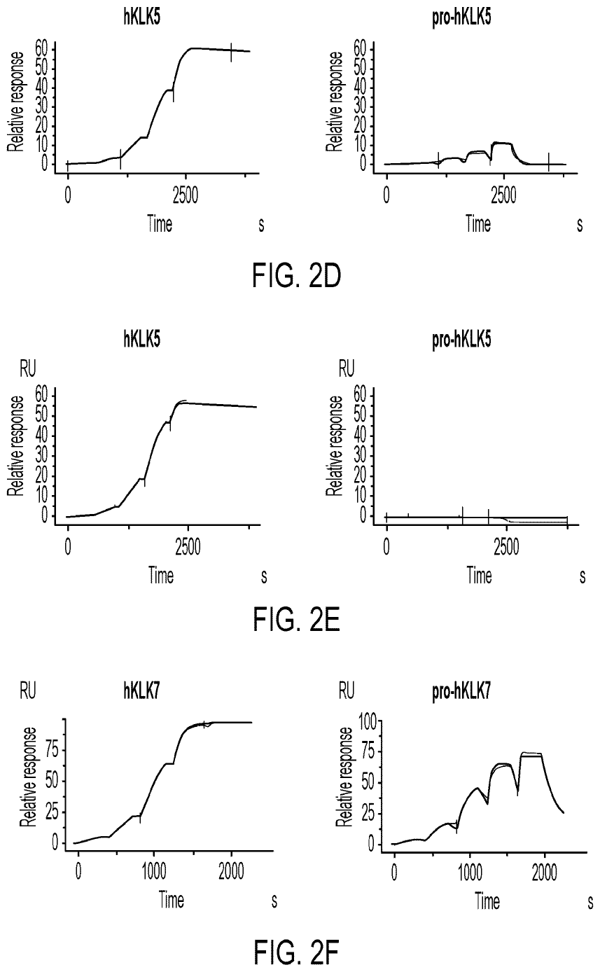

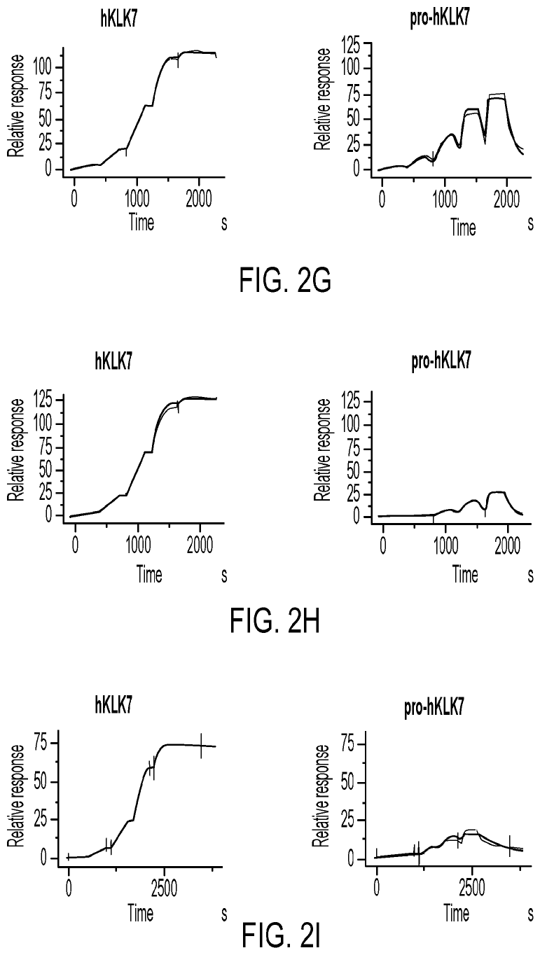

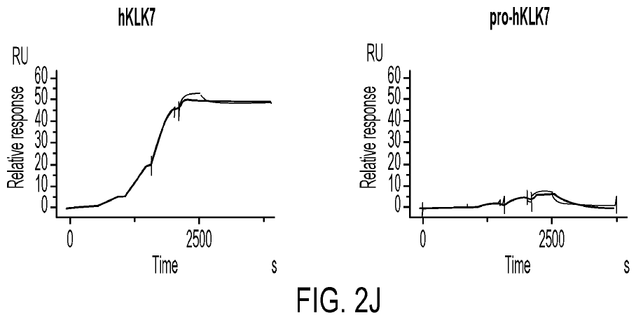

Antibodies were captured on a ProteinA chip from a 1 μg/mL stock. Either active human KLK5 (hKLK5) or human KLK7 (hKLK7), or the pro-form of hKLK5 or hKLK7 was flowed over at 30 uL/min at the following concentrations (0.195 nM, 0.78 nM, 3.125 nM, 12.5 nM or 50 nM) with a contact time of 5-7 min and a dissociation time of 5-20 min. Anti-KLK5 antibodies KLK5-L1-Ab2 (FIG. 2A), KLK5-L2-Ab3 (FIG. 2B), KLK5-L3-Ab5 (FIG. 2C), and KLK5-L2-Ab4 (FIG. 2D) were observed to bind the active form of hKLK5 but not the pro-form of hKLK5, and anti-KLK7 antibodies KLK7-L2-Ab4 (FIG. 2E), KLK7-L2-Ab5 (FIG. 2F), KLK7-L5-Ab2 (FIG. 2G), and KLK7-L1-Ab7 (FIG. 2H) were observed to bind the active form of hKLK7 but not the pro-form of hKLK7. This data is consistent with the binding of these antibodies to the active sites of hKLK5 and hKLK7, as the structure of the active site is not present in the pro-form of the protease thus blocking binding of the antibodies.

### Example 3. Anti-KLK5 and Anti-KLK7 Antibodies are not Cleaved Upon Binding their Target

A concern in developing antibodies that bind proteases is that the proteolytic activity of the antibody's target may result in cleavage of the antibody once it binds, rendering the antibody's activity moot.

To test whether the anti-KLK5 and anti-KLK7 antibodies were susceptible to cleavage by hKLK5 or hKLK7, 1 μg of hKLK5 or hKLK7 active protein was diluted to 10 L in HBS to a final concentration of 0.1 μg/μL. Activity of hKLK5 and hKLK7 was confirmed through activity assays demonstrating cleavage of peptide substrate (data not shown). 40 pM of protease was used per reaction and mixed with 40 pM of mAb. The cleavage reaction was incubated for 18-20 h at 37° C. and then analyzed by reducing SDS-PAGE gel. Cleavage of the heavy chain of KLK5-L1-Ab5 and KLK5-L3-Ab7 can be observed by gel, but KLK5-L2-Ab2 and all six anti-KLK7 antibodies tested were not cleaved by KLK5 or KLK7, respectively (FIGS. 3A-3B).

### Example 4. Anti-KLK5 and Anti-KLK7 Antibodies are Specific to KLK5 and KLK7

Members of the Kallikrein family and Kallikrein-like families are considerably similar at the gene and protein level. Thus, developing antibodies that specifically bind a select member or members of the KLK family poses a challenge.

The ability of 10 μg/mL of anti-KLK5 and anti-KLK7 antibody to inhibit plasma KLK, KLK1, KLK2, KLK4, KLK6, KLK12, KLK14, Trypsin, Chymotrypsin and Urokinase was tested using measurement of cleavage of substrate. Substrates for protease assays were as follows: plasma KLK, KLK1, KLK2 (Pro-Phe-Arg-AMC); KLK4, KLK12, KLK13 and KLK14 (Boc-VPR-AMC); KLK6 (Boc-QAR-AMC); Trypsin (MCA-RPKPVG-NVAL (DNP)-NH2); chymotrypsin (Suc-AAPF-AMC) and urokinase (Z-Gly-Gly-Arg-AMC).

The following KLK family members were screened for affinity by Biacore: hKLK3, hKLK8, hKLK9, hKLK10, hKLK11, hKLK12 and hKLK13. All screening assays were performed at 25° C. The running buffer used was 20 mM HEPES, 300 mM NaCl, 0.01% Tween-20, pH 7.5. Bispecific antibodies (1 ug/mL) were captured on Fc2-4 of a Series S ProteinA Sensor Chip (Cytiva) flowing at 10 μg/mL for 30 s. Kinetics measurements were made in single-cycle kinetics mode with 25 and 100 nM of KLK. Contact time was 120 s and dissociation time was 300 s. Flow rate of 30 μg/mL was typically used. The sensor chip was regenerated with 10 mM glycine, pH 1.5 with a contact time of 30 s flowing at 50 μg/mL.

The following concentrations of enzyme were prepared: plasma KLK (10 nM), KLK1 (10 nM), KLK2 (10 nM), KLK4 (5 nM), KLK6 (5 nM), KLK12 (2.5 nM), KLK14 (2.5 nM), Trypsin (0.25 nM), Chymotrypsin (17 nM) and Urokinase (20 nM) in assay buffer. Assay buffers were as follows: KLK1 and 2 (50 mM Tris, 0.15 M NaCl, 10 mM CaCl₂), 0.05% (w/v) Brij-35, pH 7.5), KLK3 (50 mM Tris, 1 M NaCl, pH 8.0), KLK4 (50 mM Tris, 1 M NaCl, pH 8.0), KLK6 (50 mM Tris, 1.0 M Sodium Citrate, pH 7.5), KLK12 and 13 (100 mM Tris, 150 mM NaCl, 10 mM CaCl₂), 0.05% (w/v) Brij-35, pH 7.5), KLK14 (50 mM Tris, 150 mM NaCl, 0.05% (w/v) Brij-35, pH 8.0), Trypsin (1×HBS+1 mM CaCl₂)+0.05% Tween 20, pH 7.4), Chymotrypsin (25 mM Tris, 0.5 mM CaCl₂), pH 8.0), Urokinase (50 mM Tris+0.01% Tween 20), and plasma kallikrein KLKb1 (50 mM Tris, 250 mM NaCl, pH 7.5).

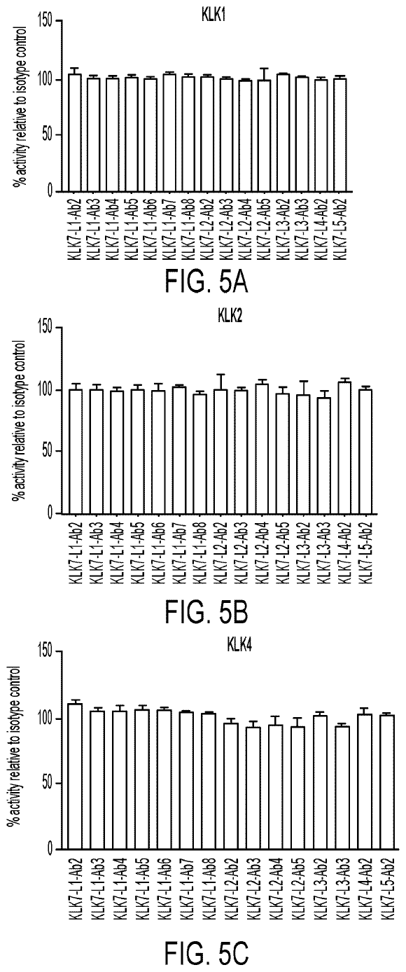

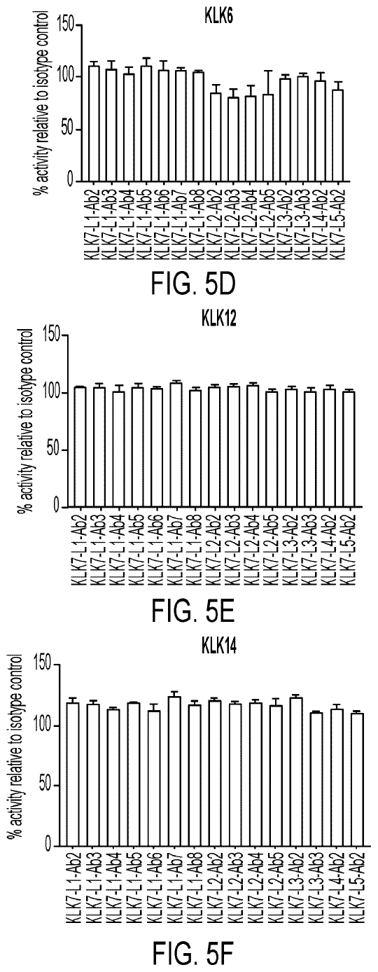

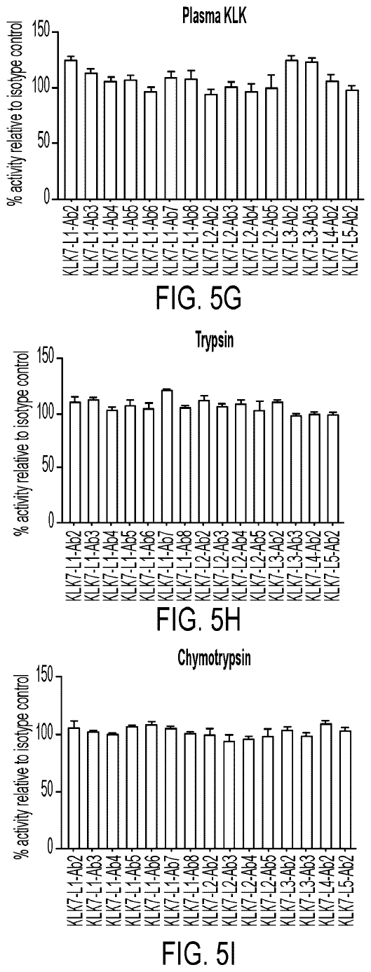

Antibodies diluted in PBS to testing concentrations were added. Substrate was then added and fluorescence was measured every min for 30 min at room temperature. Anti-KLK5 antibodies did not bind off-target proteases or other KLK family members other than KLK6, as they did not demonstrate binding above isotype control levels (FIGS. 4A-4C and 4E-4J). Follow up studies were run on KLK6 as anti-KLK5 antibodies had some binding/inhibitory activity (FIG. 4D; follow-up studies in FIG. 4K). All IC50s were at least 100-fold higher than activity against KLK5 demonstrating excellent specificity for the intended targets. Anti-KLK7 antibodies also did not bind off-target proteases or other KLK family members, as indicated by their activity compared to an isotype control (FIGS. 5A-5J).

To determine whether anti-KLK5 or anti-KLK7 antibodies bound other KLK family members or related proteases, the KLK family members hKLK3, hKLK8, hKLK9, hKLK10, hKLK11, hKLK12, hKLK13 and hKLK15, and the off-target proteases plasma KLK, KLK1, KLK2, KLK4, KLK6, KLK12, KLK14, trypsin, chymotrypsin, and urokinase were screened for binding activity by Biacore.

10 μg/mL of anti-KLK5/7 antibody was tested for inhibition of plasma KLK, KLK1, KLK2, KLK4, KLK6, KLK12, KLK14, trypsin, chymotrypsin and urokinase, using cleavage of the substrate as a readout.

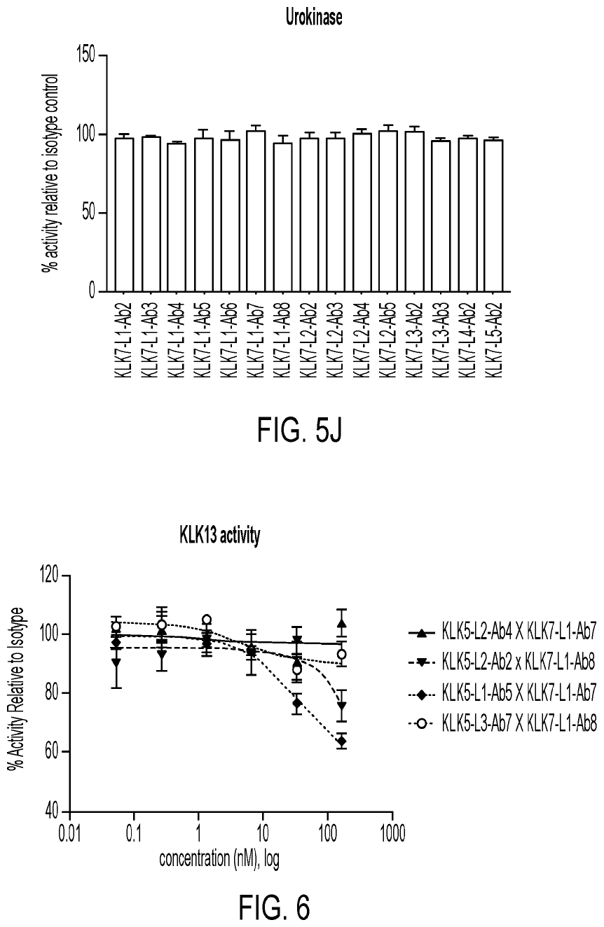

Anti-KLK5/anti-KLK7 bispecific antibodies were tested for inhibitory activity against KLK13 (FIG. 6). All IC50s were at least 100-fold higher than activity against KLK5 or KLK7, demonstrating excellent specificity for the intended targets.

The results show that anti-KLK5 antibodies described herein specifically bind to hKLK5, and do not show significant binding to other KLK family proteases or other related proteases. Similarly, anti-KLK7 antibodies described herein specifically bind to human KLK7, and do not show significant binding to other KLK family proteases or other related proteases.

### Example 5. Anti-KLK5 and Anti-KLK7 Antibodies Bind the Active Sites of KLK5 and KLK7

The inhibitory ability of an antibody is related to that antibody's ability to compete for binding of its target's active site with its target's substrate. The binding activity of anti-KLK5 and anti-KLK7 antibodies to KLK5 and KLK7 was tested in the presence of small molecule inhibitors known to bind the active sites of KLK5 and KLK7.

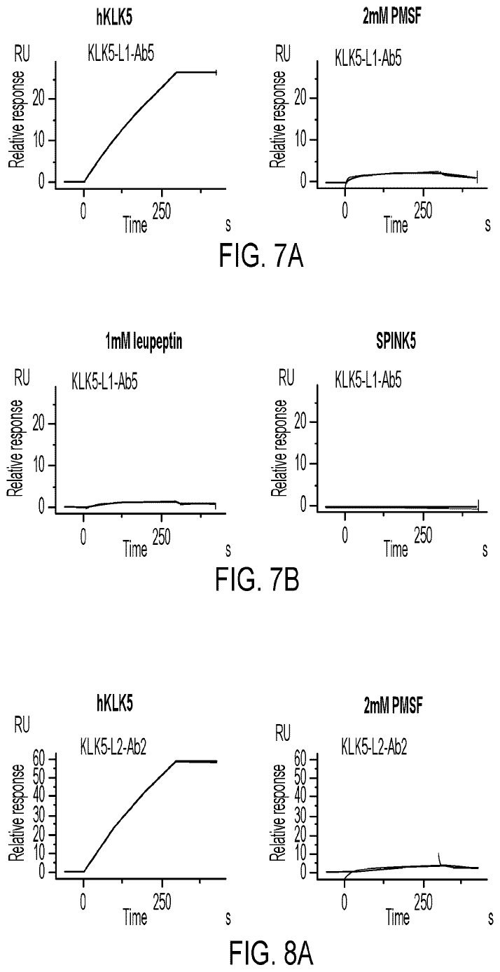

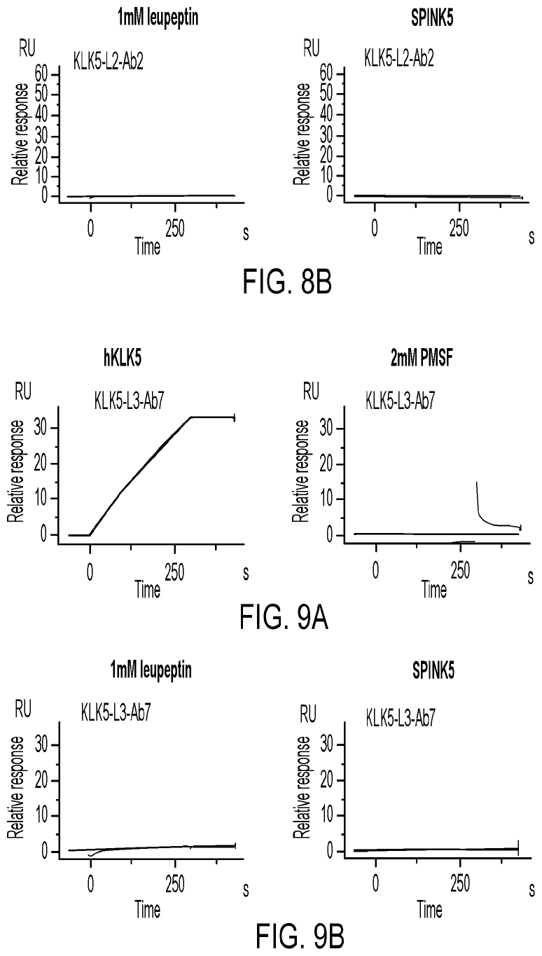

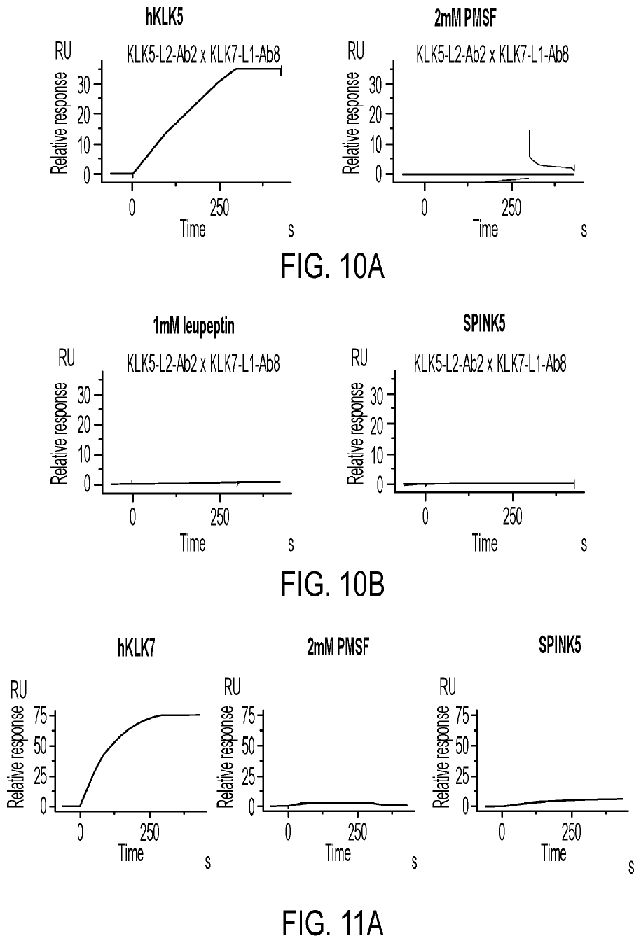

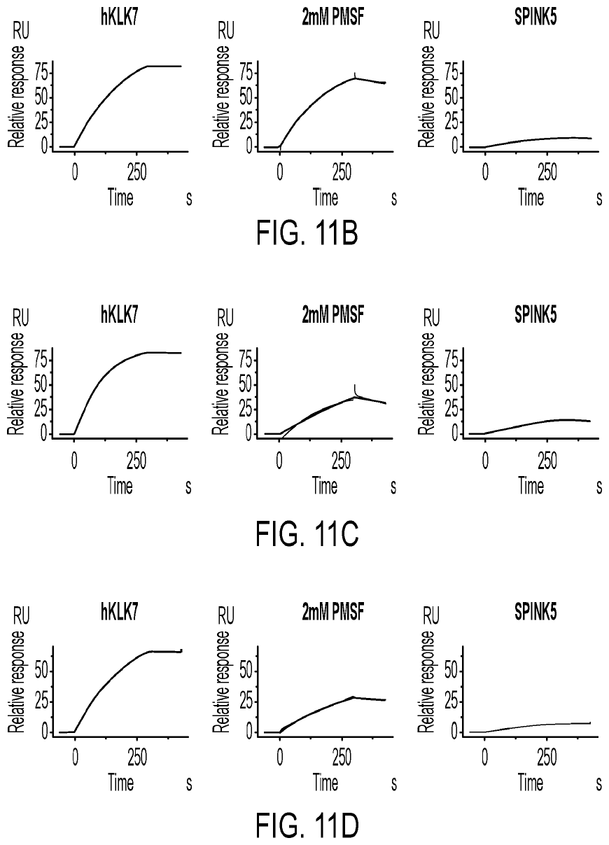

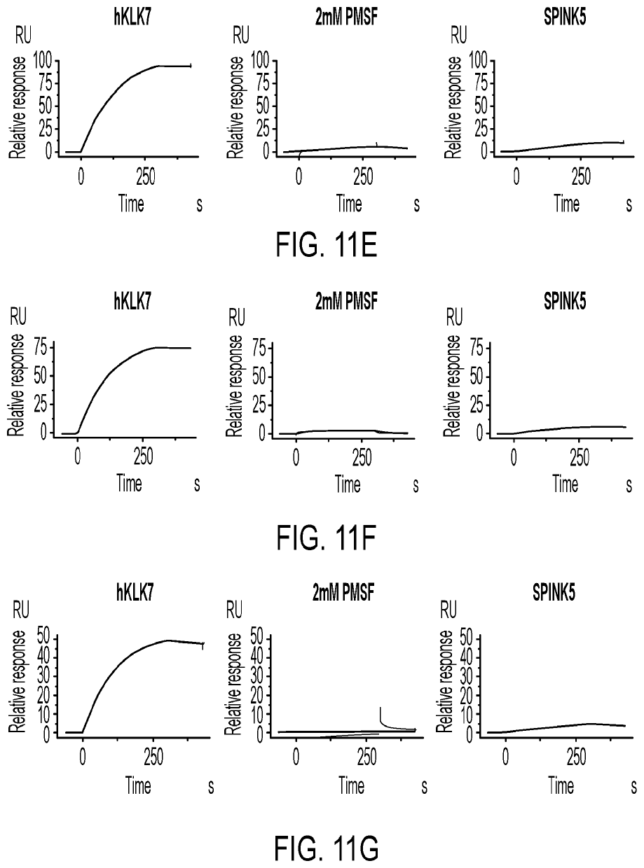

Binning was performed by capturing KLK5 and KLK7 antibodies (2 μg/mL) on a ProteinA chip surface. Flow rate was 30 μL/min and the contact time was 30 seconds. Then, either hKLK5 (FIGS. 7A-10B) or hKLK7 (FIGS. 11A-11H) was preincubated with small molecule or SPINK5 inhibitor before being applied to the chip: 20 nM of KLK5 or 7 was preincubated with 0.35 mg/mL=2 mM PMSF (from 100× stock in isopropanol), 1 mg/mL=1 mM leupeptin, or 25 μg/mL SPINK5. Flow rate during sample injection was 30 μL/min, contact time was 300 seconds and dissociation time was 120 seconds.

The data demonstrate that anti-KLK5 and anti-KLK7 antibodies compete for binding to hKLK5 and hKLK7 with the known active site inhibitor, SPINK5, indicating that the anti-KLK5 and anti-KLK7 antibodies also bind in the active site of KLK5 and KLK7. All KLK5 inhibitors and several KLK7 inhibitors also compete with small molecule inhibitors PMSF and leupeptin, which are known to bind the S1 substrate binding pocket in the active site. This indicates that these antibodies bind the S1 pocket as well.

### Example 6. Anti-KLK5 and Anti-KLK7 Antibodies Bind the Same Epitope on KLK5 and KLK7 as SPINK5

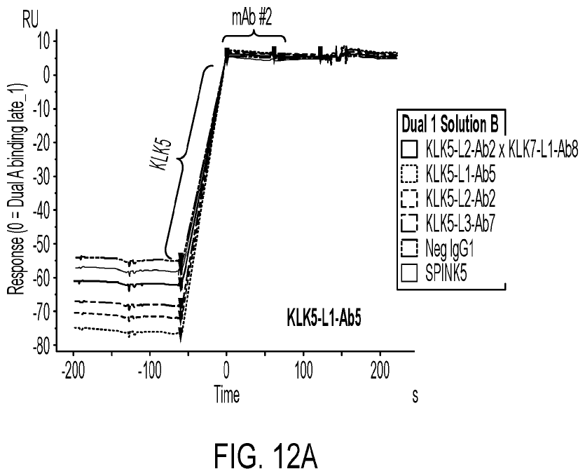

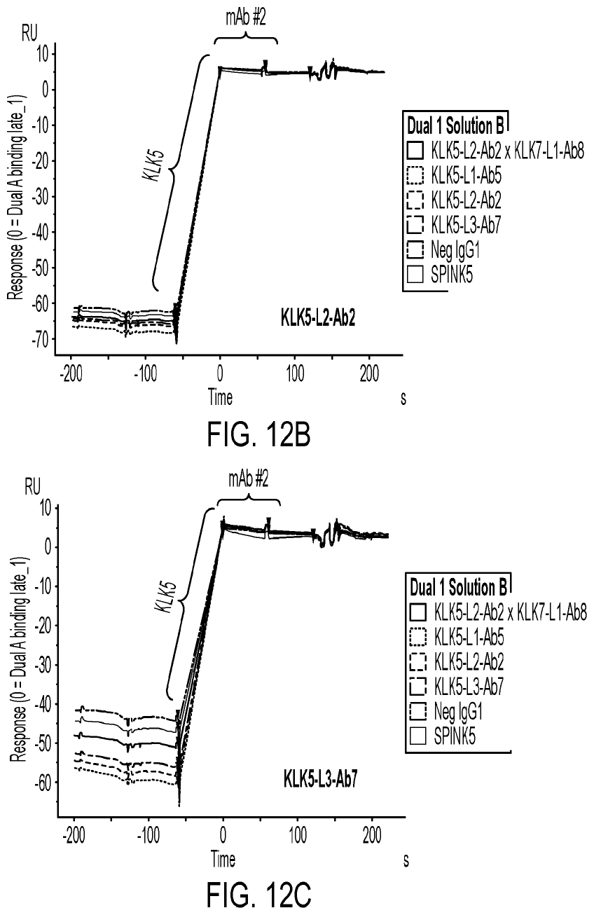

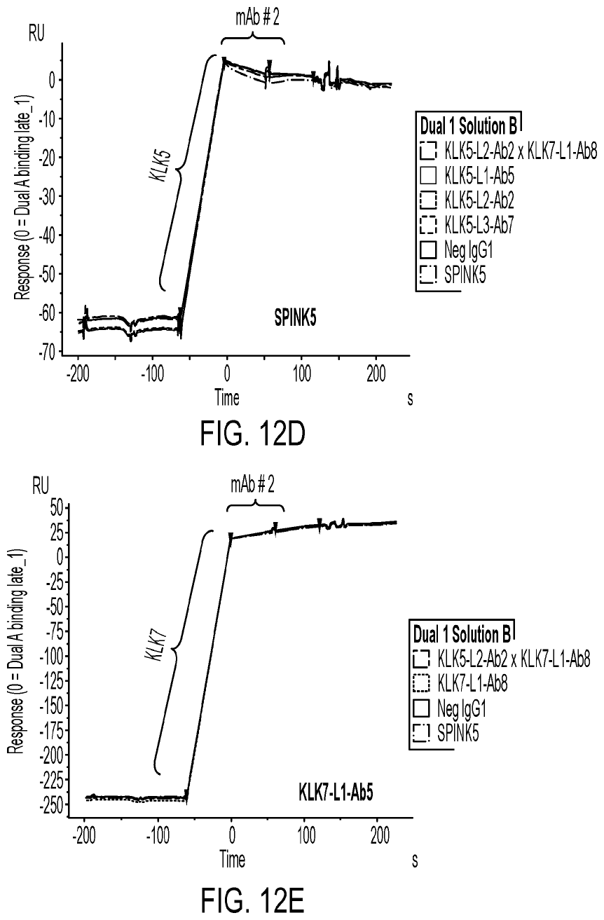

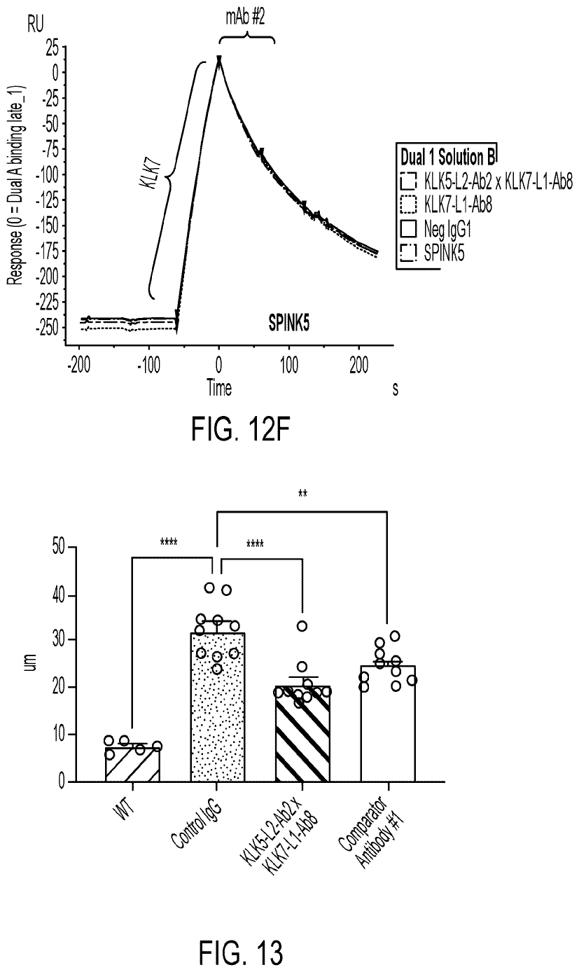

In this Example, the binding of anti-KLK5 and anti-KLK7 antibodies to the active site of KLK5 and KLK7 was further analyzed. Binning was performed in a sandwich format. The antibody indicated in each panel, or SPINK5, was covalently coupled to a CM5 surface, and then hKLK5 (FIGS. 12A-12D) or hKLK7 (FIGS. 12E-12F) was flowed over, followed by another anti-KLK5 or anti-KLK7 antibody (as indicated in the legend at 10 g/mL). Running buffer was 20 mM HEPES, 300 mM NaCl, 0.05% Tween-20, pH 7.5 Biacore was performed with a flow rate of 50 μL/min. Contact time for each antibody/protein was 60 seconds and dissociation time was 60 seconds.

The data demonstrates that anti-KLK5 and anti-KLK7 antibodies herein compete for binding to hKLK5 and hKLK7 with both each other and also with human SPINK5. As SPINK5 binds in the active site of both KLK5 and KLK7, this indicates that said antibodies also bind in the active site of KLK5 and KLK7 and that both SPINK5 and the antibodies cannot be bound to either KLK5 or KLK7 at the same time.

### Example 7. Anti-KLK5/Anti-KLK7 Bispecific Antibody Treatment Reduces Barrier Defects in the MC903 Atopic Dermatitis Mouse Model

Murine atopic dermatitis was induced in C57/BL6 mice by topical application of MC903 (40 μL (Sigma, calcipotriol hydrate, 412 ng/10 μL in ethanol) topically on the shaved area of the nape of neck once a day on days 1-8. Vehicle control, control IgG, anti-KLK5/anti-KLK7 bispecific antibody KLK5-L2-Ab2 x KLK7-L1-Ab8, or Comparator Antibody #1 (the mouse surrogate for a human anti-IL4R antibody) was injected at 30 mg/kg intraperitoneally (IP) 3 times/week starting on day −6.

On day 9, a piece of dorsal skin per mouse was preserved in 10% neutral buffered formalin (NBF). The skin was trimmed, embedded in paraffin, sectioned, and stained with H&E. Stratum corneum thickness was measured every 800 M according to a set grid in Qupath image analysis software and then averaged to generate a value for each animal.

Treatment with KLK5-L2-Ab2 x KLK7-L1-Ab8 reduced hyperkeratosis and desquamation as measured by stratum corneum thickness compared to control IgG, demonstrating the impact of KLK5/7 inhibition on barrier dysfunction (FIG. 13).

### Example 8. Treatment with Anti-KLK5/Anti-KLK7 Bispecific Antibody Reduces Disease in a Nc/Nga Atopic Dermatitis Mouse Model

Murine atopic dermatitis was induced in Nc/Nga mice (a strain with an intrinsic barrier defect due to reduced expression of ceramides) by topical application of House Dust Mite (HDM) allergen. Eight- to ten-week-old animals receive Biostir-AD ointment (120 mg/mouse), which contains mite allergens derived from Dermatophagoides farina, onto both ears and dorsal skin regions (including the neck), twice a week for two weeks, for a total of 480 mg Biostir-AD per mouse. A 150 μL SDS was applied at 2 hours before the Biostir-AD application from the second treatment (day 4). Vehicle control, control IgG, anti-KLK5/anti-KLK7 bispecific antibody KLK5-L2-Ab2 x KLK7-L1-Ab8, or Comparator Antibody #1 (a mouse surrogate for a human anti-IL4R antibody) was injected at 30 mg/kg intraperitoneally (IP) 3 times/week starting on day −6 for a total of 9 doses. The reference compound, tacrolimus ointment, was applied topically once daily (QD) starting on Day −6 for a total of 21 days (QD×21), applied 1 h after Biostir-AD application on Biostir-AD challenge days.

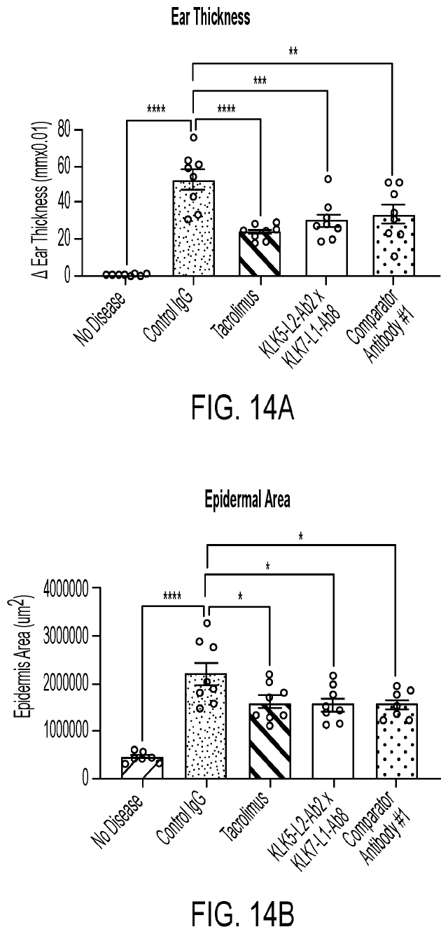

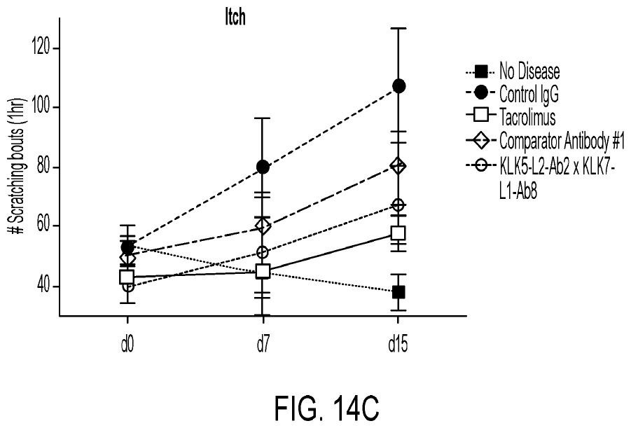

Animals were evaluated for scratching (“itch”) on days 0, 7, and 15, and ear thickness on day 15. For scratching measurement, the animals were individually placed in an observation jar (1000 mL) to acclimate for 30 min prior to start of measurements. The scratching behavior was recorded for 60 min on day 0, and at 30 minutes post-dosing on day 7 and 15 by visual observation. Ear thickness was measured with a Dyer model micrometer gauge. Treatment with KLK5-L2-Ab2 x KLK7-L1-Ab8 reduced ear thickness (FIG. 14A), epidermal area (FIG. 14B), and scratching (FIG. 14C) as compared to control IgG.

Taken together, these data indicate that inhibition of KLK5 and KLK7 can reduce inflammation, itch, and barrier dysfunction in a mouse model of atopic dermatitis in a manner exceeding or similar to that achieved with either immune suppression (e.g., using Tacrolimus) or inhibition of IL4R.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also set forth as follows:

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the application describes “a composition comprising A and B,” the application also contemplates the alternative embodiments “a composition consisting of A and B” and “a composition consisting essentially of A and B.”

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

What is claimed is:

1. An antibody specifically binds to KLK5, the antibody comprising
(a) a HC CDR1, HC CDR2 and HC CDR3 of a heavy chain variable domain having the amino acid sequence of SEQ ID NO: 3, and a LC CDR1, LC CDR2 and LC CDR3 of a light chain variable domain having the amino acid sequence of SEQ ID NO: 11;
(b) a HC CDR1, HC CDR2 and HC CDR3 of a heavy chain variable domain having the amino acid sequence of SEQ ID NO: 10, and a LC CDR1, LC CDR2 and LC CDR3 of a light chain variable domain having the amino acid sequence of SEQ ID NO: 11;
(c) a HC CDR1, HC CDR2 and HC CDR3 of a heavy chain variable domain having the amino acid sequence of SEQ ID NO: 14, and a LC CDR1, LC CDR2 and LC CDR3 of a light chain variable domain having the amino acid sequence of SEQ ID NO: 11;
(d) a HC CDR1, HC CDR2 and HC CDR3 of a heavy chain variable domain having the amino acid sequence of SEQ ID NO: 16, and a LC CDR1, LC CDR2 and LC CDR3 of a light chain variable domain having the amino acid sequence of SEQ ID NO: 11;
(e) a HC CDR1, HC CDR2 and HC CDR3 of a heavy chain variable domain having the amino acid sequence of SEQ ID NO: 18, and a LC CDR1, LC CDR2 and LC CDR3 of a light chain variable domain having the amino acid sequence of SEQ ID NO: 11;
(f) a HC CDR1, HC CDR2 and HC CDR3 of a heavy chain variable domain having the amino acid sequence of SEQ ID NO: 24, and a LC CDR1, LC CDR2 and LC CDR3 of a light chain variable domain having the amino acid sequence of SEQ ID NO: 29;
(g) a HC CDR1, HC CDR2 and HC CDR3 of a heavy chain variable domain having the amino acid sequence of SEQ ID NO: 28, and a LC CDR1, LC CDR2 and LC CDR3 of a light chain variable domain having the amino acid sequence of SEQ ID NO: 29;
(h) a HC CDR1, HC CDR2 and HC CDR3 of a heavy chain variable domain having the amino acid sequence of SEQ ID NO: 32, and a LC CDR1, LC CDR2 and LC CDR3 of a light chain variable domain having the amino acid sequence of SEQ ID NO: 37;
(i) a HC CDR1, HC CDR2 and HC CDR3 of a heavy chain variable domain having the amino acid sequence of SEQ ID NO: 36, and a LC CDR1, LC CDR2 and LC CDR3 of a light chain variable domain having the amino acid sequence of SEQ ID NO: 29;
(j) a HC CDR1, HC CDR2 and HC CDR3 of a heavy chain variable domain having the amino acid sequence of SEQ ID NO: 42, and a LC CDR1, LC CDR2 and LC CDR3 of a light chain variable domain having the amino acid sequence of SEQ ID NO: 43;
(k) a HC CDR1, HC CDR2 and HC CDR3 of a heavy chain variable domain having the amino acid sequence of SEQ ID NO: 46, and a LC CDR1, LC CDR2 and LC CDR3 of a light chain variable domain having the amino acid sequence of SEQ ID NO: 43;
(l) a HC CDR1, HC CDR2 and HC CDR3 of a heavy chain variable domain having the amino acid sequence of SEQ ID NO: 49, and a LC CDR1, LC CDR2 and LC CDR3 of a light chain variable domain having the amino acid sequence of SEQ ID NO: 43;
(m) a HC CDR1, HC CDR2 and HC CDR3 of a heavy chain variable domain having the amino acid sequence of SEQ ID NO: 51, and a LC CDR1, LC CDR2 and LC CDR3 of a light chain variable domain having the amino acid sequence of SEQ ID NO: 43;
(n) a HC CDR1, HC CDR2 and HC CDR3 of a heavy chain variable domain having the amino acid sequence of SEQ ID NO: 53, and a LC CDR1, LC CDR2 and LC CDR3 of a light chain variable domain having the amino acid sequence of SEQ ID NO: 43;
(o) a HC CDR1, HC CDR2 and HC CDR3 of a heavy chain variable domain having the amino acid sequence of SEQ ID NO: 58, and a LC CDR1, LC CDR2 and LC CDR3 of a light chain variable domain having the amino acid sequence of SEQ ID NO: 59; or
(p) a HC CDR1, HC CDR2 and HC CDR3 of a heavy chain variable domain having the amino acid sequence of SEQ ID NO: 64, and a LC CDR1, LC CDR2 and LC CDR3 of a light chain variable domain having the amino acid sequence of SEQ ID NO: 65.

2. The antibody of claim 1, wherein the antibody comprises:
(a) a HC CDR1 having the amino acid sequence of SEQ ID NO: 1, a HC CDR2 having the amino acid sequence of SEQ ID NO: 2, a HC CDR3 having the amino acid sequence of SEQ ID NO: 9, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 6:
(b) a HC CDR1 having the amino acid sequence of SEQ ID NO: 7, a HC CDR2 having the amino acid sequence of SEQ ID NO: 8, a HC CDR3 having the amino acid sequence of SEQ ID NO: 9, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 6;
(c) a HC CDR1 having the amino acid sequence of SEQ ID NO: 12, a HC CDR2 having the amino acid sequence of SEQ ID NO: 13, a HC CDR3 having the amino acid sequence of SEQ ID NO: 9, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 6;
(d) a HC CDR1 having the amino acid sequence of SEQ ID NO: 15, a HC CDR2 having the amino acid sequence of SEQ ID NO: 13, a HC CDR3 having the amino acid sequence of SEQ ID NO: 9, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 6;
(e) a HC CDR1 having the amino acid sequence of SEQ ID NO: 12, a HC CDR2 having the amino acid sequence of SEQ ID NO: 13, a HC CDR3 having the amino acid sequence of SEQ ID NO: 17, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 6;
(f) a HC CDR1 having the amino acid sequence of SEQ ID NO: 19, a HC CDR2 having the amino acid sequence of SEQ ID NO: 20, a HC CDR3 having the amino acid sequence of SEQ ID NO: 21, a LC CDR1 having the amino acid sequence of SEQ ID NO: 22, a LC CDR2 having the amino acid sequence of SEQ ID NO: 23, and a LC CDR3 having the amino acid sequence of SEQ ID NO: 34;
(g) a HC CDR1 having the amino acid sequence of SEQ ID NO: 25, a HC CDR2 having the amino acid sequence of SEQ ID NO: 26, a HC CDR3 having the amino acid sequence of SEQ ID NO: 27, a LC CDR1 having the amino acid sequence of SEQ ID NO: 22, a LC CDR2 having the amino acid sequence of SEQ ID NO: 23, and a LC CDR3 having the amino acid sequence of SEQ ID NO: 34;
(h) a HC CDR1 having the amino acid sequence of SEQ ID NO: 30, a HC CDR2 having the amino acid sequence of SEQ ID NO: 26, a HC CDR3 having the amino acid sequence of SEQ ID NO: 31, a LC CDR1 having the amino acid sequence of SEQ ID NO: 22, a LC CDR2 having the amino acid sequence of SEQ ID NO: 23, and a LC CDR3 having the amino acid sequence of SEQ ID NO: 35;
(i) a HC CDR1 having the amino acid sequence of SEQ ID NO: 30, a HC CDR2 having the amino acid sequence of SEQ ID NO: 26, a HC CDR3 having the amino acid sequence of SEQ ID NO: 33, a LC CDR1 having the amino acid sequence of SEQ ID NO: 22, a LC CDR2 having the amino acid sequence of SEQ ID NO: 23, and a LC CDR3 having the amino acid sequence of SEQ ID NO: 34;
(j) a HC CDR1 having the amino acid sequence of SEQ ID NO: 38, a HC CDR2 having the amino acid sequence of SEQ ID NO: 39, a HC CDR3 having the amino acid sequence of SEQ ID NO: 40, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 41;
(k) a HC CDR1 having the amino acid sequence of SEQ ID NO: 44, a HC CDR2 having the amino acid sequence of SEQ ID NO: 45, a HC CDR3 having the amino acid sequence of SEQ ID NO: 40, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 41;
(l) a HC CDR1 having the amino acid sequence of SEQ ID NO: 47, a HC CDR2 having the amino acid sequence of SEQ ID NO: 48, a HC CDR3 having the amino acid sequence of SEQ ID NO: 40, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 41;
(m) a HC CDR1 having the amino acid sequence of SEQ ID NO: 47, a HC CDR2 having the amino acid sequence of SEQ ID NO: 48, a HC CDR3 having the amino acid sequence of SEQ ID NO: 50, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 41;
(n) a HC CDR1 having the amino acid sequence of SEQ ID NO: 44, a HC CDR2 having the amino acid sequence of SEQ ID NO: 45, a HC CDR3 having the amino acid sequence of SEQ ID NO: 52, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 41;
(o) a HC CDR1 having the amino acid sequence of SEQ ID NO: 54, a HC CDR2 having the amino acid sequence of SEQ ID NO: 55, a HC CDR3 having the amino acid sequence of SEQ ID NO: 56, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 57; or
(p) a HC CDR1 having the amino acid sequence of SEQ ID NO: 60, a HC CDR2 having the amino acid sequence of SEQ ID NO: 61, a HC CDR3 having the amino acid sequence of SEQ ID NO: 62, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 63.

3. The antibody of claim 1 or 2, wherein the antibody comprises:
(a) a VH comprising the amino acid sequence of SEQ ID NO: 3 and a VL comprising the amino acid sequence of SEQ ID NO: 11;
(b) a VH comprising the amino acid sequence of SEQ ID NO: 10, and a VL comprising the amino acid sequence of SEQ ID NO: 11;
(c) a VH comprising the amino acid sequence of SEQ ID NO: 14, and a LC CDR1, LC CDR2 and a VL comprising the amino acid sequence of SEQ ID NO: 11;
(d) a VH comprising the amino acid sequence of SEQ ID NO: 16, and a VL comprising the amino acid sequence of SEQ ID NO: 11;
(e) a VH comprising the amino acid sequence of SEQ ID NO: 18, and a VL comprising the amino acid sequence of SEQ ID NO: 11;
(f) a VH comprising the amino acid sequence of SEQ ID NO: 24, and a VL comprising the amino acid sequence of SEQ ID NO: 29;
(g) a VH comprising the amino acid sequence of SEQ ID NO: 28, and a VL comprising the amino acid sequence of SEQ ID NO: 29;
(h) a VH comprising the amino acid sequence of SEQ ID NO: 32, and a VL comprising the amino acid sequence of SEQ ID NO: 37;
(i) a VH comprising the amino acid sequence of SEQ ID NO: 36, and a VL comprising the amino acid sequence of SEQ ID NO: 29;
(j) a VH comprising the amino acid sequence of SEQ ID NO: 42, and a VL comprising the amino acid sequence of SEQ ID NO: 43;
(k) a VH comprising the amino acid sequence of SEQ ID NO: 46, and a VL comprising the amino acid sequence of SEQ ID NO: 43;
(l) a VH comprising the amino acid sequence of SEQ ID NO: 49, and a VL comprising the amino acid sequence of SEQ ID NO: 43;
(m) a VH comprising the amino acid sequence of SEQ ID NO: 51, and a VL comprising the amino acid sequence of SEQ ID NO: 43;
(n) a VH comprising the amino acid sequence of SEQ ID NO: 53, and a VH comprising the amino acid sequence of SEQ ID NO: 43;
(o) a VH comprising the amino acid sequence of SEQ ID NO: 58, and a VH comprising the amino acid sequence of SEQ ID NO: 59; or
(p) a VH comprising the amino acid sequence of SEQ ID NO: 64, and a VH comprising the amino acid sequence of SEQ ID NO: 65.

4. An antibody specifically binds to KLK7, the antibody comprising:
(a) a HC CDR1, HC CDR2 and HC CDR3 of a heavy chain variable domain having the amino acid sequence of SEQ ID NO: 72 and a LC CDR1, LC CDR2 and LC CDR3 of a light chain variable domain having the amino acid sequence of SEQ ID NO: 73;
(b) a HC CDR1, HC CDR2 and HC CDR3 of a heavy chain variable domain having the amino acid sequence of SEQ ID NO: 199 and a LC CDR1, LC CDR2 and LC CDR3 of a light chain variable domain having the amino acid sequence of SEQ ID NO: 200;
(c) a HC CDR1, HC CDR2 and HC CDR3 of a heavy chain variable domain having the amino acid sequence of SEQ ID NO: 77 and a LC CDR1, LC CDR2 and LC CDR3 of a light chain variable domain having the amino acid sequence of SEQ ID NO: 78;
(d) a HC CDR1, HC CDR2 and HC CDR3 of a heavy chain variable domain having the amino acid sequence of SEQ ID NO: 80 and a LC CDR1, LC CDR2 and LC CDR3 of a light chain variable domain having the amino acid sequence of SEQ ID NO: 78;
(e) a HC CDR1, HC CDR2 and HC CDR3 of a heavy chain variable domain having the amino acid sequence of SEQ ID NO: 80 and a LC CDR1, LC CDR2 and LC CDR3 of a light chain variable domain having the amino acid sequence of SEQ ID NO: 82;
(f) a HC CDR1, HC CDR2 and HC CDR3 of a heavy chain variable domain having the amino acid sequence of SEQ ID NO: 84 and a LC CDR1, LC CDR2 and LC CDR3 of a light chain variable domain having the amino acid sequence of SEQ ID NO: 78;
(g) a HC CDR1, HC CDR2 and HC CDR3 of a heavy chain variable domain having the amino acid sequence of SEQ ID NO: 87 and a LC CDR1, LC CDR2 and LC CDR3 of a light chain variable domain having the amino acid sequence of SEQ ID NO: 88;
(h) a HC CDR1, HC CDR2 and HC CDR3 of a heavy chain variable domain having the amino acid sequence of SEQ ID NO: 285 and a LC CDR1, LC CDR2 and LC CDR3 of a light chain variable domain having the amino acid sequence of SEQ ID NO: 82;
(i) comprises a HC CDR1, HC CDR2 and HC CDR3 of a heavy chain variable domain having the amino acid sequence of SEQ ID NO: 98 and a LC CDR1, LC CDR2 and LC CDR3 of a light chain variable domain having the amino acid sequence of SEQ ID NO: 97;
(j) a HC CDR1, HC CDR2 and HC CDR3 of a heavy chain variable domain having the amino acid sequence of SEQ ID NO: 153 and a LC CDR1, LC CDR2 and LC CDR3 of a light chain variable domain having the amino acid sequence of SEQ ID NO: 97;
(k) a HC CDR1, HC CDR2 and HC CDR3 of a heavy chain variable domain having the amino acid sequence of SEQ ID NO: 103 and a LC CDR1, LC CDR2 and LC CDR3 of a light chain variable domain having the amino acid sequence of SEQ ID NO: 104;
(l) a HC CDR1, HC CDR2 and HC CDR3 of a heavy chain variable domain having the amino acid sequence of SEQ ID NO: 107 and a LC CDR1, LC CDR2 and LC CDR3 of a light chain variable domain having the amino acid sequence of SEQ ID NO: 108;
(m) a HC CDR1, HC CDR2 and HC CDR3 of a heavy chain variable domain having the amino acid sequence of SEQ ID NO: 111 and a LC CDR1, LC CDR2 and LC CDR3 of a light chain variable domain having the amino acid sequence of SEQ ID NO: 97;
(n) comprises a HC CDR1, HC CDR2 and HC CDR3 of a heavy chain variable domain having the amino acid sequence of SEQ ID NO: 115 and a LC CDR1, LC CDR2 and LC CDR3 of a light chain variable domain having the amino acid sequence of SEQ ID NO: 116;
(o) a HC CDR1, HC CDR2 and HC CDR3 of a heavy chain variable domain having the amino acid sequence of SEQ ID NO: 121 and a LC CDR1, LC CDR2 and LC CDR3 of a light chain variable domain having the amino acid sequence of SEQ ID NO: 122;
(p) a HC CDR1, HC CDR2 and HC CDR3 of a heavy chain variable domain having the amino acid sequence of SEQ ID NO: 127 and a LC CDR1, LC CDR2 and LC CDR3 of a light chain variable domain having the amino acid sequence of SEQ ID NO: 128;
(q) a HC CDR1, HC CDR2 and HC CDR3 of a heavy chain variable domain having the amino acid sequence of SEQ ID NO: 133 and a LC CDR1, LC CDR2 and LC CDR3 of a light chain variable domain having the amino acid sequence of SEQ ID NO: 134;
(r) a HC CDR1, HC CDR2 and HC CDR3 of a heavy chain variable domain having the amino acid sequence of SEQ ID NO: 139 and a LC CDR1, LC CDR2 and LC CDR3 of a light chain variable domain having the amino acid sequence of SEQ ID NO: 140.
(s) a HC CDR1, HC CDR2 and HC CDR3 of a heavy chain variable domain having the amino acid sequence of SEQ ID NO: 145 and a LC CDR1, LC CDR2 and LC CDR3 of a light chain variable domain having the amino acid sequence of SEQ ID NO: 146; or
(t) a HC CDR1, HC CDR2 and HC CDR3 of a heavy chain variable domain having the amino acid sequence of SEQ ID NO: 151 and a LC CDR1, LC CDR2 and LC CDR3 of a light chain variable domain having the amino acid sequence of SEQ ID NO: 152.

5. The antibody of claim 4, wherein the antibody comprises:
(a) a HC CDR1 having the amino acid sequence of SEQ ID NO: 66, a HC CDR2 having the amino acid sequence of SEQ ID NO: 67, a HC CDR3 having the amino acid sequence of SEQ ID NO: 68, a LC CDR1 having the amino acid sequence of SEQ ID NO: 69, a LC CDR2 having the amino acid sequence of SEQ ID NO: 70, and a LC CDR3 having the amino acid sequence of SEQ ID NO: 71;
(b) a HC CDR1 having the amino acid sequence of SEQ ID NO: 74, a HC CDR2 having the amino acid sequence of SEQ ID NO: 67, a HC CDR3 having the amino acid sequence of SEQ ID NO: 75, a LC CDR1 having the amino acid sequence of SEQ ID NO: 69, a LC CDR2 having the amino acid sequence of SEQ ID NO: 70, and a LC CDR3 having the amino acid sequence of SEQ ID NO: 198;
(c) a HC CDR1 having the amino acid sequence of SEQ ID NO: 284, a HC CDR2 having the amino acid sequence of SEQ ID NO: 201, a HC CDR3 having the amino acid sequence of SEQ ID NO: 68, a LC CDR1 having the amino acid sequence of SEQ ID NO: 69, a LC CDR2 having the amino acid sequence of SEQ ID NO: 70, and a LC CDR3 having the amino acid sequence of SEQ ID NO: 76;
(d) a HC CDR1 having the amino acid sequence of SEQ ID NO: 284, a HC CDR2 having the amino acid sequence of SEQ ID NO: 79, a HC CDR3 having the amino acid sequence of SEQ ID NO: 68, a LC CDR1 having the amino acid sequence of SEQ ID NO: 69, a LC CDR2 having the amino acid sequence of SEQ ID NO: 70, and a LC CDR3 having the amino acid sequence of SEQ ID NO: 76;
(e) a HC CDR1 having the amino acid sequence of SEQ ID NO: 284, a HC CDR2 having the amino acid sequence of SEQ ID NO: 79, a HC CDR3 having the amino acid sequence of SEQ ID NO: 68, a LC CDR1 having the amino acid sequence of SEQ ID NO: 69, a LC CDR2 having the amino acid sequence of SEQ ID NO: 70, and a LC CDR3 having the amino acid sequence of SEQ ID NO: 81;
(f) a HC CDR1 having the amino acid sequence of SEQ ID NO: 284, a HC CDR2 having the amino acid sequence of SEQ ID NO: 79, a HC CDR3 having the amino acid sequence of SEQ ID NO: 83, a LC CDR1 having the amino acid sequence of SEQ ID NO: 69, a LC CDR2 having the amino acid sequence of SEQ ID NO: 70, and a LC CDR3 having the amino acid sequence of SEQ ID NO: 76;
(g) a HC CDR1 having the amino acid sequence of SEQ ID NO: 284, a HC CDR2 having the amino acid sequence of SEQ ID NO: 79, a HC CDR3 having the amino acid sequence of SEQ ID NO: 85, a LC CDR1 having the amino acid sequence of SEQ ID NO: 69, a LC CDR2 having the amino acid sequence of SEQ ID NO: 70, and a LC CDR3 having the amino acid sequence of SEQ ID NO: 86;
(h) a HC CDR1 having the amino acid sequence of SEQ ID NO: 284, a HC CDR2 having the amino acid sequence of SEQ ID NO: 79, a HC CDR3 having the amino acid sequence of SEQ ID NO: 89, a LC CDR1 having the amino acid sequence of SEQ ID NO: 69, a LC CDR2 having the amino acid sequence of SEQ ID NO: 70, and a LC CDR3 having the amino acid sequence of SEQ ID NO: 81;
(i) a HC CDR1 having the amino acid sequence of SEQ ID NO: 90, a HC CDR2 having the amino acid sequence of SEQ ID NO: 91, a HC CDR3 having the amino acid sequence of SEQ ID NO: 92, a LC CDR1 having the amino acid sequence of SEQ ID NO: 93, a LC CDR2 having the amino acid sequence of SEQ ID NO: 94, and a LC CDR3 having the amino acid sequence of SEQ ID NO: 95;
(j) a HC CDR1 having the amino acid sequence of SEQ ID NO: 96, a HC CDR2 having the amino acid sequence of SEQ ID NO: 109, a HC CDR3 having the amino acid sequence of SEQ ID NO: 92, a LC CDR1 having the amino acid sequence of SEQ ID NO: 93, a LC CDR2 having the amino acid sequence of SEQ ID NO: 94, and a LC CDR3 having the amino acid sequence of SEQ ID NO: 95;
(k) a HC CDR1 having the amino acid sequence of SEQ ID NO: 99, a HC CDR2 having the amino acid sequence of SEQ ID NO: 100, a HC CDR3 having the amino acid sequence of SEQ ID NO: 101, a LC CDR1 having the amino acid sequence of SEQ ID NO: 93, a LC CDR2 having the amino acid sequence of SEQ ID NO: 94, and a LC CDR3 having the amino acid sequence of SEQ ID NO: 102;
(l) a HC CDR1 having the amino acid sequence of SEQ ID NO: 99, a HC CDR2 having the amino acid sequence of SEQ ID NO: 100, a HC CDR3 having the amino acid sequence of SEQ ID NO: 105, a LC CDR1 having the amino acid sequence of SEQ ID NO: 93, a LC CDR2 having the amino acid sequence of SEQ ID NO: 94, and a LC CDR3 having the amino acid sequence of SEQ ID NO: 106;
(m) a HC CDR1 having the amino acid sequence of SEQ ID NO: 96, a HC CDR2 having the amino acid sequence of SEQ ID NO: 109, a HC CDR3 having the amino acid sequence of SEQ ID NO: 110, a LC CDR1 having the amino acid sequence of SEQ ID NO: 93, a LC CDR2 having the amino acid sequence of SEQ ID NO: 94, and a LC CDR3 having the amino acid sequence of SEQ ID NO: 95;
(n) a HC CDR1 having the amino acid sequence of SEQ ID NO: 66, a HC CDR2 having the amino acid sequence of SEQ ID NO: 112, a HC CDR3 having the amino acid sequence of SEQ ID NO: 113, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 114;
(o) a HC CDR1 having the amino acid sequence of SEQ ID NO: 117, a HC CDR2 having the amino acid sequence of SEQ ID NO: 118, a HC CDR3 having the amino acid sequence of SEQ ID NO: 119, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 120;
(p) a HC CDR1 having the amino acid sequence of SEQ ID NO: 123, a HC CDR2 having the amino acid sequence of SEQ ID NO: 124, a HC CDR3 having the amino acid sequence of SEQ ID NO: 125, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 126;
(q) a HC CDR1 having the amino acid sequence of SEQ ID NO: 19, a HC CDR2 having the amino acid sequence of SEQ ID NO: 20, a HC CDR3 having the amino acid sequence of SEQ ID NO: 129, a LC CDR1 having the amino acid sequence of SEQ ID NO: 130, a LC CDR2 having the amino acid sequence of SEQ ID NO: 131, and a LC CDR3 having the amino acid sequence of SEQ ID NO: 132;
(r) a HC CDR1 having the amino acid sequence of SEQ ID NO: 135, a HC CDR2 having the amino acid sequence of SEQ ID NO: 136, a HC CDR3 having the amino acid sequence of SEQ ID NO: 137, a LC CDR1 having the amino acid sequence of SEQ ID NO: 130, a LC CDR2 having the amino acid sequence of SEQ ID NO: 131, and a LC CDR3 having the amino acid sequence of SEQ ID NO: 138;
(s) a HC CDR1 having the amino acid sequence of SEQ ID NO: 38, a HC CDR2 having the amino acid sequence of SEQ ID NO: 141, a HC CDR3 having the amino acid sequence of SEQ ID NO: 142, a LC CDR1 having the amino acid sequence of SEQ ID NO: 23, a LC CDR2 having the amino acid sequence of SEQ ID NO: 144, and a LC CDR3 having the amino acid sequence of SEQ ID NO: 145; or
(t) a HC CDR1 having the amino acid sequence of SEQ ID NO: 147, a HC CDR2 having the amino acid sequence of SEQ ID NO: 148, a HC CDR3 having the amino acid sequence of SEQ ID NO: 149, a LC CDR1 having the amino acid sequence of SEQ ID NO: 143, a LC CDR2 having the amino acid sequence of SEQ ID NO: 23, and a LC CDR3 having the amino acid sequence of SEQ ID NO: 150.

6. The antibody of claim 4 or 5, wherein the antibody comprises:
(a) a VH comprising the amino acid sequence of SEQ ID NO: 72 and a VL comprising the amino acid sequence of SEQ ID NO: 73;
(b) a VH comprising the amino acid sequence of SEQ ID NO: 199 and a VL comprising the amino acid sequence of SEQ ID NO: 200;
(c) a VH comprising the amino acid sequence of SEQ ID NO: 77 and a VL comprising the amino acid sequence of SEQ ID NO: 78;
(d) a VH comprising the amino acid sequence of SEQ ID NO: 80 and a VL comprising the amino acid sequence of SEQ ID NO: 78;
(e) a VH comprising the amino acid sequence of SEQ ID NO: 80 and a VL comprising the amino acid sequence of SEQ ID NO: 82;
(f) a VH comprising the amino acid sequence of SEQ ID NO: 84 and a VL comprising the amino acid sequence of SEQ ID NO: 78;
(g) a VH comprising the amino acid sequence of SEQ ID NO: 87 and a VL comprising the amino acid sequence of SEQ ID NO: 88;
(h) a VH comprising the amino acid sequence of SEQ ID NO: 285 and a VL comprising the amino acid sequence of SEQ ID NO: 82;
(i) a VH comprising the amino acid sequence of SEQ ID NO: 98 and a VL comprising the amino acid sequence of SEQ ID NO: 97;
(j) a VH comprising the amino acid sequence of SEQ ID NO: 153 and a VL comprising the amino acid sequence of SEQ ID NO: 97;
(k) a VH comprising the amino acid sequence of SEQ ID NO: 103 and a VL comprising the amino acid sequence of SEQ ID NO: 104;
(l) a VH comprising the amino acid sequence of SEQ ID NO: 107 and a VL comprising the amino acid sequence of SEQ ID NO: 108;
(m) a VH comprising the amino acid sequence of SEQ ID NO: 111 and a VL comprising the amino acid sequence of SEQ ID NO: 97;
(n) a VH comprising the amino acid sequence of SEQ ID NO: 115 and a VL comprising the amino acid sequence of SEQ ID NO: 116;
(o) a VH comprising the amino acid sequence of SEQ ID NO: 121 and a VL comprising the amino acid sequence of SEQ ID NO: 122;
(p) a VH comprising the amino acid sequence of SEQ ID NO: 127 and a VL comprising the amino acid sequence of SEQ ID NO: 128;
(q) a VH comprising the amino acid sequence of SEQ ID NO: 133 and a VL comprising the amino acid sequence of SEQ ID NO: 134;
(r) a VH comprising the amino acid sequence of SEQ ID NO: 139 and a VL comprising the amino acid sequence of SEQ ID NO: 140;
(s) a VH comprising the amino acid sequence of SEQ ID NO: 145 and a VL comprising the amino acid sequence of SEQ ID NO: 146; or
(t) a VH comprising the amino acid sequence of SEQ ID NO: 151 and a VL comprising the amino acid sequence of SEQ ID NO: 152.

7. A bispecific antibody comprising a first antigen specific binding site from an anti-KLK5 antibody selected from Table 1a and Table 1b, and second antigen specific binding site from an anti-KLK7 antibody selected from Table 2a and Table 2b.

8. The bispecific antibody of claim 7, wherein the bispecific antibody comprises pairs of anti-KLK5 antibodies and anti-KLK7 antibodies as listed in Table 4.

9. The bispecific antibody of claim 7 or 8, wherein the bispecific antibody comprises a HC CDR1, a HC CDR2, a HC CDR3, a LC CDR1, a LC CDR2, and a LC CDR3 of an anti-KLK5 antibody selected from Table 1a and Table 1b, and a HC CDR1, a HC CDR2, a HC CDR3, a LC CDR1, a LC CDR2, and a LC CDR3 of an anti-KLK7 antibody selected from Table 2a and Table 2b.

10. The bispecific antibody of any one of claims 7-9, wherein the bispecific antibody binds to the active site of KLK5.

11. The bispecific antibody of any one of claims 7-10, wherein the bispecific antibody binds to the active site of KLK7.

12. The bispecific antibody of any one of claims 7-11, wherein the bispecific antibody competes with SPINK5 and/or leupeptin for binding of the KLK5 active site.

13. The bispecific antibody of any one of claims 7-12, wherein the bispecific antibody competes with SPINK5 and/or leupeptin for binding of the KLK7 active site.

14. The bispecific antibody of any one of claims 7-13, wherein the bispecific antibody binds to the active form of KLK5 but not the inactive form of KLK5.

15. The bispecific antibody of any one of claims 7-14, wherein the bispecific antibody binds to the active form of KLK7 but not the inactive form of KLK7.

16. The bispecific antibody of any one of claims 7-15, wherein the bispecific antibody inhibits the protease activity of KLK5.

17. The bispecific antibody of any one of claims 7-16, wherein the bispecific antibody inhibits the protease activity of KLK7.

18. The bispecific antibody of any one of claims 7-17, wherein the bispecific antibody is not cleaved in the heavy chain by KLK5 on binding to KLK5.

19. The bispecific antibody of any one of claims 7-18, wherein the bispecific antibody is not cleaved in the heavy chain by KLK7 on binding to KLK7.

20. The bispecific antibody of any one of claims 7-19, wherein the bispecific antibody comprises:
(a) a first arm comprising:
(i) a HC CDR1, a HC CDR2, and a HC CDR3 of a heavy chain variable region (VH) comprising the amino acid sequence of SEQ ID NO: 28, and
(ii) a LC CDR1, a LC CDR2, and a LC CDR3 of a light chain variable region (VL) comprising the amino sequence of SEQ ID NO: 29; and
(b) a second arm comprising:
(i) a HC CDR1, a HC CDR2, and a HC CDR3 of a VH comprising the amino acid sequence of SEQ ID NO: 285, and
(ii) a LC CDR1, a LC CDR2, and a LC CDR3 of a VL comprising the amino acid sequence of SEQ ID NO: 82.

21. The bispecific antibody of any one of claims 7-20, wherein the bispecific antibody comprises:
(a) a first arm comprising a HC CDR1 comprising the amino acid sequence of SEQ ID NO: 25, a HC CDR2 comprising the amino acid sequence of SEQ ID NO: 26, a HC CDR3 comprising the amino acid sequence of SEQ ID NO: 27, a LC CDR1 comprising the amino acid sequence of SEQ ID NO: 22, a LC CDR2 comprising the amino acid sequence of SEQ ID NO: 23, and a LC CDR3 comprising the amino acid sequence of SEQ ID NO: 34; and
(b) a second arm comprising a HC CDR1 comprising the amino acid sequence of SEQ ID NO: 284, a HC CDR2 comprising the amino acid sequence of SEQ ID NO: 79, a HC CDR3 comprising the amino acid sequence of SEQ ID NO: 89, a LC CDR1 comprising the amino acid sequence of SEQ ID NO: 69, a LC CDR2 comprising the amino acid sequence of SEQ ID NO: 70, and a LC CDR3 comprising the amino acid sequence of SEQ ID NO: 81.

22. The bispecific antibody of any one of claims 7-21, wherein the bispecific antibody comprises:
(a) a first arm comprising a VH comprising the amino acid of SEQ ID NO: 28 and a VL comprising the amino acid sequence of SEQ ID NO: 29; and
(b) a second arm comprising a VH comprising the amino acid sequence of SEQ ID NO: 285 and a VL comprising the amino acid sequence of SEQ ID NO: 82.

23. A composition comprising the antibody of any one of claims 1-6, or the bispecific antibody of any one of claims 7-22, and optionally an acceptable carrier.

24. A nucleic acid encoding the antibody of any one of claims 1-6, or the bispecific antibody of any one of claims 7-22.

25. A method of treating a skin barrier defect, the method comprising administering to a subject an effective amount of the antibody of any one of claims 1-6, or the bispecific antibody of any one of claims 7-22.

26. The antibody of any one of claims 1-6, the bispecific antibody of any one of claims 7-22, or the composition of claim 10 for use in a method of treating a skin barrier defect.

27. The method of claim 26, wherein the skin barrier defect associated with Netherton syndrome, atopic dermatitis, eosinophilic esophagitis, prurigo nodularis, chronic pruritus of unknown origin (CPUO), dry skin, asthma (KLK5 specifically), ichthyosis vulgaris, or itch or chronic itch.

28. The antibody, bispecific antibody, or composition for use according to claim 27, wherein the skin barrier defect associated with Netherton syndrome, atopic dermatitis, eosinophilic esophagitis, prurigo nodularis, chronic pruritus of unknown origin (CPUO), dry skin, asthma (KLK5 specifically), ichthyosis vulgaris, or itch or chronic itch.
